(19) United States
(12) Patent Application Publication
(10) Pub. No.: US 2011/0092505 A1
(43) Pub. Date: Apr. 21, 2011

(54) ORGANIC COMPOUNDS

(76) Inventors: Robin Burgis, Cheney, WA (US); Michael Paul Capparelli, Cambridge, MA (US); Lucian Dipietro, Cambridge, MA (US); Gabriel G. Gamber, Cambridge, MA (US); Charles Francis Jewell, JR., Sudbury, MA (US); Erik Meredith, Cambridge, MA (US); Karl Miranda, Cambridge, MA (US); Lauren G. Monovich, Cambridge, MA (US); Chang Rao, Cambridge, MA (US); Nicolas Soldermann, Basel (CH); Taeyoung Yoon, Cambridge, MA (US); Qingming Zhu, Cambridge, MA (US)

(21) Appl. No.: 12/996,901

(22) PCT Filed: Jun. 12, 2009
(86) PCT No.: PCT/EP09/57298

§ 371 Date: Dec. 8, 2010

Related U.S. Application Data
(60) Provisional application No. 61/061,384, filed on Jun. 13, 2008.

(51) Int. Cl. A61K 31/535; A61K 31/4985; A61K 31/501; A61K 31/497; A61K 31/437; A61K 31/4545; C07D 417/14; C07D 487/04; C07D 413/14; C07D 471/04; C07D 401/14; A61P 17/00; A61P 9/00; A61P 35/00; A61P 37/00

(52) U.S. Cl. 514/235.5; 514/249; 514/252.02; 514/253.01; 514/253.04; 514/300; 514/318; 544/131; 544/238; 544/350; 544/362; 544/364; 546/113; 546/194

(57) ABSTRACT

The present invention provides novel organic compounds of Formula I: methods of use, and pharmaceutical compositions thereof.

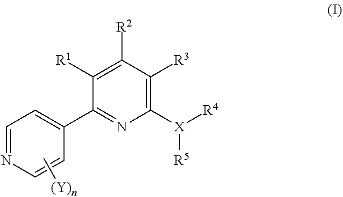

The role of protein kinase C (PKC) in cell signaling has been known for almost two decades and since then a whole family of PKC-like enzymes has been identified. In 1994, a novel PKC-related enzyme, PKCmu, otherwise known as protein kinase D (PKD), was identified. This widely expressed cytosolic serine-threonine kinase can be recruited to the trans-Golgi network and is, therefore, considered a modulator of cell trafficking. This was followed in 1999, by the identification of PKCnu (PKD3), and in 2001 by the identification of PKD2, the constitutive expression of which was largely restricted to the pancreas, heart, lung, smooth muscle, brain and rapidly proliferating tissues such as testis and colonic crypts. PKD, PKCnu and PKD2 are now accepted as a distinct PKC-related family of protein kinases, called the PKD family.

The three isoforms of the PKD family, PKD1/PKCmu PKD2 and PKD3/PKCnu, share a similar architecture with regulatory sub-domains that play specific roles in the activation, translocation and function of the enzymes. The PKD enzymes have been implicated in diverse cellular functions, including Golgi organization and plasma membrane directed transport, metastasis, immune responses, apoptosis and cell proliferation. FEBS Lett. 2003 Jul. 3; 546(1):81-6.

The PKD enzymes represent a new family of second messenger stimulated kinases, with diacylglycerol as a prime, but not the sole, mediator of activation. Their molecular architecture features a catalytic domain, unrelated to that of PKC family members; a large inhibitory, regulatory domain, comprised of two zinc fingers; and a pleckstrin homology domain. These different sub-domains play distinctive roles in the activation, translocation and biological functions of the kinase. The enzymes have been implicated in signaling mechanisms controlling cell proliferation and programmed cell death and in metastasis, immune responses, and Golgi restructuring and function. A variety of proteins specifically interact with the different sub-domains of the enzymes and directs their wide range of cellular functions. Int J Biochem Cell Biol. 2002 June; 34(6):577-81

Since its identification, PKD has been shown to play a role in growth factor signaling as well as in stress-induced signaling. It enhances expression of anti-apoptotic genes through the activation of NFkB and is activated upon treatment of cells with genotoxic chemotherapeutics. Moreover, PKD has emerged as an important regulator of plasma membrane enzymes and receptors. In some cases, it mediates cross-talk between different signaling systems.

PKD1 has been shown to play a role in proliferation of keratinocytes in skin, B and T lymphocytes and mast cells signaling. Transcriptional regulation of gene expression is tightly coupled to histone deacetylases (HDAC) and histone acetyltransferase (HAT) that modify the access of transcription factors to DNA binding sites. PKD1 has been shown to participate in nuclear export of HDAC5. HDAC5 is phosphorylated by PKD1 in cardiac myocytes, which results in the binding of 14-3-3 protein to the phosphoserine motif on HDAC5, thus leading to nuclear export through a CRM1-dependent mechanism. This results in increased transcriptional activity of hypertrophy mediating genes in myocytes. Cardiac failure is usually preceded by cardiac hypertrophy that is mediated by altered gene expression involved in myocyte contraction, calcium handling and metabolism.

The invention pertains to the compounds and methods for using them as described herein.

In another embodiment, the invention pertains, at least in part, to compounds of Formula I:

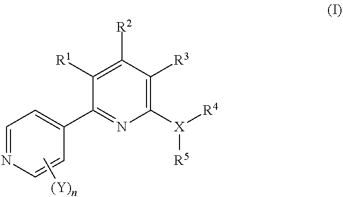

wherein

R¹, R², and R³ are each independently hydrogen, halogen, cyano, nitro, hydroxy, alkyl, alkoxy, alkoxycarbonyl, —C(O)NR⁷R⁸, hydroxycarbonyl, —NR⁹R¹⁰, alkylsulfonyl, heterocyclyl, heteroaryl, or aryl; or R² may be linked with R¹ to form a lactam ring, or R² may be linked with R³ to form a lactam ring;

X is hydrogen, nitrogen, or unsubstituted or substituted carbon;

R⁴ and R⁵ are each independently hydrogen, heterocyclyl, alkyl, or R⁴ and R⁵ are absent when X is hydrogen, or R⁴ and R⁵ are linked together to form a heterocyclic or heteroaryl ring;

R⁷ and R⁸ are each independently hydrogen, alkyl, or cycloalkyl;

R⁹ and R¹⁰ are each independently hydrogen, alkoxycarbonyl, arylaminocarbonyl, sulfonyl, acyl, or aryl;

Y is independently selected for each occurrence from halogen, cyano, nitro, hydroxy, aryl, alkyl, alkoxy, or —NR¹¹R¹², provided that at least one Y is —NR¹¹R¹²;

R¹¹ and R¹² are each independently hydrogen, cycloalkyl, heterocyclyl, aryl, arylamino, heteroaryl, or alkyl;

n is an integer selected from 0, 1, 2, 3, or 4; and pharmaceutically acceptable salts, polymorphs, rotamers, prodrugs, enantiomers, hydrates, and solvates thereof.

In another embodiment, the invention pertains, at least in part, to a method for treating a PKD associated disorder or disease in a subject by administering to the subject a therapeutically effective amount of a compound of Formula I, such that the PKD associated disorder in the subject is treated.

In yet another embodiment, the invention pertains, at least in part, to a method for treating a subject for heart failure, colorectal cancer, regulation of cell growth, autoimmune disorders, or hyperproliferative skin disorders, comprising administering to the subject a therapeutically effective amount of a compound of Formula I, such that the subject is treated.

In yet another embodiment, the invention pertains, at least in part, to pharmaceutical compositions, comprising an effective amount of a compound of Formula I and a pharmaceutical carrier, wherein said effective amount is effective to treat a PKD associated disorder or disease.

In another embodiment, the invention pertains, at least in part, to pharmaceutical compositions comprising a compound of the invention (e.g., a compound of Formula I or a compound otherwise described herein), and a pharmaceutically acceptable carrier.

The invention pertains, at least in part, to compounds, pharmaceutical compositions containing the compound and methods of use thereof. The present invention also relates to novel compounds which may be used, for example, as modulators PKD-1/2/3, or inhibitors of histone deacetylase (HDAC) phosphorylation. These compounds may, for example, be used to treat various PKD associated states such as heart failure, colorectal cancer, regulation of cell growth, autoimmune disorders, or hyperproliferative skin disorders.

PKD may be implicated in a number of clinical conditions including infectious/inflammatory disease, cancer, metabolic disease, and cardiovascular disorders. PKD has been shown to be involved in the down-stream response to receptor-antigen binding in T and B cells, neutrophils, and mast cells, and mediation of the mast cell response to a variety of cytokines. Moreover, PKD mediates the mitogenic response to a variety of biological mediators and molecules, such as for example, the biological responses elicited by PKC activation in small cell lung cancer cells, and responses that sensitize cells for apoptosis induced by genotoxic chemotherapeutics. Metabolic control may also involve PKD since it plays a role in pre-adipocyte differentiation, and the cellular location of PKD in skeletal muscle changes upon transition between the fasted and the fed state. Moreover, PKD is expressed in the myocardium and vascular smooth muscle and activated by oxidative stress. PKD is activated by key cardiovascular mediators such as angiotensin II, endothelin and PDGF. Modulation of PKD thus has the potential to modulate immune cell regulation, tumor progression, metabolic disorders and cardiovascular disease.

In particular, PKD1 may play a role in development of central tolerance in thymus gland, proliferation of pancreatic cancer cells, cardiac myocyte contraction, endothelial cell proliferation, osteoblasts differentiation, and prostate cancer cells adhesion and invasion. Furthermore, compounds that specifically modulate PKD1 may be of benefit in limiting cardiac hypertrophy.

### Compounds of the Invention

The present invention pertains, at least in part, to compounds of Formula I:

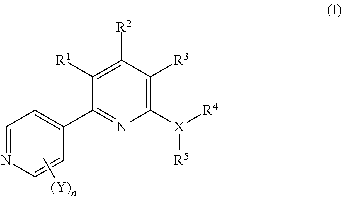

wherein

R¹, R², and R³ are each independently hydrogen, halogen, cyano, nitro, hydroxy, alkyl, alkoxy, alkoxycarbonyl, —C(O)NR⁷R⁸, hydroxycarbonyl, —NR⁹R¹⁰, alkylsulfonyl, heterocyclyl, heteroaryl, or aryl; or R² may be linked with R¹ to form a lactam ring, or R² may be linked with R³ to form a lactam ring;

X is hydrogen, nitrogen, or unsubstituted or substituted carbon;

R⁴ and R⁵ are each independently hydrogen, heterocyclyl, alkyl, or R⁴ and R⁵ are absent when X is hydrogen, or R⁴ and R⁵ are linked together to form a heterocyclic or heteroaryl ring;

R⁷ and R⁸ are each independently hydrogen, alkyl, or cycloalkyl;

R⁹ and R¹⁰ are each independently hydrogen, alkoxycarbonyl, arylaminocarbonyl, sulfonyl, acyl, or aryl;

Y is independently selected for each occurrence from halogen, cyano, nitro, hydroxy, aryl, alkyl, alkoxy, or —NR¹¹R¹², provided that at least one Y is —NR¹¹R¹²;

R¹¹ and R¹² are each independently hydrogen, cycloalkyl, heterocyclyl, aryl, arylamino, heteroaryl, or alkyl;

n is an integer selected from 0, 1, 2, 3, or 4; and pharmaceutically acceptable salts, polymorphs, rotamers, prodrugs, enantiomers, hydrates, and solvates thereof.

Examples of X include hydrogen, nitrogen, or carbon (e.g., C═O or —CR¹⁸). R¹⁸ may be hydrogen or alkyl.

In one embodiment, R¹ is hydrogen, alkyl (e.g., methyl), alkenyl, alkynyl, arylalkyl, alkoxycarbonyl (e.g., ethoxycarbonyl), aminocarbonyl, or heteroaryl (e.g., pyridinyl such as 3-pyridinyl or 4-pyridinyl). The aforementioned R¹ groups may be unsubstituted or substituted.

In another embodiment, R¹ is aryl (e.g., phenyl), which is optionally substituted with an electron withdrawing group. Examples of electron withdrawing groups include trifluoromethyl, halogen (e.g., fluorine, chlorine, iodine, or bromine), cyano, nitro, sulfonyl, and carbonyl moieties (e.g., formyl, acyl, carboxy, —C(O)-halogen, and carboxylic acid). In yet another embodiment, R¹ is alkylaminocarbonyl, such as ethylaminocarbonyl, which may be optionally substituted with cyano.

In one embodiment, R¹ and R³ are hydrogen.

Examples of R² include hydrogen, halogen (e.g., fluorine, chlorine, iodine, or bromine), cyano, nitro, hydroxy, alkyl, alkoxy (e.g., methoxy), alkoxycarbonyl (e.g., methoxycarbonyl), hydroxycarbonyl, alkylsulfonyl (e.g., methylsulfonyl), heterocyclyl, heteroaryl, or aryl (e.g., phenyl). In another embodiment, R² may also be —C(O)NR⁷R⁸, wherein R⁷ and R⁸ may independently be hydrogen, alkyl (e.g., methyl, ethyl, or isopropyl), or cycloalkyl (e.g., cyclohexyl). In yet another embodiment, R² may also be —NR⁹R¹⁰, wherein R⁹ and R¹⁰ may independently be hydrogen, alkoxycarbonyl (e.g., methoxycarbonyl), arylaminocarbonyl (e.g., phenylaminocarbonyl), sulfonyl (e.g., methylsulfonyl), acyl (e.g., —C(O)Me), or aryl (e.g., phenyl) which may be substituted with halogen (e.g., chlorine or fluorine), alkyl (e.g., methyl), or combinations thereof. This alkyl substituent may be further substituted with halogen (e.g., fluorine), and may be, for example, trifluoromethyl. Each of the aforementioned R², R⁷, R⁸, R⁹, and R¹⁰ groups may be unsubstituted or substituted.

In a further embodiment, R² is aryl, which may be substituted with cyano or halogen (e.g., fluorine).

In an additional embodiment of the present invention, R⁹ is alkyl (e.g., methyl) which is optionally substituted with aryl. This aryl substituent may be further substituted with halogen, resulting in, for example, a halo substituted benzylamino R² group.

In yet another embodiment, R² is an unsubstituted or substituted alkyl, (e.g., methyl or isopropyl). Examples of substitution on this alkyl include optionally substituted amino (e.g., methylamino). This amino may be substituted with alkyl or cyano substituted alkyl, resulting in methyl methylamino, and methyl methylamino acetonitrile, respectively as R² groups. Alternatively when R² is alkyl, this alkyl may be substituted with aminocarbonyl, resulting in, for example, —CH₂C(O)NH₂ as an R² group; hydroxy, resulting in, for example, a hydroxymethyl or a methyl hydroxyethyl R² group; cyano, resulting in, for example, a cyanomethyl R² group; azido, resulting in, for example, an azidomethyl R² group; and halogen (e.g., fluorine), resulting in, for example, a trifluoromethyl R² group.

In another embodiment, R² is heterocyclyl (e.g., 1,3,4-oxadiazolyl or oxadiazolone), which may be optionally substituted with alkyl (e.g., methyl or disubstituted methyl).

In yet another embodiment, R² is unsubstituted or substituted heteroaryl, such as pyridinyl, pyrimidinyl, oxazolyl, imidazolyl, tetrazolyl, thiazolyl, pyridazinyl or pyrazolyl. In this embodiment, heteroaryl may be substituted with alkyl (e.g., methyl), amino, alkoxycarbonyl (e.g., ethoxycarbonyl), or any other substituent that allows the compound to perform its intended function.

Another aspect of the invention includes compounds in which R³ is hydrogen; alkyl (e.g., methyl); aminocarbonyl; alkoxycarbonyl (e.g., methoxycarbonyl); or alkylaminocarbonyl (e.g., methylaminocarbonyl, ethylaminocarbonyl, or isopropylaminocarbonyl) which may be optionally substituted, e.g., with cyano or alkylamino.

In one embodiment, R⁴ and R⁵ are hydrogen. In other embodiments, R⁴ is hydrogen and R⁵ is heterocyclyl (e.g., pyrrolidinyl, piperazinyl, or morpholinyl), alkyl (e.g., methyl or ethyl), alkenyl, alkynyl, or aryl (e.g., phenyl). Each of the aforementioned R⁵ groups may be unsubstituted or substituted.

Alternatively, R⁴ and R⁵ may be linked together to form an unsubstituted or substituted heterocycle (e.g., piperazinyl).

In one embodiment, R⁴ is hydrogen and R⁵ is alkyl (e.g., methyl or ethyl) which may further be substituted, e.g., with hydroxy.

In a further embodiment, R⁴ and R⁵ are each alkyl (e.g., methyl or ethyl).

In a further embodiment, R⁴ and R⁵ are absent when X is hydrogen.

In yet another embodiment, R⁴ and R⁵ when linked together are heteroaryl, such as unsubstituted or substituted pyridinyl; or a heterocycle, such as unsubstituted or substituted piperazinyl, unsubstituted or substituted piperidinyl, unsubstituted or substituted morpholinyl, or unsubstituted or substituted pyrrolopyrazinyl. Examples of heterocycles include both spiro-heterocycles and fused heterocycles. Heterocycles of the present invention may be substituted with alkyl (e.g., methyl), amino substituted alkyl, acyl (e.g., —C(O)Me), amino, aminocarbonyl, alkylaminocarbonyl (e.g., butylaminocarbonyl or isopropylaminocarbonyl), alkylcarbonylamino, alkoxycarbonyl (e.g., methoxycarbonyl or butoxycarbonyl), hydroxycarbonyl, or any other substituent which allows the compound to perform its intended function.

In another embodiment, Y is NR¹¹R¹² and n is 1. In yet another embodiment, Y may be in the 2 position.

Examples of R¹¹ and R¹² include embodiments in which both R¹¹ and R¹² are hydrogen, or R¹¹ is hydrogen and R¹² is heterocyclyl (e.g., pyranyl).

In another embodiment, R¹¹ is hydrogen and R¹² is

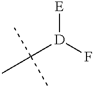

wherein:

D is aryl (e.g., phenyl)

E is alkyl (e.g., methyl) or halogen (e.g., fluorine or chlorine);

F is hydrogen; halogen; alkylaminocarbonyl (e.g., ethylaminocarbonyl or isopropylaminocarbonyl) which may be optionally substituted with heterocyclyl (e.g., morpholinyl or pyrrolidinyl) or dialkylamino (e.g., dimethylamino); heterocyclylaminocarbonyl (e.g., pyranylaminocarbonyl); or alkoxy (e.g., methoxy). The aforementioned groups D, E, and F may be unsubstituted or substituted.

In a further embodiment, R¹¹ is hydrogen and R¹² is cycloalkyl (e.g., cyclohexyl or cyclopentyl) optionally substituted with halogen, such as fluorine; or R¹¹ is hydrogen and R¹² is heteroaryl (e.g., pyrazolyl) optionally substituted with alkyl, such as methyl. In yet another embodiment, R¹¹ is hydrogen and R¹² is alkyl (e.g., methyl, isopropyl, pentyl, or ethyl) optionally substituted with alkoxy (e.g., methoxy); heteroaryl (e.g., imidizolyl); or aryl (e.g., phenyl), wherein this aryl may in turn be substituted with halogen (e.g., chlorine) or hydroxy.

Another aspect of the present invention pertains, at least in part, to compounds of Formula I, wherein R⁴ is hydrogen and R⁵ is heterocyclyl; or

R⁴ and R⁵ are linked together to form the following heterocyclic ring:

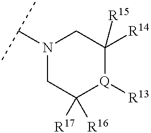

wherein

Q is nitrogen, oxygen, or —CH;

R¹³ is hydrogen, alkyl, acyl, aminocarbonyl, hydroxycarbonyl, amino, alkylaminocarbonyl, alkoxycarbonyl, or absent when Q is oxygen, or when linked with R¹⁶ may be a heterocycle; and

R¹⁴, R¹⁵, R¹⁶, and R¹⁷ are each independently hydrogen, alkyl, amino, or R¹⁴ and R¹⁵ may optionally be linked to form a ring, or R¹⁶ and R¹⁷ may optionally be linked to form a ring.

Examples of R¹³ include hydrogen, alkyl (e.g., methyl), acyl (e.g., —C(O)Me), alkoxycarbonyl (e.g., methoxycarbonyl or butoxycarbonyl, such as tertbutoxycarbonyl), aminocarbonyl, hydroxycarbonyl, amino, aminoalkyl (e.g., aminomethyl), alkylaminocarbonyl (e.g., isopropylaminocarbonyl), and acylamino.

In a further embodiment, R¹³ taken with R¹⁶ is a 5-membered heterocycle.

In yet a further embodiment, R¹⁴ and R¹⁵ or R¹⁶ and R¹⁷ are linked to form a cyclopropyl.

In another embodiment, R¹⁴ and R¹⁶ are methyl and R¹⁵ and R¹⁷ are hydrogen.

In yet another embodiment, R¹ and R³ are hydrogen; R² is hydrogen, cyano, nitro, hydroxy, —C(O)NH₂, or heteroaryl optionally substituted with —NH₂ (e.g., pyrazolyl or thiazolyl); or R² may be linked with R¹ to form a lactam ring, or R² may be linked with R³ to form a lactam ring; Y is NR¹¹R¹²; and R¹¹ and R¹² are each independently hydrogen, alkyl, cycloalkyl, heterocyclyl, aryl, or heteroaryl.

In one embodiment, R¹¹ is hydrogen.

In another embodiment, R¹² is alkyl (e.g., ethyl) optionally substituted with alkoxy (e.g., methoxy); or heterocyclyl (e.g., pyranyl). In yet another embodiment, R¹² is aryl (e.g., phenyl), which may be optionally substituted with halogen, (e.g., fluorine or chlorine); alkyl (e.g., methyl); alkylaminocarbonyl substituted alkyl, in which the alkylaminocarbonyl is, for example, ethylaminocarbonyl or isopropylaminocarbonyl and may be further substituted with heterocyclyl, such as morpholinyl or pyrrolidinyl (e.g., resulting in a heterocyclic alkylamino carbonyl alkyl R¹² group); alkoxy substituted alkyl, such as methoxy; or heterocyclylaminocarbonyl which is optionally substituted with halogen.

Additional examples of R¹² include cycloalkyl (e.g., cyclohexyl or cyclopentyl) which may optionally be substituted with halogen, such as fluorine; heteroaryl (e.g., pyrazolyl) which may be unsubstituted or substituted with alkyl (e.g., methyl); alkyl (e.g., methyl, isopropyl, pentyl, or ethyl) optionally substituted with heteroaryl, for example, imidizolyl; aryl, for example, phenyl; halogen substituted aryl, for example, chlorine substituted phenyl; hydroxy substituted aryl; or arylamino (e.g., phenylamino) which is optionally substituted with alkyl, such as methyl, alkylaminocarbonyl, such as propylaminocarbonyl which is optionally substituted with an alkylamino, such as dimethylamino.

In another embodiment, the invention pertains, at least in part, to a compound of Formula I, wherein R¹ is hydrogen; R² is hydrogen, nitro, —C(O)NH₂, or pyrazolyl; R³ is hydrogen, or R² and R³ may optionally be linked to form a lactam ring;

X is

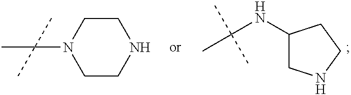

Y is —NHR¹² and Y is in the 2 position; and R¹² is isopropyl, cyclohexyl, phenyl, benzyl, pyranyl, pyrazolyl, or —C(O)(CH₂)₂.

Examples of R¹² include benzyl substituted with hydroxy; phenyl which may be unsubstituted or optionally substituted with methyl, fluorine, or methoxy; —C(O)(CH₂)₂—wherein the methylene is substituted with pyrrolidinyl; or pyrazolyl wherein the nitrogen is substituted with methyl.

Further examples of compounds of Formula I include the compounds listed in the examples and pharmaceutically acceptable salts, polymorphs, rotamers, prodrugs, enantiomers, hydrates, and solvates thereof, and are also considered to be “compounds of the invention”.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl (alicyclic) groups (cyclopropyl, cyclopentyl, cyclohexyl, cycloheptyl, cyclooctyl), alkyl substituted cycloalkyl groups, and cycloalkyl substituted alkyl groups. The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain), and more preferably 4 or fewer. Likewise, preferred cycloalkyls have from 3-8 carbon atoms in their ring structure, and more preferably have 5 or 6 carbons in the ring structure. The term C₁-C₆ includes alkyl groups containing 1 to 6 carbon atoms.

Moreover, the term alkyl includes both “unsubstituted alkyls” and “substituted alkyls,” the latter of which refers to alkyl moieties having substituents replacing a hydrogen on one or more carbons of the hydrocarbon backbone. Such substituents can include, for example, alkenyl, alkynyl, halogen, hydroxy, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids.

The term “aryl” includes groups, including 5- and 6-membered single-ring aromatic groups that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, etc. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles,” “heterocycles,” “heteroaryls” or “heteroaromatics.”

Typical heteroaryl groups include 2- or 3-thienyl, 2- or 3-furyl, 2- or 3-pyrrolyl, 2-, 4-, or 5-imidazolyl, 3-, 4-, or 5-pyrazolyl, 2-, 4-, or 5-thiazolyl, 3-, 4-, or 5-isothiazolyl, 2-, 4-, or 5-oxazolyl, 3-, 4-, or 5-isoxazolyl, 3- or 5-1,2,4-triazolyl, 4- or 5-1,2,3-triazolyl, tetrazolyl, 2-, 3-, or 4-pyridyl, 3- or 4-pyridazinyl, 3-, 4-, or 5-pyrazinyl, 2-pyrazinyl, 2-, 4-, or 5-pyrimidinyl. A heteroaryl group may be mono-, bi-, tri-, or polycyclic.

The term “heteroaryl” also refers to a group in which a heteroaromatic ring is fused to one or more aryl, cycloaliphatic, or heterocyclyl rings, where the radical or point of attachment is on the heteroaromatic ring. Non-limiting examples include but are not limited to 1-, 2-, 3-, 5-, 6-, 7-, or 8-indolizinyl, 1-, 3-, 4-, 5-, 6-, or 7-isoindolyl, 2-, 3-, 4-, 5-, 6-, or 7-indolyl, 2-, 3-, 4-, 5-, 6-, or 7-indazolyl, 2-, 4-, 5-, 6-, 7-, or 8-purinyl, 1-, 2-, 3-, 4-, 6-, 7-, 8-, or 9-quinolizinyl, 2-, 3-, 4-, 5-, 6-, 7-, or 8-quinoliyl, 1-, 3-, 4-, 5-, 6-, 7-, or 8-isoquinolinyl, 1-, 4-, 5-, 6-, 7-, or 8-phthalazinyl, 2-, 3-, 4-, 5-, or 6-naphthyridinyl, 2-, 3-, 5-, 6-, 7-, or 8-quinazolinyl, 3-, 4-, 5-, 6-, 7-, or 8-cinnolinyl, 2-, 4-, 6-, or 7-pteridinyl, 1-, 2-, 3-, 4-, 5-, 6-, 7-, or 8-4-aH carbazolyl, 1-, 2-, 3-, 4-, 5-, 6-, 7-, or 8-carbazolyl, 1-, 3-, 4-, 5-, 6-, 7-, 8-, or 9-carbolinyl, 1-, 2-, 3-, 4-, 6-, 7-, 8-, 9-, or 10-phenanthridinyl, 1-, 2-, 3-, 4-, 5-, 6-, 7-, 8-, or 9-acridinyl, 1-, 2-, 4-, 5-, 6-, 7-, 8-, or 9-perimidinyl, 2-, 3-, 4-, 5-, 6-, 8-, 9-, or 10-phenathrolinyl, 1-, 2-, 3-, 4-, 6-, 7-, 8-, or 9-phenazinyl, 1-, 2-, 3-, 4-, 6-, 7-, 8-, 9-, or 10-phenothiazinyl, 1-, 2-, 3-, 4-, 6-, 7-, 8-, 9-, or 10-phenoxazinyl, 2-, 3-, 4-, 5-, 6-, or I-, 3-, 4-, 5-, 6-, 7-, 8-, 9-, or 10-benzisoqinolinyl, 2-, 3-, 4-, or thieno[2,3-b]furanyl, 2-, 3-, 5-, 6-, 7-, 8-, 9-, 10-, or 11-7H-pyrazino[2,3-c]carbazolyl, 2-, 3-, 5-, 6-, or 7-2H-furo[3,2-b]-pyranyl, 2-, 3-, 4-, 5-, 7-, or 8-5H-pyrido[2,3-d]-o-oxazinyl, 1-, 3-, or 5-1H-pyrazolo[4,3-d]-oxazolyl, 2-, 4-, or 54H-imidazo[4,5-d]thiazolyl, 3-, 5-, or 8-pyrazino[2,3-d]pyridazinyl, 2-, 3-, 5-, or 6-imidazo[2,1-b]thiazolyl, 1-, 3-, 6-, 7-, 8-, or 9-furo[3,4-c]cinnolinyl, 1-, 2-, 3-, 4-, 5-, 6-, 8-, 9-, 10, or 11-4H-pyrido[2,3-c]carbazolyl, 2-, 3-, 6-, or 7-imidazo[1,2-b][1,2,4]triazinyl, 7-benzo[b]thienyl, 2-, 4-, 5-, 6-, or 7-benzoxazolyl, 2-, 4-, 5-, 6-, or 7-benzimidazolyl, 2-, 4-, 4-, 5-, 6-, or 7-benzothiazolyl, 1-, 2-, 4-, 5-, 6-, 7-, 8-, or 9-benzoxapinyl, 2-, 4-, 5-, 6-, 7-, or 8-benzoxazinyl, 1-, 2-, 3-, 5-, 6-, 7-, 8-, 9-, 10-, or 11-1H-pyrrolo[1,2-b][2]benzazapinyl. Typical fused heteroaryl groups include, but are not limited to 2-, 3-, 4-, 5-, 6-, 7-, or 8-quinolinyl, 1-, 3-, 4-, 5-, 6-, 7-, or 8-isoquinolinyl, 2-, 3-, 4-, 5-, 6-, or 7-indolyl, 2-, 3-, 4-, 5-, 6-, or 7-benzo[b]thienyl, 2-, 4-, 5-, 6-, or 7-benzoxazolyl, 2-, 4-, 5-, 6-, or 7-benzimidazolyl, 2-, 4-, 5-, 6-, or 7-benzothiazolyl.

The aromatic ring of an “aryl” or “heteroaryl” group can be substituted at one or more ring positions with such substituents as described above, as for example, halogen, hydroxy, alkoxy, alkylcarbonyloxy, arylcarbonyloxy, alkoxycarbonyloxy, aryloxycarbonyloxy, carboxylate, alkylcarbonyl, alkylaminocarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin).

The term “alkenyl” includes unsaturated aliphatic groups analogous in length and possible substitution to the alkyls described above, but that contain at least one double bond.

For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term alkenyl further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ or straight chain, C₃-C₆ for branched chain). Likewise, cycloalkenyl groups may have from 3-8 carbon atoms in their ring structure, and more preferably have 5 or 6 carbons in the ring structure. The term C₂-C₆ includes alkenyl groups containing 2 to 6 carbon atoms.

Moreover, the term alkenyl includes both “unsubstituted alkenyls” and “substituted alkenyls,” the latter of which refers to alkenyl moieties having substituents replacing a hydrogen on one or more carbons of the hydrocarbon backbone. Such substituents can include, for example, alkyl groups, alkynyl groups, halogens, hydroxy, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond.

For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term alkynyl further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term alkynyl includes both “unsubstituted alkynyls” and “substituted alkynyls,” the latter of which refers to alkynyl moieties having substituents replacing a hydrogen on one or more carbons of the hydrocarbon backbone. Such substituents can include, for example, alkyl groups, alkynyl groups, halogens, hydroxy, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Unless the number of carbons is otherwise specified, the term “lower alkyl” means an alkyl group, as defined above, but having from one to five carbon atoms in its backbone structure. “Lower alkenyl” and “lower alkynyl” have chain lengths of, for example, 2-5 carbon atoms.

The term “alkoxy” includes substituted and unsubstituted alkyl, alkenyl, and alkynyl groups covalently linked to an oxygen atom. Examples of alkoxy groups include methoxy, ethoxy, isopropoxy, propoxy, butoxy, and pentoxy groups. Examples of substituted alkoxy groups include halogenated alkoxy groups. The alkoxy groups can be substituted with groups such as alkenyl, alkynyl, halogen, hydroxy, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “acyl” includes compounds and moieties which contain the acyl radical (CH₃CO—) or a carbonyl group. It includes substituted acyl moieties. The term “substituted acyl” includes acyl groups where one or more of the hydrogen atoms are replaced by for example, alkyl groups, alkynyl groups, halogens, hydroxy, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein an acyl moiety is bonded to an amino group. For example, the term includes alkylcarbonylamino, arylcarbonylamino, carbamoyl and ureido groups.

The term “aroyl” includes compounds and moieties with an aryl or heteroaromatic moiety bound to a carbonyl group. Examples of aroyl groups include phenylcarboxy, naphthyl carboxy, etc. It includes substituted aroyl moieties. The term “substituted aroyl” includes aroyl groups where one or more of the hydrogen atoms are replaced by for example, alkyl groups, alkynyl groups, halogens, hydroxy, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxy, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The terms “alkoxyalkyl,” “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “carbamoyl” includes H₂NC(O)—, alkyl-NHC(O)—, (alkyl)₂NC(O)—, aryl-NHC(O)—, alkyl(aryl)-NC(O)—, heteroaryl-NHC(O)—, alkyl(heteroaryl)-NC(O)—, aryl-alkyl-NHC(O)—, alkyl(aryl-alkyl)-NC(O)—, etc. The term includes substituted carbamoyl moieties.

The term “sulfonyl” includes R—SO₂—, wherein R is hydrogen, alkyl, aryl, heteroaryl, aryl-alkyl, heteroaryl-alkyl, alkoxy, aryloxy, cycloalkyl, or heterocyclyl.

The term “sulfonamido” includes alkyl-S(O)₂—NH—, aryl-S(O)₂—NH—, aryl-alkyl-S(O)₂—NH—, heteroaryl-S(O)₂—NH—, heteroaryl-alkyl-S(O)₂—NH—, alkyl-S(O)₂—N(alkyl)-, aryl-S(O)₂—N(alkyl)-, aryl-alkyl-S(O)₂—N(alkyl)-, heteroaryl-S(O)₂—N(alkyl)-, heteroaryl-alkyl-S(O)₂—N(alkyl)-, etc. The term includes substituted carbamoyl moieties

The term “heterocyclyl” or “heterocyclo” includes an optionally substituted, saturated or unsaturated non-aromatic ring or ring system, e.g., which is a 4-, 5-, 6-, or 7-membered monocyclic, 7-, 8-, 9-, 10-, 11-, or 12-membered bicyclic or 10-, 11-, 12-, 13-, 14- or 15-membered tricyclic ring system and contains at least one heteroatom selected from O, S and N, where the N and S can also optionally be oxidized to various oxidation states. The heterocyclic group can be attached at a heteroatom or a carbon atom. The heterocyclyl can include fused or bridged rings as well as spirocyclic rings. Examples of heterocycles include tetrahydrofuran, dihydrofuran, 1,4-dioxane, morpholine, 1,4-dithiane, piperazine, piperidine, 1,3-dioxolane, imidazolidine, imidazoline, pyrroline, pyrrolidine, tetrahydropyran, dihydropyran, oxathiolane, dithiolane, 1,3-dioxane, 1,3-dithiane, oxathiane, thiomorpholine, etc.

The term “heterocyclyl” includes heterocyclic groups as defined herein substituted with 1, 2 or 3 substituents such as alkyl, hydroxy (or protected hydroxy), halo, oxo (e.g., ═O), amino, alkylamino or dialkylamino, alkoxy, cycloalkyl, carboxyl, heterocyclooxy, wherein heterocyclooxy denotes a heterocyclic group bonded through an oxygen bridge, alkyl-O—C(O)—, mercapto, nitro, cyano, sulfamoyl or sulfonamide, aryl, alkyl-C(O)—O—, aryl-C(O)—O—, aryl-S—, aryloxy, alkyl-S—, formyl (e.g., HC(O)—), carbamoyl, aryl-alkyl-, and aryl substituted with alkyl, cycloalkyl, alkoxy, hydroxy, amino, alkyl-C(O)—NH—, alkylamino, dialkylamino or halogen.

The term “sulfamoyl” includes H₂NS(O)₂—, alkyl-NHS(O)₂—, (alkyl)₂NS(O)₂—, aryl-NHS(O)₂—, alkyl(aryl)-NS(O)₂—, (aryl)₂NS(O)₂—, heteroaryl-NHS(O)₂—, (aryl-alkyl)-NHS(O)₂—, (heteroaryl-alkyl)-NHS(O)₂—, etc. The term includes substituted sulfamoyl moieties.

The term “aryloxy” includes both an —O-aryl and an —O-heteroaryl group, wherein aryl and heteroaryl are defined herein. The term includes substituted aryloxy moieties.

The term “amine” or “amino” includes compounds where a nitrogen atom is covalently bonded to at least one carbon or heteroatom. The term includes “alkyl amino” which comprises groups and compounds wherein the nitrogen is bound to at least one additional alkyl group. The term “dialkyl amino” includes groups wherein the nitrogen atom is bound to at least two additional alkyl groups. The term “arylamino” and “diarylamino” include groups wherein the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group. The term “amine” or “amino” also includes substituted moieties.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups which include aryl or heteroaryl moieties bound to an amino group which is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenylcarbonylamino,” “alkynylcarbonylamino,” and “arylcarbonylamino” are included in term “amide.” Amides also include urea groups (aminocarbonylamino) and carbamates (oxycarbonylamino). The term “amide,” “amido” or “aminocarbonyl” also includes substituted moieties.

The term “carbonyl” or “carboxy” includes compounds and moieties which contain a carbon connected with a double bond to an oxygen atom. The carbonyl can be further substituted with any moiety which allows the compounds of the invention to perform its intended function. For example, carbonyl moieties may be substituted with alkyls, alkenyls, alkynyls, aryls, alkoxy, aminos, etc. Examples of moieties which contain a carbonyl include aldehydes, ketones, carboxylic acids, amides, esters, anhydrides, etc.

The term “thiocarbonyl” or “thiocarboxy” includes compounds and moieties which contain a carbon connected with a double bond to a sulfur atom. The term also includes substituted moieties.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group. The term also includes substituted moieties.

The term “ester” includes compounds and moieties which contain a carbon or a heteroatom bound to an oxygen atom which is bonded to the carbon of a carbonyl group. The term “ester” includes alkoxycarboxy groups such as methoxycarbonyl, ethoxycarbonyl, propoxycarbonyl, butoxycarbonyl, pentoxycarbonyl, etc. The alkyl, alkenyl, or alkynyl groups are as defined above. The term also includes substituted moieties.

The term “thioether” includes compounds and moieties which contain a sulfur atom bonded to two different carbon or hetero atoms. Examples of thioethers include, but are not limited to alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and alkthioalkynyls” refer to compounds or moieties wherein an alkyl, alkenyl, or alkynyl group is bonded to a sulfur atom which is covalently bonded to an alkynyl group. The term also includes substituted moieties.

The term “hydroxy” or “hydroxyl” includes groups with an —OH or —O⁻.

The term “halogen” includes fluorine, bromine, chlorine, iodine, etc. The term “perhalogenated” generally refers to a moiety wherein all hydrogens are replaced by halogen atoms.

The terms “polycyclyl” or “polycyclic radical” refer to two or more cyclic rings (e.g., cycloalkyls, cycloalkenyls, cycloalkynyls, aryls and/or heterocyclyls) in which two or more carbons are common to two adjoining rings, e.g., the rings are “fused rings.” Rings that are joined through non-adjacent atoms are termed “bridged” rings. Each of the rings of the polycycle can be substituted with such substituents as described above, as for example, halogen, hydroxy, alkylcarbonyloxy, arylcarbonyloxy, alkoxycarbonyloxy, aryloxycarbonyloxy, carboxylate, alkylcarbonyl, alkoxycarbonyl, alkylaminocarbonyl, arylalkylaminocarbonyl, alkenylaminocarbonyl, alkylcarbonyl, arylcarbonyl, arylalkyl carbonyl, alkenylcarbonyl, aminocarbonyl, alkylthiocarbonyl, alkoxy, phosphate, phosphonato, phosphinato, cyano, amido, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkyl, alkylaryl, or an aromatic or heteroaromatic moiety.

The term “heteroatom” includes atoms of any element other than carbon or hydrogen. Preferred heteroatoms are nitrogen, oxygen, sulfur and phosphorus.

It will be noted that the structure of some of the compounds of this invention includes asymmetric carbon atoms. It is to be understood accordingly that the isomers arising from such asymmetry (e.g., all enantiomers and diastereomers) are included within the scope of this invention, unless indicated otherwise. Such isomers can be obtained in substantially pure form by classical separation techniques and by stereochemically controlled synthesis. Furthermore, the structures and other compounds and moieties discussed in this application also include all tautomers thereof.

The term “isomers” refers to different compounds that have the same molecular formula but differ in arrangement and configuration of the atoms. Moreover, the term “an optical isomer” or “a stereoisomer” refers to any of the various stereo isomeric configurations which may exist for a given compound of the present invention and includes geometric isomers. It is understood that a substituent may be attached at a chiral center of a carbon atom. Therefore, the invention includes enantiomers, diastereomers or racemates of the compound. “Enantiomers” are a pair of stereoisomers that are non-superimposable mirror images of each other. A 1:1 mixture of a pair of enantiomers is a “racemic” mixture. The term is used to designate a racemic mixture where appropriate. “Diastereoisomers” are stereoisomers that have at least two asymmetric atoms, but which are not mirror-images of each other. The absolute stereochemistry is specified according to the Cahn-Ingold-Prelog R—S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and may thus give rise to enantiomers, diastereomers, and other stereoisomeric forms that may be defined, in terms of absolute stereochemistry, as (R)-, or (S)-. The present invention is meant to include all such possible isomers, including racemic mixtures, optically pure forms and intermediate mixtures. Optically active (R)- and (S)-isomers may be prepared using chiral synthons or chiral reagents, or resolved using conventional techniques. If the compound contains a double bond, the substituent may be E or Z configuration. If the compound contains a disubstituted cycloalkyl, the cycloalkyl substituent may have a cis- or trans-configuration. All tautomeric forms are also intended to be included.

The recitation of ranges of values in the present application are merely intended to serve as a shorthand method of referring individually to each separate value falling within the range.

Any asymmetric carbon atom on the compounds of the present invention can be present in the (R)-, (S)- or (R,S)-configuration, preferably in the (R)- or (S)-configuration. Substituents at atoms with unsaturated bonds may, if possible, be present in cis-(Z)- or trans-(E)-form. Therefore, the compounds of the present invention can be in the form of one of the possible isomers or mixtures thereof, for example, as substantially pure geometric (cis or trans) isomers, diastereomers, optical isomers (antipodes), racemates or mixtures thereof.

Any resulting mixtures of isomers can be separated on the basis of the physicochemical differences of the constituents, into the pure geometric or optical isomers, diastereomers, racemates, for example, by chromatography and/or fractional crystallization.

Any resulting racemates of final products or intermediates can be resolved into the optical antipodes by known methods, e.g., by separation of the diastereomeric salts thereof, obtained with an optically active acid or base, and liberating the optically active acidic or basic compound. In particular, the imidazolyl moiety may thus be employed to resolve the compounds of the present invention into their optical antipodes, e.g., by fractional crystallization of a salt formed with an optically active acid, e.g., tartaric acid, dibenzoyl tartaric acid, diacetyl tartaric acid, di-O,O′-p-toluoyl tartaric acid, mandelic acid, malic acid or camphor-10-sulfonic acid. Racemic products can also be resolved by chiral chromatography, e.g., high pressure liquid chromatography (HPLC) using a chiral adsorbent.

Compounds of the present invention are either obtained in the free form, as a salt thereof, or as prodrug derivatives thereof.

The term “pharmaceutically acceptable salts” includes salts that retain the biological effectiveness and properties of the compounds of this invention and, which are not biologically or otherwise undesirable. In many cases, the compounds of the present invention are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, etc. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, etc. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, etc.; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, etc., specifically such as isopropylamine, trimethylamine, diethylamine, triethylamine, tripropylamine, and ethanolamine. The pharmaceutically acceptable salts of the present invention can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Lists of additional suitable salts can be found, e.g., in Remington's Pharmaceutical Sciences, 20th ed., Mack Publishing Company, Easton, Pa., (1985).

When a basic group is present in the compounds of the present invention, the compounds can be converted into acid addition salts thereof, in particular, acid addition salts with the imidazolyl moiety of the structure, preferably pharmaceutically acceptable salts thereof. These are formed, with inorganic acids or organic acids. Suitable inorganic acids include but are not limited to, hydrochloric acid, sulfuric acid, a phosphoric or hydrohalic acid. Suitable organic acids include but are not limited to, carboxylic acids, such as (C₁-C₄) alkanecarboxylic acids which, for example, are unsubstituted or substituted by halogen, e.g., acetic acid, such as saturated or unsaturated dicarboxylic acids, e.g., oxalic, succinic, maleic or fumaric acid, such as hydroxycarboxylic acids, e.g., glycolic, lactic, malic, tartaric or citric acid, such as amino acids, e.g., aspartic or glutamic acid, organic sulfonic acids, such as (C₁-C₄) alkylsulfonic acids, e.g., methanesulfonic acid; or arylsulfonic acids which are unsubstituted or substituted, e.g., by halogen. Preferred are salts formed with hydrochloric acid, methanesulfonic acid and maleic acid.

When an acidic group is present in the compounds of the present invention, the compounds can be converted into salts with pharmaceutically acceptable bases. Such salts include alkali metal salts, like sodium, lithium and potassium salts; alkaline earth metal salts, like calcium and magnesium salts; ammonium salts with organic bases, e.g., trimethylamine salts, diethylamine salts, tris(hydroxymethyl)methylamine salts, dicyclohexylamine salts and N-methyl-D-glucamine salts; salts with amino acids like arginine, lysine, etc. Salts may be formed using conventional methods, advantageously in the presence of an ethereal or alcoholic solvent, such as a lower alkanol. From the solutions of the latter, the salts may be precipitated with ethers, e.g., diethyl ether. Resulting salts may be converted into the free compounds by treatment with acids. These or other salts can also be used for purification of the compounds obtained.

When both a basic group and an acid group are present in the same molecule, the compounds of the present invention can also form internal salts.

Salts of compounds of the present invention having at least one salt-forming group may be prepared in a manner known per se. For example, salts of compounds of the present invention having acid groups may be formed, for example, by treating the compounds with metal compounds, such as alkali metal salts of suitable organic carboxylic acids, e.g. the sodium salt of 2-ethylhexanoic acid, with organic alkali metal or alkaline earth metal compounds, such as the corresponding hydroxides, carbonates or hydrogen carbonates, such as sodium or potassium hydroxide, carbonate or hydrogen carbonate, with corresponding calcium compounds or with ammonia or a suitable organic amine, stoichiometric amounts or only a small excess of the salt-forming agent preferably being used. Acid addition salts of compounds of the present invention are obtained in customary manner, e.g. by treating the compounds with an acid or a suitable anion exchange reagent. Internal salts of compounds of the present invention containing acid and basic salt-forming groups, e.g. a free carboxy group and a free amino group, may be formed, e.g. by the neutralisation of salts, such as acid addition salts, to the isoelectric point, e.g. with weak bases, or by treatment with ion exchangers.

Salts can be converted in customary manner into the free compounds; metal and ammonium salts can be converted, for example, by treatment with suitable acids, and acid addition salts, for example, by treatment with a suitable basic agent.

The present invention also provides prodrug moieties of the compounds of the present invention that convert in vivo to the compounds of the present invention. A prodrug moiety is an active or inactive compound that is modified chemically through in vivo physiological action, such as hydrolysis, metabolism, etc., into a compound of this invention following administration of the prodrug to a subject. The term “prodrug moiety” includes moieties which can be metabolized in vivo to a hydroxy group and moieties which may advantageously remain esterified in vivo. Preferably, the prodrugs moieties are metabolized in vivo by esterases or by other mechanisms to hydroxy groups or other advantageous groups. Examples of prodrugs and their uses are well known in the art (See, e.g., Berge et al. (1977) “Pharmaceutical Salts” J. Pharm. Sci. 66:1-19). The prodrugs can be prepared in situ during the final isolation and purification of the compounds, or by separately reacting the purified compound in its free acid form or hydroxy with a suitable esterifying agent. Hydroxy groups can be converted into esters via treatment with a carboxylic acid. Examples of prodrug moieties include substituted and unsubstituted, branch or unbranched lower alkyl ester moieties, (e.g., propionoic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionoic acid esters and acyl esters.

The suitability and techniques involved in making and using prodrugs are well known by those skilled in the art. Prodrugs can be conceptually divided into two non-exclusive categories, bioprecursor prodrugs and carrier prodrugs. See The Practice of Medicinal Chemistry, Ch. 31-32 (Ed. Wermuth, Academic Press, San Diego, Calif., 2001). Generally, bioprecursor prodrugs are compounds are inactive or have low activity compared to the corresponding active drug compound that contains one or more protective groups and are converted to an active form by metabolism or solvolysis. Both the active drug form and any released metabolic products should have acceptably low toxicity. Typically, the formation of active drug compound involves a metabolic process or reaction that is one of the follow types:

1. Oxidative reactions, such as oxidation of alcohol, carbonyl, and acid functions, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-dealkylation, oxidative O- and S-dealkylation, oxidative deamination, as well as other oxidative reactions.

2. Reductive reactions, such as reduction of carbonyl groups, reduction of alcoholic groups and carbon-carbon double bonds, reduction of nitrogen-containing functions groups, and other reduction reactions.

3. Reactions without change in the state of oxidation, such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improve uptake and/or localized delivery to a site(s) of action. Desirably for such a carrier prodrug, the linkage between the drug moiety and the transport moiety is a covalent bond, the prodrug is inactive or less active than the drug compound, and any released transport moiety is acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins. See, Cheng et al., US20040077595, incorporated herein by reference. Such carrier prodrugs are often advantageous for orally administered drugs. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxy groups with lipophilic carboxylic acids, or of carboxylic acid groups with alcohols, e.g., aliphatic alcohols. Wermuth, The Practice of Medicinal Chemistry, Ch. 31-32, Ed. Werriuth, Academic Press, San Diego, Calif., 2001.

Exemplary prodrugs are, e.g., esters of free carboxylic acids and S-acyl and O-acyl derivatives of thiols, alcohols or phenols, wherein acyl has a meaning as defined herein. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the -(lower alkanoyloxy, lower alkoxycarbonyl or di-lower alkylaminocarbonyl)-lower alkyl esters, such as the pivaloyloxymethyl ester, etc. conventionally used in the art. In addition, amines have been masked as arylcarbonyloxymethyl substituted derivatives which are cleaved by esterases in vivo releasing the free drug and formaldehyde (Bundgaard, J. Med. Chem. 2503 (1989)). Moreover, drugs containing an acidic NH group, such as imidazole, imide, indole, etc., have been masked with N-acyloxymethyl groups (Bundgaard, Design of Prodrugs, Elsevier (1985)). Hydroxy groups have been masked as esters and ethers. EP 039,051 (Sloan and Little) discloses Mannich-base hydroxamic acid prodrugs, their preparation and use.

In view of the close relationship between the compounds, the compounds in the form of their salts and the prodrugs, any reference to the compounds of the present invention is to be understood as referring also to the corresponding prodrugs of the compounds of the present invention, as appropriate and expedient.

Furthermore, the compounds of the present invention, including their salts, can also be obtained in the form of their hydrates, or include other solvents used for their crystallization.

Compounds of the present invention are prepared from commonly available compounds using procedures known to those skilled in the art, including any one or more of the following conditions without limitation:

Within the scope of this text, only a readily removable group that is not a constituent of the particular desired end product of the compounds of the present invention is designated a “protecting group”, unless the context indicates otherwise. The protection of functional groups by such protecting groups, the protecting groups themselves, and their cleavage reactions are described for example in standard reference works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999. A characteristic of protecting groups is that they can be removed readily (i.e. without the occurrence of undesired secondary reactions) for example by solvolysis, reduction, photolysis or alternatively under physiological conditions (e.g. by enzymatic cleavage).

Mixtures of isomers obtainable according to the invention can be separated in a manner known per se into the individual isomers; diastereoisomers can be separated, for example, by partitioning between polyphasic solvent mixtures, recrystallisation and/or chromatographic separation, for example over silica gel or by e.g. medium pressure liquid chromatography over a reversed phase column, and racemates can be separated, for example, by the formation of salts with optically pure salt-forming reagents and separation of the mixture of diastereoisomers so obtainable, for example by means of fractional crystallisation, or by chromatography over optically active column materials.

Intermediates and final products can be worked up and/or purified according to standard methods, e.g. using chromatographic methods, distribution methods, (re-) crystallization, etc. The following applies in general to all processes mentioned herein before and hereinafter.

All the above-mentioned process steps can be carried out under reaction conditions that are known per se, including those mentioned specifically, in the absence or, customarily, in the presence of solvents or diluents, including, for example, solvents or diluents that are inert towards the reagents used and dissolve them, in the absence or presence of catalysts, condensation or neutralizing agents, for example ion exchangers, such as cation exchangers, e.g. in the H+ form, depending on the nature of the reaction and/or of the reactants at reduced, normal or elevated temperature, for example in a temperature range of from about −100° C. to about 190° C., including, for example, from approximately −80° C. to approximately 150° C., for example at from −80 to −60° C., at room temperature, at from −20 to 40° C. or at reflux temperature, under atmospheric pressure or in a closed vessel, where appropriate under pressure, and/or in an inert atmosphere, for example under an argon or nitrogen atmosphere.

At all stages of the reactions, mixtures of isomers that are formed can be separated into the individual isomers, for example diastereoisomers or enantiomers, or into any desired mixtures of isomers, for example racemates or mixtures of diastereoisomers.

The solvents from which those solvents that are suitable for any particular reaction may be selected include those mentioned specifically or, for example, water, esters, such as lower alkyl-lower alkanoates, for example ethyl acetate, ethers, such as aliphatic ethers, for example diethyl ether, or cyclic ethers, for example tetrahydrofuran or dioxane, liquid aromatic hydrocarbons, such as benzene or toluene, alcohols, such as methanol, ethanol or 1- or 2-propanol, nitriles, such as acetonitrile, halogenated hydrocarbons, such as methylene chloride or chloroform, acid amides, such as dimethylformamide or dimethyl acetamide, bases, such as heterocyclic nitrogen bases, for example pyridine or N-methylpyrrolidin-2-one, carboxylic acid anhydrides, such as lower alkanoic acid anhydrides, for example acetic anhydride, cyclic, linear or branched hydrocarbons, such as cyclohexane, hexane or isopentane, or mixtures of those solvents, for example aqueous solutions, unless otherwise indicated in the description of the processes. Such solvent mixtures may also be used in working up, for example by chromatography or partitioning.

The compounds, including their salts, may also be obtained in the form of hydrates, or their crystals may, for example, include the solvent used for crystallization. Different crystalline forms may be present.

The invention relates also to those forms of the process in which a compound obtainable as an intermediate at any stage of the process is used as starting material and the remaining process steps are carried out, or in which a starting material is formed under the reaction conditions or is used in the form of a derivative, for example in a protected form or in the form of a salt, or a compound obtainable by the process according to the invention is produced under the process conditions and processed further in situ.

All starting materials, building blocks, reagents, acids, bases, dehydrating agents, solvents and catalysts utilized to synthesize the compounds of the present invention are either commercially available or can be produced by organic synthesis methods known to one of ordinary skill in the art (Houben-Weyl 4^{th} Ed. 1952, Methods of Organic Synthesis, Thieme, Volume 21).

Generally, enantiomers of the compounds of the present invention can be prepared by methods known to those skilled in the art to resolve racemic mixtures, such as by formation and recrystallization of diastereomeric salts or by chiral chromotagraphy or HPLC separation utilizing chiral stationery phases.

In starting compounds and intermediates which are converted to the compounds of the invention in a manner described herein, functional groups present, such as amino, thiol, carboxyl and hydroxy groups, are optionally protected by conventional protecting groups that are common in preparative organic chemistry. Protected amino, thiol, carboxyl and hydroxy groups are those that can be converted under mild conditions into free amino thiol, carboxyl and hydroxy groups without the molecular framework being destroyed or other undesired side reactions taking place.

The above-mentioned reactions are carried out according to standard methods, in the presence or absence of diluent, preferably, such as are inert to the reagents and are solvents thereof, of catalysts, condensing or said other agents, respectively and/or inert atmospheres, at low temperatures, room temperature or elevated temperatures, preferably at or near the boiling point of the solvents used, and at atmospheric or super-atmospheric pressure. The preferred solvents, catalysts and reaction conditions are set forth in the appended illustrative Examples.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

### ABBREVIATIONS

| | |
|---|---|
| ATP: adenosine 5′-triphosphate | BINAP: racemic 2,2′- |
| | bis(diphenylphosphino)-1,1′- |
| | binaphthyl |
| BOC: tertiary butyl carboxy | br: broad |
| bs: broad singlet | d: doublet |
| DAST: (diethylamino)sulfur trifluoride | dd: doublet of doublets |
| DCM: dichloromethane | DIEA: diethylisopropylamine |
| DME: 1,4-dimethoxyethane | DMF: N,N-dimethylformamide |
| DMSO: dimethylsulfoxide | DPPA: diphenylphosphorylazide |
| DTT: dithiothreitol | EDTA: ethylenediamine tetraacetic acid |
| ESI: electrospray ionization | EtOAc: ethyl acetate |
| FCC: flash column chromatography | H: hour(s) |
| HATU: O-(7-azobenzotriazol-1-yl)-1,1,3,3- | HOBt: 1-hydroxy-7-azabenzotriazole |
| tetramethyluroniumhexafluorophosphate | |
| HPLC: high pressure liquid chromatography | LCMS: liquid chromatography and mass |
| | spectrometry |
| MeOD: methanol-d4 | MeOH: methanol |
| MS: mass spectrometry | m: multiplet |
| min: minutes | m/z: mass to charge ratio |
| n.d.: not determined | NMR: nuclear magnetic resonance |
| ppm: parts per million | Pr: propyl |
| PyBOP: benzotriazol-1-yloxy | rt: room temperature |
| Tripyrrolidinophosphoniumhexafluorophosphate | |
| s: singlet | t: triplet |
| TFA: trifluoroacetic acid | THF: tetrahydrofuran |
| TLC: thin layer chromatography | Tris-HCl: aminotris(hydroxymethyl) |
| | methane hydrochloride |

### Methods for Synthesizing Compounds of the Invention

The compounds of the invention can be synthesized using the methods described in the following schemes, examples, and by using art recognized techniques. All compounds described herein are included in the invention as compounds.

In Scheme 1, halide 1, which can be made from a 2,6-dihalopiperidine by Buchwald coupling or by direct displacement with a nitrogen nucleophile HNR⁴R⁵, can be further elaborated to bipydridyl 2 by Suzuki coupling with a suitable pyridine boronic acid, (e.g., 2-fluoropyridine-4-boronic acid or 2-chloropyrine-4-boronic acid) and a palladium catalyst, such as Pd(PPh₃)₄. Selective 5-bromination of pyridine 2 using Br₂ yields 3. The bromide 3 may converted to an aryl group by Suzuki coupling with an aryl boronic acid to give 4. Elaboration of the chloropyridines of 2 or 4 to the corresponding aminopyridine can be effected by Buchwald amination or direct displacement with R^{a′}R^{b′}NH. Treatment with a suitable acid, such as trifluoroacetic acid, yields targets 5. Substituents present in the R⁴, R⁵, R¹, and/or R^{b′} moieties of 5 can be manipulated further by methods known in the art.

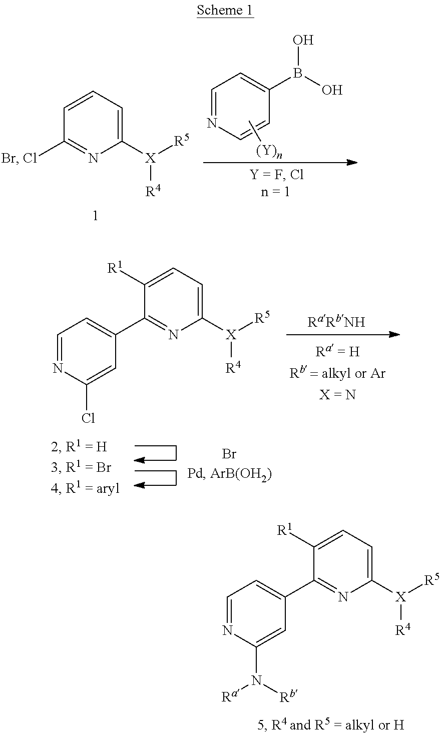

Analogs bearing a 4-substituent R² or R^{2′} on the core pyridine ring are generated from an appropriately substituted 2,6-dihalo-4-pyridine 6. Halopyridines 6, where the 4-substituent R² may be methylcarboxy, amido, tert-butylaminocarboxy, methane sulfonyl, or nitro, are generated from available 2,6-dihalo pyridines (e.g., 2,6-dichloro-4-carboxypyridine methyl ester; or 2,6-dibromo-4-nitropyridine) or 2,6-dihydroxy-4-substituted pyridines (e.g., citrazinic acid). According to Scheme 2, treatment of 6, where R² is an electron-withdrawing substituent, with a suitable nucleophile, such as a primary or secondary amine, in the presence of triethylamine in a suitable solvent, such as dioxane, with heating effects nucleophilic displacement of the halide to give amino pyridines 7. Suzuki coupling of 7 with a 2-halo-pyridine-4-boronic acids yields bipyridyls 8. Halopyridines 8 are converted to aminopyridines 9 by direct displacement of a fluoride with an amine or by Pd-catalyzed amination of the chloropyridine. Additional targets 9 can be generated by manipulation of R⁴, R⁵, and R¹ for example, where R⁴ or R⁵ contain BOC protecting groups that can be removed under acidic conditions (e.g., using TFA) or where R¹ contains an ester that can be converted to an amide under Weinreb's conditions (e.g., AlCl₃ and an appropriate amine). Alternatively, the R² moiety present in bipyridyls 8 may be converted to R^{2′} by methods known in the art to give products 10. For example, 8 (R²═CO₂Me) can be reduced with LiAlH₄ to 10 (R^{2′}═CH₂OH), which can be converted to the corresponding 11A (R^{2′}=CH₂OH). Alternatively, 8 (R²═CO₂Me) can be treated with ammonia to give 10 (R^{2′}═CONH₂) or an amine R^{a′}R^{b′}NH in the presence of AlCl₃ to yield 10 (R^{2′}═CONR^{a′}R^{b′}), which are converted to 11B (R^{2′}═CONH₂) and 11C (R^{2′}═CONR^{a′}R^{b′}), respectively. Halopyridines 10 are converted to aminopyridines 11 by direct displacement of a fluoride with an amine or by Pd-catalyzed amination of the chloropyridine. Additional targets 11 can be generated by manipulation of R⁴, R⁵, and R¹, for example, where R⁴ or R⁵ contain BOC protecting groups that can be removed under acidic conditions (e.g., using TFA). In the case of 11C (R^{2′}═CONR^{a′}R^{b′}, R^{b′}=tBu), acidic deprotection of BOC groups can occur with loss of the t-butyl group to yield targets 11B (R^{2′}═CONH₂). Substituents R²═CO₂Me of compounds 9 may also be conveniently transformed to heterocycles, for example by treatment of the ester with hydrazine and a trialkylorthoester to give 11D (R^{2′}=1,3,4-oxadiazolyl) or with hydrazine and CDI to give 11E (R^{2′}=5-oxo-4,5-dihydro-1,3,4-oxadiazolyl). Compound 9 (R²═CO₂Me) can be reacted with nucleophiles, for example methylmagnesium bromide, to yield 11F (R^{2′}═(CH₃)₂COH). Similarly, compounds 9 (R²═NO₂) can be reacted with nucleophiles, such as imidazole to yield compounds 11G (R^{2′}=1-imidazolyl); hydroxide to yield compounds 11H (R^{2′}═OH); alkoxide to yield compounds 11I (R^{2′}═OMe). Reduction of compound 9 (R²═NO₂) can be effected by ammonium formate in the presence of a palladium catalyst to afford 11J (R^{2′}═NH₂).

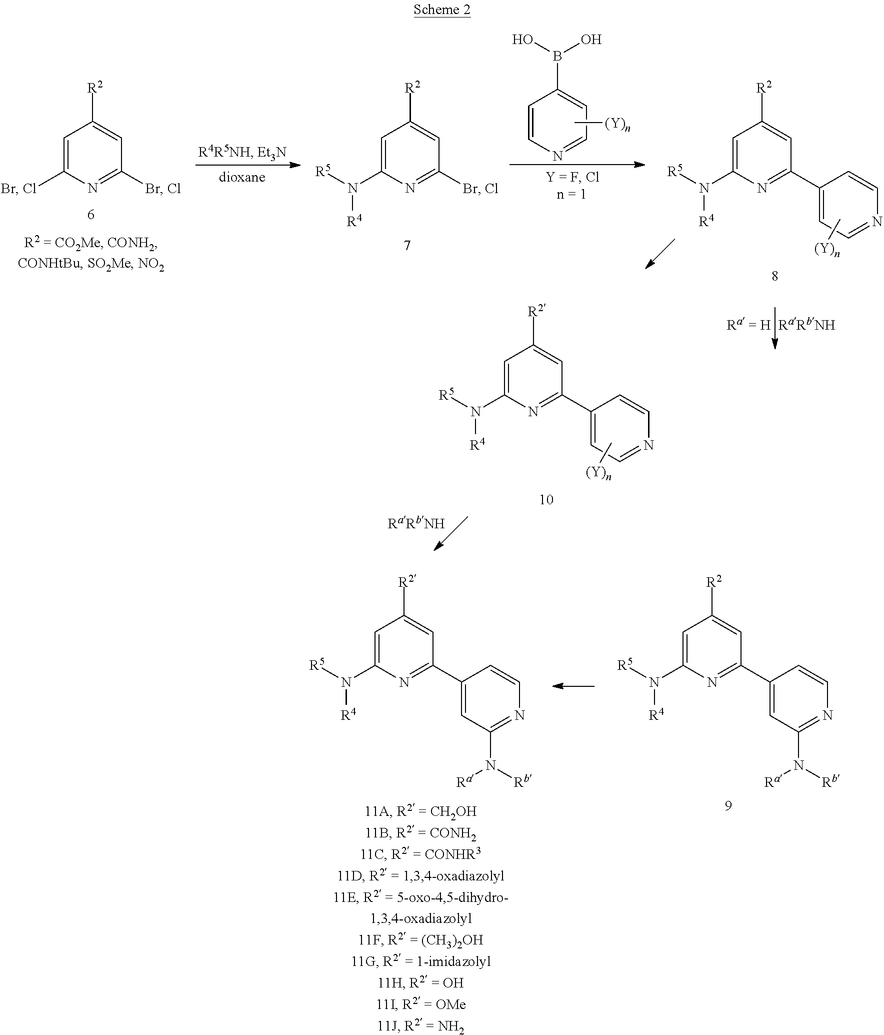

A representative product 11A described above (exemplified by R⁴ and R⁵ together comprising tert-butylcarboxypiperidine, and R^{b′}=cyclohexyl) can be used to generate additional targets by functional group transformations of the alcohol as described in Scheme 3. For example, treatment of alcohol 11A with CBr₄ and PPh₃ gives bromide 12. Bromide 12 can be displaced with a suitable nucleophile, such as NaCN, to yield nitrile 13 or NaN₃, to yield azide 14. Hydration of nitrile 13 affords amide 15. Azide 14 can be reduced to the corresponding primary amine 16 by LiAlH₄. Oxidation of 11A yields aldehyde 17. Reductive amination of aldehyde 17 produces amines 18.

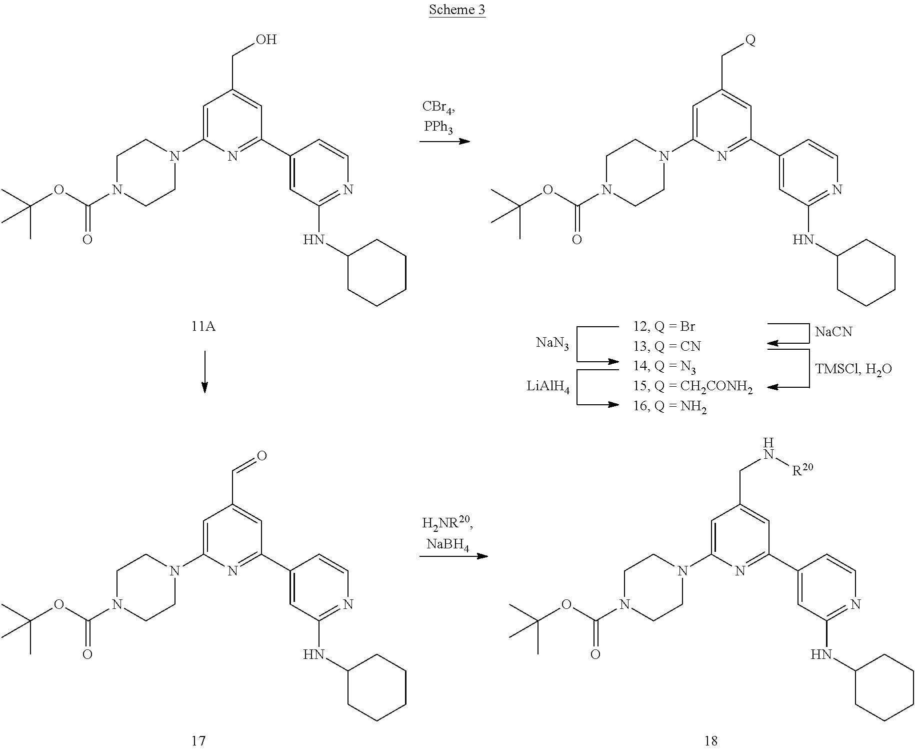

A representative product 11B described above (e.g, R⁴ and R⁵ together comprise tert-butylcarboxypiperidine, and R^{b′}=cyclohexyl) can be used to generate additional targets by functional group transformations of the amide as described in Scheme 4. For example, treatment of 11B with a suitable dehydrating agent, e.g., trifluoroacetic anhydride, produces nitrile 19. Treatment of 19 with NaN₃ yields tetrazole 20. Removal of the BOC group under acidic conditions from either 19 or 20 can be used to generate the corresponding piperidines.

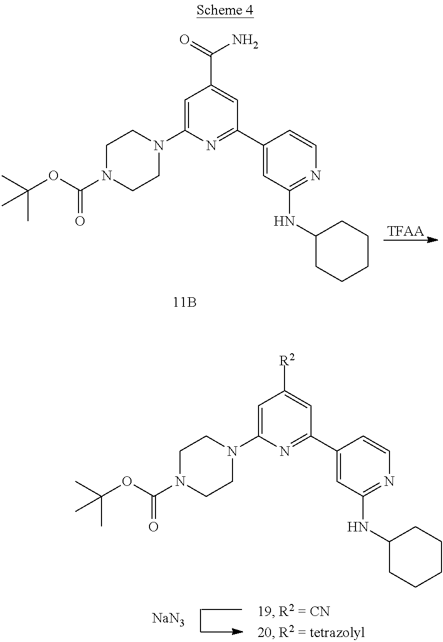

A representative product 11H described above (exemplified by R^{2′}═OH, R⁴ and R⁵ together comprise tert-butylcarboxypiperidine, and R^{b′}=cyclohexyl) can be used to generate additional targets by functional group transformations of the alcohol as described in Scheme 5. Bromination of pyridone 11H using POBr₃, followed by reprotection using BOC₂O gives 21. Alternatively, pyridone 11H can be reacted with a suitable triflating reagent to give triflate 22. Intermediates 21 or 22 can be coupled with a suitable arylmetal or arylmetalloid species in the presence of catalytic Pd and a suitable ligand to afford 4-aryl and 4-heteroaryl-substituted compounds 23.

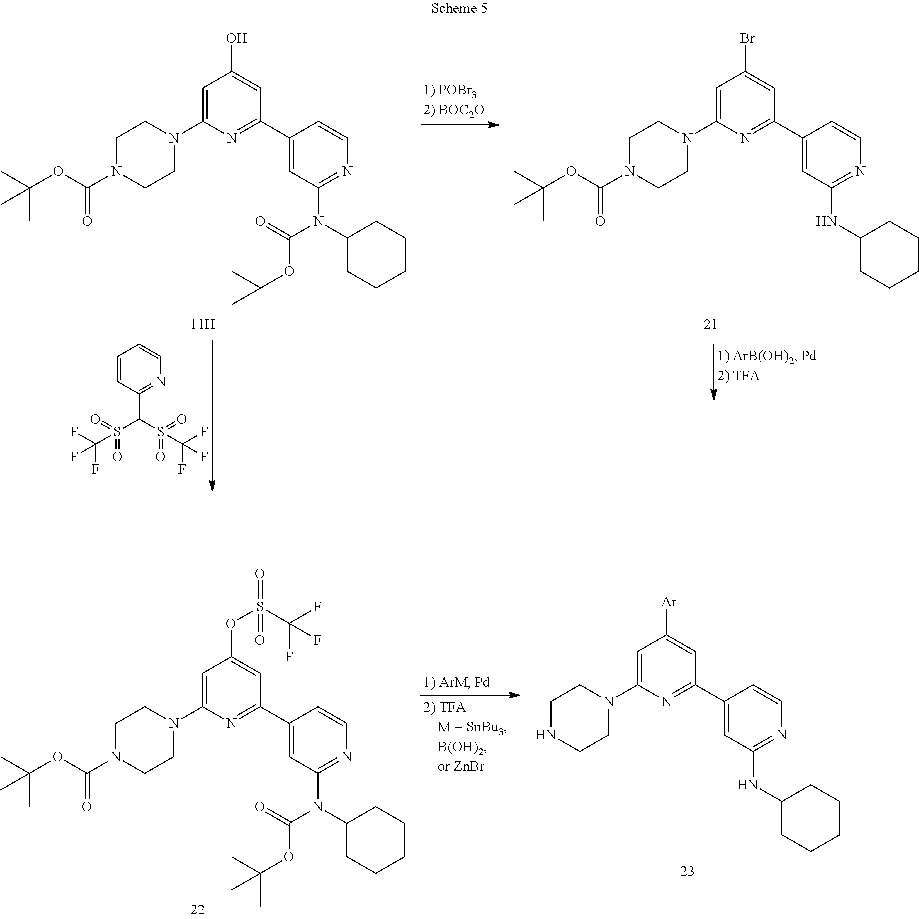

In Scheme 6, A representative product 11J described above (exemplified by R^{2′}═NH₂, R⁴ and R⁵ together comprise tert-butylcarboxypiperidine, and R^{b′}=cyclohexyl) can be used to generate additional targets by functional group transformations of the amine. For example, amine 11J can be captured by an electrophile, such as acetyl chloride or methane sulfonyl chloride to yield amide 24 and sulfonamide 25, respectively. Alternatively, amine 11J can be used to form tetrazole 26 or imidazole 27. Reductive amination of 11J produces 28. Amine 11J may also be reacted with an aryl boronic acid in the presence of a palladium catalyst to afford diarylamines 29.

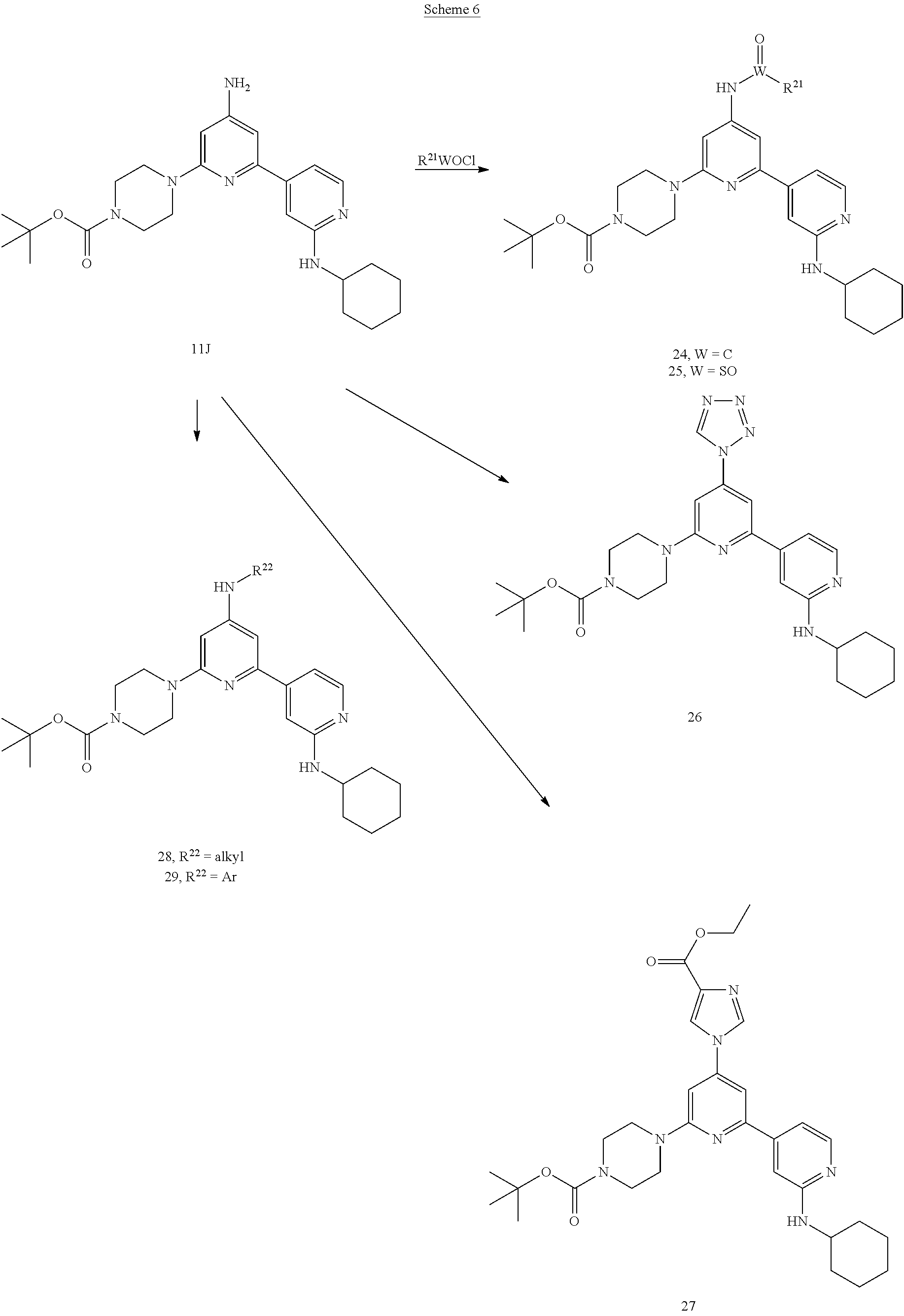

Stannane 30 of is generated as described in Scheme 7 by selective displacement of the fluoride of 2-fluoro-4-iodopyridine with a suitable nucleophile, such as cyclohexylamine, with heating. In a subsequent step, oxidative addition of a palladium catalyst, such as that generated from tetrakistriphenylphosphine palladium, to the 4-iodopyridine moiety, followed by coupling to hexamethylditin yields the desired pyridyl stannnane 30. Stille coupling of the halide 31 with 30 yields bipyridyl products 32. Bipyridyls 32 may be further elaborated to compounds 33 by Suzuki coupling with an arylboronic acid. The precursor 2,6-dichloro-4-difluoromethylpyridine needed to generate halide 31, R²═CHF₂, is generated by treatment of 2,6-dichloro-4-formylpyridine with DAST. Alternatively, halides 34 are generated by nucleophilic displacement of a halogen from an appropriately substituted 2,6-dihalo-4-pyridine 31. For example, treatment of 2,6-dichloro-4-trifluoromethylpyridine with a suitable nucleophile, such as a primary or secondary amine like BOCpiperazine, in the presence of triethylamine in a suitable solvent, such as dioxane, with heating effects nucleophilic displacement of the halide to give amino pyridines 34, where R²═CF₃. Compounds 34 can be utilized as Stille coupling partners with 30 to give products 35. The 4-substituent R² of 35 may be, for example, trifluoromethyl, difluoromethyl, or methylcarboxy. Substituents R¹, R², R⁴, and R⁵ can be further manipulated by standard methods known in the art, (e.g., by treatment of 35, where R²═CO₂Me, with ammonia to yield the carboxamide derivative. Likewise, amine substituents R¹, R⁴ and R⁵ of 35 may be manipulated by methods known in the art, for example where they contain BOC protecting groups that can be removed under acidic conditions (e.g., using TFA).

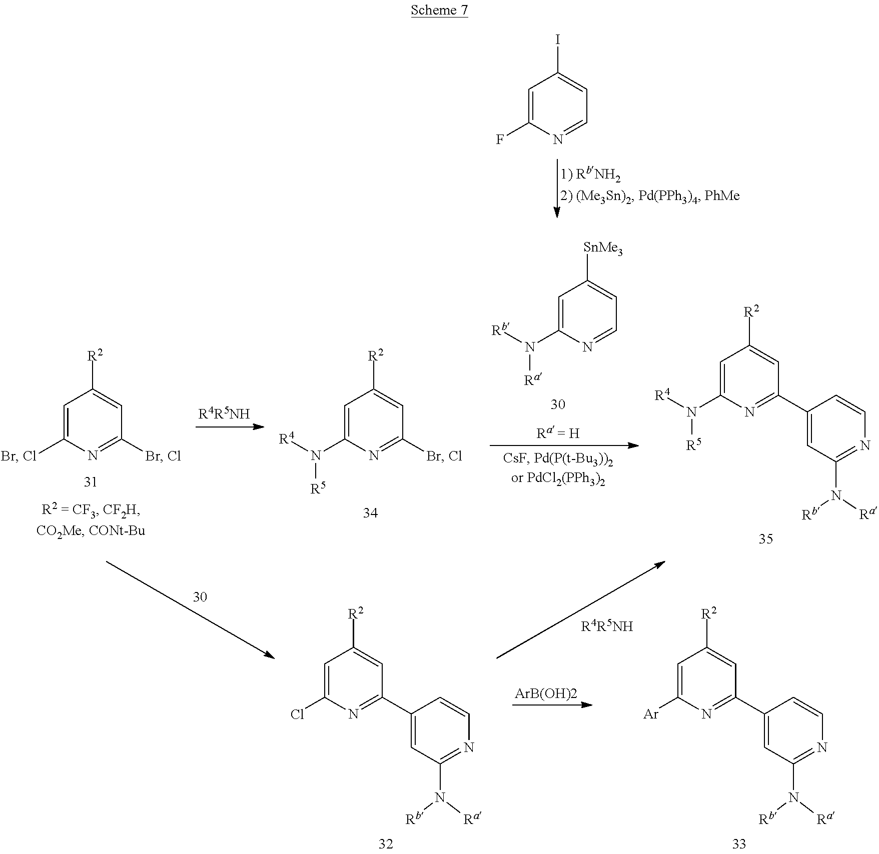

A representative analog 7, as described in Scheme 2 (exemplified by R²═CO₂Me and R⁴ and R⁵ together comprise tert-butylcarboxypiperidine and Y═Cl), undergoes smooth hydrolysis of the ester upon treatment with a suitable nucleophile, such as hydroxide as shown in Scheme 8. Treatment of the carboxylic acid 7 with DPPA and heat affords Curtius rearrangement product isocyanate intermediates that can be trapped with a suitable oxygen or nitrogen nucleophile, such as MeOH or aniline, to afford carbamate 36 and urea 37, respectively. Stille coupling of 36 or 37 is successfully achieved with pyridyl stannane 30 to afford bipyridyls 38 and 39, respectively.

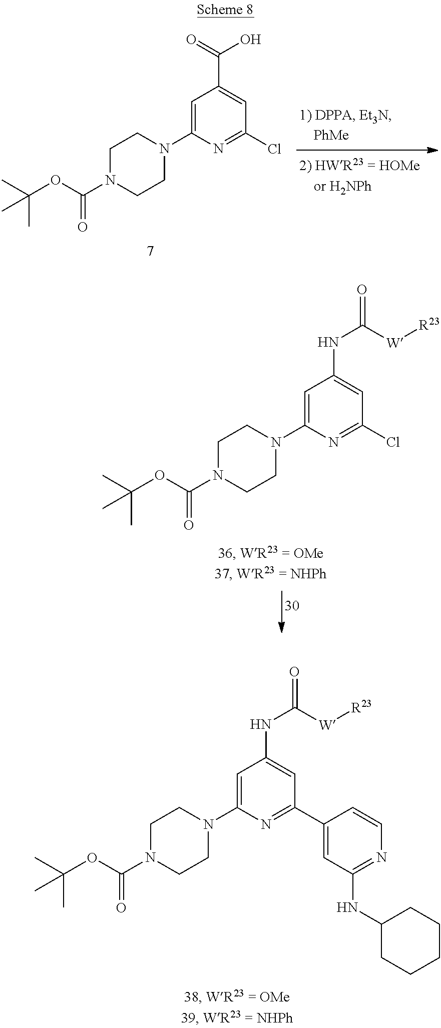

A representative analog 8, prepared according to Scheme 2, which is exemplified by the case where R²═CO₂Me and R⁴ and R⁵ together comprise tert-butylcarboxypiperidine can be selectively brominated to yield 37, as described in Scheme 9. Bromide 40 can be converted to products 41 by methods described above.

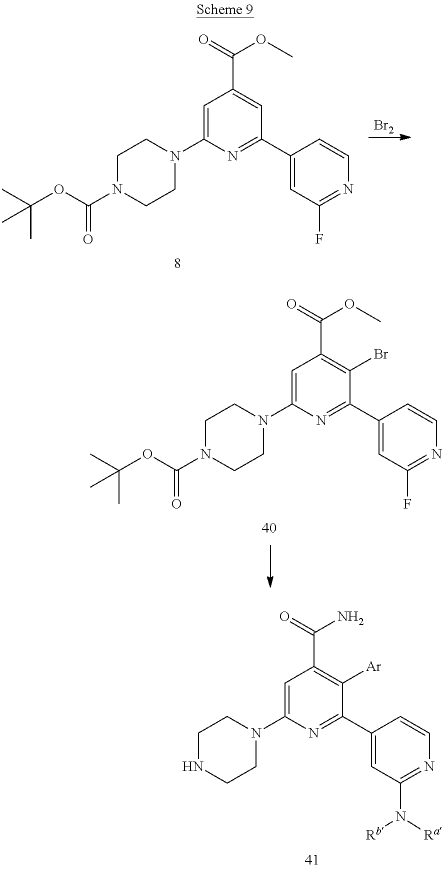

Ester 42 is prepared according to literature precedent. Displacement of chloride by BOCpiperazine yields a mixture of isomers 43 and 44, which are coupled to stannane 30 to afford products 45 and 46, respectively. Compounds 45 and 46 are separable by methods known in the art, such as HPLC purification. Compounds 45 and 46 may be converted to additional products by methods known in the art, such as removal of the BOC group under acidic conditions and conversion of the ester to an amide.

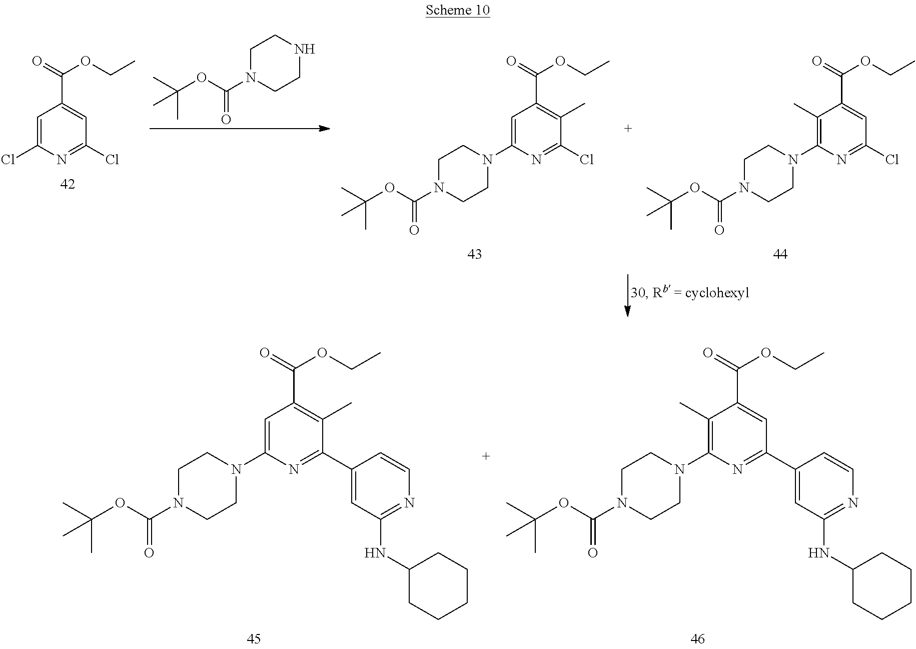

Scheme 11 illustrates that ester 42 may also be brominated under radical conditions, such as those produced when NBS is used with the radical initiator benzoyl peroxide. The resultant bromide can be displaced by ammonium hydroxide, which spontaneously forms lactam 47. Displacement of chloride 47 by BOCpiperazine yields isomers 48 and 49, which are separable by capitalizing on their different solubility properties. Compounds 48 and 49 can be coupled to stannane 30 to afford bipyridyls 50 and 51, respectively.

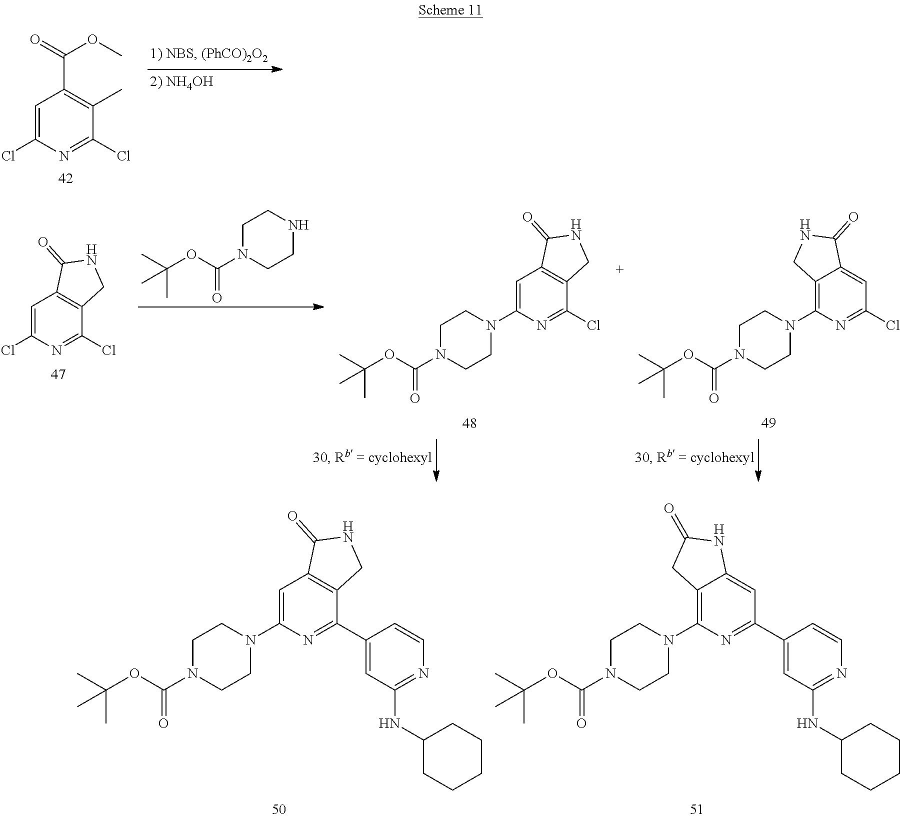

According to Scheme 12, 2,6-dichloronicotinic acid ethyl ester 52, which is prepared from the corresponding acid, reacts with BOCpiperazine to yield isomers 53 (minor) and 54 (major), which are separable by column chromatography. Chloride 53 can be coupled to a 2-halo-pyridine-4-boronic acids yielding the bipyridyl, which is further elaborated to aminopyridine 55 by direct displacement of a fluoride with cyclohexylamine. Targets 56 are produced from 55 by treatment with a source of ammonia with heating, followed by deprotection under acidic conditions. By analogy, Suzuki coupling of isomer 54, followed by conversion of the fluoropyridine to the cyclohexylaminopyridine yields 57. Ester 57 is converted to carboxamide 58 by heating with ammonia in methanol, which also produces ester 59 as a byproduct. Alternatively, ester 57 may be hydrolyzed under acidic conditions to the corresponding acid, and treated with an amine, such as isopropylamine, in the presence of a dehydrating agent, such as HATU to afford amides 60.

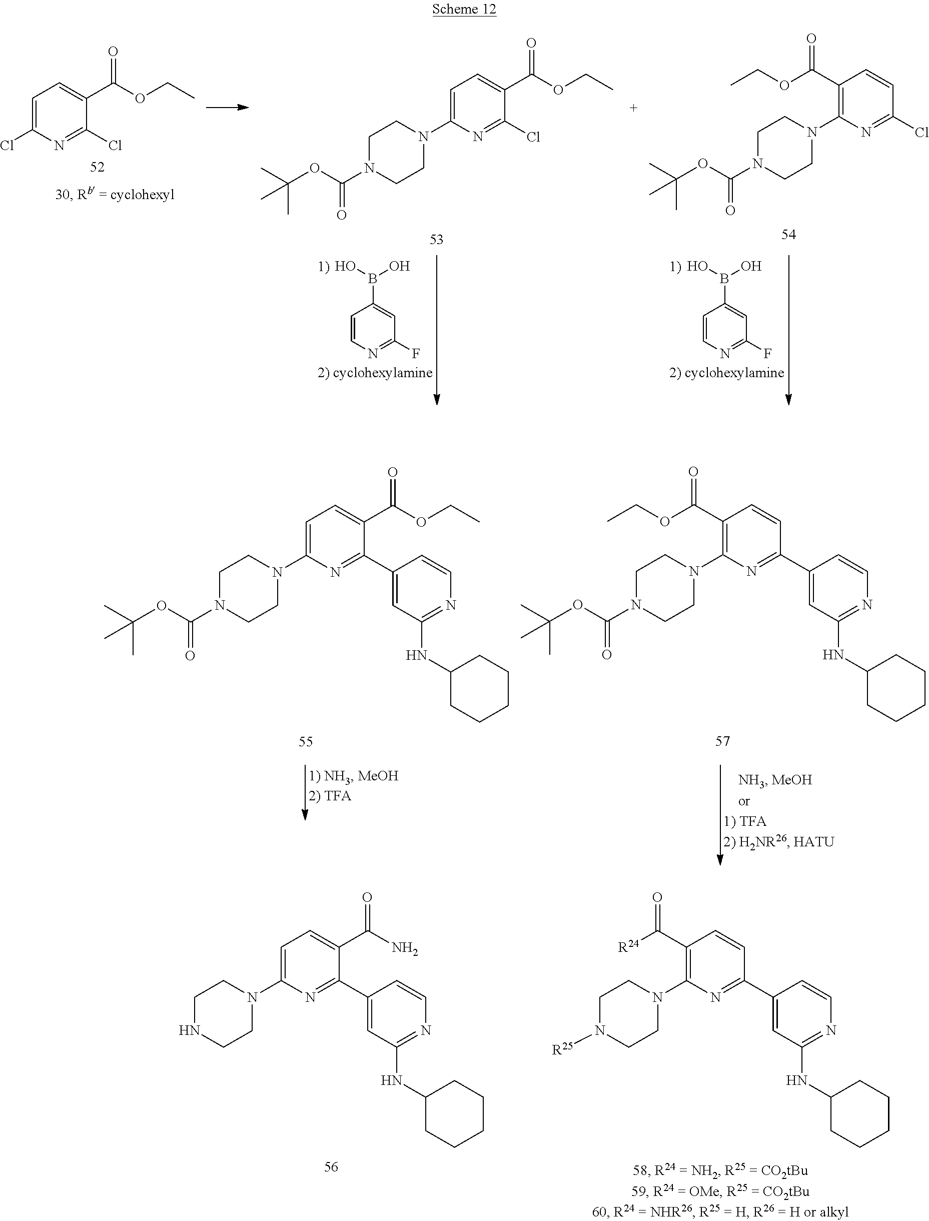

According to Scheme 13, Stille coupling of chloride 61 to stannane 30 (R^{b′}=cyclohexyl) affords bipyridyl 62. Saponification of the ester and coupling to amines H₂NR²⁶ afford targets 63.

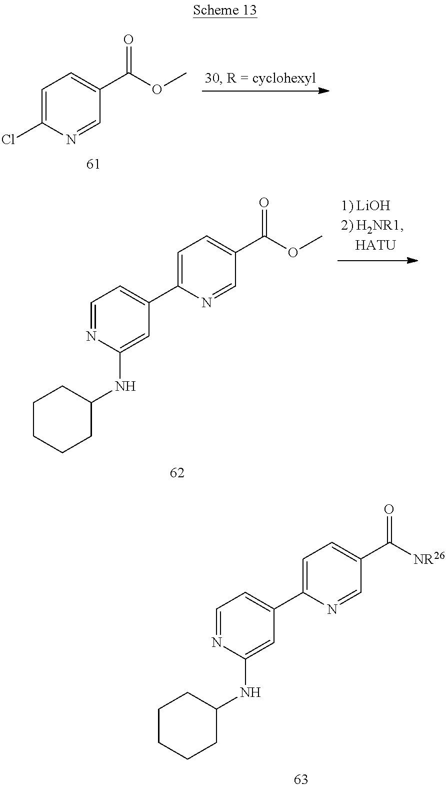

### Methods of the Invention

The invention pertains, at least in part, to methods for treating a subject for a disorder or disease, by administering to a subject a therapeutically effective amount of a compound of the invention, (e.g., a compound of Formula I or a compound otherwise described herein), such that said subject is treated for said disease or disorder.

The term “disorder” or “disease” includes any pathological condition, derangement, or abnormality of function of a part, organ, or system of an organism resulting from various causes, such as infection, genetic defect, or environmental stress, and characterized by an identifiable group of signs or symptoms; and any morbid physical or mental state. See Dorland's Illustrated Medical Dictionary, (W.B. Saunders Co. 27th ed. 1988).

In one embodiment, the disorder or disease is heart failure.

In another embodiment, the disorder or disease involves regulation of cell growth. The term “regulation of cell growth” includes mediation of cell size and cell division. Disorders involving the regulation of cell growth include cancers (e.g., breast cancer, colorectal cancer, genitourinary cancer, lung cancer, gastrointestinal cancer, epidermoid cancer, melanoma, ovarian cancer, pancreas cancer, neuroblastoma, head and/or neck cancer bladder cancer, renal cancer, brain cancer, myeloid cancer, or gastric cancer), tumors (e.g., a breast tumor; an epidermoid tumor, such as an epidermoid head and/or neck tumor or a mouth tumor; a lung tumor, for example a small cell or non-small cell lung tumor; a gastrointestinal tumor, for example, a colorectal tumor; or a genitourinary tumor, for example, a prostate tumor or a tumor that is refractory to treatment with other chemotherapeutics due to multidrug resistance), small cell lung carcinoma, large cell lung carcinoma, melanoma, prostate carcinoma, neoplasias, hyperplasias, fibrosis (e.g., pulmonary fibrosis or renal fibrosis), angiogenesis, psoriasis, atherosclerosis and smooth muscle cell proliferation in the blood vessels, such as stenosis or restenosis following angioplasty, tumors of blood and lymphatic system (e.g., Hodgkin's disease, Non-Hodgkin's lymphoma, Burkitt's lymphoma, AIDS-related lymphomas, malignant immunoproliferative diseases, multiple myeloma and malignant plasma cell neoplasms, lymphoid leukemia, acute or chronic myeloid leukemia, acute or chronic lymphocytic leukemia, monocytic leukemia, other leukemias of specified cell type, leukemia of unspecified cell type, other and unspecified malignant neoplasms of lymphoid, haematopoietic and related tissues, for example diffuse large cell lymphoma, T-cell lymphoma or cutaneous T-cell lymphoma) or a proliferative disease that is refractory to the treatment with other chemotherapeutics.

Where a tumor, a tumor disease, a carcinoma or a cancer are mentioned, metastasis in the original organ or tissue and/or in any other location are implied alternatively or in addition, whatever the location of the tumor and/or metastasis.

Examples of hyperproliferative skin disorders include psoriasis, atopic dermatitis, eczematous dermatitises, seborrhoeic dermatitis, pemphigus, and contact dermatitis (e.g., allergic contact dermatitis).

The invention also pertains to a method for treating autoimmune disorders or chronic inflammatory diseases. “Autoimmune disorders” include any of a group of disorders in which tissue injury is associated with humoral or cell-mediated responses to the body's own constituents. Such disorders may be systemic or organ-specific. Examples of autoimmune disorders or chronic inflammatory diseases include sarcoidosis, fibroid lung, idiopathic interstitial pneumonia, obstructive airways disease, including conditions such as asthma, intrinsic asthma, extrinsic asthma, dust asthma, particularly chronic or inveterate asthma (e.g., late asthma and airway hyperreponsiveness), bronchitis, including bronchial asthma, infantile asthma, rheumatoid arthritis, osteoarthritis, systemic lupus erythematosus, nephrotic syndrome lupus, Hashimoto's thyroiditis, multiple sclerosis, myasthenia gravis, type I diabetes mellitus and complications associated therewith, type II adult onset diabetes mellitus, uveitis, nephrotic syndrome, steroid dependent and steroid-resistant nephrosis, palmoplantar pustulosis, allergic encephalomyelitis, glomerulonephritis, psoriasis, psoriatic arthritis, atopic eczema (atopic dermatitis), allergic contact dermatitis, irritant contact dermatitis and further eczematous dermatitises, seborrheic dermatitis, lichen planus, pemphigus, bullous pemphigoid, epidermolysis bullosa, urticaria, angioedemas, vasculitides, erythemas, cutaneous eosinophilias, acne, alopecia areata, eosinophilic fasciitis, atherosclerosis, conjunctivitis, keratoconjunctivitis, keratitis, vernal conjunctivitis, uveitis associated with Behcet's disease, herpetic keratitis, conical cornea, dystorphia epithelialis corneae, keratoleukoma, ocular pemphigus, Mooren's ulcer, scleritis, Graves' ophthalmopathy, severe intraocular inflammation, inflammation of mucosa or blood vessels such as leukotriene B4-mediated diseases, gastric ulcers, vascular damage caused by ischemic diseases and thrombosis, cardiac hypertrophy, ischemic bowel disease, inflammatory bowel disease (e.g., Crohn's disease or ulcerative colitis), necrotizing enterocolitis, renal diseases including interstitial nephritis, Goodpasture's syndrome hemolytic uremic syndrome and diabetic nephropathy, nervous diseases selected from multiple myositis, Meniere's disease and radiculopathy, collagen disease including scleroderma, chronic autoimmune liver diseases including autoimmune hepatitis, primary biliary cirrhosis and sclerosing cholangitis), partial liver resection, acute liver necrosis (e.g., necrosis caused by toxins, viral hepatitis, shock or anoxia), cirrhosis, fulminant hepatitis, pustular psoriasis, Behcet's disease, active chronic hepatitis, Evans syndrome, pollinosis, idiopathic hypoparathyroidism, autoimmune atrophic gastritis, lupoid hepatitis, tubulointerstitial nephritis, membranous nephritis, rheumatic fever, acute disseminated encephalomyelitis, Addison's disease, ankylosing spondylitis, antiphospholipid antibody syndrome, aplastic anemia, autoimmune oophoritis, Celiac disease, hestational pemphigoid, Graves' disease, Guillain-Barré syndrome, Hashimoto's disease, idiopathic thrombocytopenic purpura, Kawasaki's Disease, mixed connective tissue disease, opsoclonus myoclonus syndrome, optic neuritis, Ord's thyroiditis, pemphigus, pernicious anaemia, polyarthritis in dogs, Reiter's syndrome, Sjögren's syndrome, Takayasu's arteritis, temporal arteritis, warm autoimmune hemolytic anemia, and Wegener's granulomatosis.

In yet another embodiment, the disorder or disease is mediated by T lymphocytes, B lymphocytes, mast cells, eosinophils or cardiomyocytes e.g., acute or chronic rejection of organ or tissue allo- or xenografts, graft-versus-host disease, host-versus-graft disease, atherosclerosis, cerebral infarction, vascular occlusion due to vascular injury such as angioplasty, restenosis, fibrosis (especially pulmonary, but also other types of fibrosis, such as renal fibrosis), angiogenesis, hypertension, heart failure, chronic obstructive pulmonary disease, CNS disease such as Alzheimer disease or amyotrophic lateral sclerosis, cancer, infectious disease such as AIDS, septic shock or adult respiratory distress syndrome, ischemia/reperfusion injury e.g., myocardial infarction, stroke, gut ischemia, renal failure or hermorrhage shock, or traumatic shock.

The invention also pertains, at least in part, to methods of modulating (e.g., inhibiting) PKD activity in a subject, by administering to a subject a therapeutically effective amount of a compound of the invention, (e.g., a compound of Formula I or a compound otherwise described herein), such that PKD activity is modulated.

Another embodiment of the present invention includes methods for treating a PKD associated state in a subject, by administering to a subject a therapeutically effective amount of a compound of the invention, (e.g., a compound of Formula I or a compound otherwise described herein), such that said subject is treated.

In certain embodiments, the compounds of the present invention may be used as modulators (e.g., PKD modulators or PKD inhibitors).

The term “PKD associated state” refers to a state, disease, or disorder which can be treated by the modulation, (e.g., inhibition) of PKD. PKD is a family of serine/threonine protein kinases (e.g., PKD1, 2 and 3) that is now classified as a subfamily of the Ca2+/calmodulin-dependent kinase (CaMK) superfamily. Reports have demonstrated the biological functions of PKD. See Wang Q J, TRENDS Pharm. Sci., 27(6): 3170323 (2006). For example, it has been found that activation of PKD regulates fission of transport carriers from the Golgi to the plasma membrane. See Liljedahl, M. et al., Cell, 104: 409-420 (2001). PKD has a major role in cell motility, invasion, and adhesion. PKD has also been demonstrated to have pro-proliferative effect in many cellular systems, as well as promotes antiapoptotic responses in tumor cells. See Prigozhina, N L et al., Curr. Biol., 14: 88-98 (2004), Rozengurt E. et al., JBC, 280(14): 13205-13208 (2005). PKD has also been found to regulate agonist-dependent cardiac hypertrophy through the nuclear export of class II histone deacetylase (HDAC5). See Vega, R B et al., Mol. Cell. Biol., 24: 8374-8385 (2004). PKD is also involved in oxidative stress response by activating the transcription factor Nf-kB to protect the cell from oxidative-stress-induced cell death. See Storz, P. and Toker, A., EMBO J., 22: 109-120 (2003). Sjoblom, T. et al. linked PKD to breast and colorectal cancers. See Sjoblom, T. et al., Science, 314:268-274 (2006). PKD has been found to regulate gene expression related to immune response and function of skin. See Matthews, S A et al., Mol. Cell. Biol., 26(4): 1569-1577 (2006), Irie, A. et al., Int. Immunology, 18(12): 1737-1747 (2006), Bollag, W B et al., Drug News Perspect, 17(2): 117 (2004), etc. Therefore, examples of PKD associated disorders include heart failure, colorectal cancer, regulation of cell growth, autoimmune disorders, and hyperproliferative skin disorders, etc.

In one embodiment, PKD associated states are characterized by an abnormal activity of PKD and/or abnormal expression of PKD. The term “abnormal” includes an activity or feature which differs from a normal activity or feature. The term “abnormal activity” includes an activity which differs from the activity of the wild-type or native gene or protein, or which differs from the activity of the gene or protein in a healthy subject. The abnormal activity can be stronger or weaker than the normal activity.

In one embodiment, the “abnormal activity” includes the abnormal (either over- or under-) production of mRNA transcribed from a gene. In another embodiment, the “abnormal activity” includes the abnormal (either over- or under-) production of polypeptide from a gene. In another embodiment, the abnormal activity refers to a level of a mRNA or polypeptide that is different from a normal level of said mRNA or polypeptide by about 15%, about 25%, about 35%, about 50%, about 65%, about 85%, about 100% or greater. Preferably, the abnormal level of the mRNA or polypeptide can be either higher or lower than the normal level of said mRNA or polypeptide. Yet in another embodiment, the abnormal activity refers to functional activity of a protein that is different from a normal activity of the wild-type protein. Abnormal activity can be stronger or weaker than the normal activity. Abnormal activity can be due to the mutations in the corresponding gene, and the mutations can be in the coding region of the gene or non-coding regions such as transcriptional promoter regions. The mutations can be substitutions, deletions, insertions.

The compounds of the present invention, as PKD modulating compounds, are useful for treatment of a disorder or disease mediated by PKD or responsive to inhibition of PKD. In particular, the compounds of the present invention are useful for treatment of a PKD associated state including heart failure, colorectal cancer, regulation of cell growth, autoimmune disorders, and hyperproliferative skin disorders, etc.

The term “PKD modulating compound” includes compounds, which modulate, e.g., inhibit, promote or otherwise alter the activity of PKD. PKD modulating compounds include PKD agonists, inverse agonists, and antagonists. This term includes, but is not limited to, compounds of Formula I and compounds listed in the examples.

The term “PKD inhibiting compound” includes compounds which reduce the activity of PKD, e.g., the ability of PKD to phosphorylate substrate (e.g., HDAC), in vivo or in vitro. In one embodiment, the PKD inhibiting compounds are PKD antagonists or inverse agonists. In another embodiment, the PKD inhibiting compounds are HDAC phosphorylation inhibiting compounds.

The term “subject” includes animals (e.g., mammals). A subject also refers to for example, primates (e.g., humans), cows, sheep, goats, horses, dogs, cats, rabbits, rats, mice, fish, birds, etc.

The term “a therapeutically effective amount” of a compound of the present invention refers to an amount of the compound of the present invention that will elicit the biological or medical response of a subject, for example, reduction or inhibition of an enzyme or a protein activity, or ameliorate symptoms, alleviate conditions, slow or delay disease progression, or prevent a disease, etc. In one non-limiting embodiment, the term “a therapeutically effective amount” refers to the amount of the compound of the present invention that, when administered to a subject, is effective to (1) at least partially alleviate, inhibit, prevent and/or ameliorate a condition, or a disorder or a disease (i) mediated by PKD, or (ii) associated with PKD activity, or (iii) characterized by abnormal activity of PKD; or (2) reduce or inhibit the activity of PKD; or (3) reduce or inhibit the expression of PKD. In another non-limiting embodiment, the term “a therapeutically effective amount” refers to the amount of the compound of the present invention that, when administered to a cell, or a tissue, or a non-cellular biological material, or a medium, is effective to at least partially reduce or inhibit the activity of PKD; or at least partially reduce or inhibit the expression of PKD.

The effective amount can vary depending on such factors as the size and weight of the subject, the type of illness, or the particular organic compound. For example, the choice of the organic compound can affect what constitutes an “effective amount.” One of ordinary skill in the art would be able to study the aforementioned factors and make the determination regarding the effective amount of the organic compound without undue experimentation.

The term “treating” or “treatment” of any disease or disorder includes curing as well as ameliorating at least one symptom of the state, disease, or disorder (e.g., the PKD associated state). The term may also include alleviating or ameliorating at least one physical parameter including those which may not be discernible by the patient; or modulating the disease or disorder, either physically, (e.g., stabilization of a discernible symptom), physiologically, (e.g., stabilization of a physical parameter), or both. The terms may also include preventing or delaying the onset or development or progression of the disease or disorder.

A further embodiment includes methods for treating a PKD associated disorder or disease in a subject by administering to a subject an effective amount of a compound of the invention (e.g., a compound of Formula I or a compound otherwise described herein) in combination with a second agent, such that the subject is treated for said PKD associated disorder.

In one embodiment the disorder or disease includes but is not limited to heart failure, colorectal cancer, regulation of cell growth, autoimmune disorders, or hyperproliferative skin disorders.

The term “in combination with” a second agent or treatment includes co-administration of the compound of the invention (e.g., a compound of Formula I or a compound otherwise described herein) with the second agent or treatment, administration of the compound of the invention first, followed by the second agent or treatment and administration of the second agent or treatment first, followed by the compound of the invention.

The term “second agent” includes any agent which is known in the art to treat, prevent, or reduce the symptoms of a disease or disorder described herein, e.g., PKD associated disorder, such as, for example, heart failure, colorectal cancer, regulation of cell growth, autoimmune disorders, and hyperproliferative skin disorders, etc. Furthermore, the second agent may be any agent of benefit to the patient when administered in combination with the administration of compound of the invention. Examples of second agents include chemotherapeutic agents, radiation therapy, and cardiovascular protective agents, etc. as described below.

The term “chemotherapeutic agent” includes chemical reagents which inhibit the growth of proliferating cells or tissues wherein the growth of such cells or tissues is undesirable or otherwise treat at least one resulting symptom of such a growth. Chemotherapeutic agents are well known in the art (see e.g., Gilman A. G., et al., The Pharmacological Basis of Therapeutics, 8^{th} Ed., Sec 12:1202-1263 (1990)), and are typically used to treat neoplastic diseases. Examples of chemotherapeutic agents include: bleomycin, docetaxel (Taxotere), doxorubicin, edatrexate, etoposide, finasteride (Proscar), flutamide (Eulexin), gemcitabine (Gemzar), goserelin acetate (Zoladex), granisetron (Kytril), irinotecan (Campto/Camptosar), ondansetron (Zofran), paclitaxel (Taxol), pegaspargase (Oncaspar), pilocarpine hydrochloride (Salagen), porfimer sodium (Photofrin), interleukin-2 (Proleukin), rituximab (Rituxan), topotecan (Hycamtin), trastuzumab (Herceptin), tretinoin (Retin-A), Triapine, vincristine, and vinorelbine tartrate (Navelbine).

Other examples of chemotherapeutic agents include alkylating drugs such as nitrogen mustards (e.g., Mechlorethamine (HN₂), cyclophosphamide, Ifosfamide, Melphalan (L-sarcolysin), Chlorambucil, etc.); ethylenimines, methylmelamines (e.g., Hexamethylmelamine, Thiotepa, etc.); alkyl sulfonates (e.g., Busulfan, etc.), nitrosoureas (e.g., Carmustine (BCNU), Lomustine (CCNU), Semustine (methyl-CCNU), Streptozocin (streptozotocin), etc.), triazenes (e.g., Decarbazine (DTIC; dimethyltriazenoimi-dazolecarboxamide)), alkylators (e.g., cis-diamminedichloroplatinum II (CDDP)), etc.

Other examples of chemotherapeutic agents include antimetabolites such as folic acid analogs (e.g., Methotrexate (amethopterin)); pyrimidine analogs (e.g., fluorouracil (5-fluorouracil; 5-FU); floxuridine (fluorode-oxyuridine); Fudr; Cytarabine (cyosine arabinoside), etc.); purine analogs (e.g., Mercaptopurine (6-mercaptopurine; 6-MP); Thioguanine (6-thioguanine; TG); and Pentostatin (2′-deoxycoformycin)), etc.

Other examples of chemotherapeutic agents also include vinca alkaloids (e.g., Vinblastin (VLB) and Vincristine); topoisomerase inhibitors (e.g., Etoposide, Teniposide, Camptothecin, Topotecan, 9-amino-campotothecin CPT-11, etc.); antibiotics (e.g., Dactinomycin (actinomycin D), adriamycin, daunorubicin, doxorubicin, bleomycin, plicamycin (mithramycin), mitomycin (mitomycin C), Taxol, Taxotere, etc.); enzymes (e.g., L-Asparaginase); and biological response modifiers (e.g., interferon-; interleukin 2, etc.). Other chemotherapeutic agents include cis-diaminedichloroplatinum II (CDDP); Carboplatin; Anthracendione (e.g., Mitoxantrone); Hydroxyurea; Procarbazine (N-methylhydrazine); and adrenocortical suppressants (e.g., Mitotane, aminoglutethimide, etc.).

Other chemotherapeutic agents include adrenocorticosteroids (e.g., Prednisone); progestins (e.g., Hydroxyprogesterone caproate, Medroxyprogesterone acetate, Megestrol acetate, etc.); estrogens (e.g., diethylstilbestrol; ethenyl estradiol, etc.); antiestrogens (e.g. Tamoxifen, etc.); androgens (e.g., testosterone propionate, Fluoxymesterone, etc.); antiandrogens (e.g., Flutamide); and gonadotropin-releasing hormone analogs (e.g., Leuprolide).

The term “radiation therapy” includes the application of a genetically and somatically safe level of x-rays, both localized and non-localized, to a subject to inhibit, reduce, or prevent symptoms or conditions associated with cancer or other undesirable cell growth. The term “x-rays” includes clinically acceptable radioactive elements and isotopes thereof, as well as the radioactive emissions therefrom. Examples of the types of emissions include alpha rays, beta rays including hard betas, high energy electrons, and gamma rays. Radiation therapy is well known in the art (see e.g., Fishbach, F., Laboratory Diagnostic Tests, 3^{rd} Ed., Ch. 10: 581-644 (1988)), and is typically used to treat neoplastic diseases.

The term “cardiovascular protective agent” includes HMG-Co-A reductase inhibitors, angiotensin II receptor antagonists, angiotensin converting enzyme (ACE) Inhibitors, calcium channel blockers (CCB), dual angiotensin converting enzyme/neutral endopeptidase (ACE/NEP) inhibitors, endothelin antagonists, renin inhibitors, diuretics, ApoA-I mimics, anti-diabetic agents, obesity-reducing agents, aldosterone receptor blockers, endothelin receptor blockers, and CETP inhibitors.

The term “HMG-Co-A reductase inhibitor” (also called beta-hydroxy-beta-methylglutaryl-co-enzyme-A reductase inhibitors) includes active agents that may be used to lower the lipid levels including cholesterol in blood. Examples include atorvastatin, cerivastatin, compactin, dalvastatin, dihydrocompactin, fluindostatin, fluvastatin, lovastatin, pitavastatin, mevastatin, pravastatin, rivastatin, simvastatin, and velostatin, or, pharmaceutically acceptable salts thereof.

The term “ACE-inhibitor” (also called angiotensin converting enzyme inhibitors) includes molecules that interrupt the enzymatic degradation of angiotensin Ito angiotensin II. Such compounds may be used for the regulation of blood pressure and for the treatment of congestive heart failure. Examples include alacepril, benazepril, benazeprilat, captopril, ceronapril, cilazapril, delapril, enalapril, enaprilat, fosinopril, imidapril, lisinopril, moveltopril, perindopril, quinapril, ramipril, spirapril, temocapril, and trandolapril, or, pharmaceutically acceptable salt thereof.

The term “calcium channel blocker (CCB)” includes dihydropyridines (DHPs) and non-DHPs (e.g., diltiazem-type and verapamil-type CCBs). Examples include amlodipine, felodipine, ryosidine, isradipine, lacidipine, nicardipine, nifedipine, niguldipine, niludipine, nimodipine, nisoldipine, nitrendipine, and nivaldipine, and is preferably a non-DHP representative selected from the group consisting of flunarizine, prenylamine, diltiazem, fendiline, gallopamil, mibefradil, anipamil, tiapamil and verapamil, or, pharmaceutically acceptable salts thereof. CCBs may be used as anti-hypertensive, anti-angina pectoris, or anti-arrhythmic drugs.

The term “dual angiotensin converting enzyme/neutral endopeptidase (ACE/NEP) inhibitor” includes omapatrilate (cf. EP 629627), fasidotril or fasidotrilate, or pharmaceutically acceptable salts thereof.

The term “endothelin antagonist” includes bosentan (cf. EP 526708 A), tezosentan (cf. WO 96/19459), or, pharmaceutically acceptable salts thereof.

The term “renin inhibitor” includes ditekiren (chemical name: [1S-[1R*,2R*,4R*(1R*,2R*)]]-1-[(1,1-dimethylethoxy)carbonyl]-L-proly I-L-phenylalanyl-N-[2-hydroxy-5-methyl-1-(2-methylpropyl)-4-[[[2-methyl-1-[[(2-pyridinylmrthyl)amino]carbonyl]butyl]amino]carbonyl]hexyl]-N-alfa-methyl-L-histidinamide); terlakiren (chemical name: [R—(R*,S*)]-N-(4-morpholinylcarbonyl)-L-phenylalanyl-N-[1-(cyclohexylmethyl)-2-hydroxy-3-(1-methylethoxy)-3-oxopropyl]-S-methyl-L-cysteineamide); and zankiren (chemical name: [1S-[1R*[R*(R*)],2S*,3R*]]—N-[1-(cyclohexylmethyl)-2,3-dihydroxy-5-methylhexyl]alfa-[[2-[[(4-methyl-1-piperazinyl)sulfonyl]methyl]-1-oxo-3-phenylpropyl]amino]-4-thiazolepropanamide), or, hydrochloride salts thereof, or, SPP630, SPP635 and SPP800 as developed by Speedel, or RO 66-1132 and RO 66-1168 of Formula (A) and (B):

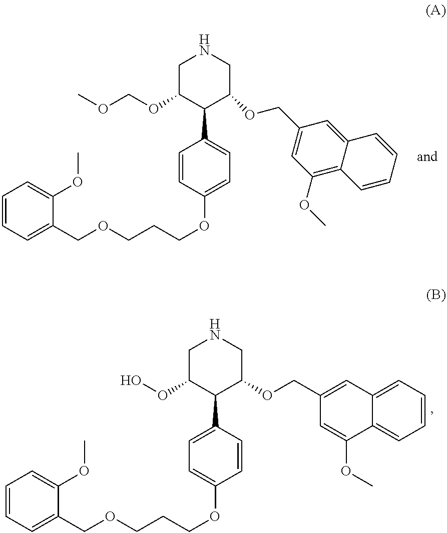

or, pharmaceutically acceptable salts thereof.

The term “diuretic” includes thiazide derivatives (e.g., chlorothiazide, hydrochlorothiazide, methylclothiazide, and chlorothalidon).

The term “ApoA-I mimic” includes D4F peptides (e.g., formula D-W-F-K-A-F-Y-D-K-V-A-E-K-F-K-E-A-F)

The term “anti-diabetic agent” includes insulin secretion enhancers that promote the secretion of insulin from pancreatic β-cells. Examples include biguanide derivatives (e.g., metformin), sulfonylureas (SU) (e.g., tolbutamide, chlorpropamide, tolazamide, acetohexamide, 4-chloro-N-[(1-pyrrolidinylamino)carbonyl]-benzensulfonamide (glycopyramide), glibenclamide (glyburide), gliclazide, 1-butyl-3-metanilylurea, carbutamide, glibonuride, glipizide, gliquidone, glisoxepid, glybuthiazole, glibuzole, glyhexamide, glymidine, glypinamide, phenbutamide, and tolylcyclamide), or pharmaceutically acceptable salts thereof. Further examples include phenylalanine derivatives (e.g., nateglinide [N-(trans-4-isopropylcyclohexylcarbonyl)-D-phenylalanine] (cf. EP 196222 and EP 526171) of the formula

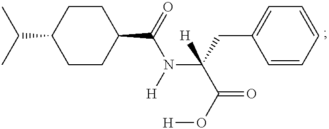

repaglinide [(S)-2-ethoxy-4-{2-[[3-methyl-1-[2-(1-piperidinyl)phenyl]butyl]amino]-2-oxoethyl}benzoic acid] (cf. EP 589874, EP 147850 A2, in particular Example 11 on page 61, and EP 207331 A1); calcium (2S)-2-benzyl-3-(cis-hexahydro-2-isoindolinlycarbonyl)-propionate dihydrate (e.g., mitiglinide (cf. EP 507534)); and glimepiride (cf. EP 31058). Further examples include DPP-IV inhibitors, GLP-1 and GLP-1 agonists.

DPP-IV is responsible for inactivating GLP-1. More particularly, DPP-IV generates a GLP-1 receptor antagonist and thereby shortens the physiological response to GLP-1. GLP-1 is a major stimulator of pancreatic insulin secretion and has direct beneficial effects on glucose disposal.

The DPP-IV inhibitor can be peptidic or, preferably, non-peptidic. DPP-IV inhibitors are in each case generically and specifically disclosed e.g. in WO 98/19998, DE 196 16 486 A1, WO 00/34241 and WO 95/15309, in each case in particular in the compound claims and the final products of the working examples, the subject-matter of the final products, the pharmaceutical preparations and the claims are hereby incorporated into the present application by reference to these publications.

GLP-1 is an insulinotropic protein which is described, e.g., by W. E. Schmidt et al. in Diabetologia, 28, 1985, 704-707 and in U.S. Pat. No. 5,705,483.

The term “GLP-1 agonists” includes variants and analogs of GLP-1(7-36)NH₂ which are disclosed in particular in U.S. Pat. No. 5,120,712, U.S. Pat. No. 5,118,666, U.S. Pat. No. 5,512,549, WO 91/11457 and by C. Orskov et al in J. Biol. Chem. 264 (1989) 12826. Further examples include GLP-1(7-37), in which compound the carboxy-terminal amide functionality of Arg³⁶ is displaced with Gly at the 37^{th} position of the GLP-1(7-36)NH₂ molecule and variants and analogs thereof including GLN⁹-GLP-1(7-37), D-GLN⁹-GLP-1(7-37), acetyl LYS⁹-GLP-1(7-37), LYS¹⁸-GLP-1(7-37) and, in particular, GLP-1(7-37)OH, VAL⁸-GLP-1(7-37), GLY⁸-GLP-1(7-37), THR⁸-GLP-1(7-37), MET⁸-GLP-1(7-37) and 4-imidazopropionyl-GLP-1. Special preference is also given to the GLP agonist analog exendin-4, described by Greig et al. in Diabetologia 1999, 42, 45-50.

Also included in the definition “anti-diabetic agent” are insulin sensitivity enhancers which restore impaired insulin receptor function to reduce insulin resistance and consequently enhance the insulin sensitivity. Examples include hypoglycemic thiazolidinedione derivatives (e.g., glitazone, (S)-((3,4-dihydro-2-(phenyl-methyl)-2H-1-benzopyran-6-yl)methyl-thiazolidine-2,4-dione (englitazone), 5-{[4-(3-(5-methyl-2-phenyl-4-oxazolyl)-1-oxopropyl)-phenyl]methyl}-thiazolidine-2,4-dione (darglitazone), 5-{[4-(1-methyl-cyclohexyl)methoxy)phenyl]methyl}-thiazolidine-2,4-dione (ciglitazone), 5-{[4-(2-(1-indolyl)ethoxy)phenyl]methyl}-thiazolidine-2,4-dione (DRF2189), 5-{4-[2-(5-methyl-2-phenyl-4-oxazolyl)-ethoxy(]benzyl}-thiazolidine-2,4-dione (BM-13.1246), 5-(2-naphthylsulfonyl)-thiazolidine-2,4-dione (AY-31637), bis{4-[(2,4-dioxo-5-thiazolidinyl)methyl]phenyl}methane (YM268), 5-{4-[2-(5-methyl-2-phenyl-4-oxazolyl)-2-hydroxyethoxy]benzyl}-thiazolidine-2,4-dione (AD-5075), 5-[4-(1-phenyl-1-cyclopropanecarbonylamino)-benzyl]-thiazolidine-2,4-dione (DN-108) 5-{[4-(2-(2,3-dihydroindol-1-yl)ethoxy)phenyl]methyl}-thiazolidine-2,4-dione, 5-[3-(4-chloro-phenyl])-2-propynyl]-5-phenylsulfonyl)thiazolidine-2,4-dione, 5-[3-(4-chlorophenyl])-2-propynyl]-5-(4-fluorophenyl-sulfonyl)thiazolidine-2,4-dione, 5-{[4-(2-(methyl-2-pyridinyl-amino)-ethoxy)phenyly]methyl}-thiazolidine-2,4-dione (rosiglitazone), 5-{[4-(2-(5-ethyl-2-pyridyl)ethoxy)phenyl]-methyl}thiazolidine-2,4-dione (pioglitazone), 5-{[4-((3,4-dihydro-6-hydroxy-2,5,7,8-tetramethyl-2H-1-benzopyran-2-yl)methoxy)-phenyl]-methyl}-thiazolidine-2,4-dione (troglitazone), 5-[6-(2-fluoro-benzyloxy)naphthalen-2-ylmethyl]-thiazolidine-2,4-dione (MCC555), 5-{[2-(2-naphthyl)-benzoxazol-5-yl]-methyl}-thiazolidine-2,4-dione (T-174) and 5-(2,4-dioxothiazolidin-5-ylmethyl)-2-methoxy-N-(4-trifluoromethyl-benzyl)benzamide (KRP297)).

Further anti-diabetic agents include, insulin signalling pathway modulators, like inhibitors of protein tyrosine phosphatases (PTPases), antidiabetic non-small molecule mimetic compounds and inhibitors of glutamine-fructose-6-phosphate amidotransferase (GFAT); compounds influencing a dysregulated hepatic glucose production, like inhibitors of glucose-6-phosphatase (G6Pase), inhibitors of fructose-1,6-bisphosphatase (F-1,6-Bpase), inhibitors of glycogen phosphorylase (GP), glucagon receptor antagonists and inhibitors of phosphoenolpyruvate carboxykinase (PEPCK); pyruvate dehydrogenase kinase (PDHK) inhibitors; inhibitors of gastric emptying; insulin; inhibitors of GSK-3; retinoid X receptor (RXR) agonists; agonists of Beta-3 AR; agonists of uncoupling proteins (UCPs); non-glitazone type PPARγ agonists; dual PPARα/PPARγ agonists; antidiabetic vanadium containing compounds; incretin hormones, like glucagon-like peptide-1 (GLP-1) and GLP-1 agonists; beta-cell imidazoline receptor antagonists; miglitol; α₂-adrenergic antagonists; and pharmaceutically acceptable salts thereof.

The term “obesity-reducing agent” includes lipase inhibitors (e.g., orlistat) and appetite suppressants (e.g., sibutramine and phentermine).

The term “aldosterone receptor blocker” includes spironolactone and eplerenone.

The term “endothelin receptor blocker” includes bosentan.

The term “CETP inhibitor” refers to a compound that inhibits the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). Examples include compounds disclosed in U.S. Pat. No. 6,140,343 and U.S. Pat. No. 6,197,786 (e.g., [2R,4S]4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib); compounds disclosed in U.S. Pat. No. 6,723,752 (e.g., (2R)-3-{[3-(4-Chloro-3-ethyl-phenoxy)-phenyl]-[[3-(1,1,2,2-tetrafluoro-ethoxy)-phenyl]-methyl]amino}-1,1,1-trifluoro-2-propanol); compounds disclosed in U.S. patent application Ser. No. 10/807,838; polypeptide derivatives disclosed in U.S. Pat. No. 5,512,548; rosenonolactone derivatives and phosphate-containing analogs of cholesteryl ester disclosed in J. Antibiot., 49(8): 815-816 (1996), and Bioorg. Med. Chem. Lett.; 6:1951-1954 (1996), respectively.

### Pharmaceutical Compositions of the Invention

The invention also pertains to pharmaceutical compositions comprising a compound of the invention, (e.g., a compound of Formula I or a compound otherwise described herein), and, optionally, one or more pharmaceutically acceptable carriers.

The pharmaceutical composition or combination of the present invention can be in unit dosage of about 1-1000 mg of active ingredients for a subject of about 50-70 kg, preferably about 1-500 mg or about 1-250 mg or about 1-150 mg or about 0.5-100 mg, or about 1-50 mg of active ingredients. The therapeutically effective dosage of a compound, the pharmaceutical composition, or the combinations thereof, is dependent on the species of the subject, the body weight, age and individual condition, the disorder or disease or the severity thereof being treated. A physician, clinician or veterinarian of ordinary skill can readily determine the effective amount of each of the active ingredients necessary to prevent, treat or inhibit the progress of the disorder or disease.

The above-cited dosage properties are demonstrable in vitro and in vivo tests using advantageously mammals, e.g., mice, rats, dogs, monkeys or isolated organs, tissues and preparations thereof. The compounds of the present invention can be applied in vitro in the form of solutions, e.g., preferably aqueous solutions, and in vivo either enterally, parenterally, advantageously intravenously, e.g., as a suspension or in aqueous solution. The dosage in vitro may range between about 10⁻³ molar and about 10⁻⁹ molar concentrations, or between about 10⁻⁶ molar and about 10⁻⁹ molar concentrations.

The activities of a compound according to the present invention can be assessed by both in vitro and in vivo methods, such as the DSS rat model as described in Journal of Hypertension (2005) 23, 87, the mouse pressure overload model Circulation (1999) 84, 735.

The term “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, wetting agents, emulsifiers, buffers, disintegration agents, lubricants, coatings, sweetening agents, flavoring agents, dyes, such like materials and combinations thereof, as would be known to one of ordinary skill in the art (see, for example, Remington's Pharmaceutical Sciences, 18^{th} Ed. Mack Printing Company, 1990, pp. 1289-1329, incorporated herein by reference). Except insofar as any conventional carrier is incompatible with the active ingredient, its use in the therapeutic or pharmaceutical compositions is contemplated. Suitable pharmaceutically acceptable carriers include but are not limited to water, salt solutions, alcohol, vegetable oils, polyethylene glycols, gelatin, lactose, amylase, magnesium stearate, talc, silicic aid, viscous paraffin, perfume oil, fatty acid monoglycerides and diglycerides, petroethral fatty acid esters, hydroxymethyl-cellulose, polyvinylpyrrolidone, etc.

The pharmaceutical compositions of the invention can be formulated for particular routes of administration such as oral administration, parenteral administration, and rectal administration, etc. In addition, the pharmaceutical compositions of the present invention can be made up in a solid form including capsules, tablets, pills, granules, powders or suppositories, or in a liquid form including solutions, suspensions or emulsions. The pharmaceutical compositions can be subjected to conventional pharmaceutical operations such as sterilization and/or can contain conventional inert diluents, lubricating agents, or buffering agents, as well as adjuvants, such as preservatives, stabilizers, wetting agents, emulsifiers and buffers, etc.

In certain embodiments, the pharmaceutical compositions are tablets and gelatin capsules comprising the active ingredient together with
a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and/or polyvinylpyrrolidone; if desired
d) disintegrants, e.g., starches, agar, alginic acid or its sodium salt, or effervescent mixtures; and/or
e) absorbents, colorants, flavors and sweeteners.

Tablets may be either film coated or enteric coated according to methods known in the art.

Suitable compositions for oral administration include an effective amount of a compound of the invention in the form of tablets, lozenges, aqueous or oily suspensions, dispersible powders or granules, emulsion, hard or soft capsules, or syrups or elixirs. Compositions intended for oral use are prepared according to any method known in the art for the manufacture of pharmaceutical compositions and such compositions can contain one or more agents selected from the group consisting of sweetening agents, flavoring agents, coloring agents and preserving agents in order to provide pharmaceutically elegant and palatable preparations. Tablets contain the active ingredient in admixture with nontoxic pharmaceutically acceptable excipients which are suitable for the manufacture of tablets. These excipients are, for example, inert diluents, such as calcium carbonate, sodium carbonate, lactose, calcium phosphate or sodium phosphate; granulating and disintegrating agents, for example, corn starch, or alginic acid; binding agents, for example, starch, gelatin or acacia; and lubricating agents, for example magnesium stearate, stearic acid or talc. The tablets are uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Injectable compositions are preferably aqueous isotonic solutions or suspensions, and suppositories are advantageously prepared from fatty emulsions or suspensions. Said compositions may be sterilized and/or contain adjuvants, such as preserving, stabilizing, wetting or emulsifying agents, solution promoters, salts for regulating the osmotic pressure, and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable compositions for transdermal application include an effective amount of a compound of the invention with carrier. Advantageous carriers include absorbable pharmacologically acceptable solvents to assist passage through the skin of the host. For example, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

Suitable compositions for topical application, e.g., to the skin and eyes, include aqueous solutions, suspensions, ointments, creams, gels or sprayable formulations, e.g., for delivery by aerosol or the like. Such topical delivery systems will in particular be appropriate for dermal application, e.g., for the treatment of skin cancer, e.g., for prophylactic use in sun creams, lotions, sprays, etc. They are thus particularly suited for use in topical, including cosmetic, formulations well-known in the art. Such may contain solubilizers, stabilizers, tonicity enhancing agents, buffers and preservatives.

The present invention further provides anhydrous pharmaceutical compositions and dosage forms comprising the compounds of the present invention as active ingredients, since water can facilitate the degradation of some compounds. Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions are packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

The invention further provides pharmaceutical compositions and dosage forms that comprise one or more agents that reduce the rate by which the compound of the present invention as an active ingredient will decompose. Such agents, which are referred to herein as “stabilizers,” include, but are not limited to, antioxidants such as ascorbic acid, pH buffers, or salt buffers, etc.

One embodiment of the invention includes pharmaceutical compositions comprising an effective amount of a compound of the present invention, (e.g., a compound of Formula I or a compound otherwise described herein), in combination with a second agent and a pharmaceutical carrier.

In yet another embodiment, the invention pertains to compounds of the present invention, (e.g., a compound of Formula I or a compound otherwise described herein), for use in therapy.

Another embodiment of the invention includes a formulation comprising an effective amount of a compound of the present invention, (e.g., a compound of Formula I or a compound otherwise described herein), and a pharmaceutically acceptable excipient or carrier.

Another embodiment of the invention pertains to kits comprising,

(a) a pharmaceutical composition comprising tablets, each comprising a compound of the present invention, (e.g., a compound of Formula I or a compound otherwise described herein) and optionally a pharmaceutically acceptable carrier,

(b) a packaging material enclosing a pharmaceutical composition, and transferred instructions for use of a pharmaceutical composition in the treatment of a PKD associated disorder in a subject in need thereof.

### EXEMPLIFICATION OF THE INVENTION

The following examples are intended to illustrate the compounds of the invention and are not to be construed as being limitations thereon. Temperatures are given in degrees centrigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

### Example 1

### A. 4-(2′-Chloro-[2,4′]bipyridinyl-6-yl)-piperazine-1-carboxylic acid tert-butyl ester

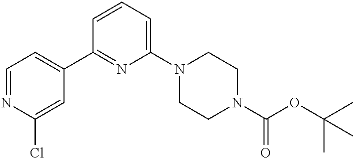

A mixture of 4-(6-bromopyridin-2-yl)-piperazine-1-carboxylic acid tert-butyl ester (1.98 g, 5.78 mmol), 2-chloropyridine-4-boronic acid (1.0 g, 6.35 mmol), Pd(Ph₃P)₄ (0.330 g, 0.289 mmol), aqueous solution of Na₂CO₃ (5.7 mL, 2.0 M) and CH₃CN (10 mL) is sparged with argon for 10 min. The vessel is then sealed and the contents heated to 90° C. for 4 h. The mixture is then allowed to cool followed by concentration. The residue is taken up in CH₂Cl₂ and washed with H₂O. The aqueous layer is further extracted with CH₂Cl₂ (2×50 mL). The combined organic layers are then dried (Na₂SO₄), filtered and concentrated. The residue is then separated via flash chromatography (SiO₂, 20-30% EtOAc/hexanes gradient) to give the title compound 4-(2′-chloro-[2,4′]bipyridinyl-6-yl)piperazine-1-carboxylic acid tert-butyl ester. MS (ESI) m/z 375.0, 376.9 (M+1). ¹H NMR (400 MHz, CDCl₃) δ ppm 8.44 (d, J=5.3 Hz, 1H), 7.93 (s, 1H), 7.78 (dd, J=5.1, 1.5 Hz, 1H), 7.61 (dd, J=8.5, 7.5 Hz, 1H), 7.16 (d, J=7.3 Hz, 1H), 6.73 (d, J=8.6 Hz, 1H), 3.55-3.69 (m, 8H), 1.50 (s, 9H).

### B. 4-(2′-Cyclohexylamino-[2,4′]bipyridinyl-6-yl)-piperazine-1-carboxylic acid tert-butyl ester

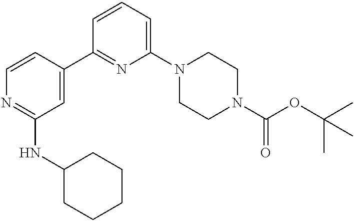

A mixture of 4-(2′-chloro-[2,4′]bipyridinyl-6-yl)piperazine-1-carboxylic acid tert-butyl ester (0.300 g, 0.801 mmol), Pd(t-Bu₃P)₂ (0.041 g, 0.080 mmol), NaOtBu (0.115 g, 1.20 mmol), cyclohexylamine (0.18 mL, 1.60 mmol) and 1,4-dioxane (4 mL) is sparged with argon for 10 min. The vessel is then sealed and the contents heated to 130° C. for 8 h. The mixture is then allowed to cool followed by concentration. The residue is then separated via flash chromatography (SiO₂, EtOAc/hexanes gradient) to give the title compound 4-(2′-cyclohexylamino-[2,4′]bipyridinyl-6-yl)-piperazine-1-carboxylic acid tert-butyl ester. MS (ESI) m/z 438.0 (M+1). ¹H NMR (400 MHz, DMSO-d₆) δ ppm 7.99 (d, J=5.3 Hz, 1H), 7.61-7.69 (m, 1H), 7.19 (d, J=7.6 Hz, 1H), 7.15 (s, 1H), 7.01 (d, J=5.6 Hz, 1H), 6.88 (d, J=8.6 Hz, 1 H), 6.32-6.61 (m, 1H), 3.68-3.78 (m, 1H), 3.59 (d, J=10.4 Hz, 4H), 3.42-3.50 (m, 4H), 1.94 (d, J=15.9 Hz, 2H), 1.73 (d, J=19.5 Hz, 2H), 1.60 (d, J=20.5 Hz, 1H), 1.43 (s, 9H), 1.26-1.40 (m, 2H), 1.11-1.26 (m, 3H).

### C. Cyclohexyl-(6-piperazin-1-yl-[2,4′]bipyridinyl-2′-yl)-amine

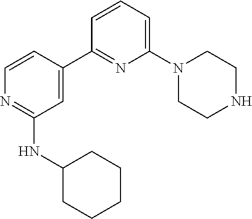

To a solution of 4-(2′-cyclohexylamino-[2,4′]bipyridinyl-6-yl)-piperazine-1-carboxylic acid tert-butyl ester (0.220 g, 0.503 mmol) and CH₂Cl₂ (7 mL) is added TFA (5 mL). After stirring for 1 h the solution is concentrated. The residue is taken up in CH₂Cl₂ (50 mL) and washed with a saturated aqueous solution of Na₂CO₃. The aqueous layer is further extracted with CH₂Cl₂ (2×50 mL). The combined organic layers are then dried (Na₂SO₄), filtered and concentrated. The residue is then separated via semi-prep HPLC (10-90% CH₃CN/H₂O gradient with 0.1% NH₄OH) to give the title compound cyclohexyl-(6-piperazin-1-yl-[2,4′]bipyridinyl-2′-yl)-amine. MS (ESI) m/z 338.2 (M+1). ¹H NMR (400 MHz, DMSO-d₆) δ ppm 7.99 (d, J=5.3 Hz, 1H), 7.61 (t, J=8.0 Hz, 1H), 7.12 (s, 1H), 7.10-7.17 (m, 1H), 6.98 (d, J=5.6 Hz, 1H), 6.82 (d, J=8.6 Hz, 1H), 6.43 (d, J=7.8 Hz, 1H), 3.66-3.80 (m, 1H), 3.44-3.55 (m, 4H), 3.31 (s, 1H), 2.77-2.86 (m, 4H), 1.87-1.98 (m, 2H), 1.67-1.78 (m, 2H), 1.55-1.65 (m, 1H), 1.26-1.39 (m, 2H), 1.12-1.25 (m, 3H).

Compounds D and E of Example 1 can be prepared by a similar method as those above.

### D. Isopropyl-(6-piperazin-1-yl-[2,4′]bipyridinyl-2′-yl)-amine

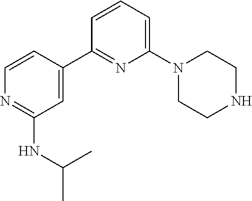

MS (ESI) m/z 298.1 (M+1). ¹H NMR (400 MHz, DMSO-d₆) δ ppm 8.00 (d, J=5.3 Hz, 1H), 7.58-7.65 (m, 1H), 7.14 (d, J=7.3 Hz, 1H), 7.10 (s, 1H), 6.99 (dd, J=5.4, 1.4 Hz, 1 H), 6.83 (d, J=8.6 Hz, 1H), 6.40 (d, J=7.6 Hz, 1H), 3.97-4.11 (m, 1H), 3.47-3.55 (m, 4 H), 2.79-2.88 (m, 4H), 1.15 (d, J=6.6 Hz, 6H).

### E. ((R)-1-Phenyl-ethyl)-(6-piperazin-1-yl-[2,4′]bipyridinyl-2′-yl)-amine

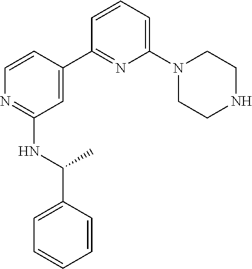

MS (ESI) m/z 360.1 (M+1). ¹H NMR (400 MHz, DMSO-d₆) δ ppm 7.95 (d, J=5.6 Hz, 1H), 7.60 (dd, J=8.6, 7.6 Hz, 1H), 7.36-7.42 (m, 2H), 7.28 (t, J=7.6 Hz, 2H), 7.05-7.21 (m, 4H), 7.00 (dd, J=5.4, 1.4 Hz, 1H), 6.81 (d, J=8.3 Hz, 1H), 4.96-5.08 (m, 1H), 3.42-3.50 (m, 4H), 2.76-2.85 (m, 4H), 1.44 (d, J=7.1 Hz, 3H).

### F. (6-piperazin-1-yl-[2,4′]bipyridinyl-2′-yl)-(tetrahydro-pyran-4-yl)-amine

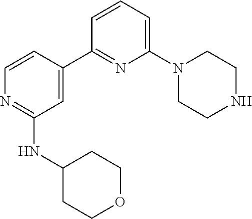

MS (ESI) m/z 340.1 (M+1). ¹H NMR (400 MHz, DMSO-d₆) δ ppm 8.01 (d, J=5.3 Hz, 1H), 7.57-7.65 (m, 1H), 7.10-7.17 (m, 2H), 7.02 (dd, J=5.4, 1.4 Hz, 1H), 6.83 (d, J=8.3 Hz, 1H), 6.58 (d, J=7.3 Hz, 1H), 3.91-4.03 (m, 1H), 3.82-3.92 (m, 2H), 3.46-3.54 (m, 4 H), 3.36-3.46 (m, 2H), 2.77-2.86 (m, 4H), 1.83-1.95 (m, 2H), 1.36-1.52 (m, 2H).

### G. Phenyl-(6-piperazin-1-yl-[2,4′]bipyridinyl-2′-yl)-amine

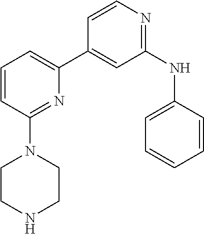

MS (ESI) m/z 332.1 (M+1). ¹H NMR (400 MHz, CDCl₃) δ ppm 8.27 (d, J=5.3 Hz, 1 H), 7.54-7.60 (m, 2H), 7.39-7.44 (m, 2H), 7.30-7.38 (m, 3H), 7.10 (d, J=7.6 Hz, 1H), 7.02-7.08 (m, 1H), 6.68 (d, J=8.3 Hz, 1H), 6.61 (br. s., 1H), 3.58-3.63 (m, 4H), 2.99-3.05 (m, 4H).

### H. (1-Methyl-1H-pyrazol-3-yl)-(6-piperazin-1-yl-[2,4′]bipyridinyl-2′-yl)-amine

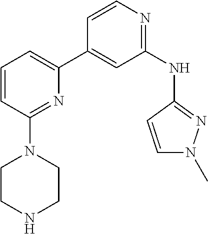

MS (ESI) m/z 336.0 (M+1). ¹H NMR (400 MHz, DMSO-d₆) δ ppm 9.24 (s, 1H), 8.15 (d, J=5.3 Hz, 1H), 8.09 (br. s., 1H), 7.65 (dd, J=8.5, 7.5 Hz, 1H), 7.51 (d, J=2.0 Hz, 1H), 7.27 (dd, J=5.3, 1.5 Hz, 1H), 7.21 (d, J=7.3 Hz, 1H), 6.86 (d, J=8.6 Hz, 1H), 6.28 (d, J=2.3 Hz, 1H), 3.75 (s, 3H), 3.52-3.57 (m, 4H), 2.82-2.86 (m, 4H).

### I. (2-Methyl-2H-pyrazol-3-yl)-(6-piperazin-1-yl-[2,4′]bipyridinyl-2′-yl)-amine

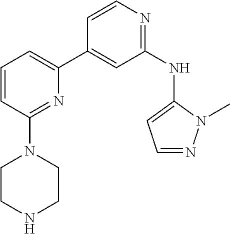

MS (ESI) m/z 336.1 (M+1). ¹H NMR (400 MHz, MeOD) δ ppm 8.08-8.15 (m, 1H), 7.60-7.68 (m, 1H), 7.42-7.48 (m, 2H), 7.36-7.41 (m, 1H), 7.19-7.24 (m, 1H), 6.82-6.89 (m, 1H), 6.23-6.27 (m, 1H), 3.74 (s, 3H), 3.59-3.64 (m, 3H), 2.91-2.99 (m, 3H), 2.19-2.23 (m, 1H), 1.92-1.95 (m, 1H).

### Example 2

### A. 4-[2′-(3-Methoxycarbonyl-2-methyl-phenylamino)-[2,4′]bipyridinyl-6-yl]-piperazine-1-carboxylic acid tert-butyl ester

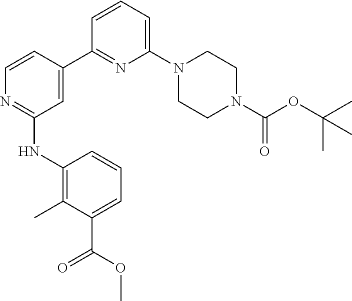

The title compound is prepared in similar method to Example 1B. ¹H NMR (400 MHz, CDCl₃) δ ppm 8.25 (d, J=5.3 Hz, 1H), 7.61-7.73 (m, 2H), 7.49-7.59 (m, 1H), 7.23-7.31 (m, 3H), 7.07 (d, J=7.6 Hz, 1H), 6.66 (d, J=8.3 Hz, 1H), 6.37 (s, 1H), 3.92 (s, 3H), 3.56 (q, J=5.4 Hz, 8H), 2.51 (s, 3H), 1.50 (s, 9H).

### B. 2-Methyl-3-(6-piperazin-1-yl-[2,4′]bipyridinyl-2′-ylamino)-N-(2-pyrrolidin-1-yl-ethyl)-benzamide

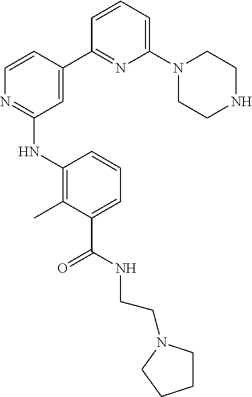

To a solution of toluene (6 mL) and AIMe₃ (1.3 mL, 2.62 mmol) is added 2-pyrrolidin-1-yl-ethylamine (0.33 mL, 2.62 mmol). After 5 min 4-[2′-(3-methoxycarbonyl-2-methyl-phenylamino)-[2,4′]bipyridinyl-6-yl]-piperazine-1-carboxylic acid tert-butyl ester (0.220 g, 0.437 mmol) is added in toluene (4 mL) and the resulting solution heated to 100° C. After 2 h the solution is carefully diluted with 1 M HCl (10 mL) and vigorously stirred for 5 min. The mixture is then basified with 8 M NaOH (2 mL), further diluted with H₂O and extracted with CH₂Cl₂. The combined organic layers are then dried (Na₂SO₄), filtered and concentrated. The residue is then taken on without further purification.

The residue from above is taken up in CH₂Cl₂ (5 mL) and treated with TFA (3 mL). After 1 h the solution is concentrated. The residue is taken up in CH₂Cl₂ (50 mL) and washed with a saturated aqueous solution of Na₂CO₃. The aqueous layer is further extracted with CH₂Cl₂ (2×50 mL). The combined organic layers are then dried (Na₂SO₄), filtered and concentrated. The residue is then separated via semi-preparative HPLC (10-90% CH₃CN/H₂O gradient with 0.1% TFA) followed by conversion to the free base to give the title compound 2-methyl-3-(6-piperazin-1-yl-[2,4′]bipyridinyl-2′-ylamino)-N-(2-pyrrolidin-1-yl-ethyl)-benzamide. MS (ESI) m/z 486.2 (M+1). ¹H NMR (400 MHz, DMSO-d₆) δ ppm 8.34 (s, 1H), 8.13-8.20 (m, 1H), 8.10 (d, J=5.3 Hz, 1H), 7.56-7.67 (m, 2H), 7.43 (s, 1H), 7.25 (dd, J=5.3, 1.3 Hz, 1H), 7.13-7.21 (m, 2H), 6.99 (dd, J=7.6, 1.0 Hz, 1H), 6.86 (d, J=8.6 Hz, 1H), 3.46-3.54 (m, 4H), 3.31-3.39 (m, 7H), 2.79-2.87 (m, 4H), 2.53-2.60 (m, 2H), 2.22 (s, 3H), 1.62-1.74 (m, 4H).

Compounds C and D of Example 2 can be prepared by a similar method as those above.

### C. 2-Methyl-N-(2-morpholin-4-yl-ethyl)-3-(6-piperazin-1-yl-[2,4′]bipyridinyl-2′-ylamino)-benzamide

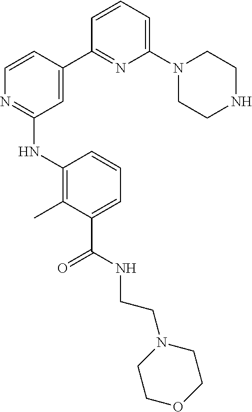

MS (ESI) m/z 502.2 (M+1). ¹H NMR (400 MHz, DMSO-d₆) δ ppm 8.34 (s, 1H), 8.11-8.17 (m, 1H), 8.10 (d, J=5.3 Hz, 1H), 7.59-7.67 (m, 2H), 7.44 (s, 1H), 7.25 (dd, J=5.3, 1.5 Hz, 1H), 7.13-7.21 (m, 2H), 7.00 (dd, J=7.3, 1.0 Hz, 1H), 6.85 (d, J=8.6 Hz, 1H), 3.53-3.60 (m, 4H), 3.45-3.52 (m, 4H), 3.32-3.40 (m, 2H), 2.77-2.84 (m, 4H), 2.43-2.48 (m, 2H), 2.38-2.44 (m, 4H), 2.24 (s, 3H).

### D. N-(3-Dimethylaminopropyl)-2-methyl-3-(6-piperazin-1-yl-[2,4′]bipyridinyl-2′-ylamino)-benzamide

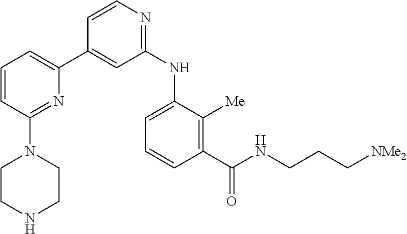

MS (ESI) m/z 474.2 (M+1). ¹H NMR (400 MHz, DMSO-d₆) δ ppm 8.33 (s, 1H), 8.20-8.28 (m, 1H), 8.10 (d, J=5.3 Hz, 1H), 7.55-7.67 (m, 2H), 7.44 (s, 1H), 7.25 (dd, J=5.4, 1.4 Hz, 1H), 7.13-7.20 (m, 2H), 7.00 (d, J=7.6 Hz, 1H), 6.85 (d, J=8.3 Hz, 1H), 3.45-3.52 (m, 4H), 3.24 (app q, J=6.7 Hz, 2H), 2.77-2.83 (m, 4H), 2.26 (app t, J=7.2 Hz, 2H), 2.21 (s, 3H), 2.13 (s, 6H), 1.56-1.70 (m, 2H).

### Example 3

### A. 4-(3-Bromo-2′-chloro-[2,4′]bipyridinyl-6-yl)-piperazine-1-carboxylic acid tert-butyl ester

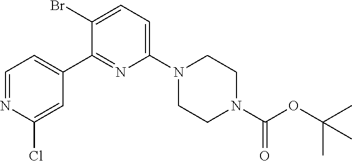

To a solution of 4-(2′-chloro-[2,4′]bipyridinyl-6-yl)-piperazine-1-carboxylic acid tert-butyl ester Example 1A (5.15 g, 13.8 mmol) in CH₂Cl₂ (100 mL) is added bromine (0.74 mL, 14.5 mmol). After 10 min the excess bromine is quenched by the addition of saturated aqueous Na₂S₂O₃ (20 mL) and saturated aqueous NaHCO₃ (20 mL). The mixture is then diluted further with CH₂Cl₂ (100 mL) and H₂O (200 mL). The aqueous layer is further extracted with CH₂Cl₂ (2×100 mL). The combined organic layers are then dried (Na₂SO₄), filtered and concentrated. The residue is then separated via flash chromatography (SiO₂, 10-30% EtOAc/hexanes gradient) to give the title compound 4-(3-Bromo-2′-chloro-[2,4]bipyridinyl-6-yl)-piperazine-1-carboxylic acid tert-butyl ester MS (ESI) m/z 452.9, 454.9, 456.8 (M+1). ¹H NMR (400 MHz, CDCl₃) δ ppm 8.45 (dd, J=5.2, 0.6 Hz, 1H), 7.71 (d, J=8.8 Hz, 1H), 7.64-7.67 (m, 1H), 7.57 (dd, J=5.1, 1.5 Hz, 1H), 6.58 (d, J=9.1 Hz, 1H), 3.55 (br. s., 8H), 1.48 (s, 9H).

### B. 4-[2′-Chloro-3-(4-fluorophenyl)-[2,4′]bipyridinyl-6-yl]-piperazine-1-carboxylic acid tert-butyl ester

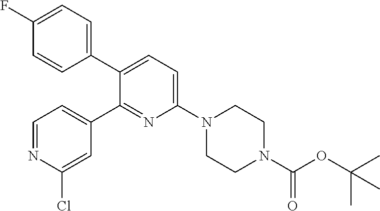

A suspension of 4-(3-bromo-2′-chloro-[2,4′]bipyridinyl-6-yl)-piperazine-1-carboxylic acid tert-butyl ester (0.300 g, 0.661 mmol), 4-fluorobenzene boronic acid (0.139 g, 0.992 mmol), Pd(dppf)Cl₂.CH₂Cl₂ (0.054 g, 0.066 mmol), aqueous solution of Na₂CO₃ (0.66 mL, 2.0 M) and DME (8 mL) is heated in a microwave reactor at 120° C. for 0.5 h. The mixture is then allowed to cool followed by concentration. The residue is taken up in CH₂Cl₂ (50 mL) and washed with brine (50 mL). The aqueous layer is further extracted with CH₂Cl₂ (2×50 mL). The combined organic layers are then dried (Na₂SO₄), filtered and concentrated. The residue is then separated via flash chromatography (SiO₂, 5-20% EtOAc/hexanes gradient) to give the title compound 4-[2′-chloro-3-(4-fluorophenyl)-[2,4′]bipyridinyl-6-yl]-piperazine-1-carboxylic acid tert-butyl ester. MS (ESI) m/z 469.0, 470.8 (M+1). ¹H NMR (400 MHz, DMSO-d₆) δ ppm 8.28 (d, J=5.1 Hz, 1H), 7.67 (d, J=8.8 Hz, 1H), 7.33-7.36 (m, 1H), 7.17 (d, J=10.4 Hz, 5H), 7.04 (d, J=8.8 Hz, 1H), 3.57-3.65 (m, 4H), 3.42-3.52 (m, 4H), 1.43 (s, 9H).

### C. 4-[2′-Cyclohexylamino-3-(4-fluorophenyl)-[2,4′]bipyridinyl-6-yl]-piperazine-1-carboxylic acid tert-butyl ester

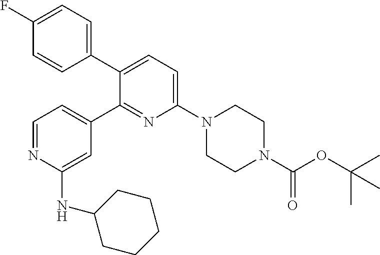

A mixture of 4-[2′-chloro-3-(4-fluoro-phenyl)-[2,4′]bipyridinyl-6-yl]-piperazine-1-carboxylic acid tert-butyl ester (0.290 g, 0.801 mmol), Pd(t-Bu₃P)₂ (0.032 g, 0.080 mmol), NaOtBu (0.178 g, 1.86 mmol), cyclohexylamine (0.21 mL, 1.86 mmol) and 1,4-dioxane (6 mL) is heated in a microwave reactor at 130° C. for 1 h. The mixture is then allowed to cool followed by concentration. The residue is then taken up in CH₂Cl₂ (50 mL) and washed with brine (50 mL). The aqueous layer is further extracted with CH₂Cl₂ (2×50 mL). The combined organic layers are then dried (Na₂SO₄), filtered and concentrated. The residue is then separated via flash chromatography (SiO₂, 20-60% EtOAc/heptane gradient) to give the title compound 4-[2′-cyclohexylamino-3-(4-fluorophenyl)-[2,4′]bipyridinyl-6-yl]-piperazine-1-carboxylic acid tert-butyl ester. MS (ESI) m/z 532.3 (M+1).

### D. Cyclohexyl-[3-(4-fluorophenyl)-6-piperazin-1-yl-[2,4′]bipyridinyl-2′-yl]-amine

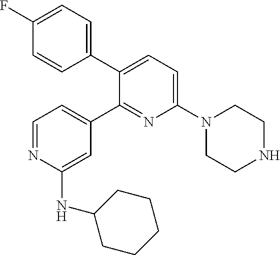

To a solution of 4-[2′-cyclohexylamino-3-(4-fluorophenyl)-[2,4′]bipyridinyl-6-yl]-piperazine-1-carboxylic acid tert-butyl ester (0.175 g, 0.329 mmol) and CH₂Cl₂ (3 mL) is added TFA (1 mL). After stirring for 1 h, the solution is concentrated. The residue is then separated via semi-preparative HPLC (10-90% CH₃CN/H₂O gradient with 0.1% NH₄OH) to give the title compound cyclohexyl-(6-piperazin-1-yl-[2,4′]bipyridinyl-2′-yl)-amine. MS (ESI) m/z 432.0 (M+1). ¹H NMR (400 MHz, CDCl₃) δ ppm 7.95 (d, J=5.1 Hz, 1H), 7.51 (d, J=2.5 Hz, 1H), 7.49 (s, 1H), 7.09-7.16 (m, 2H), 6.92-7.01 (m, 2H), 6.70 (d, J=8.6 Hz, 1H), 6.58-6.64 (m, 1H), 6.26 (s, 1H), 3.55-3.64 (m, 4H), 3.06-3.23 (m, 1H), 2.98-3.04 (m, 4H), 1.60-1.83 (m, 5H), 1.17-1.32 (m, 3H), 1.00-1.15 (m, 2H).

Compounds E-L of Example 3 can be prepared by a similar method as those above.

### E. Cyclohexyl-(3-phenyl-6-piperazin-1-yl-[2,4′]bipyridinyl-2′-yl)-amine

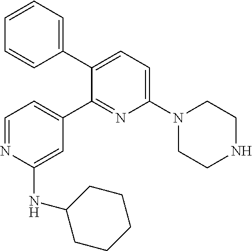

MS (ESI) m/z 414.1 (M+1). ¹H NMR (400 MHz, CDCl₃) δ ppm 7.93 (d, J=5.3 Hz, 1H), 7.54 (d, J=8.6 Hz, 1H), 7.19-7.30 (m, 4H), 7.14-7.19 (m, 2H), 6.71 (d, J=8.8 Hz, 1H), 6.65 (dd, J=5.3, 1.3 Hz, 1H), 6.28 (s, 1H), 4.27-4.57 (m, 1H), 3.61 (d, J=10.1 Hz, 4H), 3.05-3.15 (m, 1H), 2.99-3.04 (m, 4H), 1.72-1.80 (m, 2H), 1.61-1.70 (m, 3H), 1.12-1.30 (m, 3H), 0.98-1.10 (m, 2H).

### F. Cyclohexyl-[6-piperazin-1-yl-3-(3-trifluoromethyl-phenyl)-[2,4′]bipyridinyl-2′-yl]-amine

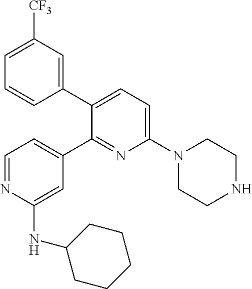

MS (ESI) m/z 482.1 (M+1). ¹H NMR (400 MHz, DMSO-d₆) δ ppm 7.78 (d, J=5.1 Hz, 1H), 7.64 (d, J=8.6 Hz, 1H), 7.55-7.60 (m, 1H), 7.51 (app t, J=7.7 Hz, 1H), 7.39-7.46 (m, 2H), 6.91 (d, J=8.6 Hz, 1H), 6.35 (s, 1H), 6.24 (d, J=7.8 Hz, 1H), 6.20 (dd, J=5.3, 1.3 Hz, 1H), 3.46-3.54 (m, 4H), 3.34-3.46 (m, 1H), 2.74-2.85 (m, 4H), 2.34-2.48 (m, 1H), 1.71-1.81 (m, 2H), 1.61-1.70 (m, 2H), 1.51-1.59 (m, 1H), 1.14-1.31 (m, 2H), 0.99-1.14 (m, 3H).

### G. Cyclohexyl-(6′-piperazin-1-yl-[4,2′,3′,4″]terpyridin-2-yl)-amine

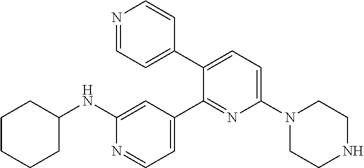

MS (ESI) m/z 415.2 (M+1). ¹H NMR (400 MHz, MeOH-d₄) δ ppm 8.29-8.33 (m, 2 H), 7.72 (d, J=5.6 Hz, 1H), 7.57 (d, J=8.8 Hz, 1H), 7.12-7.17 (m, 2H), 6.82 (d, J=8.6 Hz, 1H), 6.41 (dd, J=5.6, 1.5 Hz, 1H), 6.28 (s, 1H), 3.52-3.61 (m, 4H), 3.23-3.31 (m, 1H), 2.81-2.88 (m, 4H), 1.49-1.75 (m, 5H), 1.03-1.30 (m, 5H).

### H. Cyclohexyl-[6-piperazin-1-yl-3-(4-trifluoromethyl-phenyl)-[2,4′]bipyridinyl-2′-yl]-amine

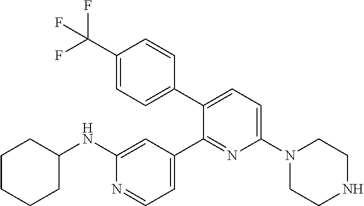

(ESI) m/z 482.1 (M+1). ¹H NMR (400 MHz, MeOH-d₄) δ ppm 7.70 (d, J=5.8 Hz, 1H), 7.54 (d, J=8.6 Hz, 1H), 7.48 (d, J=8.3 Hz, 2H), 7.26 (d, J=7.8 Hz, 2H), 6.81 (d, J=8.6 Hz, 1H), 6.42 (dd, J=5.4, 1.4 Hz, 1H), 6.26 (s, 1H), 3.49-3.60 (m, 4H), 3.10-3.19 (obs m, 1H), 2.80-2.90 (m, 4H), 1.40-1.74 (m, 5H), 0.92-1.31 (m, 5H).

### I. Cyclohexyl-[6-piperazin-1-yl-3-(3-fluoro-phenyl)-[2,4′]bipyridinyl-2′-yl]-amine

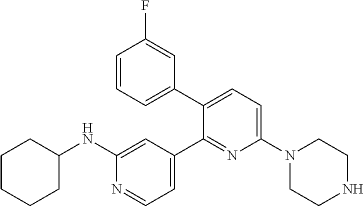

MS (ESI) m/z 432.1 (M+1). ¹H NMR (400 MHz, MeOH-d₄) δ ppm 7.68 (d, J=4.8 Hz, 1H), 7.49 (d, J=8.6 Hz, 1H), 7.11-7.24 (m, 1H), 6.68-6.93 (m, 4H), 6.42 (dd, J=5.4, 1.4 Hz, 1H), 6.31 (s, 1H), 3.43-3.59 (m, 4H), 3.10-3.21 (obs m, 1H), 2.75-2.90 (m, 4H), 1.45-1.79 (m, 5H), 0.94-1.35 (m, 5H).

### J. Cyclohexyl-[6-piperazin-1-yl-3-(3-cyano-phenyl)-[2,4′]bipyridinyl-2′-yl]-amine

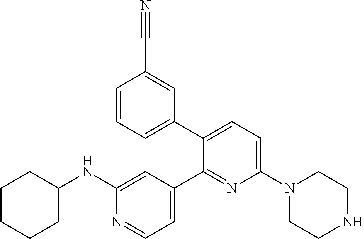

MS (ESI) m/z 439.2 (M+1). ¹H NMR (400 MHz, MeOH-d₄) δ ppm 7.70 (d, J=6.1 Hz, 1H), 7.43-7.57 (m, 3H), 7.27-7.37 (m, 2H), 6.81 (d, J=8.8 Hz, 1H), 6.38 (dd, J=5.4, 1.4 Hz, 1H), 6.26 (s, 1H), 3.47-3.61 (m, 4H), 2.78-2.89 (m, 4H), 1.48-1.78 (m, 5H), 0.92-1.32 (m, 5H).

### K. Cyclohexyl-[6-piperazin-1-yl-3-(4-cyano-phenyl)-[2,4′]bipyridinyl-2′-yl]-amine

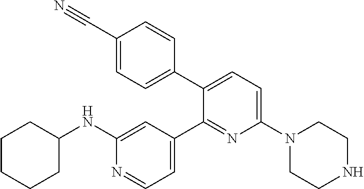

MS (ESI) m/z 439.2 (M+1). ¹H NMR (400 MHz, MeOH-d₄) δ ppm 7.70 (d, J=5.3 Hz, 1H), 7.47-7.59 (m, 3H), 7.18-7.31 (m, 2H), 6.81 (d, J=8.8 Hz, 1H), 6.40 (dd, J=5.4, 1.4 Hz, 1H), 6.25 (s, 1H), 3.49-3.63 (m, 4H), 2.79-2.91 (m, 4H), 1.47-1.77 (m, 5H), 0.91-1.31 (m, 5H).

### L. Cyclohexyl-(6′-piperazin-1-yl-[3,3′;2′,4″]terpyridin-2″-yl)-amine

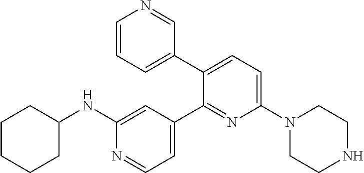

MS (ESI) m/z 415.1 (M+1). ¹H NMR (400 MHz, MeOH-d₄) δ ppm 8.29 (dd, J=4.9, 1.6 Hz, 1H), 8.22 (d, J=3.0 Hz, 1H), 7.69 (d, J=6.1 Hz, 1H), 7.50-7.63 (m, 2H), 7.28 (dd, J=7.6, 4.5 Hz, 1H), 6.83 (d, J=8.8 Hz, 1H), 6.34 (dd, J=5.3, 1.5 Hz, 1H), 6.31 (s, 1H), 3.48-3.60 (m, 4H), 3.24-3.32 (m, 1H), 2.81-2.89 (m, 4H), 1.68-1.79 (m, 2H), 1.58-1.68 (m, 2H), 1.48-1.58 (m, 1H), 0.93-1.33 (m, 5H).

### Example 4

### A. 2,6-Dibromo-isonicotinic acid methyl ester

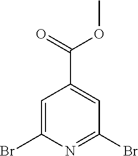

A mixture of citrazinic acid (5.0 g, 32.2 mmol) and POBr₃ (27.5 g, 96.8 mmol) is heated at 130° C. Upon completion of the reaction, the thick slurry is cooled to 0° C. and the reaction is carefully quenched with MeOH (250 mL). The reaction mixture is concentrated in vacuo and then extracted between dichloromethane and sat. aq. NaHCO₃. Organic layer is dried over anhydrous Na₂SO₄ and concentrated in vacuo to give a tan solid that is clean enough by NMR/LCMS to use further (7.5 g, 79%). (ESI) m/z 295.8 (M+1). ¹H NMR (400 MHz, CD₂Cl₂) δ ppm 8.10 (s, 2H), 4.05 (s, 3H).

### B. 4-(6-Bromo-4-methoxycarbonyl-pyridin-2-yl)-piperazine-1-carboxylic acid tert-butyl ester

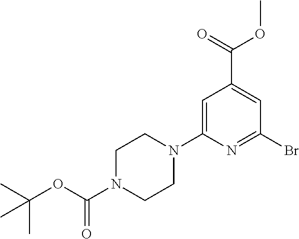

2,6-Dibromo-isonicotinic acid methyl ester (5.0 g, 17.0 mmol), piperazine-1-carboxylic acid tert-butyl ester (3.2 g, 17.0 mmol) and Et₃N (3.5 mL, 25.5 mmol) are stirred in 1,4-dioxane (75 mL) at 110° C. in a 150 mL pressure vessel until reaction is complete by LCMS. The reaction vessel is cooled to room temperature and the reaction mixture is concentrated in vacuo to afford a residue that is taken up in ACN/water (1:9). A tan solid precipitates out. The mixture is filtered and dried to give the above product that is clean enough by NMR/LCMS to use further (5.4 g, 80%). MS (ESI) m/z 402.0 (M+1). ¹H-NMR (400 MHz, CDCl₃) δ ppm 7.21 (s, 1H), 7.03 (s, 1H), 3.85 (s, 3H), 3.50-3.55 (m, 4H), 3.44-3.49 (m, 4H), 1.41 (s, 9H).

### C. 6-(4-tert-Butoxycarbonyl-piperazin-1-yl)-2′-chloro-[2,4′]-bipyridinyl-4-carboxylic acid methyl ester

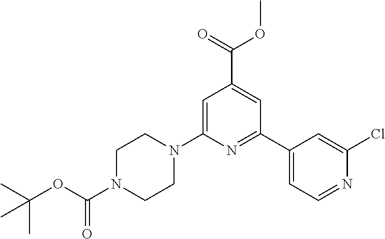

Stirred 4-(6-bromo-4-methoxycarbonyl-pyridin-2-yl)-piperazine-1-carboxylic acid tert-butyl ester (1.5 g, 3.76 mmol) and 2-chloro-4-pyridine boronic acid (0.71 g, 4.51 mmol) in DME (25 mL). To this is added 2.0 M Na₂CO₃ solution (6.0 mL, 11.28 mmol) and Pd(dppf)Cl₂.CH₂Cl₂ (0.31 g, 0.37 mmol). This above suspension is heated to 80° C. for 4 h. Reaction is diluted with EtOAc (25 mL) and extracted between organic and saturated NaHCO₃ (×2). The organic layer is washed with brine, dried over anhydrous Na₂SO₄ and evaporated under reduced pressure to provide a crude residue that is purified via flash chromatography (SiO₂, EtOAc/heptanes gradient) to afford the compound as a pale yellow solid (1.40 g, 87%). MS (ESI) m/z 433.2 (M+1). ¹H-NMR (400 MHz, CD₂Cl₂) δ ppm 8.37 (d, J=4.5 Hz, 1H), 7.92 (d, J=1.5 Hz, 1H), 7.79 (dd, J=5.1, 1.5 Hz, 1H), 7.60 (s, 1H), 7.26 (s, 1 H), 3.86 (s, 3H), 3.57-3.68 (m, 4H), 3.41-3.53 (m, 4H), 1.39 (s, 9H).

### D. 4-(4-tert-Butylcarbamoyl-2′-chloro-[2,4′]bipyridinyl-6-yl)-piperazine-1-carboxylic acid tert-butyl ester

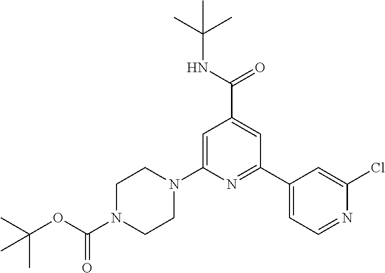

To a solution of toluene (60 mL) and trimethylaluminum (23.1 mL, 46.3 mmol) is added tert-butylamine (4.9 mL, 46.3 mmol). This solution is stirred at room temperature for 10 minutes before 6-(4-tert-butoxycarbonyl-piperazin-1-yl)-2′-chloro-[2,4′]-bipyridinyl-4-carboxylic acid methyl ester (2.5 g, 5.78 mmol) is added portion-wise. The resulting suspension is heated at 110° C. until LCMS indicated complete reaction. The reaction is cooled to ambient temperature and quenched carefully with MeOH. The gelatinous suspension is filtered and the filter cake washed well with MeOH. The organic is concentrated in vacuo and the residue purified via flash chromatography (SiO₂, EtOAc/heptanes gradient) to afford the compound as a yellow solid (2.05 g, 75%). MS (ESI) m/z 474.1 (M+1). ¹H-NMR (400 MHz, CD₂Cl₂) δ ppm 8.35 (d, J=5.1 Hz, 1H), 7.88 (s, 1H), 7.76 (dd, J=5.3, 1.5 Hz, 1H), 7.21 (s, 1H), 6.93 (s, 1H), 5.95 (br. s., 1H), 3.55-3.65 (m, 4 H), 3.44-3.51 (m, 4H), 1.39 (s, 18H).

### E. 4-[4-tert-Butylcarbamoyl-2′-(tetrahydropyran-4-ylamino)-[2,4′]bipyridinyl-6-yl]-piperazine-1-carboxylic acid tert-butyl ester

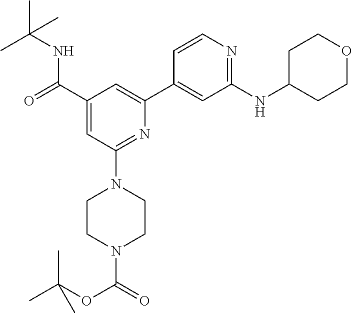

A mixture of 4-(4-tert-butylcarbamoyl-2′-chloro-[2,4′]bipyridinyl-6-yl)-piperazine-1-carboxylic acid tert-butyl ester (225.0 mg, 0.47 mmol), Pd(tBu₃P)₂ (24.0 mg, 0.047 mmol), NaOtBu (141.0 mg, 1.41 mmol), 4-aminotetrahydropyran (0.14 mL, 1.41 mmol) and 1,4-dioxane (5 mL) is sparged with argon for 10 min. The vessel is sealed and the contents heated to 130° C. for 2 h. The mixture is allowed to cool followed by concentration. The residue is separated via flash chromatography (SiO₂, EtOAc/hexanes gradient) to give the title compound (150 mg, 59%). MS (ESI) m/z 539.2 (M+1). ¹H NMR (400 MHz, CD₂Cl₂) δ ppm 8.13 (d, J=5.3 Hz, 1H), 7.26 (s, 1H), 7.13 (dd, J=5.3, 1.5 Hz, 1H), 7.07 (s, 1H), 7.01 (s, 1H), 6.07 (br. s., 1H), 4.76 (br. s., 1H), 3.93-4.16 (m, 1H), 3.65-3.76 (m, 4H), 3.58 (dd, J=6.3, 4.0 Hz, 4H), 1.50 (s, 18H), 1.29 (d, J=6.6 Hz, 6H).

### F. 6-piperazin-1-yl-2′-(tetrahydropyran-4-ylamino)-[2,4′]bipyridinyl-4-carboxylic acid amide

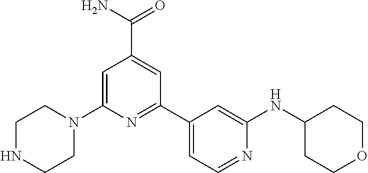

4-[4-Carbamoyl-2′-(tetrahydropyran-4-ylamino)-[2,4′]bipyridinyl-6-yl]-piperazine-1-carboxylic acid tertbutyl ester (115.0 mg, 0.21 mmol) and TFA (8 mL) are stirred in a microwave at 120° C. for 2 h. After stirring for 2 h the solution is concentrated. The residue is then separated via semi-preparative HPLC (10-90% CH₃CN/H₂O gradient with 0.1% NH₄OH) to give the title compound (95.0 mg, 75%). MS (ESI) m/z 383.1 (M+1). ¹H NMR (400 MHz, MeOH-d₄) δ ppm 7.90 (d, J=5.6 Hz, 1H), 7.46 (s, 1H), 7.16 (s, 1H), 7.13 (s, 1H), 7.07 (dd, J=5.6, 1.5 Hz, 1H), 3.80-3.95 (m, 3H), 3.55-3.66 (m, 4H), 3.41-3.53 (m, 2H), 2.78-2.94 (m, 4H), 1.82-1.98 (m, 2H), 1.33-1.54 (m, 2H).

Compounds G-V of Example 4 can be prepared by a similar method as those above.

### G. 2′-(4-Methoxy-2-methylphenylamino)-6-piperazin-1-yl-[2,4′]bipyridinyl-4-carboxylic acid amide

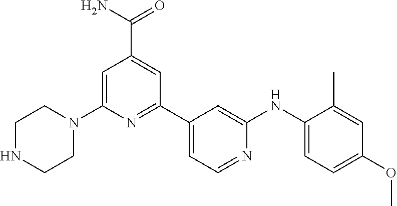

MS (ESI) m/z 419.1 (M+1). ¹H NMR (400 MHz, MeOH-d₄) δ ppm 8.02 (d, J=5.6 Hz, 1H), 7.51 (s, 1H), 7.25 (dd, J=5.4, 1.4 Hz, 1H), 7.22 (app d, J=2.0 Hz, 1H), 7.20 (app d, J=2.3 Hz, 1H), 6.87 (app d, J=2.8 Hz, 1H), 6.80 (dd, J=8.6, 2.8 Hz, 1H), 3.80 (s, 3H), 3.57-3.65 (m, 4H), 2.87-2.96 (m, 4H), 2.23 (s, 3H).

### H. 2′-Ethylamino-6-piperazin-1-yl-[2,4′]bipyridinyl-4-carboxylic acid amide

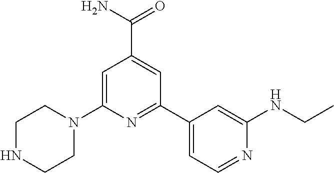

MS (ESI) m/z 327.1 (M+1). ¹H NMR (400 MHz, DMSO-d₆) δ ppm 8.18 (s, 1H), 8.04 (d, J=5.3 Hz, 1H), 7.60 (s, 1H), 7.53 (s, 1H), 7.20 (s, 1H), 7.13 (s, 1H), 7.07 (dd, J=5.4, 1.5 Hz, 1H), 6.56 (t, J=5.4 Hz, 1H), 3.49-3.59 (m, 4H), 3.24-3.36 (obs q, 2H), 2.73-2.87 (m, 4H), 2.38 (br. s., 1H), 1.15 (t, J=7.1 Hz, 3H).

### I. 2′-(2-Methoxyethylamino)-6-piperazin-1-yl-[2,4′]bipyridinyl-4-carboxylic acid amide

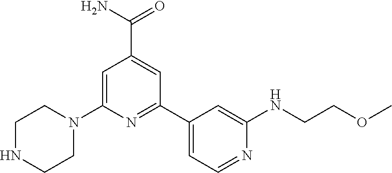

MS (ESI) m/z 357.2 (M+1). ¹H NMR (400 MHz, MeOH-d₄) δ ppm 8.00 (d, J=5.6 Hz, 1H), 7.55 (s, 1H), 7.27 (s, 1H), 7.21 (s, 1H), 7.19 (dd, J=5.7, 1.5 Hz, 1H), 3.66-3.72 (m, 4H), 3.60 (t, 2H), 3.52 (t, 2H), 3.39 (s, 3H), 2.93-2.99 (m, 4H).

### J. 2′-(2,2-Dimethylpropylamino)-6-piperazin-1-yl-[2,4′]bipyridinyl-4-carboxylic acid amide

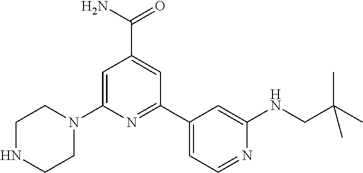

MS (ESI) m/z 369.2 (M+1). ¹H NMR (400 MHz, MeOH-d₄) δ ppm 7.96 (d, J=6.2 Hz, 1H), 7.55 (s, 1H), 7.31 (s, 1H), 7.21 (s, 1H), 7.14 (dd, J=5.6, 1.5 Hz, 1H), 3.60-3.78 (m, 4H), 3.19 (s, 2H), 2.90-2.99 (m, 4H), 1.00 (s, 9H).

### K. 2′-(2-Fluoro-4-methoxyphenylamino)-6-piperazin-1-yl-[2,4′]bipyridinyl-4-carboxylic acid amide

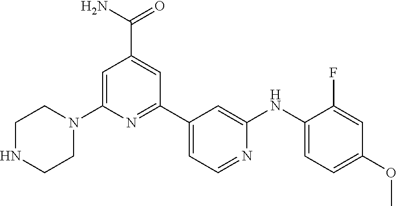

MS (ESI) m/z 423.1 (M+1). ¹H NMR (400 MHz, MeOH-d₄) δ ppm 8.07 (d, J=5.6 Hz, 1H), 7.49-7.60 (m, 2H), 7.43 (s, 1H), 7.33 (dd, J=5.6, 1.5 Hz, 1H), 7.21 (s, 1H), 6.66-6.86 (m, 2H), 3.81 (s, 3H), 3.56-3.70 (m, 4H), 2.89-3.01 (m, 4H).

### L. 4′-tert-Butylcarbamoyl-2″-isopropylamino-3,4,5,6-tetrahydro-2H-[4,2′;6′,4″]terpyridine-1-carboxylic acid tert-butyl ester

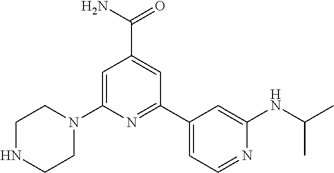

MS (ESI) m/z 341.1 (M+1). ¹H NMR (400 MHz, MeOH-d₄) δ ppm 7.89 (d, J=6.1 Hz, 1H), 7.45 (d, J=1.0 Hz, 1H), 7.09-7.15 (m, 2H), 7.05 (dd, J=5.7, 1.6 Hz, 1H), 3.82-4.01 (m, 1H), 3.51-3.65 (m, 4H), 2.79-2.92 (m, 4H), 1.15 (d, J=6.6 Hz, 6H).

### M. 2′-(2-Chlorophenylamino)-6-piperazin-1-yl-[2,4′]bipyridinyl-4-carboxylic acid amide

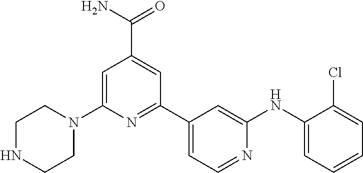

MS (ESI) m/z 409.1 (M+1). ¹H NMR (400 MHz, MeOH-d₄) δ ppm 8.16 (d, J=5.4 Hz, 1H), 7.88 (dd, J=8.1, 1.5 Hz, 1H), 7.66 (s, 1H), 7.59 (s, 1H), 7.44 (d, J=6.8 Hz, 2H), 7.28 (dt, 1H), 7.23 (s, 1H), 7.05 (dt, J=7.7, 1.5 Hz, 1H), 3.61-3.73 (m, 4H), 2.89-3.01 (m, 4H).

### N. 2′-(3-Imidazol-1-yl-propylamino)-6-piperazin-1-yl-[2,4′]bipyridinyl-4-carboxylic acid amide

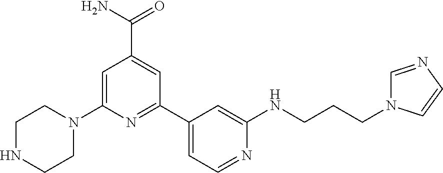

MS (ESI) m/z 407.2 (M+1). ¹H NMR (400 MHz, MeOH-d₄) δ ppm 8.01 (d, J=5.6 Hz, 1H), 7.67 (s, 1H), 7.55 (s, 1H), 7.20-7.26 (m, 2H), 7.11-7.20 (m, 2H), 6.97 (s, 1H), 4.16 (t, J=7.0 Hz, 2H), 3.62-3.75 (m, 4H), 3.34 (t, 2H), 2.87-3.02 (m, 4H), 2.04-2.19 (m, 2 H).

### O. 2′-(2-Methyl-3-trifluoromethyl-phenylamino)-6-piperazin-1-yl-[2,4′]bipyridinyl-4-carboxylic acid amide

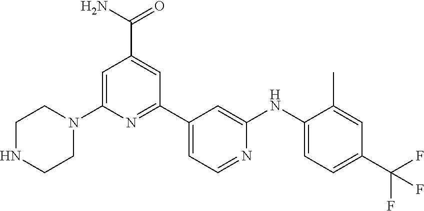

MS (ESI) m/z 457.2 (M+1). ¹H NMR (400 MHz, MeOH-d₄) δ ppm 8.09 (d, J=5.6 Hz, 1H), 7.67 (d, J=7.8 Hz, 1H), 7.58 (s, 1H), 7.49 (d, J=7.7 Hz, 1H), 7.43 (s, 1H), 7.32-7.39 (m, 2H), 7.22 (s, 1H), 3.56-3.71 (m, 4H), 2.86-2.97 (m, 4H), 2.39 (s, 3H).

### P. 2′-(4-Chloro-2-fluorophenylamino)-6-piperazin-1-yl-[2,4′]bipyridinyl-4-carboxylic acid amide

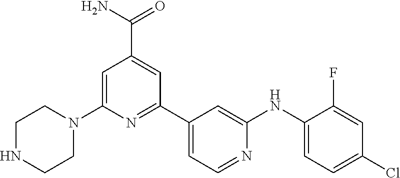

MS (ESI) m/z 427.1 (M+1). ¹H NMR (400 MHz, MeOH-d₄) δ ppm 8.18 (d, J=5.6 Hz, 1H), 8.10 (t, J=8.8 Hz, 1H), 7.63 (s, 1H), 7.59 (s, 1H), 7.43 (dd, J=5.4, 1.5 Hz, 1H), 7.17-7.25 (m, 2H), 7.08-7.17 (m, 1H), 3.62-3.74 (m, 4H), 2.88-3.00 (m, 4H).

### Q. 2′-(4-Chlorophenylamino)-6-piperazin-1-yl-[2,4′]bipyridinyl-4-carboxylic acid amide

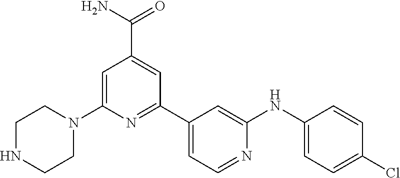

MS (ESI) m/z 409.2 (M+1). ¹H NMR (400 MHz, MeOH-d₄) δ ppm 8.18 (d, J=5.3 Hz, 1H), 7.50-7.64 (m, 4H), 7.38 (dd, J=5.6, 1.5 Hz, 1H), 7.22-7.29 (m, 3H), 3.63-3.78 (m, 4 H), 2.92-3.07 (m, 4H).

### R. 2′-(3-Chlorophenylamino)-6-piperazin-1-yl-[2,4′]bipyridinyl-4-carboxylic acid amide

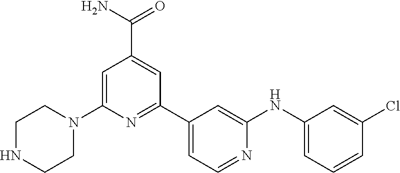

MS (ESI) m/z 409.1 (M+1). ¹H NMR (400 MHz, MeOH-d₄) δ ppm 8.22 (d, J=6.1 Hz, 1H), 7.80 (t, J=2.0 Hz, 1H), 7.56-7.66 (m, 2H), 7.34-7.44 (m, 2H), 7.16-7.27 (m, 2H), 6.83-6.95 (m, 1H), 3.64-3.75 (m, 4H), 2.94-3.05 (m, 4H).

### S. 2′-(2-Fluoro-4-trifluoromethyl-phenylamino)-6-piperazin-1-yl-[2,4′]bipyridinyl-4-carboxylic acid amide

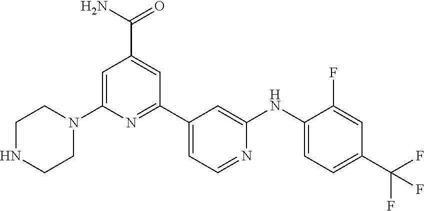

MS (ESI) m/z 461.1 (M+1). ¹H NMR (400 MHz, MeOH-d₄) δ ppm 8.57 (t, J=8.5 Hz, 1H), 8.28 (d, J=5.6 Hz, 1H), 7.78 (s, 1H), 7.62 (s, 1H), 7.52 (dd, J=5.4, 1.4 Hz, 1H), 7.34-7.46 (m, 2H), 7.25 (s, 1H), 3.61-3.79 (m, 4H), 2.89-3.02 (m, 4H).

### T. 6-piperazin-1-yl-2′-(piperidin-4-ylamino)-[2,4′]bipyridinyl-4-carboxylic acid amide

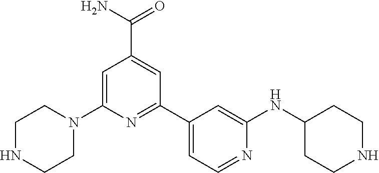

MS (ESI) m/z 382.1 (M+1). ¹H NMR (400 MHz, MeOH-d₄) δ ppm 7.91 (d, J=5.6 Hz, 1H), 7.45 (s, 1H), 7.16 (s, 1H), 7.12 (s, 1H), 7.08 (dd, J=5.8, 1.5 Hz, 1H), 3.73-3.88 (m, 1 H), 3.54-3.64 (m, 4H), 3.06-3.17 (m, 2H), 2.84-2.91 (m, 4H), 2.71-2.84 (m, 2H), 1.96-2.08 (m, 2H), 1.37-1.53 (m, 2H).

### U. 2′-[2-(3,4-Dichlorophenyl)-ethylamino]-6-piperazin-1-yl-[2,4′]bipyridinyl-4-carboxylic acid amide

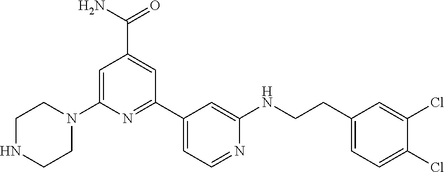

MS (ESI) m/z 471.0 (M+1). ¹H NMR (400 MHz, MeOH-d₄) δ ppm 7.93 (d, J=5.6 Hz, 1H), 7.47 (s, 1H), 7.36 (d, J=2.0 Hz, 1H), 7.33 (d, J=8.3 Hz, 1H), 7.08-7.14 (m, 4H), 3.55-3.63 (m, 4H), 3.51 (t, J=7.2 Hz, 2H), 2.85-2.90 (m, 4H), 2.83 (t, J=7.1 Hz, 2H).

### V. 2′-(4,4-Difluorocyclohexylamino)-6-piperazin-1-yl-[2,4′]bipyridinyl-4-carboxylic acid amide

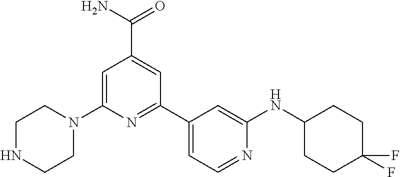

MS (ESI) m/z 417.1 (M+1). ¹H NMR (400 MHz, MeOH-d₄) δ ppm 8.00 (d, J=5.6 Hz, 1H), 7.54 (d, J=0.9 Hz, 1H), 7.25 (s, 1H), 7.21 (d, J=0.9 Hz, 1H), 7.16 (dd, J=5.6, 1.6 Hz, 1H), 3.81-3.95 (m, 1H), 3.64-3.73 (m, 4H), 2.91-3.00 (m, 4H), 2.02-2.17 (m, 4H), 1.85-2.01 (m, 2H), 1.55-1.70 (m, 2H).

### Example 5

### A. 4-[4-tert-Butylcarbamoyl-2′-(3-methoxycarbonyl-2-methylphenylamino)-[2,4′]bipyridinyl-6-yl]-piperazine-1-carboxylic acid tert-butyl ester

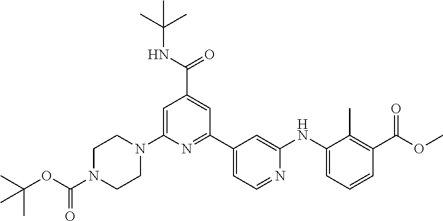

The title compound is prepared in similar method to Example 4E. MS (ESI) m/z 603.4 (M+1). ¹H NMR (400 MHz, CD₂Cl₂) δ ppm 8.26 (d, J=5.3 Hz, 1H), 7.75 (d, J=7.8 Hz, 1H), 7.66 (d, J=7.6 Hz, 1H), 7.28-7.38 (m, 3H), 7.24 (s, 1H), 7.00 (s, 1H), 6.70 (br. s., 1 H), 6.01 (br. s., 1H), 3.93 (s, 3H), 3.61-3.70 (m, 4H), 3.52-3.59 (m, 4H), 2.52 (s, 3H), 1.51 (s, 9H), 1.49 (s, 9H).

### B. 4-[4-tert-Butylcarbamoyl-2′-(3-isopropylcarbamoyl-2-methylphenylamino)-[2,4′]bipyridinyl-6-yl]-piperazine-1-carboxylic acid tert-butyl ester

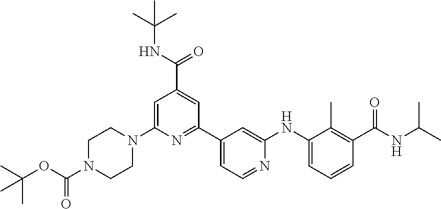

To a solution of toluene (10.0 mL) and AIMe₃ (1.5 mL, 2.99 mmol) is added isopropylamine (0.26 mL, 2.99 mmol). After 5 min 4-[4-tert-butylcarbamoyl-2′-(3-methoxycarbonyl-2-methyl-phenylamino)-[2,4′]bipyridinyl-6-yl]-piperazine-1-carboxylic acid tert-butyl ester (225 mg, 0.37 mmol) is added in toluene (4 mL) and the resulting solution heated to 110° C. After 2 h the solution is carefully diluted with MeOH and the resulting gelatinous suspension is filtered. The filterate is concentrated in vacuo and purified via flash chromatography (SiO₂, EtOAc/heptanes gradient) to give a yellow solid (130 mg, 55%). (ESI) m/z 630.5 (M+1). ¹H NMR (400 MHz, DMSO-d₆) δ ppm 8.35 (s, 1H), 8.10-8.16 (m, 2H), 7.99 (s, 1H), 7.64 (d, J=7.3 Hz, 1H), 7.50 (d, J=12.9 Hz, 2H), 7.34 (dd, 1H), 7.12-7.22 (m, 2H), 6.98 (d, J=7.3 Hz, 1H), 3.96-4.12 (m, 1H), 3.58-3.70 (m, 4H), 3.52-3.48 (m, 4 H), 2.22 (s, 3H), 1.44 (s, 9H), 1.40 (s, 6H), 1.15 (d, J=6.6 Hz, 6H).

### C. 2′-(3-Isopropylcarbamoyl-2-methylphenylamino)-6-piperazin-1-yl-[2,4 ]bipyridinyl-4-carboxylic acid amide

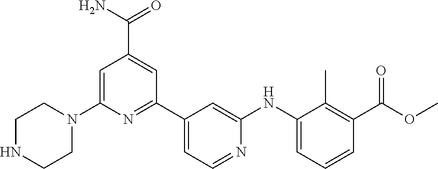

The title compound is prepared in similar method to Example 4F. MS (ESI) m/z 474.3 (M+1). ¹H NMR (400 MHz, MeOH-d₄) δ ppm 8.07 (d, J=5.6 Hz, 1H), 7.55 (s, 1H), 7.40-7.51 (m, 2H), 7.35 (dd, J=5.6, 1.5 Hz, 1H), 7.19-7.29 (m, 2H), 7.08-7.16 (m, 1H), 4.07-4.25 (m, 1H), 3.58-3.72 (m, 4H), 2.87-3.01 (m, 4H), 2.28 (s, 3H), 1.25 (d, J=6.6 Hz, 6H).

### Example 6

### A. 4-(4-Carbamoyl-2′-fluoro-[2,4′]bipyridinyl-6-yl)-piperazine-1-carboxylic acid tert-butyl ester

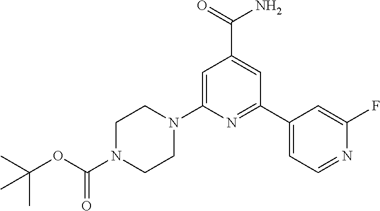

6-(4-tert-Butoxycarbonyl-piperazin-1-yl)-2′-fluoro-[2,4′]-bipyridinyl-4-carboxylic acid methyl ester (435.0 mg, 1.00 mmol) (prepared in similar fashion to Example 4C employing 2-fluoropyridine-4-boronic acid) is dissolved in 7.0M NH₃/MeOH solution (25 mL) and heated at 90° C. in a sealed pressure vessel until reaction is complete. The reaction is concentrated in vacuo and the residue obtained is used without further purification (398.0 mg, 95%). (ESI) m/z 402.1 (M+1). ¹H NMR (400 MHz, CD₃CN) δ ppm 8.30 (d, J=5.3 Hz, 1H), 7.91-7.98 (m, 1H), 7.71 (s, 1H), 7.58 (d, J=1.0 Hz, 1H), 7.21 (d, J=1.0 Hz, 1H), 6.98 (s, 1H), 6.23 (s, 1 H), 3.64-3.76 (m, 4H), 3.48-3.59 (m, 4H), 1.48 (s, 9H).

### B. 4-(4-Carbamoyl-2′-cyclohexylamino-[2,4′]bipyridinyl-6-yl)-piperazine-1-carboxylic acid tert-butyl ester

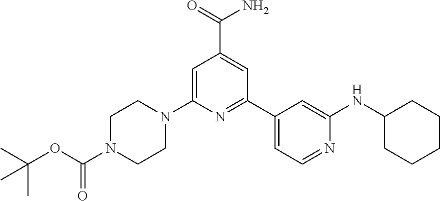

4-(4-Carbamoyl-2′-fluoro-[2,4′]bipyridinyl-6-yl)-piperazine-1-carboxylic acid tert-butyl ester (160.0 mg, 0.39 mmol) is dissolved in neat cyclohexylamine (8 mL) and heated at 130° C. in a sealed pressure vessel until the reaction is complete. The reaction is concentrated in vacuo and the residue purified via semi-preparative HPLC (5-50% CH₃CN/H₂O gradient with 0.1% NH₄OH) to give the title compound (95.0 mg, 50%). (ESI) m/z 481.4 (M+1). ¹H NMR (400 MHz, CD₂Cl₂) δ ppm 8.01 (d, J=5.6 Hz, 1H), 7.53 (s, 1H), 7.18 (s, 1H), 7.02 (dd, J=5.3, 1.3 Hz, 1H), 6.97 (s, 1H), 4.60 (br. s., 1H), 3.55-3.65 (m, 4H), 3.44-3.51 (m, 4H), 1.91-2.05 (m, 2H), 1.63-1.76 (m, 2H), 1.53-1.63 (m, 2H), 1.39 (s, 9H), 1.10-1.25 (m, 4H).

### C. 2′-(1-Ethylpentylamine)-6-piperazin-1-yl-[2,4′]bipyridinyl-4-carboxylic acid amide

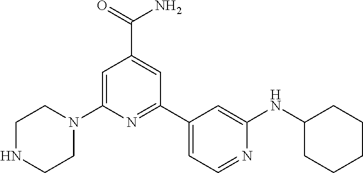

To a solution of 4-(4-carbamoyl-2′-cyclohexylamino-[2,4′]bipyridinyl-6-yl)-piperazine-1-carboxylic acid tert-butyl ester (96.0 mg, 0.20 mmol) and CH₂Cl₂ (5.0 mL) is added TFA (5.0 mL). After stirring for 2 h the solution is concentrated. The residue is taken up in CH₂Cl₂ (10 mL) and washed with a saturated aqueous solution of NaHCO₃. The aqueous layer is further extracted with CH₂Cl₂ (2×10 mL). The combined organic layers are then dried (Na₂SO₄), filtered and concentrated. The residue is then separated via semi-preparative HPLC (10-90% CH₃CN/H₂O gradient with 0.1% NH₄OH) to give the title compound (35.0 mg, 46%). MS (ESI) m/z 381.2 (M+1). ¹H NMR (400 MHz, DMSO-d₆) δ ppm 8.17 (s, 1H), 8.02 (d, J=5.3 Hz, 1H), 7.60 (s, 1H), 7.52 (s, 1H), 7.20 (s, 1H), 7.16 (s, 1H), 7.03 (dd, J=5.4, 1.4 Hz, 1H), 6.47 (d, J=7.8 Hz, 1H), 3.67-3.84 (m, 1H), 3.48-3.63 (m, 4H), 2.76-2.92 (m, 4H), 1.93 (dd, J=11.7, 2.4 Hz, 2H), 1.67-1.80 (m, 2H), 1.53-1.66 (m, 1H), 1.10-1.41 (m, 5H).

Compound D of Example 6 can be prepared by a similar method as those above.

### D. 2′-Cyclopentylamino-6-piperazin-1-yl-[2,4′]bipyridinyl-4-carboxylic acid amide

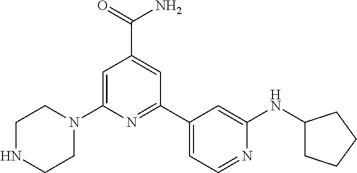

MS (ESI) m/z 367.2 (M+1). ¹H NMR (400 MHz, DMSO-d₆) δ ppm 8.18 (s, 1H), 8.04 (d, J=5.6 Hz, 1H), 7.61 (s, 1H), 7.54 (s, 1H), 7.22 (s, 1H), 7.15 (s, 1H), 7.05 (dd, J=5.3, 1.5 Hz, 1H), 6.58 (d, J=6.8 Hz, 1H), 4.09-4.24 (m, 1H), 3.52-3.65 (m, 4H), 2.80-2.98 (m, 4H), 1.85-1.98 (m, 2H), 1.63-1.78 (m, 2H), 1.37-1.62 (m, 4H).

### E. 2′-Amino-6-piperazin-1-yl-[2,4′]bipyridinyl-4-carboxylic acid amide

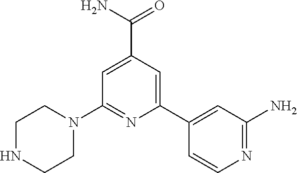

To a solution of 4-[4-carbamoyl-2′-fluoro-[2,4′]bipyridinyl-6-yl]-piperazine-1-carboxylic acid tert-butyl ester (100 mg, 0.249 mmol) in 1-methylpyrrolidin-2-one is added 3,4-dimethoxybenzylamine (0.188 ml, 1.25 mmol) and DIPEA (0.131 ml, 0.747 mmol). The reaction mixture is stirred at 5 days at 120° C. The mixture is evaporated and the residue is purified by preparative reverse phase HPLC (Gilson) to yield the title compound (100 mg, 0.182 mmol) as a yellow powder. MS (ESI) m/z 549.3 (M+1).

To a solution of 4-[4-carbamoyl-2′-(3,4-dimethoxybenzylamino)-[2,4′]bipyridinyl-6-yl]-piperazine-1-carboxylic acid tert-butyl ester (100 mg, 0.182 mmol) in CH₂Cl₂ is added thioanisole (0.643 ml, 5.47 mmol) and trifluoroacetic acid (2.3 ml). The reaction mixture is stirred at rt for 16 h. The mixture is evaporated and the residue is purified by preparative reverse phase HPLC (Gilson) to yield the title compound (10 mg, 0.024 mmol, 2×TFA salt) as a yellow powder. ¹H-NMR (400 MHz, DMSO-d₆, 298 K): δ ppm 3.24-3.28 (m, 4H) 3.86-3.93 (m, 4H) 7.49 (d, 1H) 7.67 (s, 1H) 7.80 (s, 1H) 7.84-7.96 (m, 2H) 8.06 (d, J=6.85 Hz, 1H) 8.27 (s, 1H) 8.90 (s, 2H). MS (ESI) m/z 299.1 (M+1).

### F. 2′-Benzylamino-6-piperazin-1-yl-[2,4′]bipyridinyl-4-carboxylic acid amide

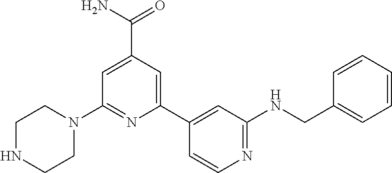

A solution of 4-[4-carbamoyl-2′-fluoro-[2,4′]bipyridinyl-6-yl]-piperazine-1-carboxylic acid tert-butyl ester (80 mg, 0.199 mmol), benzylamine (109 μL, 0.996 mmol) and ethyldiisopropylamine (104 μL, 0.598 mmol) in 1-methylpyrrolidin-2-one (1 mL) is heated to 120° C. in a sealed tube for 3.5 days. The reaction mixture is cooled to rt, the solvent removed by distillation under vacuum. The crude residue is purified by preparative reverse phase HPLC (Gilson) to yield the BOC-protected intermediate 4-{4-carbamoyl-2′-[2-(4-hydroxyphenyl)ethylamino]-[2,4′]bipyridinyl-6-yl}-piperazine-1-carboxylic acid tert-butyl ester which is directly dissolved in CH₂Cl₂ (4 mL) and trifluoroacetic acid (2 mL, 23.9 mmol) is added at 0° C. The reaction mixture is stirred for 10 minutes at 0° C. and for 1 h at rt. The solvent and the excess of trifluoroacetic acid are evaporated and the crude residue is purified by preparative reverse phase HPLC (Gilson) to yield the title compound (29 mg, 0.047 mmol, 2×TFA salt) as a yellow lyophilized powder. ¹H-NMR (400 MHz, MeOD, 298 K): δ ppm 3.35-3.40 (m, 4H), 3.97-4.01 (m, 4H), 4.69 (s, 2H), 7.39 (d, J=6.60 Hz, 1H), 7.42-7.48 (m, 4H), 7.51 (s, 1H), 7.59 (d, J=6.85 Hz, 1H), 7.80-7.84 (m, 2H), 7.97 (d, J=6.85, 1H). MS (ESI) 389.1 (M+1).

### G. 2′-(2-Chlorobenzylamino-6-piperazin-1-yl-[2,4′]bipyridinyl-4-carboxylic acid amide

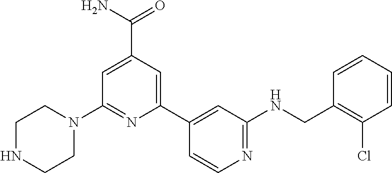

A solution of 4-[4-carbamoyl-2′-fluoro-[2,4′]bipyridinyl-6-yl]-piperazine-1-carboxylic acid tert-butyl ester (80 mg, 0.199 mmol), 2-chlorobenzylamine (174 μL, 1.39 mmol) and ethyldiisopropylamine (104 μL, 0.598 mmol) in 1-methyl-pyrrolidin-2-one (1 mL) is heated to 120° C. in a sealed tube for 5 days. The reaction mixture is cooled to rt. and the solvent is removed by distillation under vacuum. The crude residue is purified by preparative reverse phase HPLC (Gilson) to yield the BOC-protected intermediate 4-{4-carbamoyl-2′-[2-(4-hydroxy-phenyl)ethylamino]-[2,4′]bipyridinyl-6-yl}-piperazine-1-carboxylic acid tert-butyl ester which is directly dissolved in CH₂Cl₂ (4 mL) and trifluoroacetic acid (2 mL, 23.9 mmol) is added at 0° C. The reaction mixture is stirred for 10 minutes at 0° C. and for 1 h at rt. The solvent and the excess of trifluoroacetic acid are evaporated and the crude residue is purified by preparative reverse phase HPLC (Gilson) to yield the title compound (10 mg, 0.015 mmol, 2×TFA salt) as a yellow lyophilized powder. ¹H-NMR (400 MHz, MeOD, 298 K): δ ppm 3.23-3.29 (m, 4H), 3.83-3.90 (m, 4H), 4.67 (s, 2H), 7.24-7.31 (m, 2H), 7.38-7.49 (m, 4H), 7.65 (s, 1H), 7.72 (s, 1H), 7.88 (d, J=6.60 Hz, 1H). MS (ESI) m/z 423.3 (M+1).

### H. 2′-[2-(4-Hydroxyphenyl)ethylamino]-6-piperazin-1-yl-[2,4′]bipyridinyl-4-carboxylic acid amide

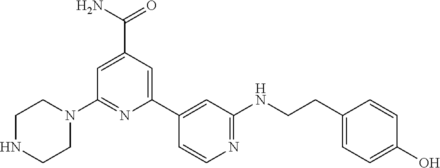

A solution of 4-[4-carbamoyl-2′-fluoro-[2,4′]bipyridinyl-6-yl]-piperazine-1-carboxylic acid tert-butyl ester (80 mg, 0.199 mmol), tyramine (137 mg, 0.996 mmol) and ethyldiisopropylamine (104 μL, 0.598 mmol) in 1-methylpyrrolidin-2-one (1 mL) is heated to 120° C. in a sealed tube for 2 days. The reaction mixture is cooled to rt, the solvent removed by distillation under vacuum. The crude BOC-protected intermediate 4-{4-carbamoyl-2′-[2-(4-hydroxyphenyl)ethylamino]-[2,4′]bipyridinyl-6-yl}piperazine-1-carboxylic acid tert-butyl ester is directly dissolved in CH₂Cl₂ (4 mL) and trifluoroacetic acid (1.83 mL, 23.9 mmol) is added at 0° C. The reaction mixture is stirred for 10 minutes at 0° C. and for 1 h at rt. The solvent and the excess of trifluoroacetic acid are evaporated and the crude residue is purified by preparative reverse phase HPLC (Gilson) to yield the title compound (10 mg, 0.015 mmol, 2×TFA salt) as a yellow lyophilized powder. ¹H-NMR (400 MHz, DMSO-d₆, 298 K): δ ppm 2.83 (s, 2H), 3.26 (s, 4H), 3.89 (s, 4H), 6.71 (d, J=3.91 Hz, 2H), 7.11 (d, J=4.16 Hz, 2H), 7.48 (bs, 2H), 7.70 (s, 1H), 7.80 (s, 2H), 8.02 (s, 1H), 8.27 (s, 1H), 8.96 (s, 2H), 9.27 (s, 1H). MS (ESI) m/z 419.3 (M+1).

### I. 2′-(1-Methyl-1H-pyrazol-3-ylamino)-6-piperazin-1-yl-[2,4′]bipyridinyl-4-carboxylic acid amide

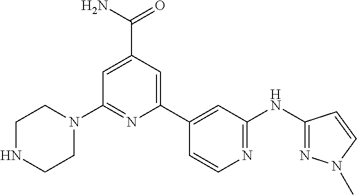

A solution of NaHMDS (0.5 mL, 0.48 mmol, 1.0M THF) is added to a solution of 3-amino-1-methylpyrazole in THF (3 mL) at ambient temperature before adding 4-(4-carbamoyl-2′-fluoro-[2,4′]bipyridinyl-6-yl)-piperazine-1-carboxylic acid tert-butyl ester (50 mg, 0.12 mmol). The reaction mixture is sealed and heated to 80° C. for 3 h. Reaction is quenched with iPrOH and concentrated in vacuo. The residue is purified by flash chromatography (2 to 10% MeOH/DCM) to afford 4-(4-Carbamoyl-2′-(2-methyl-2H-pyrazol-3-yl)-piperazine-1-carboxylic acid tert-butyl ester. MS (ESI) m/z 479.3 (M+1).

4-(4-Carbamoyl-2′-(2-methyl-2H-pyrazol-3-yl)-piperazine-1-carboxylic acid tert-butyl ester (175 mg, 0.37 mmol) and TFA (5 mL) are stirred in DCM (5 mL) at 25° C. for 2 h. After stirring for 2 h the solution is concentrated. The residue is then separated via semi-preparative HPLC (10-90% CH₃CN/H₂O gradient with 0.1% NH₄OH) to give 2′-(1-Methyl-1H-pyrazol-3-ylamino)-6-piperazin-1-yl-[2,4′]bipyridinyl-4-carboxylic acid amide. MS (ESI) m/z 379.2 (M+1). ¹H NMR (400 MHz, DMSO-d₆) ppm 8.94 (br. s., 2H), 8.20-8.37 (m, 2H), 8.12 (s, 1H), 7.80 (s, 1H), 7.76 (s, 1H), 7.71 (s, 1H), 7.57 (d, J=5.4 Hz, 1H), 7.46 (s, 1H), 6.24 (d, J=2.1 Hz, 1H), 3.88-3.98 (m, 4H), 3.85 (s, 3H), 3.28 (br. s., 4H).

### Example 7

### A. Cyclohexyl-(4-trimethylstannanylpyridin-2-yl)amine

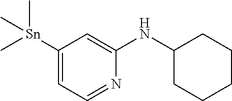

The title compound is prepared from 2-fluoro-4-iodopyridine (4.0 g, 17.9 mmol) and cyclohexylamine (5.1 mL, 44.8 mmol). The two reaction components are sealed in a pressure vessel and heated to 120° C. for 3 h. After cooling, the reaction is concentrated under reduced pressure. The residue is purified by flash chromatography (10 to 20 to 30% EtOAc/hexanes) to yield 5.1 g of 2-cyclohexylamino-4-iodopyridine.

To a reaction vessel containing 2-cyclohexylamino-4-iodo-pyridine prepared above (4.9 g, 16.2 mmol) dissolved in toluene (175 mL) is added Me₃SnSnMe₃ (7.93 g, 24.2 mmol). The solution is degassed with N₂ for 10 min, Pd(PPh₃)₄ (1.87 g, 1.6 mmol) is added, and the reaction is heated to 100° C. on. Upon cooling, the reaction is filtered over Celite®, concentrated under reduced pressure, and partitioned between EtOAc and a saturated aqueous solution of KF. The separated organic phase is washed with a saturated aqueous solution of NaCl, dried (Na₂SO₄), and concentrated in vacuo. The residue is purified by flash chromatography (10 to 25 to 30% EtOAc/hexanes) to afford the product (3.8 g, 69%) as a white solid: ¹H NMR (400 MHz, CDCl₃) δ ppm 0.29 (s, 7.54H), 0.29 (d, J=55.7 Hz, 0.77H), 0.29 (d, J=53.3 Hz, 0.69H), 1.14-1.31 (m, 3H), 1.35-1.50 (m, 2H), 1.60-1.66 (m, 1H), 1.71-1.80 (m, 2H), 1.99-2.09 (m, 2H), 3.55-3.68 (m, 1H), 4.25-4.34 (d, J=7.7 Hz, 1H), 6.45 (s, 0.84H), 6.45 (d, J=49.6 Hz, 0.16 Hz), 6.61 (d, J=4.8 Hz, 0.84H), 6.61 (dd, J=39.8, 4.8 Hz, 0.16H), 7.96-8.03 (m, 1H).

### B. (Tetrahydropyran-4-yl)-(4-trimethylstannanylpyridin-2-yl)amine

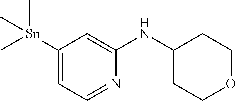

The title compound is prepared from 4-aminotetrahydropyran by analogy to cyclohexyl-(4-trimethylstannanylpyridin-2-yl)amine Example 7A. ¹H NMR (400 MHz, DMSO-d₆) δ ppm 0.23 (d, J=56.6 Hz, 0.8H), 0.23 (s, 7.5H), 0.23 (d, J=54.1 Hz, 0.7H), 1.32-1.44 (m, 2H), 1.83 (dd, J=12.6, 2.5 Hz, 2H), 3.34-3.42 (m, 2H), 3.81-3.87 (m, 2H), 3.87-3.95 (m, 1H), 6.30 (d, J=7.6 Hz, 1H), 6.45-6.55 (m, 0.15H), 6.51 (dd, J=4.8, 0.8 Hz, 0.85H), 6.45-6.64 (m, 0.08H), 6.57 (t, J=0.8 Hz, 0.85H), 6.45-6.64 (m, 0.07H), 7.81-7.88 (m, 0.08H), 7.84 (dd, J=4.8, 0.8 Hz, 0.85H), 7.81-7.88 (m, 0.07H).

### C. 2,6-Dichloro-4-difluoromethylpyridine

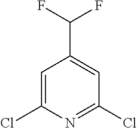

To a solution of commercially available 2,6-dichloropyridine-4-carbaldehyde (0.3 g, 1.7 mmol) in CH₂Cl₂ (34 mL) under N₂ at −78° C. is added DAST (0.67 mL, 5.1 mmol). The reaction is allowed to warm to room temperature, stirred 1 h, and poured into cold water. The separated aqueous layer is extracted with fresh CH₂Cl₂. The combined organic layers are dried (Na₂SO₄), concentrated, and purified by flash chromatography (10% EtOAc/heptanes) to yield an orange oil: ¹H NMR (400 MHz, CDCl₃) δ ppm 6.60 (t, J=55.1 Hz, 1H), 7.40 (s, 2H).

### D. 3,5-Dichloro-6-methyl-[1,4]oxazin-2-one

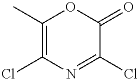

To a 0° C. solution of oxalyl chloride (179 g, 1.41 mol) in toluene (303 mL) under nitrogen in a 3-necked round-bottom flask, is added a solution of DL-lactonitrile (25.0 g, 352 mmol) in toluene (118 mL) dropwise over 20 min. The reaction is stirred at 0° C. for 50 minutes, then placed in a 70° C. oil bath, which is warmed to about 95° C. Triethylamine hydrochloride (16.8 g, 176 mmol) is added very carefully, as the reaction is prone to exotherm. After the addition, the reaction is stirred at 100° C. for 18 h. The solvents are then removed by rotary evaporation over a 60° C. water bath. Diethyl ether (about 2 L) is used to extract desired product and contaminants from the crude solid. Additional diethyl ether (2 L) is used to extract nearly pure desired product from the solid. The two solutions are concentrated down to dryness separately by rotary evaporation. The contaminated product is cooled to 0° C. until a yellow solid is formed which is isolated by decanting the dark red oil. This yellow solid is combined with that obtained from the second ether extraction (45.5 g, 253 mmol, 72%). ¹H NMR (400 MHz, DMSO-d₆) δ ppm 2.30 (s, 3H).

### E. 2,6-Dichloro-3-methyl-isonicotinic acid ethyl ester

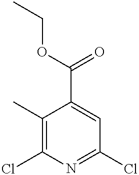

A mixture of 3,5-dichloro-6-methyl-[1,4]oxazin-2-one (45.5 g, 253 mmol) and ethyl propiolate (74.4 g, 758 mmol) in toluene (135 mL) is stirred under nitrogen at 80° C. for 23 h. The reaction is then cooled down to room temperature and the solvents are removed by rotary evaporation. The residue is treated with hexanes (400 mL), and the somewhat cloudy solution is decanted from a dark red residue containing undesired impurities. The hexanes are removed by rotary evaporation. The crude is then cooled to 0° C., and the flask is swirled occasionally until the crude oil solidifies. The solid is then washed with small amounts of pentane. The cooled filtrate is filtered again, resulting in removal of additional yellow solid, which is the undesired regioisomer (16 g). The filtrate is then concentrated down to dryness by rotary evaporation and purified by silica gel chromatography (9:1 heptane/dichloromethane) to yield a clear liquid which solidifies upon standing (15.4 g, 65.8 mmol, 26%). MS (ESI) m/z 234.16 (M+1). ¹H NMR (400 MHz, CDCl₃) δ ppm 7.55 (s, 1H), 4.39 (q, J=7.07 Hz, 2H), 2.53 (s, 3H), 1.39 (t, J=7.14 Hz, 3H).

### F. N-tert-Butyl-2,6-dichloroisonicotinamide

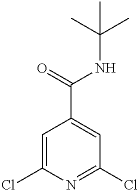

To toluene (48 mL) at 0° C. is added a solution of Me₃Al (19 mL of 2.0 M in hexanes, 38.8 mmol), followed by dropwise addition of tert-butylamine (4.1 mL, 38.8 mmol). The reaction is warmed to rt before 2,6-dichloro-4-methoxycarbonylpyridine (1.0 g, 4.8 mmol) is added. The reaction is heated to 110° C. for 2 h, cooled to 0° C., and quenched by the slow addition of 1 N HCl. After addition of 30 mL of 1 N NaOH, the reaction is extracted three times with CH₂Cl₂. The combined organic phases are washed with brine, dried (Na₂SO₄), and concentrated in vacuo. The residue is purified by flash chromatography (5 to 30% EtOAc/heptanes) to yield 1.1 g (93%) as a white solid: ¹H NMR (400 MHz, CDCl₃) δ ppm 1.46 (s, 9H), 5.83 (s, 1H), 7.51 (s, 2H).

### Example 8

### A. 4-(6-chloro-4-difluoromethylpyridin-2-yl)piperazine-1-carboxylic acid tert-butyl ester

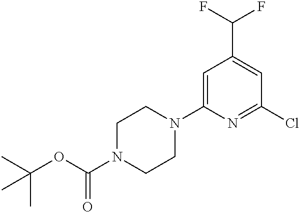

A solution of 2,6-dichloro-4-difluoromethylpyridine (0.1 g, 0.5 mmol), piperazine-1-carboxylic acid tert-butyl ester (0.94 g, 0.5 mmol), Et₃N (0.28 mL, 2.0 mmol) in dioxane (3 mL) is heated in a sealed tube at 90° C. for 48 h. After cooling, the reaction is concentrated in vacuo. The residue is purified by flash chromatography (10 to 30% EtOAc/heptanes) to give the title compound as a clear oil: ¹H NMR (400 MHz, CDCl₃) δ ppm 1.48 (s, 9H), 3.49-3.63 (m, 8H), 6.47 (t, J=55.7 Hz, 1H), 6.56 (s, 1H), 6.72 (s, 1H).

### B. 4-(6-chloro-4-trifluoromethylpyridin-2-yl)piperazine-1-carboxylic acid tert-butyl ester

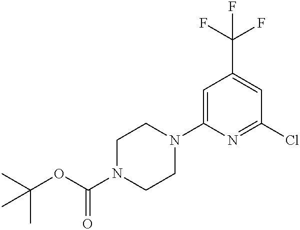

Prepared from commercial 2,6-dichloro-4-trifluoromethylpyridine by analogy to the method described above for the preparation of 4-(6-chloro-4-difluoromethylpyridin-2-yl)piperazine-1-carboxylic acid tert-butyl ester. ¹H NMR (400 MHz, CDCl₃) δ ppm 1.49 (s, 9H), 3.51-3.64 (m, 8H), 6.64 (s, 1H), 6.79 (s, 1H).

### C. 4-(6-Chloro-4-methoxycarbonyl-pyridin-2-yl)-piperazine-1-carboxylic acid tert-butyl ester

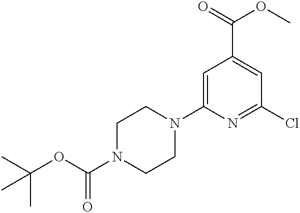

The title compound is prepared from commercial 2,6-dichloro-4-methoxycarbonyl pyridine by analogy to the method described above for the preparation of 4-(6-chloro-4-difluoromethylpyridin-2-yl)piperazine-1-carboxylic acid tert-butyl ester. ¹H NMR (400 MHz, MeOD) δ ppm 1.48 (s, 9H), 3.50-3.57 (m, 4H), 3.57-3.64 (m, 4H), 3.91 (s, 3H), 7.07 (d, J=0.9 Hz, 2H), 7.20 (d, J=0.9 Hz, 2H).

### D. (S)-4-(6-Chloro-4-methoxycarbonylpyridin-2-yl)-2-methyl-piperazine-1-carboxylic acid tert-butyl ester

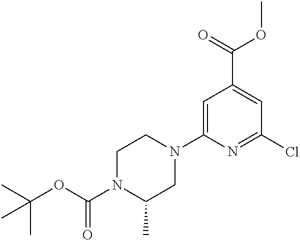

The title compound is prepared from commercial 2,6-dichloro-4-methoxycarbonyl pyridine by analogy to the method described above for the preparation of 4-(6-chloro-4-difluoromethylpyridin-2-yl)piperazine-1-carboxylic acid tert-butyl ester. ¹H NMR (400 MHz, CDCl₃) δ ppm 1.17 (d, J=6.7 Hz, 3H), 1.48 (s, 9H), 3.01-3.11 (m, 1H), 3.20-3.35 (m, 2 H), 3.89-4.03 (m, 5H), 4.10-4.18 (m, 1H), 4.27-4.38 (m, 1H), 7.04 (d, J=0.9 Hz, 1H), 7.10 (d, J=0.9 Hz, 1H).

### E. (R)-4-(6-Chloro-4-methoxycarbonylpyridin-2-yl)-2-methyl-piperazine-1-carboxylic acid tert-butyl ester

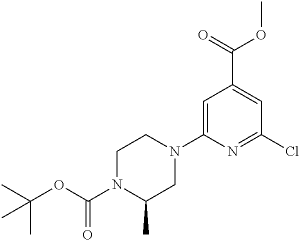

The title compound is prepared from commercial 2,6-dichloro-4-methoxycarbonyl pyridine by analogy to the method described above for the preparation of 4-(6-chloro-4-difluoromethylpyridin-2-yl)piperazine-1-carboxylic acid tert-butyl ester. ¹H NMR (400 MHz, MeOD) δ ppm 1.17 (d, J=6.8 Hz, 3H), 1.47 (s, 9H), 2.99-3.09 (m, 1H), 3.21-3.28 (m, 2 H), 3.86-3.95 (m, 4H), 4.06-4.21 (m, 2H), 4.27-4.35 (m, 1H), 7.04 (s, 1H), 7.17 (s, 1 H).

### F. 2-Chloro-6-(3,3-dimethylpiperazin-1-yl)isonicotinic acid methyl ester

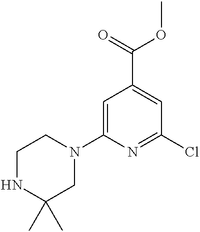

The title compound is prepared from commercial 2,6-dichloro-4-methoxycarbonyl pyridine by analogy to the method described above for the preparation of 4-(6-chloro-4-difluoromethylpyridin-2-yl)piperazine-1-carboxylic acid tert-butyl ester. ¹H NMR (400 MHz, CDCl₃) δ ppm 1.17 (s, 6H), 2.98-3.04 (m, 2H), 3.35 (s, 2H), 3.53-3.59 (m, 2H), 3.92 (s, 3H), 7.05 (s, 2H).

### G. 7-(6-Chloro-4-methoxycarbonylpyridin-2-yl)-4,7-diaza-spiro[2.5]octane

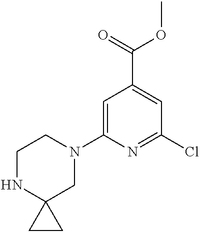

The title compound is prepared from commercial 2,6-dichloro-4-methoxycarbonyl pyridine by analogy to the method described above for the preparation of 4-(6-chloro-4-difluoromethylpyridin-2-yl)piperazine-1-carboxylic acid tert-butyl ester. ¹H NMR (400 MHz, CDCl₃) δ ppm 0.60-0.68 (m, 4H), 3.03-3.08 (m, 2H), 3.43 (s, 2H), 3.55-3.59 (m, 2H), 7.01-7.03 (m, 1H), 7.06-7.09 (m, 1H).

### H. 2-Chloro-6-morpholin-4-yl-isonicotinic acid methyl ester

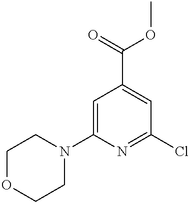

The title compound is prepared from commercial 2,6-dichloro-4-methoxycarbonyl pyridine by analogy to the method described above for the preparation of 4-(6-chloro-4-difluoromethylpyridin-2-yl)piperazine-1-carboxylic acid tert-butyl ester. ¹H NMR (400 MHz, CDCl₃) δ ppm 3.55-3.60 (m, 4H), 3.77-3.82 (m, 4H), 3.92 (s, 3H), 7.07 (d, J=0.9 Hz, 1H), 7.14 (d, J=0.9 Hz, 1H).

### I. 2-Chloro-6-[(2-hydroxyethyl)methylamino]isonicotinic acid methyl ester

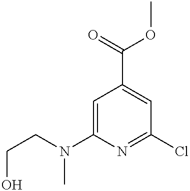

The title compound is prepared from commercial 2,6-dichloro-4-methoxycarbonyl pyridine by analogy to the method described above for the preparation of 4-(6-chloro-4-difluoromethylpyridin-2-yl)piperazine-1-carboxylic acid tert-butyl ester. ¹H NMR (400 MHz, CDCl₃) δ ppm 2.77-2.85 (m, 1H), 3.13 (s, 3H), 3.73-3.79 (m, 2H), 3.85-3.90 (m, 2H), 3.92 (s, 3H), 7.00 (d, J=1.0 Hz, 1H), 7.07 (d, J=1.0 Hz, 1H).

### J. 6′-Chloro-3,4,5,6-tetrahydro-2H-[1,2′]bipyridinyl-4,4′-dicarboxylic acid 4-tert-butyl ester 4′-methyl ester

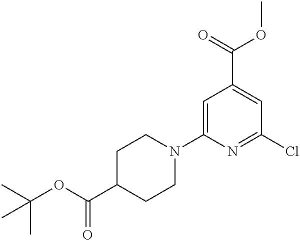

The title compound is prepared from commercial 2,6-dichloro-4-methoxycarbonyl pyridine by analogy to the method described above for the preparation of 4-(6-chloro-4-difluoromethylpyridin-2-yl)piperazine-1-carboxylic acid tert-butyl ester. ¹H NMR (400 MHz, MeOD) δ ppm 1.45 (s, 9H), 1.56-1.69 (m, 2H), 1.88-2.01 (m, 2H), 2.47-2.57 (m, 1H), 3.00-3.10 (m, 2H), 3.91 (s, 3H), 4.21-4.29 (m, 2H), 7.00 (s, 1H), 7.18 (s, 1H).

### K. 4-(6-Chloro-4-ethoxycarbonyl-5-methylpyridin-2-yl)piperazine-1-carboxylic acid tert-butyl ester and 4-(6-chloro-4-ethoxycarbonyl-3-methylpyridin-2-yl)piperazine-1-carboxylic acid tert-butyl ester

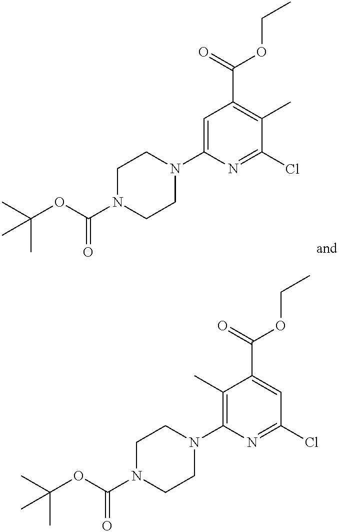

The title compounds are prepared from 2,6-dichloro-3-methylisonicotinic acid ethyl ester above and piperazine-1-carboxylic acid tert-butyl ester by analogy to the method described above for the preparation of 4-(6-chloro-4-difluoromethylpyridin-2-yl)piperazine-1-carboxylic acid tert-butyl ester. ¹H NMR (400 MHz, CDCl₃) δ ppm 1.39 (t, J=7.2 Hz, 3H), 1.48 (s, 9H), 2.39 (s, 3H), 3.10-3.14 (m, 2.5H), 3.48-3.60 (m, 5.5H), 4.32-4.42 (m, 2H), 6.82 (s, 0.35H), 7.28 (s, 0.65H).

### Example 9

### A. 4-(4-Carboxy-6-chloropyridin-2-yl)piperazine-1-carboxylic acid tert-butyl ester

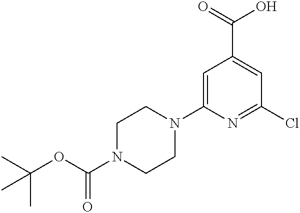

4-(6-Chloro-4-methoxycarbonyl-pyridin-2-yl)-piperazine-1-carboxylic acid tert-butyl ester prepared above (8 g, 22.5 mmol), LiOH.H₂O (1.9 g, 45.1 mmol) in THF (200 mL) and water (100 mL) are stirred at rt for 2 h. The THF is removed under reduced pressure and the pH of the residue is adjusted to 4 with concentrated aqueous HCl. The resulting solid is isolated by filtration and dried in vacuo to yield the title acid as a white solid. ¹H NMR (400 MHz, MeOD) δ ppm 1.46 (s, 9H), 3.48-3.65 (m, 8H), 7.08 (s, 1H), 7.21 (s, 1H).

### B. 4-(6-Chloro-4-methoxycarbonylaminopyridin-2-yl)piperazine-1-carboxylic acid tert-butyl ester

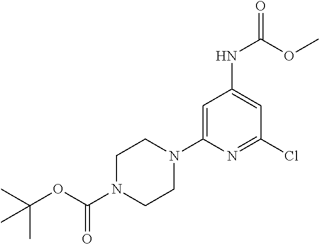

To a solution of 4-(4-carboxy-6-chloropyridin-2-yl)piperazine-1-carboxylic acid tert-butyl ester prepared above (1.0 g, 2.9 mmol) and Et₃N (1.7 mL, 4.1 mmol) in toluene (70 mL) is added DPPA (2.5 mL, 11.6 mmol). The reaction is stirred at rt for 30 min and at reflux for 30 min. To the cooled reaction is added MeOH (10 mL) before reheating to 100° C. for 3 h. The reaction is cooled and concentrated to ½ volume under reduced pressure and diluted with EtOAc. The organic phase is washed with brine, washed with a saturated aqueous solution of NaHCO₃, dried (Na₂SO₄), and concentrated under reduced pressure. Purification by flash chromatography (20 to 60% EtOAc/heptanes affords the title compound as a white solid. ¹H NMR (400 MHz, MeOD) δ ppm 1.47 (s, 9H), 3.45-3.56 (m, 9H), 3.75 (s, 3H), 6.78 (d, J=1.4 Hz, 1H), 6.81 (d, J=1.4 Hz, 1H).

### C. 4-[6-Chloro-4-(3-phenylureido)pyridin-2-yl]piperazine-1-carboxylic acid tert-butyl ester

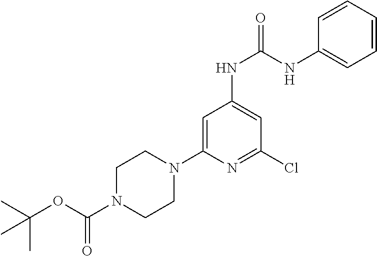

By analogy to the preparation of 4-(6-chloro-4-methoxycarbonylaminopyridin-2-yl)piperazine-1-carboxylic acid tert-butyl ester above, the isocyanate derived from 4-(4-carboxy-6-chloropyridin-2-yl)piperazine-1-carboxylic acid tert-butyl ester prepared above is trapped by aniline to afford the title compound as a white solid. ¹H NMR (400 MHz, CDCl₃) δ ppm 1.48 (s, 9H), 3.50 (s, 8H), 6.43 (d, J=1.5 Hz, 1H), 6.74 (br. s., 1H), 6.86 (br. s., 1H), 6.94 (d, J=1.5 Hz, 1H), 7.16-7.22 (m, 1H), 7.30-7.41 (m, 4H).

### Example 10

### A. 6-(4-tert-Butoxycarbonylpiperazin-1-yl)-2′-cyclohexylamino-[2,4′]bipyridinyl-4-carboxylic acid methyl ester

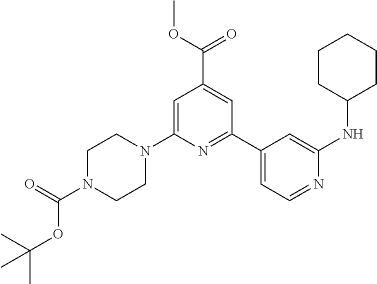

To an argon-degassed mixture of 4-(6-chloro-4-methoxycarbonyl-pyridin-2-yl)-piperazine-1-carboxylic acid tert-butyl ester (1.03 g, 2.88 mmol), cyclohexyl-(4-trimethylstannanyl-pyridin-2-yl)-amine (1.17 g, 3.46 mmol), and cesium fluoride (1.01 g, 6.63 mmol) in dioxane (100 mL) is added bis(tri-tert-butylphosphine)palladium(0) (0.118 g, 0.231 mmol). The reaction mixture is stirred at 100° C. for 20 h. The room temperature reaction mixture is then filtered through celite and concentrated to dryness. The filtrate residue is purified by silica gel chromatography (dichloromethane, then 98:2 dichloromethane/methanol). The fractions containing desired product and contaminants are concentrated to dryness by rotary evaporation and then treated with a mixture of petroleum ether and a very small amount of diethyl ether (less than 10 mL of the solvent mix). A yellow powder is isolated by filtration. The procedure is repeated on the filtrate after concentrating to dryness to yield additional yellow powder (1.12 g, 2.25 mmol, 78%). MS (ESI) m/z 496.26 (M+1). ¹H NMR (400 MHz, DMSO-d₆) δ ppm 8.03 (d, J=5.30 Hz, 1H), 7.52 (d, J=0.76 Hz, 1H), 7.27 (d, J=0.76 Hz, 1H), 7.19-7.15 (m, 1H), 7.03 (dd, J=5.43, 1.64 Hz, 1H), 6.49 (d, J=7.58 Hz, 1H), 3.90 (s, 3H), 3.80-3.70 (m, 1H), 3.66 (dd, J=6.44, 4.17 Hz, 4H), 3.48 (dd, J=6.44, 3.16 Hz, 4H), 1.97-1.88 (m, 2H), 1.77-1.67 (m, 2H), 1.63-1.55 (m, 1H), 1.43 (s, 9H), 1.40-1.26 (m, 2H), 1.26-1.13 (m, 3H).

### B. 6-(4-tert-Butoxycarbonylpiperazin-1-yl)-2′-cyclohexylamino-[2,4′]bipyridinyl-4-difluoromethane

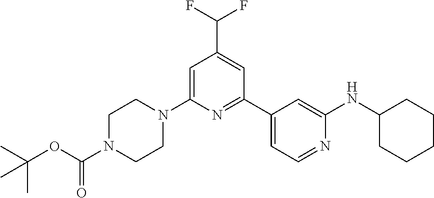

A solution of 4-(6-chloro-4-difluoromethylpyridin-2-yl)piperazine-1-carboxylic acid tert-butyl ester (0.06 g, 0.17 mmol), cyclohexyl-(4-trimethylstannanylpyridin-2-yl)amine (0.065 g, 0.19 mmol) in dioxane (4 mL) is degassed with N₂. CsF (0.059 g, 0.39 mmol) and Pd(P(t-Bu)₃)₂ are added. The reaction is heated under N₂ to 100° C. for 5 h. The reaction is cooled to rt, filtered through Celite®, rinsed with fresh dioxane, and concentrated. The residue is purified by flash chromatography (10-50% EtOAc/heptanes) to give the title compound as a white foam ¹H NMR (400 MHz, CDCl₃) δ ppm 1.18-1.32 (m, 2H), 1.36-1.53 (m, 11H), 1.59-1.71 (m, 2H), 1.73-1.83 (m, 2H), 2.05-2.14 (m, 2H), 3.53-3.73 (m, 9H), 4.50-4.58 (m, 1H), 6.59 (t, J=56.0 Hz, 1H), 6.71-6.77 (m, 1H), 6.98 (s, 1H), 7.07 (dd, J=5.3, 1.3 Hz, 1H), 7.15 (s, 1H), 8.14 (d, J=5.3 Hz, 1H).

### C. 6-(4-tert-Butoxycarbonylpiperazin-1-yl)-2′-cyclohexylamino-[2,4′]bipyridinyl-4-trifluoromethane

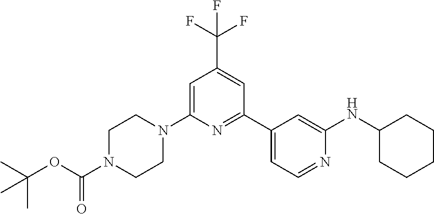

The title compound is prepared from 4-(6-chloro-4-trifluoromethylpyridin-2-yl)piperazine-1-carboxylic acid tert-butyl ester and cyclohexyl-(4-trimethylstannanylpyridin-2-yl)amine by analogy to the Stille coupling method outlined above. ¹H NMR (400 MHz, CDCl₃) δ ppm 1.17-1.32 (m, 3H), 1.38-1.52 (m, 11H), 1.61-1.71 (m, 1H), 1.73-1.84 (m, 2H), 2.05-2.13 (m, 2H), 3.55-3.74 (m, 9H), 4.53 (d, J=8.3 Hz, 1H), 6.81 (s, 1H), 6.96 (s, 1H), 7.06 (dd, J=5.3, 1.5 Hz, 1H), 7.23 (s, 1H), 8.15 (d, J=5.3 Hz, 1H).

### D. 6-(4-tert-Butoxycarbonylpiperazin-1-yl)-2′-(4″-tetrahydropyranyl)-[2,4′]bipyridinyl-4-trifluoromethane

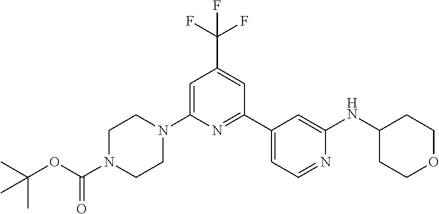

The title compound is prepared from 4-(6-chloro-4-trifluoromethylpyridin-2-yl)piperazine-1-carboxylic acid tert-butyl ester and (tetrahydropyran-4-yl)-(4-trimethylstannanylpyridin-2-yl)amine by analogy to the Stille coupling method outlined above. ¹H NMR (400 MHz, CDCl₃) δ ppm 1.50 (s, 9H), 1.52-1.61 (m, 2H), 2.05-2.13 (m, 2H), 3.56-3.63 (m, 6H), 3.66-3.72 (m, 4H), 3.97-4.06 (m, 3H), 4.42-4.49 (m, 1H), 6.82 (s, 1H), 6.98 (s, 1H), 7.11 (dd, J=5.4, 1.3 Hz, 1H), 7.22 (s, 1H), 8.18 (d, J=5.4 Hz, 1H).

### E. 6-((S)-4-tert-Butoxycarbonyl-3-methyl-piperazin-1-yl)-2′-cyclohexylamino-[2,4′]bipyridinyl-4-carboxylic acid methyl ester

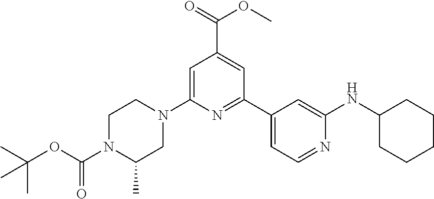

The title compound is prepared from (S)-4-(6-chloro-4-methoxycarbonylpyridin-2-yl)-2-methyl-piperazine-1-carboxylic acid tert-butyl ester by analogy to the Stille coupling method outlined above. MS (ESI) m/z 510.4 (M+1); ¹H NMR (400 MHz, CDCl₃) δ ppm 1.19-1.23 (m, 4H) 1.35-1.53 (m, 12H) 1.61-1.71 (m, 2H) 1.72-1.85 (m, 2H) 2.05-2.15 (m, 2H) 3.04-3.15 (m, 1H) 3.23-3.36 (m, 2H) 3.60-3.73 (m, 1H) 3.94-4.02 (m, 4H) 4.15-4.22 (m, 1H) 4.27-4.42 (m, 2H) 4.52-4.57 (m, 1H) 7.02 (s, 1H) 7.11 (dd, J=5.31, 1.26 Hz, 1H) 7.21 (s, 1H) 7.58 (s, 1H) 8.15 (d, J=5.31 Hz, 1H).

### F. 6-((R)-4-tert-Butoxycarbonyl-3-methylpiperazin-1-yl)-2′-cyclohexylamino-[2,4′]bipyridinyl-4-carboxylic acid methyl ester

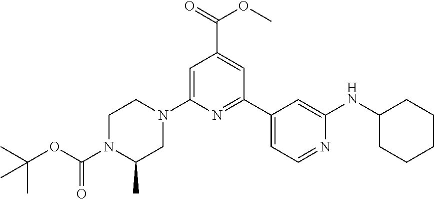

The title compound is prepared from (R)-4-(6-chloro-4-methoxycarbonylpyridin-2-yl)-2-methyl-piperazine-1-carboxylic acid tert-butyl ester by analogy to the Stille coupling method outlined above. ¹H NMR (400 MHz, CDCl₃) δ ppm 1.22 (d, J=6.7 Hz, 3H), 1.36-1.61 (m, 13H), 1.61-1.70 (m, 3H), 1.73-1.83 (m, 2H), 2.05-2.14 (m, 2H), 3.05-3.14 (m, 1H), 3.23-3.36 (m, 2H), 3.62-3.73 (m, 1H), 3.93-4.02 (m, 4H), 4.15-4.21 (m, 1H), 4.26-4.42 (m, 2H), 4.48-4.54 (m, 1H), 7.08-7.13 (m, 1H), 7.20 (s, 1H), 7.57 (s, 1H), 8.15 (d, J=5.3 Hz, 1H).

### Example 11

### A. 6-(3,3-dimethylpiperazin-1-yl)-2′-cyclohexylamino-[2,4′]bipyridinyl-4-carboxylic acid methyl ester

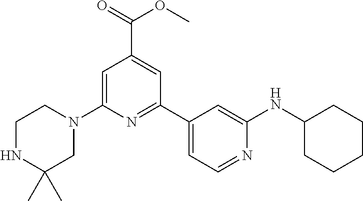

The title compound is prepared from 2-chloro-6-(3,3-dimethylpiperazin-1-yl)isonicotinic acid methyl ester by Stille coupling similar to the method outlined above for the preparation of 6-(4-tert-butoxycarbonylpiperazin-1-yl)-2′-cyclohexylamino-[2,4′]bipyridinyl-4-difluoromethane. 2-Chloro-6-(3,3-dimethylpiperazin-1-yl)isonicotinic acid methyl ester (70 mg, 0.25 mmol) and cyclohexyl-(4-trimethylstannanylpyridin-2-yl)amine (100 mg, 0.3 mmol) in toluene (5 mL) are degassed with N₂ before PdCl₂(PPh₃)₂ (18 mg, 0.025 mmol) is added. The reaction is concentrated under reduced pressure and purified by flash chromatography (2 to 4% MeOH/NH₄OH/CH₂Cl₂) to afford the title compound. ¹H NMR (400 MHz, MeOD) δ ppm 1.16-1.33 (m, 9H), 1.38-1.52 (m, 2H), 1.63-1.72 (m, 1H), 1.75-1.84 (m, 2H), 1.99-2.09 (m, 2H), 2.96-3.03 (m, 2H), 3.50 (s, 2H), 3.61-3.71 (m, 3H), 3.94 (s, 3H), 7.08 (dd, J=5.6, 1.5 Hz, 1H), 7.18-7.21 (m, 1H), 7.30 (s, 1H), 7.56 (s, 1H), 7.97 (d, J=5.7 Hz, 1H).

### B. 2′-Cyclohexylamino-6-(4,7-diazaspiro[2.5]oct-7-yl)-[2,4′]bipyridinyl-4-carboxylic acid methyl ester

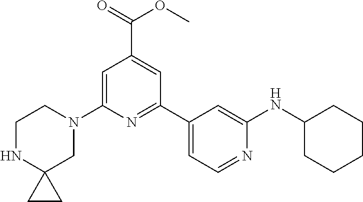

The title compound is prepared from 7-(6-chloro-4-methoxycarbonylpyridin-2-yl)-4,7-diaza-spiro[2.5]octane by analogy to the Stille coupling method outlined above. ¹H NMR (400 MHz, CDCl₃) δ ppm 1.22-1.49 (m, 8H), 1.60-1.85 (m, 4H), 2.05-2.12 (m, 2H), 3.09-3.14 (m, 2H), 3.54 (s, 2H), 3.58-3.71 (m, 3H), 3.88-3.99 (m, 4H), 7.07 (s, 1H), 7.09-7.14 (m, 1H), 7.20-7.23 (m, 1H), 7.56-7.60 (m, 1H), 8.07 (d, J=5.6 Hz, 1H)

### C. 2′-Cyclohexylamino-6-morpholin-4-yl-[2,4′]bipyridinyl-4-carboxylic acid methyl ester

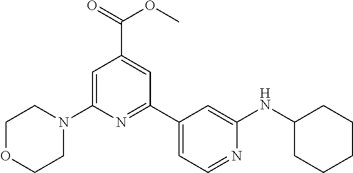

The title compound is prepared from 2-chloro-6-morpholin-4-yl-isonicotinic acid methyl ester by analogy to the Stille coupling method outlined above. ¹H NMR (400 MHz, CDCl₃) δ ppm 1.18-1.34 (m, 2H), 1.36-1.50 (m, 3H), 1.62-1.70 (m, 1H), 1.73-1.82 (m, 2H), 2.04-2.13 (m, 2H), 3.62-3.70 (m, 5H), 3.83-3.89 (m, 4H), 3.96 (s, 3H), 4.49-4.55 (m, 1H), 7.00-7.02 (m, 1H), 7.11 (dd, J=5.4, 1.5 Hz, 1H), 7.24 (d, J=0.9 Hz, 1H), 7.63 (d, J=1.0 Hz, 1H), 8.15 (dd, J=5.3, 0.6 Hz, 1H).

### D. 6-(4-tert-Butoxycarbonylpiperazin-1-yl)-2′-cyclohexylamino-3-methyl-[2,4′]bipyridinyl-4-carboxylic acid ethyl ester

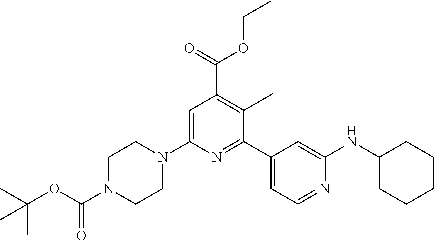

The title compound is prepared from a mixture of 4-(6-chloro-4-ethoxycarbonyl-5-methylpyridin-2-yl)piperazine-1-carboxylic acid tert-butyl ester and 4-(6-chloro-4-ethoxycarbonyl-3-methylpyridin-2-yl)piperazine-1-carboxylic acid tert-butyl ester by analogy to the Stille coupling to cyclohexyl-(4-trimethylstannanylpyridin-2-yl)amine using the method outlined above. The regioisomers are separated by HPLC to yield the title compound as the first eluting isomer. ¹H NMR (400 MHz, CDCl₃) δ ppm 1.16-1.31 (m, 3H), 1.36-1.44 (m, 5 H), 1.48 (s, 9H), 1.61-1.69 (m, 1H), 1.70-1.80 (m, 2H), 2.02-2.10 (m, 2H), 2.30 (s, 3 H), 3.54 (s, 9H), 4.40 (q, J=7.1 Hz, 2H), 4.46-4.52 (m, 1H), 6.42 (s, 1H), 6.61 (dd, J=5.3, 1.3 Hz, 1H), 6.95 (s, 1H), 8.11 (d, J=5.3 Hz, 1H).

### E. 6-(4-tert-Butoxycarbonylpiperazin-1-yl)-2′-cyclohexylamino-5-methyl-[2,4′]bipyridinyl-4-carboxylic acid ethyl ester

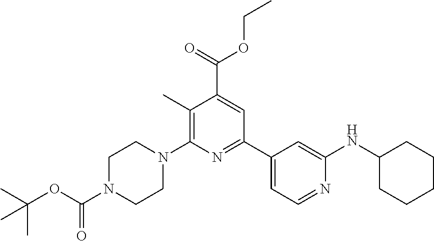

The title compound is prepared from a mixture of 4-(6-chloro-4-ethoxycarbonyl-5-methylpyridin-2-yl)piperazine-1-carboxylic acid tert-butyl ester and 4-(6-chloro-4-ethoxycarbonyl-3-methylpyridin-2-yl)piperazine-1-carboxylic acid tert-butyl ester by analogy to the Stille coupling to cyclohexyl-(4-trimethylstannanylpyridin-2-yl)amine using the method outlined above. The regioisomers are separated by HPLC to yield the title compound as the second eluting isomer. ¹H NMR (400 MHz, CDCl₃) δ ppm 1.19-1.33 (m, 3H), 1.37-1.47 (m, 5H), 1.50 (s, 9H), 1.61-1.71 (m, 1H), 1.73-1.83 (m, 2H), 2.04-2.14 (m, 2H), 2.44-2.51 (m, 3H), 3.18-3.24 (m, 4H), 3.59-3.65 (m, 4H), 3.65-3.73 (m, 1H), 4.42 (q, J=7.3 Hz, 2H), 4.48-4.54 (m, 1H), 7.05 (s, 1H), 7.10 (dd, J=5.3, 1.3 Hz, 1H), 7.72 (s, 1H), 8.15 (d, J=5.3 Hz, 1H).

### F. 6-Chloro-2′-cyclohexylamino-[2,4′]bipyridinyl-4-carboxylic acid methyl ester

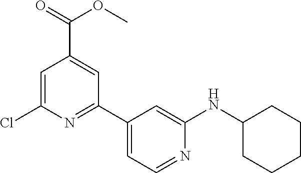

The title compound is prepared from 2,6-dichloroisonicotinic acid methyl ester and cyclohexyl-(4-trimethylstannanylpyridin-2-yl)amine by analogy to the Stille coupling method outlined above. ¹H NMR (400 MHz, CDCl₃) δ ppm 1.17-1.32 (m, 2H), 1.38-1.52 (m, 3H), 1.60-1.70 (m, 1H), 1.71-1.82 (m, 2H), 2.02-2.11 (m, 2H), 3.69-3.80 (m, 1H), 4.00 (s, 3H), 4.58-4.68 (m, 1H), 7.03 (s, 1H), 7.05-7.10 (m, 1H), 7.87 (d, J=1.1 Hz, 1H), 8.16-8.21 (m, 2H).

### G. 4-(2′-Cyclohexylamino-4-methoxycarbonylamino-[2,4′]bipyridinyl-6-yl)-piperazine-1-carboxylic acid tert-butyl ester

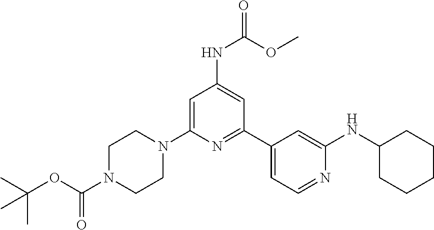

The title compound is prepared from 4-(6-chloro-4-methoxycarbonylaminopyridin-2-yl)piperazine-1-carboxylic acid tert-butyl ester and cyclohexyl-(4-trimethylstannanylpyridin-2-yl)amine by analogy to the Stille coupling method outlined above. ¹H NMR (400 MHz, CDCl₃) δ ppm 1.19-1.32 (m, 3H), 1.36-1.46 (m, 2H), 1.49 (s, 9H), 1.61-1.69 (m, 1H), 1.72-1.81 (m, 2H), 2.05-2.13 (m, 2H), 3.52-3.68 (m, 9H), 3.81 (s, 3H), 4.54 (br. s., 1H), 6.72 (s, 1H), 6.92-7.04 (m, 4H), 8.11 (d, J=5.6 Hz, 1H).

### H. 4-[2′-Cyclohexylamino-4-(3-phenyl-ureido)-[2,4′]bipyridinyl-6-yl]-piperazine-1-carboxylic acid tert-butyl ester

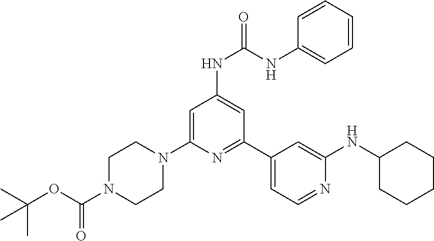

The title compound is prepared from 4-[6-chloro-4-(3-phenylureido)pyridin-2-yl]piperazine-1-carboxylic acid tert-butyl ester and cyclohexyl-(4-trimethylstannanylpyridin-2-yl)amine by analogy to the Stille coupling method outlined above. MS (ESI) m/z 572.2 (M+1).

### Example 12

### A. 6-((R)-1-tert-Butoxycarbonyl-pyrrolidin-3-ylamino)-2′-cyclohexylamino-[2,4′]bipyridinyl-4-carboxylic acid methyl ester

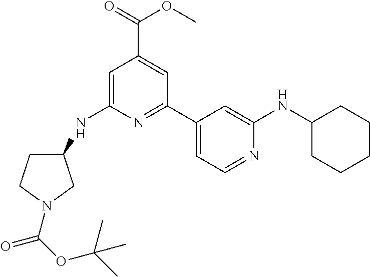

6-Chloro-2′-cyclohexylamino-[2,4′]bipyridinyl-4-carboxylic acid methyl ester (120 mg, 0.35 mmol), (R)-3-amino-pyrrolidine-1-carboxylic acid tert-butyl ester (65 mg, 0.35 mmol), Pd(OAc)₂ (7 mg, 0.032 mmol), BINAP (30 mg, 0.048 mmol), Cs₂CO₃ (140 mg, 0.42 mmol) in toluene (2 mL) are heated in a sealed tube under N₂. After cooling, the reaction is filtered through Celite® and rinsed with EtOAc. The filtrate is concentrated in vacuo. The residue is purified by flash chromatography (10 to 60% EtOAc/heptanes) to afford to title compound as a yellow oil. MS (ESI) m/z 496.2 (M+1).

### B. 2′-Cyclohexylamino-6-((S)-pyrrolidin-3-ylamino)-[2,4′]bipyridinyl-4-carboxylic acid methyl ester

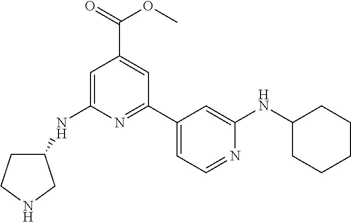

By analogy to the above coupling, 6-chloro-2′-cyclohexylamino-[2,4′]bipyridinyl-4-carboxylic acid methyl ester (120 mg, 0.35 mmol) is reacted with (S)-3-amino-pyrrolidine-1-carboxylic acid tert-butyl ester. In this case, the product of the coupling is observed to undergo hydrolysis of the ester to the corresponding carboxylic acid. After cooling, the reaction is filtered through Celite® and rinsed with MeOH. The filtrate is concentrated under reduced pressure and the residue is partitioned between water (adjusted to pH 3 with 1 N HCl) and a 10:1 mixture of CH₂Cl₂/EtOH. The separated organic phase is dried (Na₂SO₄) and concentrated in vacuo. The residue is triturated with MeOH. The MeOH soluble portion is isolated by filtration and reconcentrated. The residue is purified by reverse-phase HPLC eluting with a mixture of CH₃CN in aqueous ammonia to yield 6-((S)-1-tert-butoxycarbonyl-pyrrolidin-3-ylamino)-2′-cyclohexylamino-[2,4′]bipyridinyl-4-carboxylic acid.

The desired methyl ester is generated by Fisher esterification of the above acid. Acetyl chloride (0.25 mL) is added to MeOH (25 mL) at room temperature. After 5 min, the carboxylic acid product above is added and the reaction is heated at 65° C. for 6 h. The reaction is cooled and concentrated to the title compound as the HCl salt. MS (ESI) m/z 396.2 (M+1).

### Example 13

### A. 2′-Fluoro-6-[(2-hydroxyethyl)methylamino]-[2,4′]bipyridinyl-4-carboxylic acid

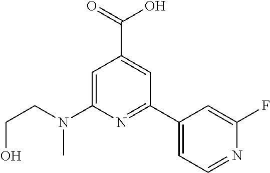

2-Chloro-6-[(2-hydroxyethyl)methylamino]isonicotinic acid methyl ester prepared above (150 mg, 0.61 mmol), a 2 M aqueous solution of Na₂CO₃ (1.5 mL), and n-butanol (8 mL) are placed in a microwave vial and degassed with N₂. 2-Fluoropyridinyl-4-boronic acid (260 mg, 1.8 mmol), PdCl₂(PPh₃)₂ (64 mg, 0.09 mmol) are added and the reaction is heated to 145° C. in the microwave for 30 min. The reaction is cooled and concentrated in vacuo. The residue is purified by flash chromatography eluting with 2% MeOH/CH₂Cl₂, then 5% MeOH/1% NH₄OH/CH₂Cl₂ to give the title compound. ¹H NMR (400 MHz, DMSO-d₆) δ ppm 3.13 (s, 3H), 3.52-3.70 (m, 4H), 4.72 (br. s., 1H), 7.14 (s, 1H), 7.61 (s, 1H), 7.69 (s, 1H), 7.92-7.95 (m, 1H), 8.29 (d, J=5.3 Hz, 1H).

### B. 2′-Fluoro-6-[(2-hydroxyethyl)methylamino]-[2,4′]bipyridinyl-4-carboxylic acid amide

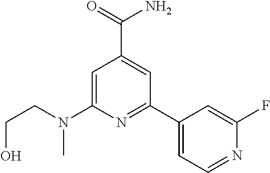

The 2′-fluoro-6-[(2-hydroxyethyl)methylamino]-[2,4′]bipyridinyl-4-carboxylic acid prepared above (50 mg, 0.17 mmol), NH₄Cl (90 mg, 1.7 mmol), HATU (130 mg, 0.34 mmol), and i-Pr₂EtNH (35 uL, 0.2 mmol) are stirred in DMF (2 mL) at rt. Upon completion, the reaction is diluted with CH₂Cl₂, washed with a saturated aqueous solution of NaHCO₃, washed with a saturated aqueous solution of NaCl, dried (Na₂SO₄), and concentrated in vacuo. The residue is purified by flash chromatography (10% MeOH/CH₂Cl₂) to yield the title amide. ¹H NMR (400 MHz, MeOD) δ ppm 3.24 (s, 3H), 3.34 (s, 1H), 3.79-3.85 (m, 4H), 7.16 (d, J=1.0 Hz, 1H), 7.61 (d, J=1.0 Hz, 1H), 7.73 (s, 1H), 8.26 (d, J=5.3 Hz, 1H).

### C. 2′-Cyclohexylamino-6-[(2-hydroxyethyl)methylamino]-[2,4′]bipyridinyl-4-carboxylic acid amide

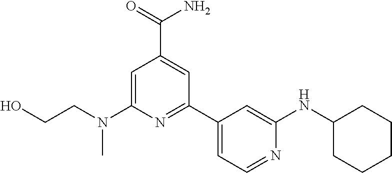

2′-Fluoro-6-[(2-hydroxyethyl)methylamino]-[2,4′]bipyridinyl-4-carboxylic acid amide (110 mg, 0.31 mmol) and cyclohexylamine (2 mL) are placed in a sealed tube and heated to 120° C. for 48 h. After cooling, the reaction is concentrated under reduced pressure and the residue is purified by reverse-phase HPLC eluting with 10 to 100% CH₃CN in dilute aqueous ammonia to afford an off-white solid. MS (ESI) m/z 370.2 (M+1). ¹H NMR (400 MHz, MeOD) d ppm 1.20-1.33 (m, 3H), 1.39-1.52 (m, 2H), 1.63-1.73 (m, 1H), 1.75-1.85 (m, 2H), 2.04 (s, 2H), 3.22 (s, 3H), 3.62-3.71 (m, 1H), 3.82 (s, 4H), 7.06-7.08 (m, 1H), 7.13 (dd, J=5.6, 1.5 Hz, 1H), 7.20-7.22 (m, 1H), 7.46 (d, J=1.0 Hz, 1H), 7.96 (dd, J=5.6, 0.5 Hz, 1H).

### Example 14

### A. 6-Chloro-[2,4′]bipyridinyl-4-carboxylic acid tert-butylamide

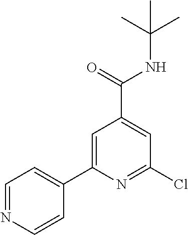

N-tert-Butyl-2,6-dichloroisonicotinamide prepared above (110 mg, 0.4 mmol), 4-pyridylboronic acid (50 mg, 0.4 mmol), a 10:1 solution of toluene/EtOH (2 mL), and a 1 M solution of Na₂CO₃ in water (0.3 mL) are placed in a vessel and degassed with N₂. The catalyst Pd(dppb)₂Cl₂ (24 mg, 0.04 mmol) is added and the sealed reaction vessel is heated at 100° C. on. The reaction is cooled and filtered. The filter cake is washed with EtOAc and the resulting filtrate is concentrated in vacuo. The residue is purified by flash chromatography (20 to 60% EtOAc/heptanes) to afford the product as a white solid. ¹H NMR (400 MHz, MeOD) δ ppm 1.48 (s, 9H), 7.74-7.81 (m, 1H), 8.10-8.15 (m, 2H), 8.25-8.29 (m, 1H), 8.66-8.72 (m, 2H).

### B. 2-Cyclohexylamino-[4,2′;6′4″]terpyridine-4′-carboxylic acid tert-butylamide

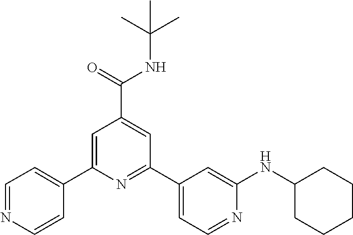

The title compound is prepared from 6-chloro-[2,4′]bipyridinyl-4-carboxylic acid tert-butylamide above and cyclohexyl-(4-trimethylstannanylpyridin-2-yl)amine according to the method outlined in the preparation of 6-(4-tert-butoxycarbonylpiperazin-1-yl)-2′-cyclohexylamino-[2,4′]bipyridinyl-4-difluoromethane. ¹H NMR (400 MHz, CDCl₃) δ ppm 1.21-1.34 (m, 2H), 1.41-1.50 (m, 2H), 1.54 (s, 9H), 1.62-1.72 (m, 2H), 1.74-1.84 (m, 2H), 2.07-2.15 (m, 2H), 3.69-3.80 (m, 1H), 4.57-4.63 (m, 1H), 6.06 (br. s., 1H), 7.15 (s, 1 H), 7.18-7.21 (m, 1H), 7.97 (d, J=1.3 Hz, 1H), 8.02-8.07 (m, 3H), 8.22 (d, J=5.4 Hz, 1H), 8.75-8.81 (m, 2H).

### C. 2-Cyclohexylamino-[4,2′;6′,4″]terpyridine-4′-carboxylic acid amide

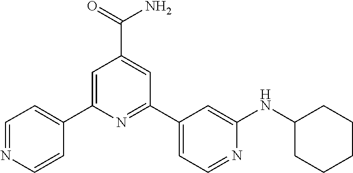

2-Cyclohexylamino-[4,2′;6′,4″]terpyridine-4′-carboxylic acid tert-butylamide prepared above (73 mg, 0.17 mmol) and TFA (5 mL) are heated at 120° C. for 2 h. The reaction is concentrated under reduced pressure. The residue is taken up in 7 N NH₃ in MeOH and reconcentrated under reduced pressure. The residue is purified by reverse-phase HPLC using 10 to 100% CH₃CN in dilute aqueous ammonia to afford the title compound. MS (ESI) m/z 374.2 (M+1). ¹H NMR (400 MHz, DMSO-d₆) δ ppm 1.12-1.43 (m, 5H), 1.55-1.66 (m, 1H), 1.68-1.79 (m, 2H), 1.89-2.01 (m, 2H), 3.74-3.85 (m, 1H), 6.61 (d, J=7.3 Hz, 1H), 7.22 (dd, J=5.6, 1.5 Hz, 1H), 7.37 (s, 1H), 7.90 (s, 1H), 8.12 (d, J=5.3 Hz, 1H), 8.21-8.23 (m, 2H), 8.33 (d, J=1.0 Hz, 1H), 8.46 (s, 1H), 8.50 (d, J=1.0 Hz, 1H), 8.77-8.80 (m, 2H).

### Example 15

### A. Cyclohexyl-(4-difluoromethyl-6-piperazin-1-yl-[2,4′]bipyridinyl-2′-yl)-amine

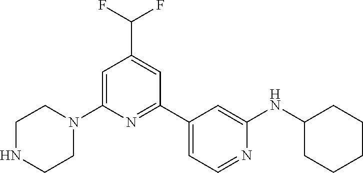

TFA is added dropwise to a solution of 6-(4-tert-butoxycarbonylpiperazin-1-yl)-2′-cyclohexylamino-[2,4′]bipyridinyl-4-difluoromethane (110 mg, 0.23 mmol) in CH₂Cl₂ (5 mL) until TLC showed complete consumption of the starting material. A 3 N solution of NH₃ in MeOH is added and the reaction is concentrated under reduced pressure. The residue is purified by reverse phase HPLC to yield the title compound as a white solid: MS (ESI) m/z 388.2 (M+1). ¹H NMR (400 MHz, MeOD) δ ppm 1.19-1.33 (m, 3H), 1.37-1.53 (m, 2H), 1.68 (m, 1H), 1.75-1.85 (m, 2H), 1.98-2.08 (m, 2H), 2.92-3.00 (m, 4H), 3.62-3.73 (m, 5H), 6.74 (t, J=55.8 Hz, 1H), 6.93 (s, 1H), 7.09 (dd, J=5.6, 1.5 Hz, 1H), 7.20 (s, 1H), 7.26 (s, 1H), 7.96 (dd, J=5.6, 0.76 Hz, 1H).

### B. Cyclohexyl-(6-piperazin-1-yl-4-trifluoromethyl-[2,4′]bipyridinyl-2′-yl)amine

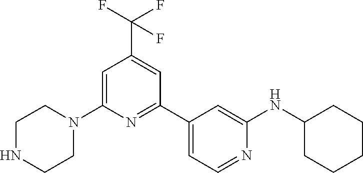

The title compound is prepared by TFA deprotection of 6-(4-tert-butoxycarbonylpiperazin-1-yl)-2′-cyclohexylamino-[2,4′]bipyridinyl-4-trifluoromethane. MS (ESI) m/z 406.2 (M+1). ¹H NMR (400 MHz, MeOD) δ ppm 1.19-1.33 (m, 3H), 1.38-1.51 (m, 2H), 1.63-1.72 (m, 1H), 1.74-1.84 (d, 2H), 1.99-2.08 (m, 2H), 2.94-2.99 (m, 4H), 3.68-3.73 (m, 5H), 7.02 (s, 1H), 7.09 (dd, J=5.6, 1.5 Hz, 1H), 7.18-7.20 (m, 1H), 7.31 (s, 1H), 7.98 (dd, J=5.6, 0.8 Hz, 1H).

### C. (6-piperazin-1-yl-4-trifluoromethyl-[2,4′]bipyridinyl-2′-yl)-(tetrahydropyran-4-yl)-amine

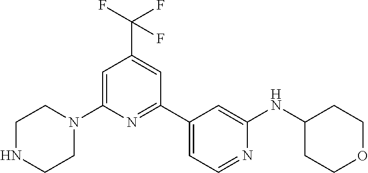

The title compound is prepared by TFA deprotection of 6-(4-tert-butoxycarbonylpiperazin-1-yl)-2′-(4″-tetrahydropyranyl)-[2,4′]bipyridinyl-4-trifluoromethane. MS (ESI) m/z 408.2 (M+1). ¹H NMR (400 MHz, MeOD) δ ppm 1.49-1.61 (m, 2H), 1.94-2.05 (m, 2H), 2.92-2.99 (m, 4H), 3.52-3.61 (m, 2H), 3.67-3.74 (m, 4H), 3.94-4.02 (m, 3H), 7.02 (s, 1H), 7.13 (dd, J=5.6, 1.52 Hz, 1H), 7.22-7.26 (m, 1H), 7.31 (s, 1H), 8.01 (d, J=5.6 Hz, 1H).

### D. 2′-Cyclohexylamino-6-((S)-3-methylpiperazin-1-yl)-[2,4′]bipyridinyl-4-carboxylic acid methyl ester

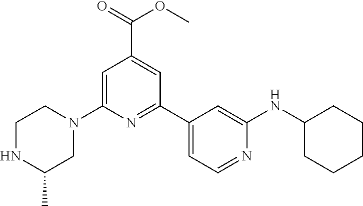

The title compound is prepared by TFA deprotection of 6-((S)-4-tert-butoxycarbonyl-3-methyl-piperazin-1-yl)-2′-cyclohexylamino-[2,4′]bipyridinyl-4-carboxylic acid methyl ester.

### E. 2′-Cyclohexylamino-6-((R)-3-methylpiperazin-1-yl)-[2,4′]bipyridinyl-4-carboxylic acid methyl ester

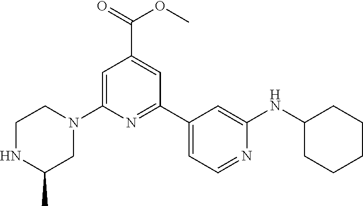

The title compound is prepared by TFA deprotection of 6-((R)-4-tert-butoxycarbonyl-3-methyl-piperazin-1-yl)-2′-cyclohexylamino-[2,4′]bipyridinyl-4-carboxylic acid methyl ester. MS (ESI) m/z 410.2 (M+1).

### F. 2′-Cyclohexylamino-6-((R)-pyrrolidin-3-ylamino)-[2,4′]bipyridinyl-4-carboxylic acid methyl ester

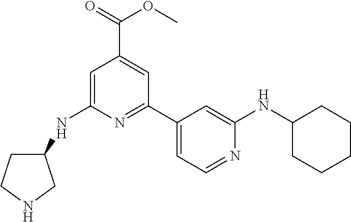

The title compound is prepared by TFA deprotection of 6-((R)-1-tert-butoxycarbonyl-pyrrolidin-3-ylamino)-2′-cyclohexylamino-[2,4′]bipyridinyl-4-carboxylic acid methyl ester. MS (ESI) m/z 396.2 (M+1).

### G. 6-(piperazin-1-yl)-2′-cyclohexylamino-3-methyl-[2,4′]bipyridinyl-4-carboxylic acid ethyl ester

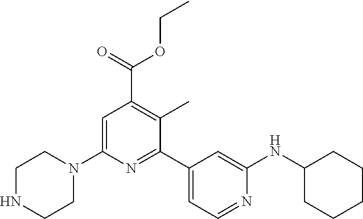

The title compound is prepared by TFA deprotection of 6-(4-tert-butoxycarbonylpiperazin-1-yl)-2′-cyclohexylamino-3-methyl-[2,4′]bipyridinyl-4-carboxylic acid ethyl ester. MS (ESI) m/z 424.2 (M+1).

### H. 6-(piperazin-1-yl)-2′-cyclohexylamino-5-methyl-[2,4′]bipyridinyl-4-carboxylic acid ethyl ester

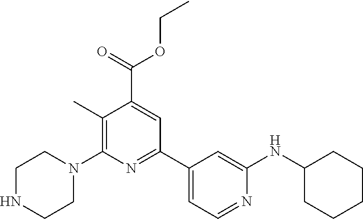

The title compound is prepared by TFA deprotection of 6-(4-tert-butoxycarbonylpiperazin-1-yl)-2′-cyclohexylamino-5-methyl-[2,4′]bipyridinyl-4-carboxylic acid ethyl ester. MS (ESI) m/z 424.2 (M+1).

### I. (2′-Cyclohexylamino-6-piperazin-1-yl-[2,4′]bipyridinyl-4-yl)-carbamic acid methyl ester

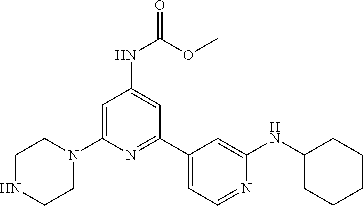

The title compound is prepared by TFA deprotection of 4-(2′-cyclohexylamino-4-methoxycarbonylamino-[2,4′]bipyridinyl-6-yl)-piperazine-1-carboxylic acid tert-butyl ester. MS (ESI) m/z 411.2 (M+1). ¹H NMR (400 MHz, CDCl₃) δ ppm 1.16-1.31 (m, 3H), 1.36-1.48 (m, 2H), 1.60-1.69 (m, 1H), 1.70-1.81 (m, 2H), 2.08 (dd, J=14.3, 4.4 Hz, 2H), 2.97-3.03 (m, 4H), 3.58-3.64 (m, 4H), 3.64-3.71 (m, 1H), 3.81 (s, 3H), 4.47 (d, J=8.8 Hz, 1H), 6.71 (s, 1H), 6.94 (d, J=1.5 Hz, 1H), 6.98 (s, 2H), 7.03 (dd, J=5.3, 1.5 Hz, 1H), 8.11 (d, J=5.6 Hz, 1H).

### J. 1-(2′-Cyclohexylamino-6-piperazin-1-yl-[2,4′]bipyridinyl-4-yl)-3-phenylurea

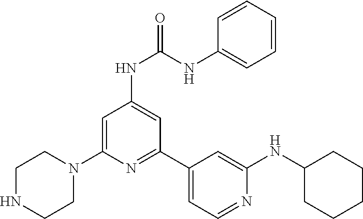

The title compound is prepared by TFA deprotection of 4-[2′-cyclohexylamino-4-(3-phenylureido)-[2,4′]bipyridinyl-6-yl]-piperazine-1-carboxylic acid tert-butyl ester. MS (ESI) m/z 472.2 (M+1). ¹H NMR (400 MHz, MeOD) δ ppm 1.20-1.34 (m, 3H), 1.38-1.52 (m, 2 H), 1.67 (d, J=11.6 Hz, 1H), 1.80 (d, J=13.9 Hz, 2H), 2.04 (d, J=14.4 Hz, 2H), 2.96-3.01 (m, 4H), 3.63 (m, 5H), 7.02-7.08 (m, 3H), 7.15 (s, 1H), 7.22 (s, 1H), 7.31 (t, J=8.0 Hz, 2 H), 7.42-7.47 (m, 2H), 7.94 (d, J=5.6 Hz, 1H).

### Example 16

### A. 2′-Cyclohexylamino-6-(3,3-dimethylpiperazin-1-yl)-[2,4′]bipyridinyl-4-carboxylic acid amide

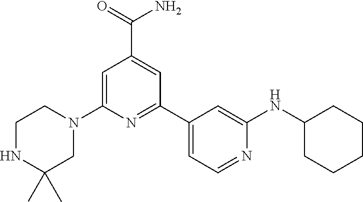

6-(3,3-dimethylpiperazin-1-yl)-2′-cyclohexylamino-[2,4′]bipyridinyl-4-carboxylic acid methyl ester (61 mg, 0.14 mmol) and a solution of 7 M NH₃ in MeOH (10 mL) are placed in a pressure vessel and heated to 90° C. on. The reaction is cooled and concentrated under reduced pressure. The residue is purified by HPLC to give the title compound. MS (ESI) m/z 409.2 (M+1). ¹H NMR (400 MHz, MeOD)) δ ppm 1.23-1.24 (s, 6H), 1.25-1.32 (m, 2H), 1.37-1.53 (m, 2H), 1.68 (d, J=13.9 Hz, 1H), 1.80 (d, J=13.9 Hz, 2H), 2.05 (d, J=10.9 Hz, 2 H), 2.96-3.03 (m, 2H), 3.52 (s, 2H), 3.64-3.72 (m, 3H), 7.12 (dd, J=5.7, 1.6 Hz, 1H), 7.20 (d, J=1.8 Hz, 2H), 7.50 (s, 1H), 7.97 (d, J=5.6 Hz, 1H).

### B. 2′-Cyclohexylamino-6-(4,7-diazaspiro[2.5]oct-7-yl)-[2,4′]bipyridinyl-4-carboxylic acid amide

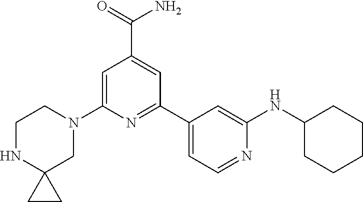

The title compound is prepared from 2′-cyclohexylamino-6-(4,7-diazaspiro[2.5]oct-7-yl)-[2,4′]bipyridinyl-4-carboxylic acid methyl ester by analogy to the aminolysis method outlined above. MS (ESI) m/z 407.2 (M+1). ¹H NMR (400 MHz, MeOD) δ ppm 0.62-0.71 (m, 4H), 1.20-1.33 (m, 3H), 1.37-1.52 (m, 2H), 1.64-1.73 (m, 1H), 1.74-1.84 (m, 2H), 1.98-2.09 (m, 2H), 2.97-3.06 (m, 2H), 3.59 (s, 2H), 3.62-3.75 (m, 3H), 7.11 (dd, J=5.7, 1.5 Hz, 1H), 7.17-7.20 (m, 2H), 7.51-7.58 (m, 2H), 7.61-7.67 (m, 1H), 7.96 (dd, J=5.7, 0.6 Hz, 1H).

Compounds C, D and F-I of Example 16 can be prepared by a similar method as those above.

### C. 2′-Cyclohexylamino-6-((S)-3-methylpiperazin-1-yl)-[2,4′]bipyridinyl-4-carboxylic acid amide

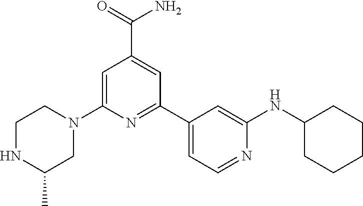

MS (ESI) m/z 395.2 (M+1). ¹H NMR (400 MHz, MeOD)) δ ppm 1.19 (d, J=6.3 Hz, 3 H), 1.23-1.33 (m, 3H), 1.39-1.52 (m, 2H), 1.68 (d, J=11.9 Hz, 1H), 1.80 (d, J=13.1 Hz, 2 H), 2.04 (d, J=12.4 Hz, 2H), 2.57-2.65 (m, 1H), 2.84-3.00 (m, 3H), 3.10 (d, J=10.9 Hz, 1H), 3.63-3.71 (m, 1H), 4.38 (d, J=10.9 Hz, 2H), 7.12 (dd, J=5.7, 1.6 Hz, 1H), 7.22 (d, J=3.8 Hz, 2H), 7.53 (s, 1H), 7.97 (d, J=5.6 Hz, 1H).

### D. 2′-Cyclohexylamino-6-((R)-3-methylpiperazin-1-yl)-[2,4′]bipyridinyl-4-carboxylic acid amide

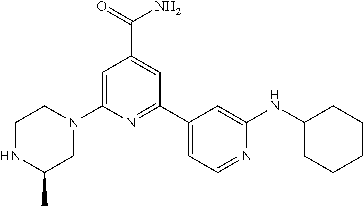

MS (ESI) m/z 395.2 (M+1). ¹H NMR (400 MHz, MeOD) δ ppm 1.20 (d, J=6.32 Hz, 3 H), 1.24-1.33 (m, 3H), 1.39-1.48 (m, 2H), 1.65-1.72 (m, 1H), 1.80 (d, J=13.9 Hz, 2H), 2.04 (d, J=9.9 Hz, 2H), 2.58-2.66 (m, 1H), 2.85-2.98 (m, 3H), 3.07-3.15 (m, 1H), 3.62-3.72 (m, 1H), 4.39 (d, J=12.9 Hz, 2H), 7.12 (dd, J=5.7, 1.4 Hz, 1H), 7.22 (d, J=6.8 Hz, 2 H), 7.54 (s, 1H), 7.97 (d, J=5.6 Hz, 1H).

### E. 2′-Cyclohexylamino-6-morpholin-4-yl-[2,4′]bipyridinyl-4-carboxylic acid amide

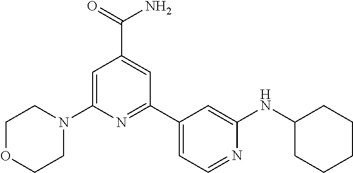

The title compound is prepared from 2′-cyclohexylamino-6-morpholin-4-yl-[2,4′]bipyridinyl-4-carboxylic acid methyl ester by analogy to the aminolysis method outlined above. MS (ESI) m/z 382.2 (M+1). ¹H NMR (400 MHz, DMSO-d₆) δ ppm 1.12-1.26 (m, 3 H), 1.27-1.40 (m, 2H), 1.54-1.66 (m, 1H), 1.68-1.78 (m, 2H), 1.87-1.98 (m, 2H), 3.56-3.63 (m, 4H), 3.71-3.78 (m, 5H), 6.47 (d, J=7.8 Hz, 1H), 7.04 (dd, J=5.3, 1.5 Hz, 1H), 7.16 (s, 1H), 7.24 (s, 1H), 7.58 (s, 1H), 7.63 (s, 1H), 8.02 (d, J=5.3 Hz, 1H), 8.18 (s, 1H).

### F. 2′-Cyclohexylamino-6-((R)-pyrrolidin-3-ylamino)-[2,4′]bipyridinyl-4-carboxylic acid amide

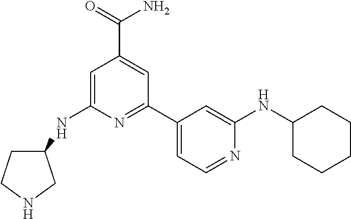

MS (ESI) m/z 381.2 (M+1). ¹H NMR (400 MHz, MeOD) δ ppm 1.27 (q, J=12.9 Hz, 3 H), 1.39-1.52 (m, 2H), 1.69 (d, J=14.2 Hz, 1H), 1.80 (d, J=13.4 Hz, 2H), 1.85-1.94 (m, 1 H), 2.05 (d, J=12.4 Hz, 2H), 2.29 (dd, J=12.5, 7.7 Hz, 1H), 2.96-3.02 (m, 1H), 3.04-3.12 (m, 1H), 3.16-3.25 (m, 1H), 3.36 (dd, J=11.8, 6.19 Hz, 1H), 3.63-3.71 (m, 1H), 4.48-4.57 (m, 1H), 6.93 (d, J=1.0 Hz, 1H), 7.13 (dd, J=5.6, 1.5 Hz, 1H), 7.18 (s, 1H), 7.43 (d, J=1.3 Hz, 1H), 7.96 (d, J=5.6 Hz, 1H).

### G. 2′-Cyclohexylamino-6-((S)-pyrrolidin-3-ylamino)-[2,4′]bipyridinyl-4-carboxylic acid amide

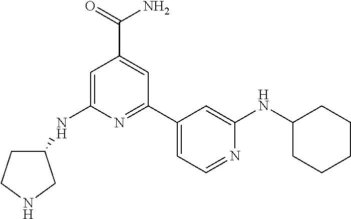

MS (ESI) m/z 381.2 (M+1). ¹H NMR (400 MHz, MeOD) δ ppm 1.21-1.33 (m, 3H), 1.46 (d, J=12.4 Hz, 2H), 1.68 (d, J=10.4 Hz, 1H), 1.75-1.84 (m, 2H), 1.84-1.93 (m, 1H), 2.05 (d, J=11.6 Hz, 2H), 2.23-2.34 (m, 1H), 2.97 (dd, J=12.0, 4.4 Hz, 1H), 3.02-3.11 (m, 1H), 3.14-3.23 (m, 1H), 3.36-3.39 (m, 1H), 3.63-3.71 (m, 1H), 4.48-4.57 (m, 1H), 6.93 (d, J=1.3 Hz, 1H), 7.13 (dd, J=5.6, 1.5 Hz, 1H), 7.18 (d, J=2.3 Hz, 1H), 7.43 (d, J=1.0 Hz, 1H), 7.96 (dd, J=5.7, 0.6 Hz, 1H).

### H. 2′-Cyclohexylamino-3-methyl-6-piperazin-1-yl-[2,4′]bipyridinyl-4-carboxylic acid amide

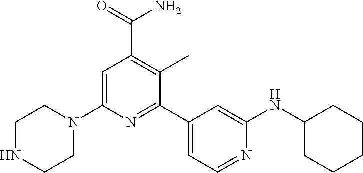

MS (ESI) m/z 395.2 (M+1). ¹H NMR (400 MHz, MeOD) δ ppm 1.16-1.33 (m, 2H), 1.37-1.50 (m, 2H), 1.67 (d, J=12.4 Hz, 1H), 1.79 (d, J=12.6 Hz, 2H), 2.03 (d, J=13.1 Hz, 2 H), 2.22 (s, 3H), 2.91-2.97 (m, 4H), 3.50-3.58 (m, 4H), 3.64 (m, 1H), 6.56 (s, 1H), 6.59 (dd, J=5.3, 1.5 Hz, 1H), 6.79 (s, 1H), 7.94 (dd, J=5.3, 0.8 Hz, 1H).

### I. 2′-Cyclohexylamino-5-methyl-6-piperazin-1-yl-[2,4′]bipyridinyl-4-carboxylic acid amide

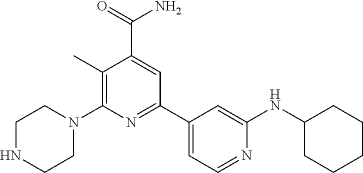

MS (ESI) m/z 395.2 (M+1). ¹H NMR (400 MHz, MeOD)) δ ppm 1.17-1.34 (m, 3H), 1.36-1.53 (m, 2H), 1.69 (d, J=14.4 Hz, 1H), 1.80 (d, J=16.2 Hz, 2H), 2.04 (d, J=12.1 Hz, 2 H), 2.37 (s, 3H), 3.01-3.07 (m, 4H), 3.25 (d, J=9.9 Hz, 4H), 3.61-3.72 (m, 1H), 7.14 (dd, J=5.7, 1.6 Hz, 1H), 7.22 (s, 1H), 7.50 (s, 1H), 7.96 (d, J=5.1 Hz, 1H).

### Example 17

### A. 4′-Carbamoyl-2″-cyclohexylamino-3,4,5,6-tetrahydro-2H-[1,2′;6′,4″]terpyridine-4-carboxylic acid

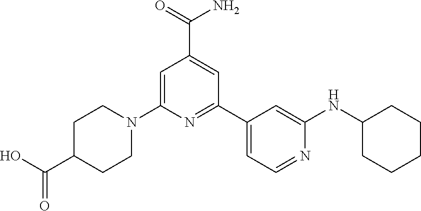

4′-Carbamoyl-2″-cyclohexylamino-3,4,5,6-tetrahydro-2H-[1,2′;6′,4″]terpyridine-4-carboxylic acid tert-butyl ester prepared above (105 mg, 0.22 mmol), Et₃SiH (0.1 mL, 0.55 mmol), TFA (0.2 mL, 2.86 mmol), and CH₂Cl₂ (0.45 mL) are combined in a flask and stirred. After 1 h, an additional aliquot of TFA (0.1 mL) is added. The reaction is stirred at rt on and concentrated under reduced pressure. The residue is triturated with Et₂O and filtered. The solids are dissolved in MeOH, filtered, and isolated from the filtrate to give the product as a TFA salt. Purification by reverse-phase HPLC using 10 to 100% CH₃CN in dilute aqueous ammonia affords the title compound. MS (ESI) m/z 424.2 (M+1). ¹H NMR (400 MHz, DMSO-d₆) δ ppm 1.11-1.26 (m, 3H), 1.31 (t, J=12.9 Hz, 2H), 1.59 (t, J=10.1 Hz, 3H), 1.72 (d, J=12.1 Hz, 2H), 1.93 (d, J=12.4 Hz, 4H), 3.05 (t, J=12.1 Hz, 2H), 3.67-3.81 (m, 1H), 4.36 (d, J=13.9 Hz, 2H), 6.47 (d, J=7.8 Hz, 1H), 7.02 (d, J=6.8 Hz, 1H), 7.15 (s, 1H), 7.24 (s, 1H), 7.50 (s, 1H), 7.60 (br s, 1H), 8.02 (d, J=5.6 Hz, 1H), 8.17 (br s, 1H).

### B. 2″-Cyclohexylamino-3,4,5,6-tetrahydro-2H-[1,2′;6′,4″]terpyridine-4,4′-dicarboxylic acid diamide

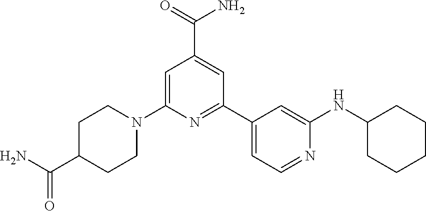

4′-Carbamoyl-2″-cyclohexylamino-3,4,5,6-tetrahydro-2H-[1,2′;6′,4″]terpyridine-4-carboxylic acid prepared above (70 mg, 0.16 mmol), NH₄Cl (85 mg, 1.6 mmol), HATU (120 mg, 0.32 mmol), i-Pr₂EtN (30 uL, 0.19 mmol) are stirred in DMF (2 mL) at rt on. The reaction is concentrated under reduced pressure and purified by reverse-phase HPLC using 10 to 100% CH₃CN in dilute aqueous ammonia to afford the title compound. MS (ESI) m/z 423.2 (M+1). ¹H NMR (400 MHz, DMSO-d₆) δ ppm 1.12-1.26 (m, 3H), 1.31 (t, J=12.8 Hz, 2H), 1.49-1.64 (m, 3H), 1.73 (d, J=12.6 Hz, 2H), 1.81 (d, J=12.4 Hz, 2H), 1.94 (d, J=12.1 Hz, 2 H), 2.34-2.45 (m, 1H), 2.93 (m, 2H), 3.66-3.80 (m, 1H), 4.47 (d, J=13.1 Hz, 2H), 6.48 (d, J=8.1 Hz, 1H), 6.78 (s, 1H), 7.02 (dd, J=5.3, 1.5 Hz, 1H), 7.15 (s, 1H), 7.24 (s, 1H), 7.30 (s, 1H), 7.50 (s, 1H), 7.60 (s, 1H), 8.02 (d, J=5.3 Hz, 1H), 8.17 (s, 1H).

### Example 18

### A. 4-(2′-Cyclohexylamino-4-[1,3,4]oxadiazol-2-yl-[2,4′]bipyridinyl-6-yl)-piperazine-1-carboxylic acid tert-butyl ester

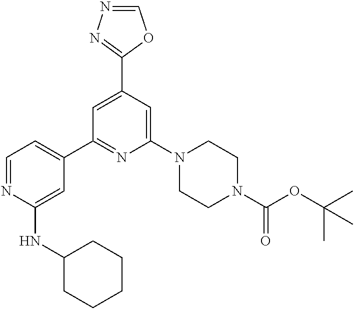

To a stirred solution of 6-(4-tert-Butoxycarbonyl-piperazin-1-yl)-2′-cyclohexylamino-[2,4′]bipyridinyl-4-carboxylic acid methyl ester Example 10A (0.136 g, 0.2745 mmol) and MeOH (2 mL) is added hydrazine (0.09 mL, 2.754 mmol). After 2 h, an additional 10 equivalents of hydrazine are added. A third aliquot of hydrazine (1 mL) is added after 1 h along with MeOH (2 mL). The mixture is then stirred overnight. Concentration then provides crude 4-(2′-cyclohexylamino-4-hydrazinocarbonyl-[2,4′]bipyridinyl-6-yl)-piperazine-1-carboxylic acid tert-butyl ester that is taken on without further purification.

A solution of 4-(2′-cyclohexylamino-4-hydrazinocarbonyl-[2,4′]bipyridinyl-6-yl)-piperazine-1-carboxylic acid tert-butyl ester (0.274 mmol) and triethylorthoformate (4 mL) is heated to 130° C. After 6 h, the solution is concentrated. The residue is then separated via flash chromatography (SiO₂, 50-100% EtOAc/heptane gradient) to give the title compound 4-(2′-cyclohexylamino-4-[1,3,4]oxadiazol-2-yl-[2,4′]bipyridinyl-6-yl)-piperazine-1-carboxylic acid tert-butyl ester. MS (ESI) m/z 506.1 (M+1). ¹H NMR (400 MHz, CDCl₃) δ ppm 8.54 (s, 1H), 8.17 (d, J=5.1 Hz, 1H), 7.68 (d, J=1.0 Hz, 1H), 7.31 (s, 1H), 7.12 (dd, J=5.3, 1.5 Hz, 1H), 7.03 (s, 1H), 4.50-4.61 (m, 1H), 3.71-3.77 (m, 4H), 3.65-3.71 (m, 1H), 3.57-3.64 (m, 4H), 2.04-2.14 (m, 2H), 1.73-1.84 (m, 2H), 1.62-1.71 (m, 1H), 1.50 (s, 9H), 1.38-1.48 (m, 2H), 1.18-1.33 (m, 3H)

### B. Cyclohexyl-(4-[1,3,4]oxadiazol-2-yl-6-piperazin-1-yl-[2,4′]bipyridinyl-2′-yl)-amine

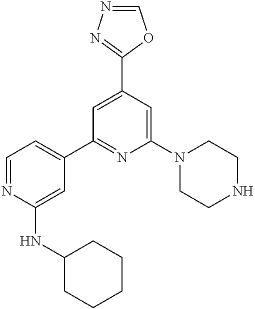

To a solution of 4-(2′-cyclohexylamino-4-[1,3,4]oxadiazol-2-yl-[2,4′]bipyridinyl-6-yl)-piperazine-1-carboxylic acid tert-butyl ester (0.076 g, 0.150 mmol) and CH₂Cl₂ (1 mL) is added TFA (0.5 mL). After stirring for 1 h the solution is concentrated. The residue is then separated via semi-preparative HPLC (10-90% CH₃CN/H₂O gradient with 0.1% NH₄OH) to give the title compound cyclohexyl-(4-[1,3,4]oxadiazol-2-yl-6-piperazin-1-yl-[2,4′]bipyridinyl-2′-yl)-amine. MS (ESI) m/z 406.0 (M+1). ¹H NMR (400 MHz, CDCl₃) δ ppm 8.53 (s, 1H), 8.17 (d, J=5.8 Hz, 1H), 7.65 (d, J=0.8 Hz, 1H), 7.31 (d, J=1.0 Hz, 1H), 7.13 (dd, J=5.4, 1.4 Hz, 1H), 7.04 (s, 1H), 4.50-4.59 (m, 1H), 3.60-3.76 (m, 5H), 3.00-3.07 (m, 4H), 2.05-2.15 (m, 2H), 1.73-1.83 (m, 2H), 1.56-1.72 (m, 1H), 1.44 (d, J=43.2 Hz, 2H), 1.19-1.33 (m, 3H).

### C. Cyclohexyl-[4-(5-methyl-[1,3,4]oxadiazol-2-yl)-6-piperazin-1-yl-[2,4′]bipyridinyl-2′-yl]-amine

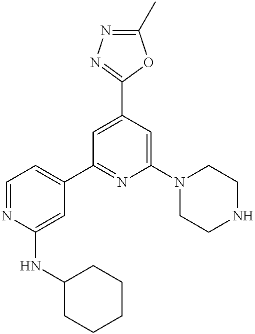

The title compound is prepared by a similar method to Example 18B using trimethylorthoacetate in place of triethylorthoformate. MS (ESI) m/z 419.5 (M+1). ¹H NMR (400 MHz, DMSO-d₆) δ ppm 8.04 (d, J=5.3 Hz, 1H), 7.55 (s, 1H), 7.24 (s, 1H), 7.18 (s, 1 H), 7.05 (dd, J=5.3, 1.5 Hz, 1H), 6.53 (d, J=7.6 Hz, 1H), 3.70-3.84 (m, 1H), 3.55-3.65 (m, 4H), 2.78-2.87 (m, 4H), 2.62 (s, 3H), 1.88-1.98 (m, 2H), 1.67-1.79 (m, 2H), 1.53-1.64 (m, 1H), 1.27-1.41 (m, 2H), 1.09-1.26 (m, 3H).

### Example 19

### A. 2,6-Dibromo-4-methanesulfonyl-pyridine

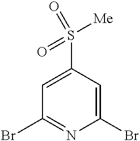

Methanesulfinic acid sodium salt (1.66 g, 16.3 mmol) is added to a solution of 2,6-dibromo-4-nitropyridine (0.917 g, 3.253 mmol) and DMF (15 mL). After 1 h the DMF is removed in vacuo and the residue is taken up in CH₂Cl₂ (150 mL) and brine (150 mL). The layers are mixed and then separated. The aqueous layer is extracted further with CH₂Cl₂ (2×150 mL). The combined organic layers are then dried (Na₂SO₄), filtered and concentrated. The residue is then separated via flash chromatography (SiO₂, 5-30% EtOAc/heptane gradient) to give the title compound 2,6-dibromo-4-methanesulfonylpyridine as a white powder. MS (ESI) m/z 316.2 (M+1). ¹H NMR (400 MHz, CDCl₃) δ ppm 7.94 (s, 2H), 3.12 (s, 3H).

### B. 4-(6-Bromo-4-methanesulfonyl-pyridin-2-yl)-piperazine-1-carboxylic acid tert-butyl ester

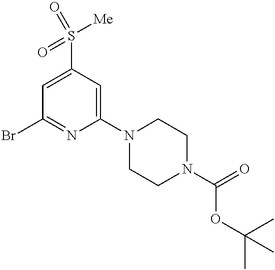

A solution of 2,6-dibromo-4-methanesulfonyl-pyridine (0.720 g, 2.28 mmol), Et₃N (1.0 mL, 6.86 mmol), tert-butyl-1-piperazine carboxylate (0.639 g, 3.43 mmol) and 1,4-dioxane (15 mL) is warmed to reflux for 3 h. After cooling to rt the solution is diluted with CH₂Cl₂ (150 mL) and NaHCO₃ (150 mL). The layers are agitated and then allowed to separate. The aqueous layer is extracted further with fresh CH₂Cl₂ (2×150 mL). The combined organic layers are then dried (Na₂SO₄), filtered and concentrated. The residue is then purified via flash chromatography (SiO₂, 10-50% EtOAc/heptane gradient) to give the title compound 4-(6-bromo-4-methanesulfonyl-pyridin-2-yl)-piperazine-1-carboxylic acid tert-butyl ester. MS (ESI) m/z 420.0 (M+1). ¹H NMR (400 MHz, CDCl₃) δ ppm 7.15 (s, 1H), 6.96 (s, 1H), 3.61-3.73 (m, 4H), 3.47-3.60 (m, 4H), 3.06 (s, 3H), 1.49 (s, 9H).

### C. 4-(2′-Chloro-4-methanesulfonyl-[2,4′]bipyridinyl-6-yl)-piperazine-1-carboxylic acid tert-butyl ester

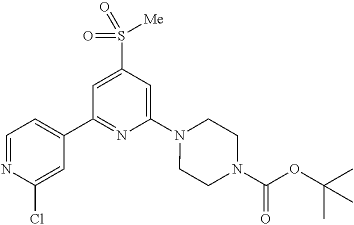

A mixture of 4-(6-bromo-4-methanesulfonyl-pyridin-2-yl)-piperazine-1-carboxylic acid tert-butyl ester (0.900 g, 2.14 mmol), Pd(dppf)₂Cl₂.CH₂Cl₂ (0.087 g, 0.107 mmol), 2-chloropyridine boronic acid (0.506 g, 3.21 mmol), aqueous solution of Na₂CO₃ (3.2 mL, 2.0 M) and DME (15 mL) is sparged with argon for 5 min. The mixture is then heated to 90° C. for 2 h. The mixture is then allowed to cool followed by concentration. The residue is taken up in CH₂Cl₂ (150 mL) and washed with brine (150 mL). The aqueous layer is further extracted with CH₂Cl₂ (2×150 mL). The combined organic layers are then dried (Na₂SO₄), filtered and concentrated. The residue is then separated via flash chromatography (SiO₂, 10-50% EtOAc/hexanes gradient) to give the title compound 4-(2′-chloro-4-methanesulfonyl-[2,4′]bipyridinyl-6-yl)-piperazine-1-carboxylic acid tert-butyl ester. MS (ESI) m/z 453.0, 455.0 (M+1, M+2). ¹H NMR (400 MHz, CDCl₃) δ ppm 8.50 (d, J=5.8 Hz, 1H), 7.93 (d, J=0.8 Hz, 1H), 7.80 (dd, J=5.3, 1.5 Hz, 1H), 7.50 (d, J=1.0 Hz, 1H), 7.17 (d, J=0.8 Hz, 1H), 3.70-3.80 (m, 4H), 3.56-3.66 (m, 4H), 3.11 (s, 3H), 1.50 (s, 9H).

### D. 4-(2′-Cyclohexylamino-4-methanesulfonyl-[2,4′]bipyridinyl-6-yl)-piperazine-1-carboxylic acid tert-butyl ester

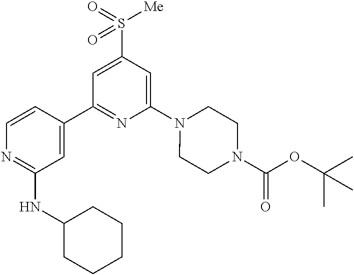

A mixture of 4-(2′-chloro-4-methanesulfonyl-[2,4′]bipyridinyl-6-yl)-piperazine-1-carboxylic acid tert-butyl ester (0.200 g, 0.442 mmol), Pd(t-Bu₃P)₂ (0.023 g, 0.044 mmol), NaOtBu (0.085 g, 0.884 mmol), cyclohexylamine (0.10 mL, 0.884 mmol) and 1,4-dioxane (4 mL) is sparged with argon for 10 min. The vessel is then sealed and the contents heated to 130° C. for 2 h. The mixture is then allowed to cool followed by concentration. The residue is taken up in CH₂Cl₂ and washed with saturated aqueous NH₄Cl. The aqueous layer is further extracted with CH₂Cl₂ (2×). The combined organic layers are then dried (Na₂SO₄), filtered and concentrated. The residue is then separated via flash chromatography (SiO₂, 60% EtOAc/heptane) to give the title compound 4-(2′-cyclohexylamino-4-methanesulfonyl-[2,4′]bipyridinyl-6-yl)-piperazine-1-carboxylic acid tert-butyl ester. MS (ESI) m/z 516.2 (M+1). ¹H NMR (400 MHz, CDCl₃) δ ppm 8.16 (d, J=5.3 Hz, 1H), 7.45 (s, 1H), 7.10 (s, 1H), 7.07 (dd, J=5.4, 1.4 Hz, 1H), 6.97 (s, 1H), 4.67 (br. s., 1H), 3.71-3.77 (m, 4H), 3.63-3.70 (m, 1H), 3.59 (d, J=10.1 Hz, 4H), 3.09 (s, 3H), 2.03-2.13 (m, 2H), 1.73-1.82 (m, 2H), 1.61-1.70 (m, 1H), 1.50 (s, 9H), 1.40-1.47 (m, 2H), 1.19-1.34 (m, 3H).

### E. Cyclohexyl-(4-methanesulfonyl-6-piperazin-1-yl-[2,4′]bipyridinyl-2′-yl)-amine

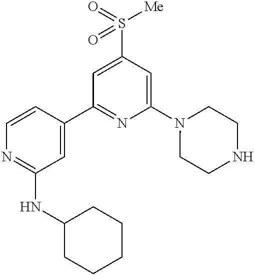

To a solution of 4-(2′-cyclohexylamino-[2,4′]bipyridinyl-6-yl)-piperazine-1-carboxylic acid tert-butyl ester (0.055 g, 0.107 mmol) and CH₂Cl₂ (2 mL) is added TFA (0.5 mL). After stirring for 1 h the solution is concentrated. The residue is then separated via semi-prep HPLC (10-90% CH₃CN/H₂O gradient with 0.1% NH₄OH) to give the title compound cyclohexyl-(4-methanesulfonyl-6-piperazin-1-yl-[2,4′]bipyridinyl-2′-yl)-amine. MS (ESI) m/z 416.2 (M+1). ¹H NMR (400 MHz, DMSO-d₆CDCl₃) δ ppm 8.04 (d, J=5.3 Hz, 1H), 7.46 (d, J=1.0 Hz, 1H), 7.18 (s, 1H), 7.16 (s, 1H), 7.05 (dd, J=5.3, 1.5 Hz, 1H), 6.54 (d, J=7.8 Hz, 1H), 3.69-3.82 (m, 1H), 3.57-3.65 (m, 4H), 3.33 (s, 3H), 2.78-2.86 (m, 4H), 1.87-1.98 (m, 2H), 1.66-1.77 (m, 2H), 1.54-1.64 (m, 1H), 1.26-1.39 (m, 2H), 1.12-1.26 (m, 3H).

### Example 20

### A. 4-[2′-Cyclohexylamino-4-(1-hydroxy-1-methylethyl)-[2,4′]bipyridinyl-6-yl]-piperazine-1-carboxylic acid tert-butyl ester

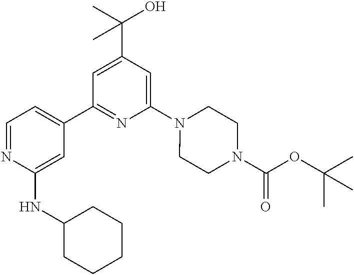

To a stirred solution of 6-(4-tert-butoxycarbonyl-piperazin-1-yl)-2′-cyclohexylamino-[2,4′]bipyridinyl-4-carboxylic acid methyl ester Example 10A (0.100 g, 0.202 mmol) and THF (2 mL) at 0° C. is added methylmagnesium bromide (0.34 mL, 3.0 M). The solution is then allowed to warm to rt and stir for an additional 0.5 h. The solution is then poured into 25 mL of saturated NaHCO₃ and extracted with CH₂Cl₂ (3×25 mL). The combined organic layers are then dried (Na₂SO₄), filtered and concentrated. The residue is then separated via flash chromatography (SiO₂, 10-100% EtOAc/heptane) to give the title compound 4-[2′-cyclohexylamino-4-(1-hydroxy-1-methyl-ethyl)-[2,4′]bipyridinyl-6-yl]-piperazine-1-carboxylic acid tert-butyl ester. MS (ESI) m/z 496.2 (M+1). ¹H NMR (400 MHz, CDCl₃) δ ppm 8.12 (d, J=5.3 Hz, 1H), 7.15 (s, 1H), 7.06 (dd, J=5.3, 1.3 Hz, 1H), 7.01 (s, 1H), 6.84 (d, J=1.0 Hz, 1H), 4.46-4.54 (m, 1H), 3.66-3.74 (m, 1H), 3.62-3.67 (m, 4H), 3.52-3.61 (m, 4H), 2.04-2.13 (m, 2H), 1.92 (br. s., 1H), 1.72-1.81 (m, 2H), 1.61-1.70 (m, 1H), 1.58 (s, 6H), 1.49 (s, 9H), 1.35-1.47 (m, 2H), 1.24 (d, J=46.0 Hz, 3H).

### B. 2-(2′-Cyclohexylamino-6-piperazin-1-yl-[2,4′]bipyridinyl-4-yl)-propan-2-ol

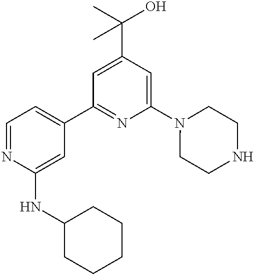

To a solution of 4-[2′-cyclohexylamino-4-(1-hydroxy-1-methyl-ethyl)-[2,4′]bipyridinyl-6-yl]-piperazine-1-carboxylic acid tert-butyl ester (0.072 g, 0.145 mmol) and CH₂Cl₂ (2 mL) is added TFA (0.5 mL). After stirring for 1 h, the solution is concentrated. The residue is then separated via semi-preparative HPLC (10-90% CH₃CN/H₂O gradient with 0.1% NH₄OH) to give the title compound 2-(2′-cyclohexylamino-6-piperazin-1-yl-[2,4′]bipyridinyl-4-yl)-propan-2-01. MS (ESI) m/z 396.0 (M+1). ¹H NMR (400 MHz, CDCl₃) δ ppm 8.11 (d, J=5.3 Hz, 1H), 7.13 (d, J=1.0 Hz, 1H), 7.07 (dd, J=5.4, 1.4 Hz, 1H), 7.03 (s, 1H), 6.83 (s, 1H), 4.47-4.55 (m, 1H), 3.64-3.73 (m, 1H), 3.60-3.64 (m, 4H), 2.98-3.05 (m, 4H), 2.03-2.14 (m, 2H), 1.81-1.94 (m, 2H), 1.70-1.81 (m, 2H), 1.60 (none, 1H), 1.60-1.69 (m, 1H), 1.58 (s, 6 H), 1.35-1.50 (m, 2H), 1.15-1.31 (m, 3H).

### Example 21

### A. 4-[2′-Cyclohexylamino-4-(5-oxo-4,5-dihydro-[1,3,4]oxadiazol-2-yl)-[2,4′]bipyridinyl-6-yl]-piperazine-1-carboxylic acid tert-butyl ester

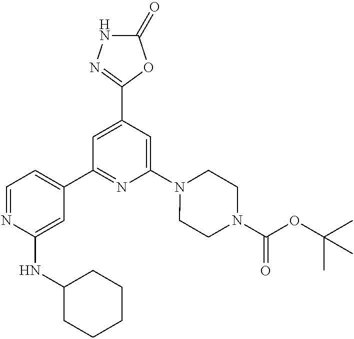

To a mixture of 4-(2′-cyclohexylamino-4-hydrazinocarbonyl-[2,4′]bipyridinyl-6-yl)-piperazine-1-carboxylic acid tert-butyl ester (prepared in Example 4A) (0.100 g, 0.202 mmol) and THF (2 mL) is added carbonyl diimidazole (0.039 g, 0.242 mmol) and Et₃N (0.05 mL, 0.404 mmol). After 15 min the mixture is poured into 25 mL of H₂O and extracted with CH₂Cl₂ (3×25 mL). The combined organic layers are then dried (Na₂SO₄), filtered and concentrated. The residue, 4-[2′-cyclohexylamino-4-(5-oxo-4,5-dihydro-[1,3,4]oxadiazol-[2-yl)-2,4′]bipyridinyl-6-yl]-piperazine-1-carboxylic acid tert-butyl ester, is taken on without further purification. MS (ESI) m/z 522.0 (M+1)

### B. 5-(2′-Cyclohexylamino-6-piperazin-1-yl-[2,4′]bipyridinyl-4-yl)-3H-[1,3,4]oxadiazol-2-one

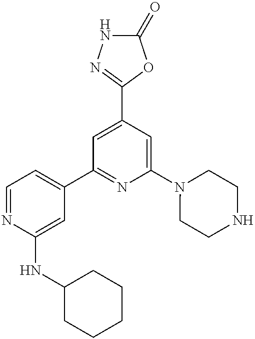

To a solution of 4-[2′-cyclohexylamino-4-(5-oxo-4,5-dihydro-[1,3,4]oxadiazol-2-yl)-[2,4′]bipyridinyl-6-yl]-piperazine-1-carboxylic acid tert-butyl ester (0.202 mmol) and CH₂Cl₂ (5 mL) is added TFA (2.5 mL). After stirring for 1 h, the solution is concentrated. The residue is then separated via semi-prep HPLC (10-90% CH₃CN/H₂O gradient with 0.1% NH₄OH) to give the title compound 5-(2′-cyclohexylamino-6-piperazin-1-yl-[2,4′]bipyridinyl-4-yl)-3H-[1,3,4]oxadiazol-2-one. MS (ESI) m/z 422.1 (M+1). ¹H NMR (400 MHz, DMSO-d₆) δ ppm 8.02 (d, J=5.3 Hz, 1H), 7.39 (s, 1H), 7.15 (s, 1H), 7.05 (s, 1H), 7.03 (dd, J=5.6, 1.5 Hz, 1H), 6.48 (d, J=7.6 Hz, 1H), 3.70-3.81 (m, 1H), 3.58-3.67 (m, 4H), 2.85-2.94 (m, 4H), 1.88-1.97 (m, 2H), 1.67-1.76 (m, 2H), 1.54-1.64 (m, 1H), 1.26-1.40 (m, 2H), 1.12-1.26 (m, 3H).

### Example 22

### A. 6-Bromopyridine-2-carboxylic acid

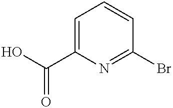

To a solution of 6-bromo-pyridine-2-carboxylic acid methyl ester (2.4 g, 10.5 mmol) in THF/water (30 mL, 2:1) is added LiOH.H₂O (2.2 g, 52.0 mmol) and the suspension is stirred at room temperature until reaction is complete by LCMS. Reaction is quenched to pH 5 with concentrated HCl and then evaporated to dryness in vacuo. This crude residue is used without purification for the next step. (ESI) m/z 203.9 (M+1).

### B. 4-(6-Bromopyridine-2-carbonyl)-piperazine-1-carboxylic acid tert-butyl ester

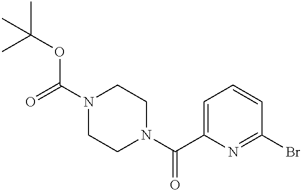

6-bromo-pyridine-2-carboxylic acid (2.5 g, 10.5 mmol), piperazine-1-carboxylic acid tert-butyl ester (3.9 g, 21.1 mmol), PyBOP (10.9 g, 21.1 mmol), HOBt.H₂O (3.2 g, 21.1 mmol) and Hunig's base (8.7 mL, 52.5 mmol) in DMF (20 mL) are stirred at ambient temperature for 16 h. Reaction is diluted with DCM (50 mL) and extracted between DCM and sat. aq. NaHCO₃ (×2). Organic is washed with brine, dried over anhydrous Na₂SO₄ and concentrated in vacuo. Crude residue is purified via flash chromatography (SiO₂, EtOAc/heptanes gradient) to give a yellow solid as the title compound (1.41 g, 36%). (ESI) m/z 372.2 (M+1). ¹H NMR (400 MHz, CD₂Cl₂) δ ppm 7.57-7.63 (m, 1H), 7.45-7.54 (m, 2 H), 3.58-3.67 (m, 2H), 3.40-3.48 (m, 4H), 3.33-3.39 (m, 2H), 1.37 (s, 9H).

### C. 4-(2′-Fluoro-[2,4′]bipyridinyl-6-carbonyl)-piperazine-1-carboxylic acid tert-butyl ester

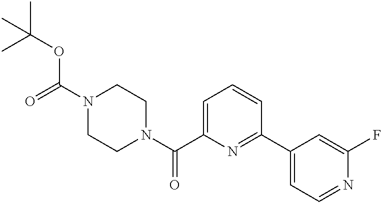

4-(6-bromopyridine-2-carbonyl)-piperazine-1-carboxylic acid tert-butyl ester (500 mg, 1.35 mmol), 2-fluoro-4-pyridine boronic acid (286 mg, 2.03 mmol) and 2.0 M Na₂CO₃ solution (2.0 mL, 4.06 mmol) in DME (15.0 mL) are stirred. Added Pd(PPh₃)₄ (156 mg, 0.13 mmol) and heated the reaction in a sealed pressure vessel at 110° C. until reaction is complete. Reaction is then diluted with EtOAc (15 mL) and extracted between organic and saturated NaHCO₃ (×2). The organic layer is washed with brine, dried over anhydrous Na₂SO₄ and then evaporated under reduced pressure to provide a crude residue that is purified via flash chromatography (SiO₂, EtOAc/heptanes gradient) to afford the compound as a pale yellow solid (418 mg, 80%). MS (ESI) m/z 387.1 (M+1). ¹H-NMR (400 MHz, CD₂Cl₂) δ ppm 8.36 (d, J=5.3 Hz, 1H), 7.98-8.05 (m, 1H), 7.94 (dd, 1H), 7.85 (dt, J=5.3, 1.6 Hz, 1H), 7.76 (dd, J=7.6, 1.0 Hz, 1H), 7.61 (s, 1H), 3.76-3.84 (m, 2H), 3.56-3.67 (m, 4H), 3.48-3.55 (m, 2H), 1.49 (s, 9H).

### D. 4-(2′-Cyclohexylamino-[2,4′]bipyridinyl-6-carbonyl)-piperazine-1-carboxylic acid tert-butyl ester

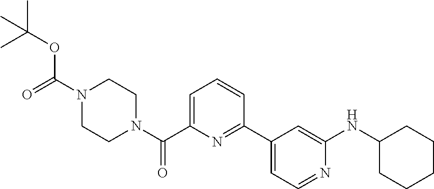

4-(2′-Fluoro-[2,4′]bipyridinyl-6-carbonyl)-piperazine-1-carboxylic acid tert-butyl ester (300 mg, 0.77 mmol) and neat cyclohexylamine (6.0 mL) are heated to 120° C. in a sealed pressure tube. After reaction is complete, the residue is separated via semi-prep HPLC (10-90% CH₃CN/H₂O gradient with 0.1% NH₄OH). A white solid is obtained (170 mg, 36%). MS (ESI) m/z 466.2 (M+1). ¹H-NMR (400 MHz, CD₂Cl₂) δ ppm 8.06 (d, J=5.6 Hz, 1H), 7.99 (t, J=7.8 Hz, 1H), 7.89 (dd, J=7.8, 1.0 Hz, 1H), 7.74 (d, J=7.1 Hz, 1H), 7.22 (s, 1H), 7.17 (dd, J=5.8, 1.5 Hz, 1H), 3.76-3.85 (m, 2H), 3.57-3.70 (m, 4H), 3.48-3.56 (m, 2H), 2.03-2.15 (m, 2H), 1.77-1.90 (m, 2H), 1.62-1.73 (m, 1H), 1.24-1.53 (m, 16H).

### E. (2′-Cyclohexylamino-[2,4′]bipyridinyl-6-yl)-piperazin-1-yl-methanone

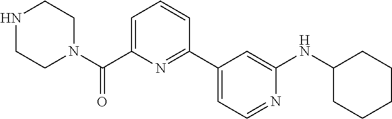

4-(2′-cyclohexylamino-[2,4′]bipyridinyl-6-carbonyl)-piperazine-1-carboxylic acid tert-butyl ester (50 mg, 0.10 mmol) in TFA/DCM 1:1 (4 mL) is stirred for 1 h. The mixture is evaporated to dryness in vacuo and purified via semi-preparative HPLC (10-90% CH₃CN/H₂O gradient with 0.1% NH₄OH). A white solid is obtained (26 mg, 67%). MS (ESI) m/z 366.4 (M+1). ¹H-NMR (400 MHz, CD₂Cl₂) δ ppm 8.16 (d, J=5.3 Hz, 1H), 7.93 (t, J=7.7 Hz, 1H), 7.83 (d, J=8.8 Hz, 1H), 7.64 (d, J=6.8 Hz, 1H), 7.11 (dd, J=5.4, 1.4 Hz, 1H), 7.07 (s, 1H), 4.73 (d, J=7.8 Hz, 1H), 3.76-3.85 (m, 2H), 3.58-3.76 (m, 3H), 2.97-3.06 (m, 2 H), 2.89-2.97 (m, 2H), 2.05-2.16 (m, 2H), 1.76-1.86 (m, 2H), 1.63-1.75 (m, 1H), 1.39-1.54 (m, 2H), 1.20-1.38 (m, 4H).

### Example 23

### A. 4-(2′-Cyclohexylamino-[2,4′]bipyridinyl-6-ylmethyl)-piperazine-1-carboxylic acid tert-butyl ester

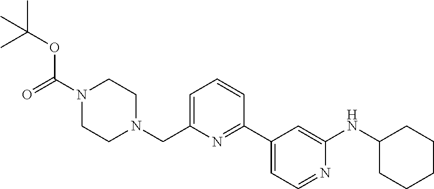

Stirred 4-(2′-cyclohexylamino-[2,4′]bipyridinyl-6-carbonyl)-piperazine-1-carboxylic acid tert-butyl ester, Example 22D, (130 mg, 0.28 mmol) in THF (5.0 mL) at 0° C. To this is added 1.0 M THF solution of DIBAL (2.80 mL, 2.80 mmol). After 1 h, the reaction is diluted with EtOAc and then extracted between EtOAc and a saturated solution of Rochelle's salt (×2), followed by brine (×1). The organic layer is dried over anhydrous Na₂SO₄ and evaporated in vacuo and used without purification further. MS (ESI) m/z 452.5 (M+1).

### B. Cyclohexyl-(6-piperazin-1-ylmethyl-[2,4′]bipyridinyl-2′-yl)-amine

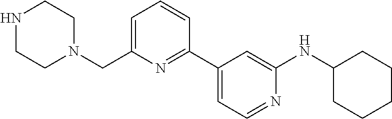

Stirred crude 4-(2′-cyclohexylamino-[2,4′]bipyridinyl-6-ylmethyl)-piperazine-1-carboxylic acid tert-butyl ester (126 mg, 0.28 mmol) in TFA/DCM 1:1 (4 mL) for 1 h. Evaporate to dryness in vacuo and separated via semi-prep HPLC (10-90% CH₃CN/H₂O gradient with 0.1% NH₄OH). A white solid is obtained (30 mg, 30%). MS (ESI) m/z 352.2 (M+1). ¹H-NMR (400 MHz, CD₂Cl₂) δ ppm 8.02 (d, J=4.8 Hz, 1H), 7.67 (t, J=7.7 Hz, 1H), 7.52 (d, J=7.1 Hz, 1H), 7.36 (d, J=7.6 Hz, 1H), 6.98 (dd, J=5.3, 1.5 Hz, 1H), 6.94 (s, 1H), 4.52 (d, J=7.8 Hz, 1H), 3.62 (s, 2H), 3.53-3.69 (obs m, 1H), 2.77-2.88 (m, 4H), 2.38-2.52 (m, 4H), 2.24 (br. s., 2H), 1.90-2.04 (m, 2H), 1.62-1.76 (m, 2H), 1.49-1.62 (m, 1 H), 1.27-1.43 (m, 2H), 1.06-1.26 (m, 4H).

### Example 24

### A. 6-piperazin-1-yl-2′-(tetrahydro-pyran-4-ylamino)-[2,4′]bipyridinyl-4-carbonitrile

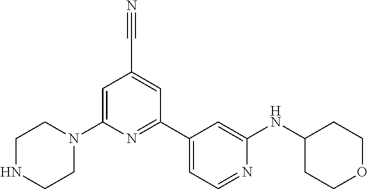

Stir 6-piperazin-1-yl-2′-(tetrahydro-pyran-4-ylamino)-[2,4′]bipyridinyl-4-carboxylic acid amide (257 mg, 0.67 mmol), Example 4F, in DCM (10.0 mL) and Et₃N (1.40 mL, 10.1 mmol) at room temperature before adding trifluoroacetic acid anhydride (0.47 mL, 3.36 mmol). After 2 h, the intermediate bis-triflamide nitrile product is observed via LCMS. The reaction is diluted with DCM (10.0 mL) and extracted with saturated NaHCO₃ solution. The organic layer is dried over anhydrous Na₂SO₄, filtered, and concentrated. This is used without further purification. MS (ESI) m/z 557.1 (M+1).

The residue from above is dissolved in MeOH (10.0 mL) and treated with NaBH₄ (128 mg, 3.36 mmol). After 2 h, the reaction is evaporated and separated via semi-prep HPLC (10-90% CH₃CN/H₂O gradient with 0.1% NH₄OH) to give the title compound (105 mg, 43%). MS (ESI) m/z 365.2 (M+1). ¹H NMR (400 MHz, MeOH-d₄) δ ppm 8.00 (d, J=5.6 Hz, 1H), 7.37 (s, 1H), 7.20 (s, 1H), 7.07-7.15 (m, 2H), 3.91-4.03 (m, 3H), 3.64-3.72 (m, 4H), 3.56 (dt, J=11.6, 2.0 Hz, 2H), 2.88-2.99 (m, 4H), 1.95-2.07 (m, 2H), 1.47-1.62 (m, 2H).

Compounds B-F of Example 24 can be prepared by a similar method.

### B. 2′-Phenylamino-6-piperazin-1-yl-[2,4′]bipyridinyl-4-carbonitrile

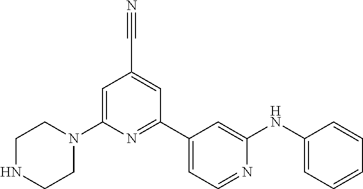

MS (ESI) m/z 357.2 (M+1). ¹H NMR (400 MHz, MeOH-d₄) δ ppm 8.16 (d, J=5.6 Hz, 1H), 7.51-7.57 (m, 2H), 7.48-7.52 (m, 1H), 7.39 (s, 1H), 7.24-7.35 (m, 3H), 7.14 (s, 1 H), 6.91-7.03 (m, 1H), 3.57-3.74 (m, 4H), 2.86-3.00 (m, 4H).

### C. 2′-Cyclohexylamino-6-piperazin-1-yl-[2,4′]bipyridinyl-4-carbonitrile

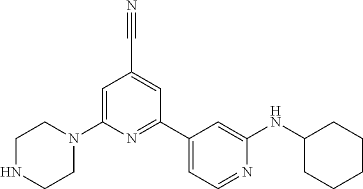

MS (ESI) m/z 363.3 (M+1). ¹H NMR (400 MHz, DMSO-d₆) δ ppm 8.03 (d, J=5.3 Hz, 1H), 7.45 (s, 1H), 7.30 (s, 1H), 7.15 (s, 1H), 7.03 (dd, J=5.4, 1.4 Hz, 1H), 6.50 (d, J=7.7 Hz, 1H), 3.67-3.81 (m, 1H), 3.51-3.65 (m, 4H), 2.73-2.88 (m, 4H), 1.85-1.99 (m, 2H), 1.66-1.78 (m, 2H), 1.54-1.66 (m, 1H), 1.09-1.41 (m, 6H).

### D. 2′-(1-Methyl-1H-pyrazol-3-ylamino)-6-piperazin-1-yl-[2,4′]bipyridinyl-4-carbonitrile

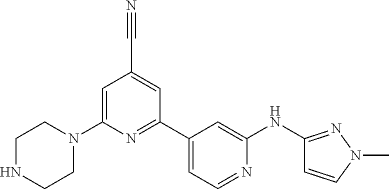

MS (ESI) m/z 361.2 (M+1). ¹H NMR (400 MHz, MeOH-d₄) δ ppm 8.16 (d, J=5.3 Hz, 1H), 7.92 (s, 1H), 7.46 (d, J=2.0 Hz, 1H), 7.40 (s, 1H), 7.32 (dd, J=5.3, 1.5 Hz, 1H), 7.14 (s, 1H), 6.25 (d, J=2.3 Hz, 1H), 3.82 (s, 3H), 3.64-3.73 (m, 4H), 2.89-3.01 (m, 4H).

### E. 2′-Isopropylamino-6-piperazin-1-yl-[2,4′]bipyridinyl-4-carbonitrile

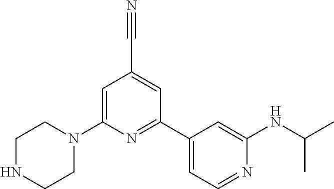

MS (ESI) m/z 323.2 (M+1). ¹H NMR (400 MHz, MeOH-d₄) δ ppm 7.99 (d, J=5.1 Hz, 1H), 7.36 (s, 1H), 7.15 (s, 1H), 7.12 (s, 1H), 7.09 (dd, J=5.7, 1.6 Hz, 1H), 3.94-4.09 (m, 1H), 3.60-3.72 (m, 4H), 2.87-2.99 (m, 4H), 1.23 (d, J=6.6 Hz, 6H).

### F. 6-piperazin-1-yl-2′-(piperidin-4-ylamino)-[2,4′]bipyridinyl-4-carbonitrile

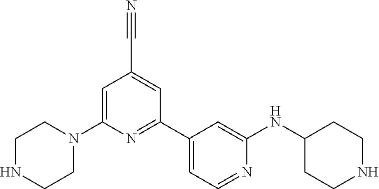

MS (ESI) m/z 364.1 (M+1). ¹H NMR (400 MHz, MeOH-d₄) δ ppm 7.91 (d, J=5.1 Hz, 1H), 7.27 (s, 1H), 7.10 (s, 1H), 6.97-7.06 (m, 2H), 3.70-3.87 (m, 1H), 3.49-3.64 (m, 4 H), 3.00-3.11 (m, 2H), 2.79-2.89 (m, 4H), 2.62-2.78 (m, 2H), 1.94-2.02 (m, 2H), 1.30-1.48 (m, 2H).

### Example 25

### A. 6-(4-tert-Butoxycarbonyl-piperazin-1-yl)-2′-fluoro-[2,4′]-bipyridinyl-4-carboxylic acid methyl ester

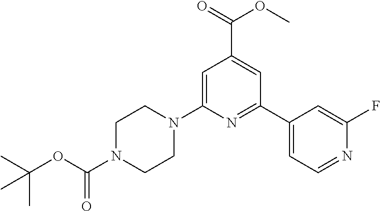

4-(6-Bromo-4-methoxycarbonyl-pyridin-2-yl)-piperazine-1-carboxylic acid tert-butyl (2.0 g, 5.01 mmol), Example 4B, and 2-fluoro-4-pyridine boronic acid (0.85 g, 6.01 mmol) are dissolved in DME (40 mL). To this is added 2.0 M Na₂CO₃ solution (7.5 mL, 15.03 mmol) and Pd(dppf)Cl₂.CH₂Cl₂ (0.41 g, 0.50 mmol). This above suspension is heated to 80° C. for 4 h. Reaction is diluted with EtOAc (25.0 mL) and extracted between organic and saturated NaHCO₃ (×2). The organic layer is washed with brine, dried over anhydrous Na₂SO₄ and then evaporated under reduced pressure to provide a crude residue that is purified via flash chromatography (SiO₂, EtOAc/heptanes gradient) to afford the title compound as a pale yellow solid (1.80 g, 90%). MS (ESI) m/z 417.1 (M+1). ¹H-NMR (400 MHz, CDCl₃) δ ppm 8.32 (d, J=5.3 Hz, 1H), 7.81 (dt, J=5.2, 1.5 Hz, 1H), 7.70 (s, 1H), 7.60 (s, 1H), 7.35 (s, 1 H), 4.00 (s, 3H), 3.70-3.78 (m, 4H), 3.58-3.67 (m, 4H), 1.52 (s, 9H).

### B. 3-Bromo-6-(4-tert-butoxycarbonyl-piperazin-1-yl)-2′-fluoro-[2,4′]bipyridinyl-4-carboxylic acid methyl ester

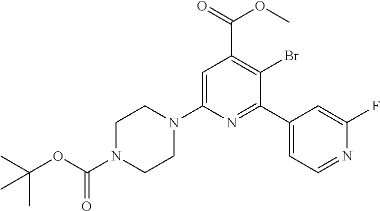

6-(4-tert-butoxycarbonyl-piperazin-1-yl)-2′-fluoro-[2,4′]-bipyridinyl-4-carboxylic acid methyl ester (100 mg, 0.24 mmol) is dissolved in 3.0 mL of DCM. Solid KOAc (141.0 mg, 1.44 mmol) is then added to the solution. The solution is cooled to 0° C. and a solution of Br₂ (13.0 uL, 0.25 mmol) in 1.0 mL DCM is added. After 20 min, reaction is poured into a 1:1 Na₂S₂O₃/NaHCO₃ solution and extracted. The organic layer dried over anhydrous Na₂SO₄ and then evaporated under reduced pressure to provide a crude residue that is purified via flash chromatography (SiO₂, EtOAc/heptanes gradient) to afford the title compound as a pale yellow solid (83.0 mg, 70%). MS (ESI) m/z 496.8 (M+1). ¹H-NMR (400 MHz, CDCl₃) δ ppm 8.19 (d, J=5.1 Hz, 1H), 7.35-7.42 (m, 1H), 7.12 (s, 1H), 6.77 (s, 1H), 3.86 (s, 3H), 3.46-3.53 (m, 4H), 3.40-3.45 (m, 4H), 1.37 (s, 9H).

### C. 6-(4-tert-Butoxycarbonyl-piperazin-1-yl)-2′-fluoro-3-phenyl-[2,4′]bipyridinyl-4-carboxylic acid methyl ester

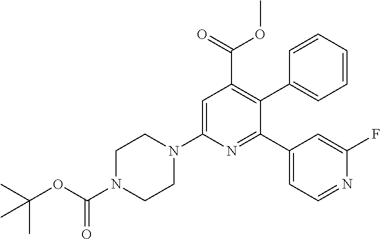

3-bromo-6-(4-tert-butoxycarbonyl-piperazin-1-yl)-2′-fluoro-[2,4′]bipyridinyl-4-carboxylic acid methyl ester (100.0 mg, 0.20 mmol), phenyl boronic acid (39.0 mg, 0.30 mmol), Pd(dppf)Cl₂.DCM (8.0 mg, 10.1 umol) and 2.0 M Na₂CO₃ solution (0.20 mL, 0.40 mmol) is heated in DME (2.0 mL) in a microwave reactor at 130° C. for 45 min. Reaction is diluted with DCM and extracted between organic and saturated NaHCO₃ (×2). The organic layer is washed with brine, dried over anhydrous Na₂SO₄ and then evaporated under reduced pressure to provide a crude residue that is purified via flash chromatography (SiO₂, EtOAc/heptanes gradient) to afford the title compound as a pale yellow solid (90.2 mg, 91%). MS (ESI) m/z 493.2 (M+1).

### D. 6-(4-tert-Butoxycarbonyl-piperazin-1-yl)-2′-fluoro-3-phenyl-[2,4′]bipyridinyl-4-carboxylic acid

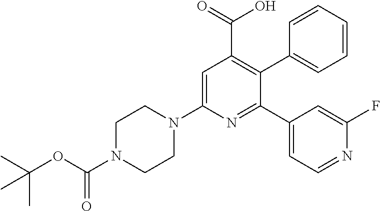

To a solution of 6-(4-tert-butoxycarbonyl-piperazin-1-yl)-2′-fluoro-3-phenyl-[2,4′]bipyridinyl-4-carboxylic acid methyl ester (150.0 mg, 0.30 mmol) in THF:water (4.0 mL, 3:1) is added LiOH.H₂O (64.0 mg, 1.50 mmol) and the suspension is stirred at room temperature until reaction is complete by LCMS. Reaction is quenched to pH 6 with 1.0 N HCl and then evaporated to dryness in vacuo. This crude residue is used without purification for the next step. (ESI) m/z 479.0 (M+1).

### E. 4-(4-Carbamoyl-2′-fluoro-3-phenyl-[2,4′]bipyridinyl-6-yl)-piperazine-1-carboxylic acid tert-butyl ester

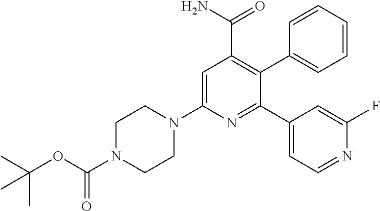

6-(4-tert-butoxycarbonyl-piperazin-1-yl)-2′-fluoro-3-phenyl-[2,4′]bipyridinyl-4-carboxylic acid (146.0 mg, 0.30 mmol), HATU (460.0 mg, 1.20 mmol), Hunig's base (0.50 mL, 3.04 mmol) and NH₄Cl (162.0 mg, 3.04 mmol) are combined in anhydrous DMF (10.0 mL). Reaction is monitored via LCMS and is evaporated in vacuo on completion. Reaction is extracted between DCM and saturated sodium bicarbonate solution. The organic layer is dried over anhydrous Na₂SO₄ and concentrated. The residue is purified via flash chromatography (SiO₂, EtOAc/heptanes gradient) to afford the title compound as a pale yellow solid (74.1 mg, 51%). MS (ESI) m/z 478.1 (M+1).

### F. 4-(4-Carbamoyl-2′-cyclohexylamino-3-phenyl-[2,4′]bipyridinyl-6-yl)-piperazine-1-carboxylic acid tert-butyl ester

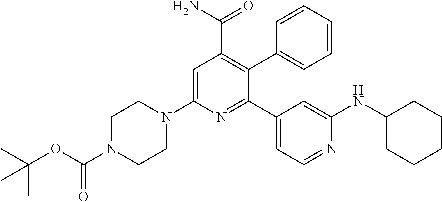

4-(4-carbamoyl-2′-fluoro-3-phenyl-[2,4′]bipyridinyl-6-yl)-piperazine-1-carboxylic acid tert-butyl ester (75.0 mg, 0.15 mmol), CuF₂ (32.0 mg, 0.31 mmol) and cyclohexylamine (2.0 mL, excess) are combined in a pressure vessel. The vessel is then sealed and the contents heated to 150° C. for 12 h. The mixture is then allowed to cool followed by concentration. The residue is then separated via flash chromatography (SiO₂, EtOAc/hexanes gradient) to give the title compound (65.0 mg, 75%). MS (ESI) m/z 557.2 (M+1).

### G. 2′-Cyclohexylamino-3-phenyl-6-piperazin-1-yl-[2,4′]bipyridinyl-4-carboxylic acid amide

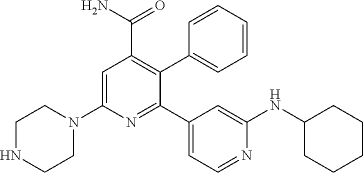

To a solution of 4-(4-carbamoyl-2′-cyclohexylamino-3-phenyl-[2,4′]bipyridinyl-6-yl)-piperazine-1-carboxylic acid tert-butyl ester (65.0 mg, 0.12 mmol) and CH₂Cl₂ (5.0 mL) is added TFA (5.0 mL). After stirring for 1 h the solution is concentrated. The residue is taken up in CH₂Cl₂ (10.0 mL) and washed with a saturated aqueous solution of NaHCO₃. The aqueous layer is further extracted with CH₂Cl₂ (2×10 mL). The combined organic layers are then dried (Na₂SO₄), filtered and concentrated. The residue is separated via semi-prep HPLC (10-90% CH₃CN/H₂O gradient with 0.1% NH₄OH) to give the title compound (37.0 mg, 70%). MS (ESI) m/z 457.2 (M+1). ¹H NMR (400 MHz, MeOH-d₄) δ ppm 7.60 (d, J=6.1 Hz, 1H), 7.13-7.19 (m, 3H), 7.05-7.12 (m, 2H), 6.79 (s, 1H), 6.33 (dd, J=5.4, 1.4 Hz, 1H), 6.23 (s, 1H), 3.51-3.60 (m, 4H), 3.06-3.18 (m, 1H), 2.79-2.90 (m, 4H), 1.49-1.76 (m, 5H), 0.93-1.32 (m, 5H).

### Example 26

### A. 4-(2′-Fluoro-4-hydroxymethyl-[2,4′]bipyridinyl-6-yl)-piperazine-1-carboxylic acid tert-butyl ester

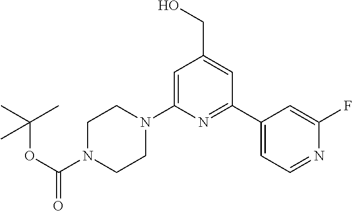

6-(4-tert-butoxycarbonyl-piperazin-1-yl)-2′-fluoro-[2,4′]-bipyridinyl-4-carboxylic acid methyl ester (3.00 g, 7.21 mmol), Example 25A, is stirred in THF (100 mL) at 0° C. before added 1.0 M Et₂O solution of LiAlH₄ (8.60 mL, 8.65 mmol). When the reaction is complete, it is quenched with 9.0 mL water followed by 18.0 mL 1 N NaOH and finally 9.0 mL water. This is transferred to a separatory funnel and extracted between DCM and saturated aqueous NH₄Cl and brine. The organic layer is dried over anhydrous Na₂SO₄ and concentrated to give a crude residue that is purified via flash chromatography (SiO₂, EtOAc/hexanes gradient) to give the title compound (2.50 g, 89%). MS (ESI) m/z 389.2 (M+1). ¹H NMR (400 MHz, CD₂Cl₂) δ ppm 8.16 (d, J=5.3 Hz, 1H), 7.63-7.74 (m, 1H), 7.49 (s, 1H), 7.09 (s, 1H), 6.70 (s, 1H), 4.64 (s, 2H), 3.52-3.61 (m, 4H), 3.42-3.50 (m, 4H), 1.39 (s, 9H).

### B. 4-(2′-Cyclohexylamino-4-hydroxymethyl-[2,4′]bipyridinyl-6-yl)-piperazine-1-carboxylic acid tert-butyl ester

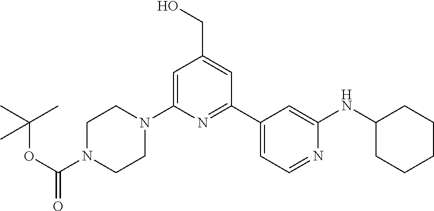

4-(2′-fluoro-4-hydroxymethyl-[2,4′]bipyridinyl-6-yl)-piperazine-1-carboxylic acid tert-butyl ester (1.00 g, 2.60 mmol) is stirred along with CuF₂ (0.26 mg, 2.60 mmol) in neat cyclohexylamine (25 mL) at 150° C. in a sealed pressure vessel for 16 h. When the reaction is complete, it is filtered to remove salts and concentrated to give a crude residue that is purified via column chromatography (SiO₂, EtOAc/hexanes gradient) to give the title compound (0.64 g, 54%). A side-product, Example 26A, is also obtained (0.26 g, 15%). MS (ESI) m/z 468.2 (M+1). ¹H NMR (400 MHz, CD₂Cl₂) δ ppm 7.97 (d, J=5.3 Hz, 1H), 6.97-7.04 (m, 2H), 6.95 (s, 1H), 6.64 (s, 1H), 4.61 (s, 2H), 4.55 (br. s., 1H), 3.51-3.58 (m, 4 H), 3.39-3.49 (m, 4H), 1.94-2.04 (m, 2H), 1.64-1.75 (m, 2H), 1.49-1.62 (m, 1H), 1.27-1.41 (m, 12H), 1.08-1.25 (m, 3H).

### C. (2′-Cyclohexylamino-6-piperazin-1-yl-[2,4′]bipyridinyl-4-yl)-methanol

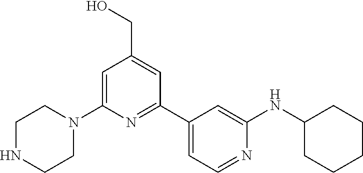

To a solution of 4-(2′-cyclohexylamino-4-hydroxymethyl-[2,4′]bipyridinyl-6-yl)-piperazine-1-carboxylic acid tert-butyl ester (50.0 mg, 0.11 mmol) and CH₂Cl₂ (5.0 mL) is added TFA (5.0 mL). After stirring for 1 h the solution is concentrated. The residue is taken up in CH₂Cl₂ (10.0 mL) and washed with a saturated aqueous solution of NaHCO₃. The aqueous layer is further extracted with CH₂Cl₂ (2×10 mL). The combined organic layers are then dried (Na₂SO₄), filtered and concentrated. The residue is then separated via semi-prep HPLC (10-90% CH₃CN/H₂O gradient with 0.1% NH₄OH) to give the title compound (25.1 mg, 64%). MS (ESI) m/z 368.1 (M+1). ¹H NMR (400 MHz, MeOH-d₄) δ ppm 7.84 (d, J=5.6 Hz, 1H), 7.08 (s, 2H), 6.99 (dd, J=5.6, 1.5 Hz, 1H), 6.72 (s, 1H), 4.53 (s, 2H), 3.50-3.58 (m, 4H), 2.83-2.92 (m, 4H), 1.88-1.99 (m, 2H), 1.64-1.75 (m, 2H), 1.52-1.63 (m, 1H), 1.27-1.42 (m, 2H), 1.06-1.24 (m, 3H).

### Example 27

### A. 4-(4-Bromomethyl-2′-cyclohexylamino-[2,4′]bipyridinyl-6-yl)-piperazine-1-carboxylic acid tert-butyl ester

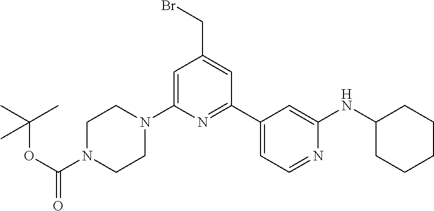

4-(2′-cyclohexylamino-4-hydroxymethyl-[2,4′]bipyridinyl-6-yl)-piperazine-1-carboxylic acid tert-butyl ester (0.60 g, 1.29 mmol), Example 26B, PPh₃ (0.67 g, 2.58 mmol) and CBr₄ (0.64 g, 1.94 mmol) are stirred in THF (12 mL) at room temperature until reaction is complete after which the reaction is filtered, concentrated and purified via flash chromatography (SiO₂, EtOAc/heptanes gradient) to give the title compound (0.38 g, 56%). MS (ESI) m/z 531.9 (M+1). ¹H NMR (400 MHz, CD₂Cl₂) δ ppm 7.99 (d, J=5.3 Hz, 1H), 7.04 (s, 1H), 6.98 (dd, J=5.4, 1.4 Hz, 1H), 6.93 (s, 1H), 6.60 (s, 1H), 4.58 (br. s., 1H), 4.33 (s, 2H), 3.52-3.58 (m, 4H), 3.43-3.50 (m, 4H), 1.94-2.03 (m, 2H), 1.64-1.75 (m, 2H), 1.50-1.62 (m, 1H), 1.39 (s, 9H), 1.28-1.35 (m, 2H), 1.09-1.25 (m, 4H).

### B. 4-(4-Cyanomethyl-2′-cyclohexylamino-[2,4′]bipyridinyl-6-yl)-piperazine-1-carboxylic acid tert-butyl ester

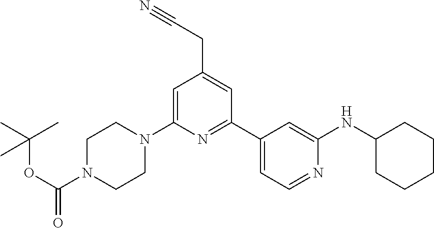

4-(4-bromomethyl-2′-cyclohexylamino-[2,4′]bipyridinyl-6-yl)-piperazine-1-carboxylic acid tert-butyl ester (380.0 mg, 0.72 mmol) and NaCN (53.0 mg, 1.10 mmol) are stirred in DMSO (10 mL) and warmed the reaction to 80° C. for 1 h. The reaction is concentrated and partitioned between DCM and saturated aqueous NaHCO₃ and brine. The organic layer is dried over anhydrous Na₂SO4 and concentrated to give a crude residue that is purified via flash chromatography (SiO₂, EtOAc/hexanes gradient) to give the title compound (150.0 mg, 44%). MS (ESI) m/z 477.2 (M+1). ¹H NMR (400 MHz, CD₂Cl₂) δ ppm 7.97 (d, J=5.3 Hz, 1H), 6.94-7.01 (m, 3H), 6.57 (s, 1H), 3.67 (s, 2H), 3.52-3.61 (m, 4H), 3.42-3.51 (m, 4H), 1.88-2.04 (m, 2H), 1.64-1.77 (m, 2H), 1.52-1.63 (m, 1H), 1.39 (s, 9H), 1.26-1.35 (m, 2H), 1.08-1.26 (m, 5H).

### C. (2′-Cyclohexylamino-6-piperazin-1-yl-[2,4′]bipyridinyl-4-yl)-acetonitrile

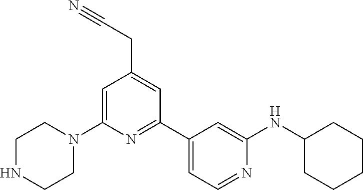

To a solution of 4-(4-cyanomethyl-2′-cyclohexylamino-[2,4′]bipyridinyl-6-yl)-piperazine-1-carboxylic acid tert-butyl ester (50.0 mg, 0.10 mmol) and CH₂Cl₂ (5.0 mL) is added TFA (5.0 mL). After stirring for 1 h the solution is concentrated. The residue is taken up in CH₂Cl₂ (10.0 mL) and washed with a saturated aqueous solution of NaHCO₃. The aqueous layer is further extracted with CH₂Cl₂ (2×10 mL). The combined organic layers are then dried (Na₂SO₄), filtered and concentrated. The residue is then separated via semi-preparative HPLC (10-90% CH₃CN/H₂O gradient with 0.1% NH₄OH) to give the title compound (15.1 mg, 38%). MS (ESI) m/z 377.1 (M+1). ¹H NMR (400 MHz, MeOH-d₄) δ ppm 7.86 (d, J=5.1 Hz, 1H), 7.09 (s, 1H), 7.07 (s, 1H), 6.99 (dd, J=5.6, 1.5 Hz, 1H), 6.71 (s, 1H), 3.50-3.65 (m, 5H), 2.78-2.92 (m, 4H), 1.88-2.01 (m, 2H), 1.65-1.78 (m, 2H), 1.53-1.64 (m, 1H), 1.27-1.43 (m, 2H), 1.07-1.25 (m, 3H).

### Example 28

### A. 2-(2′-Cyclohexylamino-6-piperazin-1-yl-[2,4′]bipyridinyl-4-yl)-acetamide

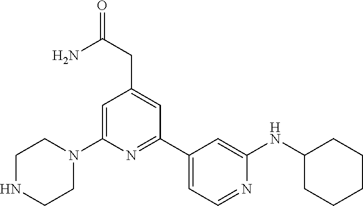

4-(4-cyanomethyl-2′-cyclohexylamino-[2,4′]bipyridinyl-6-yl)-piperazine-1-carboxylic acid tert-butyl ester (40.0 mg, 84.0 umol), Example 27B, is stirred in THF (2.0 mL) and TMSCl (15.0 mL, 8.40 mmol) is added followed by water (60.0 uL, 8.40 mmol). After 4 h, the reaction is concentrated and purified via semi-preparative HPLC (10-90% CH₃CN/H₂O gradient with 0.1% NH₄OH) to give the title compound (15.0 mg, 45%). MS (ESI) m/z 395.1 (M+1). ¹H NMR (400 MHz, MeOH-d₄) δ ppm 7.84 (d, J=5.6 Hz, 1H), 7.08 (s, 1H), 7.05 (s, 1 H), 6.98 (dd, J=5.6, 1.5 Hz, 1H), 6.67 (s, 1H), 3.51-3.58 (m, 4H), 3.43 (s, 2H), 2.81-2.91 (m, 4H), 1.89-1.99 (m, 2H), 1.65-1.76 (m, 2H), 1.53-1.63 (m, 1H), 1.28-1.42 (m, 2H), 1.10-1.26 (m, 4H).

### Example 29

### A. 4-(4-Azidomethyl-2′-cyclohexylamino-[2,4′]bipyridinyl-6-yl)-piperazin-1-carboxylic acid tert-butyl ester

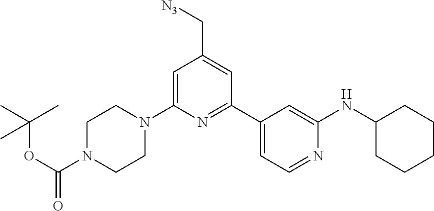

4-(4-bromomethyl-2′-cyclohexylamino-[2,4′]bipyridinyl-6-yl)-piperazine-1-carboxylic acid tert-butyl ester (285.0 mg, 0.54 mmol), Example 27A, and NaN₃ (53.0 mg, 0.81 mmol) are stirred in DMSO (5 mL)/DCM (2 mL) at room temperature for 3 h. The reaction is concentrated and extracted between DCM and brine. The organic layer is dried over anhydrous Na₂SO4 and concentrated to give a crude residue that is purified via flash chromatography (SiO₂, EtOAc/hexanes gradient) to give the title compound (230.0 mg, 87%). MS (ESI) m/z 493.3 (M+1).

### B. (4-Azidomethyl-6-piperazin-1-yl-[2,4′]bipyridinyl-2′-yl)-cyclohexyl-amine

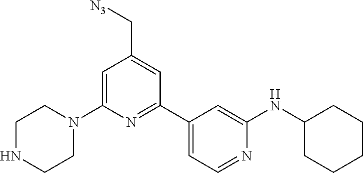

To a solution of 4-(4-azidomethyl-2′-cyclohexylamino-[2,4′]bipyridinyl-6-yl)-piperazin-1-carboxylic acid tert-butyl ester (80.0 mg, 0.16 mmol) and CH₂Cl₂ (5.0 mL) is added TFA (5.0 mL). After stirring for 2 h the solution is concentrated. The residue is taken up in CH₂Cl₂ (10.0 mL) and washed with a saturated aqueous solution of NaHCO₃. The aqueous layer is further extracted with CH₂Cl₂ (2×10 mL). The combined organic layers are then dried (Na₂SO₄), filtered and concentrated. The residue is then separated via semi-prep HPLC (10-90% CH₃CN/H₂O gradient with 0.1% NH₄OH) to give the title compound (15.5 mg, 24%). MS (ESI) m/z 393.1 (M+1). ¹H NMR (400 MHz, MeOH-d₄) δ ppm 7.85 (d, J=5.7 Hz, 1H), 7.09 (s, 1H), 7.05 (s, 1H), 6.99 (dd, J=5.6, 1.5 Hz, 1H), 6.69 (s, 1H), 4.32 (s, 2H), 3.47-3.67 (m, 5H), 2.86 (dd, J=6.0, 4.4 Hz, 4H), 1.85-2.01 (m, 2H), 1.65-1.76 (m, 2H), 1.50-1.63 (m, 1H), 1.28-1.43 (m, 2H), 1.09-1.25 (m, 3H).

### Example 30

### A. 4-(4-Aminomethyl-2′-cyclohexylamino-[2,4′]bipyridinyl-6-yl)-piperazin-1-carboxylic acid tert-butyl ester

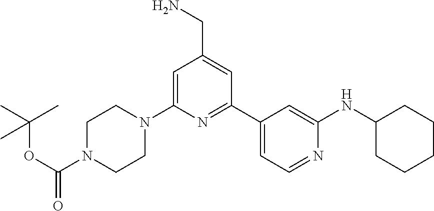

4-(4-azidomethyl-2′-cyclohexylamino-[2,4′]bipyridinyl-6-yl)-piperazin-1-carboxylic acid tert-butyl ester (170 mg, 0.34 mmol), Example 29B, is stirred in THF (5 mL) at 0° C. then a 1.0 M THF solution of LiAlH₄ (0.36 mL, 0.36 mmol) is added. When the reaction is complete, it is quenched with 1.0 mL water followed by 2.0 mL 1 N NaOH and finally 1.0 mL water. This is transferred to a separatory funnel and extracted between DCM and saturated aqueous NH₄Cl and brine. The organic layer is dried over anhydrous Na₂SO₄ and concentrated to give a crude residue that is purified via flash chromatography (SiO₂, EtOAc/hexanes gradient) to give the title compound (161.0 mg, 99%). MS (ESI) m/z 467.2 (M+1).

### B. (4-Aminomethyl-6-piperazin-1-yl-[2,4′]bipyridinyl-2′-yl)-cyclohexyl-amine

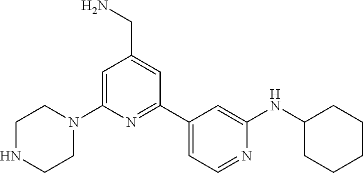

To a solution of 4-(4-aminomethyl-2′-cyclohexylamino-[2,4′]bipyridinyl-6-yl)-piperazin-1-carboxylic acid tert-butyl ester (161.0 mg, 0.34 mmol) and CH₂Cl₂ (5.0 mL) is added TFA (5.0 mL). After stirring for 2 h the solution is concentrated. The residue is taken up in CH₂Cl₂ (10.0 mL) and washed with a saturated aqueous solution of NaHCO₃. The aqueous layer is further extracted with CH₂Cl₂ (2×10 mL). The combined organic layers are then dried (Na₂SO₄), filtered and concentrated. The residue is then separated via semi-prep HPLC (10-90% CH₃CN/H₂O gradient with 0.1% NH₄OH) to give the title compound (30.0 mg, 21%). MS (ESI) m/z 367.1 (M+1). ¹H NMR (400 MHz, MeOH-d₄) δ ppm 7.84 (d, J=5.1 Hz, 1H), 7.10 (s, 2H), 7.01 (dd, J=5.6, 1.5 Hz, 1H), 6.72 (s, 1H), 3.71 (s, 2H), 3.47-3.60 (m, 5 H), 2.76-2.92 (m, 4H), 1.85-2.01 (m, 2H), 1.65-1.77 (m, 2H), 1.52-1.64 (m, 1H), 1.27-1.43 (m, 2H), 1.07-1.26 (m, 3H).

### Example 31

### A. 4-(2′-Cyclohexylamino-4-formyl-[2,4′]bipyridinyl-6-yl)-piperazine-1-carboxylic acid tert-butyl ester

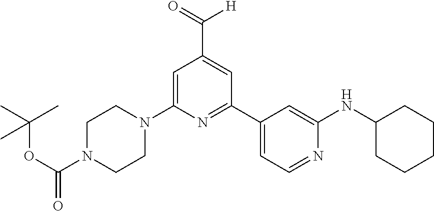

The title compound is obtained as a side-product of reaction to synthesize Example 26B. MS (ESI) m/z 466.2 (M+1). ¹H NMR (400 MHz, DMSO-d₆) δ ppm 10.04 (s, 1H), 8.04 (d, J=5.3 Hz, 1H), 7.51 (s, 1H), 7.37 (s, 1H), 7.20 (s, 1H), 7.06 (dd, J=5.4, 1.4 Hz, 1H), 3.64-3.73 (m, 4H), 3.44-3.53 (m, 4H), 1.86-2.01 (m, 2H), 1.47-1.55 (m, 3H), 1.44 (s, 9H), 1.19-1.37 (m, 3H), 1.00-1.12 (m, 4H).

### B. 4-{4-[Cyanomethylamino)-methyl]2′-cyclohexylamino-[2,4′]bipyridinyl-6-yl}-piperazine-1-carboxylic acid tert-butyl ester

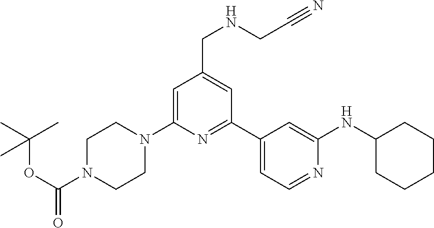

4-(2′-cyclohexylamino-4-formyl-[2,4′]bipyridinyl-6-yl)-piperazine-1-carboxylic acid tert-butyl ester (100.0 mg, 0.21 mmol) and aminoacetonitrile (18.0 uL, 0.32 mmol) are stirred in 1,2-dichoroethane (5 mL) at room temperature before adding sodium triactoxyborohydride (136.0 mg, 0.64 mmol). After 8 h, reaction is not complete. To the reaction is added MeOH (5 mL), followed by sodium borohydride (24.0 mg, 63.0 mmol). After stirring for 2 h, the solution is concentrated. The residue is taken up in CH₂Cl₂ (5 mL) and washed with a saturated aqueous solution of NaHCO₃. The aqueous layer is further extracted with CH₂Cl₂ (2×10 mL). The combined organic layers are then dried (Na₂SO₄), filtered and concentrated. The residue is used without further purification. MS (ESI) m/z 506.2 (M+1).

### C. [(2′-Cyclohexylamino-6-piperazin-1-yl-[2,4′]bipyridinyl-4-ylmethyl)-amino]-acetonitrile

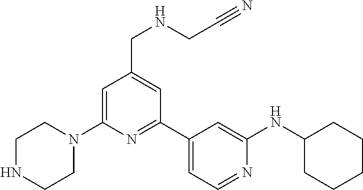

To a solution of 4-{-4-[cyanomethyl-amino)-methyl]2′-cyclohexylamino-[2,4′]bipyridinyl-6-yl}-piperazine-1-carboxylic acid tert-butyl ester (80.0 mg, 0.16 mmol) and CH₂Cl₂ (5.0 mL) is added TFA (5.0 mL). After stirring for 2 h the solution is concentrated. The residue is taken up in CH₂Cl₂ (10 mL) and washed with a saturated aqueous solution of NaHCO₃. The aqueous layer is further extracted with CH₂Cl₂ (2×10 mL). The combined organic layers are then dried (Na₂SO₄), filtered and concentrated. The residue is then separated via semi-preparative HPLC (10-90% CH₃CN/H₂O gradient with 0.1% NH₄OH) to give the title compound (25.0 mg, 39%). MS (ESI) m/z 406.1 (M+1). ¹H NMR (400 MHz, MeOH-d₄) δ ppm 7.84 (d, J=5.7 Hz, 1H), 7.10 (d, J=6.9 Hz, 2H), 7.00 (dd, J=5.6, 1.5 Hz, 1H), 6.73 (s, 1 H), 3.78 (s, 2H), 3.49-3.59 (m, 7H), 2.79-2.91 (m, 4H), 1.87-2.01 (m, 2H), 1.64-1.76 (m, 2H), 1.51-1.64 (m, 1H), 1.28-1.43 (m, 2H), 1.07-1.24 (m, 3H).

### Example 32

### A. 6-(4-tert-Butoxycarbonyl-piperazin-1-yl)-2′-cyclohexylamino-[2,4′]bipyridinyl-4-carboxylic acid methyl ester

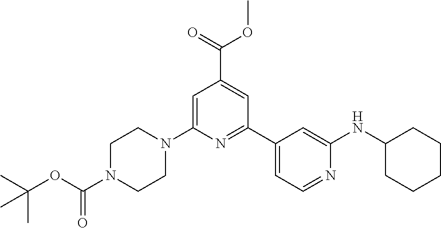

A mixture of 6-(4-tert-butoxycarbonyl-piperazin-1-yl)-2′-chloro-[2,4′]bipyridinyl-4-carboxylic acid methyl ester (75 mg, 0.17 mmol), Example 4C, Pd(tBu₃P)₂ (9.0 mg, 20.0 umol), Cs₂CO₃ (226.0 mg, 0.69 mmol), cyclohexylamine (40.0 uL, 0.34 mmol) and 1,4-dioxane (3.0 mL) is sparged with argon for 10 min. The vessel is then sealed and the contents heated to 100° C. for 3 h. The mixture is then allowed to cool followed by concentration. The residue is then separated via flash chromatography (SiO₂, EtOAc/hexanes gradient) to give the title compound (22.0 mg, 25%). MS (ESI) m/z 496.0 (M+1). ¹H NMR (400 MHz, CD₂Cl₂) δ ppm 8.01 (d, J=5.6 Hz, 1H), 7.53 (s, 1H), 7.18 (s, 1 H), 7.02 (dd, J=5.3, 1.3 Hz, 1H), 6.97 (s, 1H), 4.60 (br. s., 1H), 3.85 (s, 3H), 3.55-3.65 (m, 4H), 3.44-3.51 (m, 4H), 1.91-2.05 (m, 2H), 1.63-1.76 (m, 2H), 1.53-1.63 (m, 2H), 1.39 (s, 9H), 1.10-1.25 (m, 4H).

### B. 2′-Cyclohexylamino-6-piperazin-1-yl-[2,4′]bipyridinyl-4-carboxylic acid methyl ester

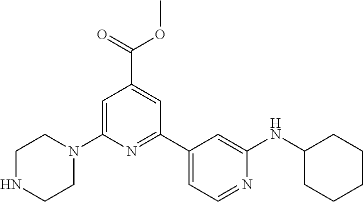

To a solution of 6-(4-tert-butoxycarbonyl-piperazin-1-yl)-2′-cyclohexylamino-[2,4′]bipyridinyl-4-carboxylic acid methyl ester (125.0 mg, 0.25 mmol) and CH₂Cl₂ (5.0 mL) is added TFA (5.0 mL). After stirring for 2 h the solution is concentrated. The residue is taken up in CH₂Cl₂ (10 mL) and washed with a saturated aqueous solution of NaHCO₃. The aqueous layer is further extracted with CH₂Cl₂ (2×10 mL). The combined organic layers are then dried (Na₂SO₄), filtered and concentrated. The residue is then separated via semi-prep HPLC (10-90% CH₃CN/H₂O gradient with 0.1% NH₄OH) to give the title compound (28.0 mg, 28%). MS (ESI) m/z 396.2 (M+1). ¹H NMR (400 MHz, MeOH-d₄) δ ppm 7.87 (d, J=5.6 Hz, 1H), 7.52 (s, 1H), 7.23 (s, 1H), 7.11 (s, 1H), 7.00 (dd, J=5.6, 1.5 Hz, 1H), 3.85 (s, 3H), 3.53-3.65 (m, 5H), 2.81-2.93 (m, 4H), 1.86-1.99 (m, 2H), 1.65-1.76 (m, 2H), 1.53-1.64 (m, 1H), 1.27-1.42 (m, 2H), 1.09-1.25 (m, 3H).

### C. 2′-Cyclohexylamino-6-piperazin-1-yl-[2,4′]bipyridinyl-4-carboxylic acid

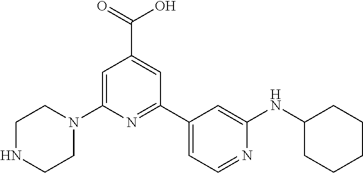

To a solution of 2′-cyclohexylamino-6-piperazin-1-yl-[2,4′]bipyridinyl-4-carboxylic acid methyl ester (56.0 mg, 0.14 mmol) in THF:water (5 mL, 4:1) is added LiOH.H₂O (59.0 g, 1.41 mmol) and the suspension is stirred at 100° C. in a pressure tube until reaction is complete by LCMS. Reaction is quenched to pH 5 with concentrated HCl and then evaporated to dryness in vacuo. This crude residue is separated via semi-prep HPLC (5-50% CH₃CN/H₂O gradient with 0.1% NH₄OH) to give the title compound (40.0 mg, 75%). (ESI) m/z 382.2 (M+1). ¹H NMR (400 MHz, DMSO-d₆) δ ppm 9.31 (br. s., 1H), 8.00 (d, J=5.3 Hz, 1H), 7.63 (s, 1H), 7.31 (s, 1H), 7.15 (s, 1H), 7.01 (d, J=5.3 Hz, 1H), 6.48 (d, J=7.7 Hz, 1H), 3.81 (br. s., 4H), 3.75 (br. s., 1H), 3.11 (br. s., 4H), 1.85-2.00 (m, 2H), 1.67-1.79 (m, 2H), 1.53-1.66 (m, 1H), 1.07-1.44 (m, 6H).

### Example 33

### A. 6-(4-tert-Butoxycarbonyl-piperazin-1-yl)-2′-phenylamino-[2,4′]bipyridinyl-4-carboxylic acid methyl ester

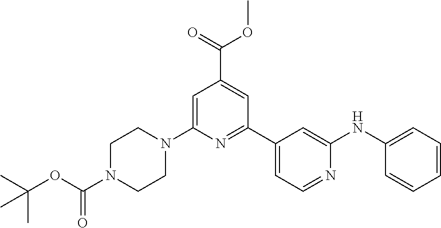

The title compound is prepared via the same procedure as Example 32A with aniline as the reacting amine. (ESI) m/z 490.3 (M+1). ¹H NMR (400 MHz, CD₂Cl₂) δ ppm 8.19 (d, J=5.3 Hz, 1H), 7.55 (s, 1H), 7.48 (s, 1H), 7.35-7.44 (m, 2H), 7.23-7.30 (m, 3H), 7.20 (s, 1H), 6.96 (t, J=7.5 Hz, 1H), 6.69 (br. s., 1H), 3.85 (s, 3H), 3.53-3.65 (m, 4H), 3.44-3.51 (m, 4H), 1.39 (s, 9H).

### B. 2′-Phenylamino-6-piperazin-1-yl-[2,4′]bipyridinyl-4-carboxylic acid methyl ester

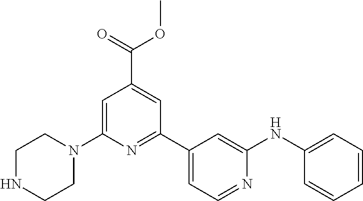

The title compound is prepared via the same procedure as Example 32B. (ESI) m/z 390.2 (M+1). ¹H NMR (400 MHz, CD₂Cl₂) δ ppm 8.18 (d, J=5.3 Hz, 1H), 7.54 (s, 1H), 7.48 (s, 1H), 7.36-7.44 (m, 2H), 7.25-7.30 (m, 3H), 7.21 (s, 1H), 6.96 (t, J=7.5 Hz, 1H), 6.69 (br. s., 1H), 3.83 (s, 3H), 3.53-3.65 (m, 4H), 3.44-3.51 (m, 4H).

### C. 2′-Phenylamino-6-piperazin-1-yl-[2,4′]bipyridinyl-4-carboxylic acid methyl amide

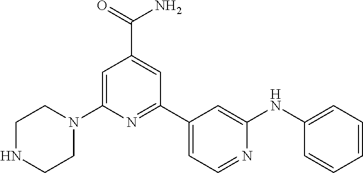

2′-phenylamino-6-piperazin-1-yl-[2,4′]bipyridinyl-4-carboxylic acid methyl ester (167.0 mg, 0.43 mmol) is dissolved in 7.0M NH₃/MeOH (10 mL) solution. Sealed the pressure vial and heated at 90° C. until reaction is complete. The reaction is concentrated in vacuo and the residue obtained is triturated with Et₂O/DCM (4:1) to give a white solid as the title compound (92.0 mg, 58%). (ESI) m/z 375.1 (M+1). ¹H NMR (400 MHz, DMSO-d₆) δ ppm 8.18 (d, J=5.4 Hz, 1H), 7.62 (d, J=8.0 Hz, 2H), 7.53 (d, J=8.5 Hz, 2H), 7.38 (dd, J=5.6, 1.5 Hz, 1H), 7.31 (app t, 2H), 7.26 (s, 1H), 7.00 (t, 1H), 3.66-3.83 (m, 4H), 2.95-3.10 (m, 4H).

### Example 34

### A. 4-(4-Cyano-2′-cyclohexylamino-[2,4′]bipyridinyl-6-yl)-piperazine-1-carboxylic acid tert-butyl ester

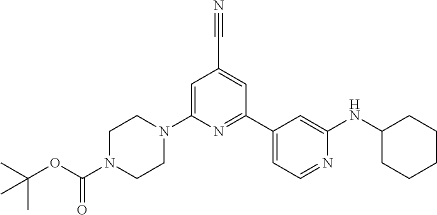

4-(4-Carbamoyl-2′-cyclohexylamino-[2,4′]bipyridinyl-6-yl)-piperazine-1-carboxylic acid tert-butyl ester (290.0 mg, 0.60 mmol), Example 6B, is dissolved in DCM (20 mL) and Et₃N (0.42 mL, 3.00 mmol) at room temperature before adding trifluoroacetic acid anhydride (0.25 mL, 1.81 mmol). After 2 h, the intermediate bis-triflamide nitrile product is observed via LCMS. The reaction is diluted with DCM (10.0 mL) and extracted with saturated NaHCO₃ solution. The organic layer is dried over anhydrous Na₂SO₄ and concentrated down. This is used without further purification. MS (ESI) m/z 559.2 (M+1).

The residue from above is dissolved in MeOH (20 mL) and treated with K₂CO₃ (828.0 mg, 6.00 mmol). After 0.5 h, the reaction is evaporated and separated via flash chromatography (SiO₂, EtOAc/heptanes gradient) to give the title compound (126.0 mg, 45%). MS (ESI) m/z 463.2 (M+1).

### B. 4-[2′-Cyclohexylamino-4-(1H-tetrazol-5-yl)-[2,4′]bipyridinyl-6-yl]-piperazine-1-carboxylic acid tert-butyl ester

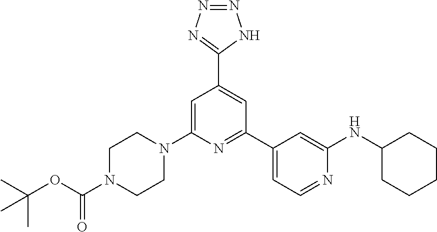

4-(4-cyano-2′-cyclohexylamino-[2,4′]bipyridinyl-6-yl)-piperazine-1-carboxylic acid tert-butyl ester (126.0 mg, 0.27 mmol), NaN₃ (18.0 mg, 0.27 mmol) and NH₄Cl (14.0 mg, 0.27 mmol) are heated in DMF (5 mL) at 120° C. After 16 h, the reaction is concentrated and used without further purification. MS (ESI) m/z 506.2 (M+1).

### C. Cyclohexyl-[6-piperazin-1-yl-4-(1H-tetrazol-5-yl)-[2,4′]bipyridinyl-2′-yl]-amine

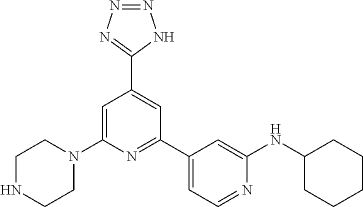

To a solution of 4-[2′-cyclohexylamino-4-(1H-tetrazol-5-yl)-[2,4′]bipyridinyl-6-yl]-piperazine-1-carboxylic acid tert-butyl ester (137.0 mg, 0.27 mmol) and CH₂Cl₂ (5.0 mL) is added TFA (5.0 mL). After stirring for 2 h, the solution is concentrated. The residue is taken up in CH₂Cl₂ (10 mL) and washed with a saturated aqueous solution of NaHCO₃. The aqueous layer is further extracted with CH₂Cl₂ (2×10 mL). The combined organic layers are then dried (Na₂SO₄), filtered and concentrated. The residue is then separated via semi-prep HPLC (10-90% CH₃CN/H₂O gradient with 0.1% NH₄OH) to give the title compound (38.1 mg, 35%). MS (ESI) m/z 406.2 (M+1). ¹H NMR (400 MHz, MeOH-d₄) δ ppm 7.90-8.01 (m, 2H), 7.54 (s, 1H), 7.16-7.27 (m, 2H), 3.87-4.01 (m, 4H), 3.61-3.77 (m, 1H), 3.31-3.37 (m, 4H), 1.97-2.11 (m, 2H), 1.75-1.88 (m, 2H), 1.60-1.74 (m, 1H), 1.38-1.55 (m, 2H), 1.19-1.37 (m, 3H).

### Example 35

### A. 4-(2′-Chloro-4-isopropylcarbamoyl-[2,4′]bipyridinyl-6-yl)-piperazine-1-carboxylic acid tert-butyl ester

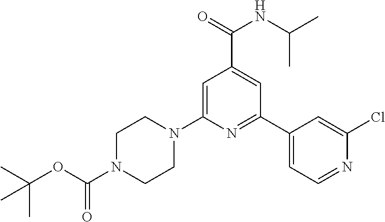

To a solution of toluene (10 mL) and trimethylaluminum (3.00 mL, 5.55 mmol) is added isopropylamine (0.50 mL, 5.55 mmol). This solution is stirred at room temperature for 10 minutes before 6-(4-tert-butoxycarbonyl-piperazin-1-yl)-2′-chloro-[2,4′]-bipyridinyl-4-carboxylic acid methyl ester (300 mg, 0.69 mmol) is added portion-wise. The resulting suspension is heated at 110° C. until LCMS indicated complete reaction. Reaction is cooled to ambient temperature and quenched carefully with MeOH. The gelatinous suspension is filtered and the filter cake washed well with MeOH. The organic is concentrated in vacuo and the residue purified via column chromatography (SiO₂, EtOAc/heptanes gradient) to afford the compound as a yellow solid (270 mg, 84%). MS (ESI) m/z 460.2 (M+1). ¹H-NMR (400 MHz, DMSO-d₆) δ ppm 8.59 (d, J=5.3 Hz, 1H), 8.50 (d, J=7.6 Hz, 1H), 8.18 (s, 1H), 8.15 (dd, J=5.3, 1.5 Hz, 1H), 7.81 (s, 1H), 7.35 (s, 1H), 4.14-4.25 (m, 1H), 3.68-3.76 (m, 4 H), 3.50-3.59 (m, 4H), 1.50 (s, 9H), 1.27 (d, J=6.6 Hz, 6H).

### B. 4-[4-Isopropylcarbamoyl-2′-(1-methyl-1H-pyrazol-3-ylamine)-[2,4′]bipyridinyl-6-yl]-piperazine-1-carboxylic acid tert-butyl ester

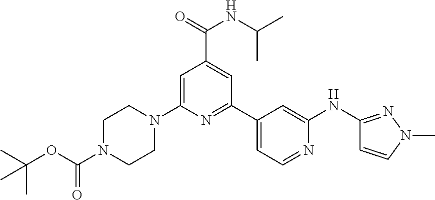

A mixture of 4-(2′-chloro-4-isopropylcarbamoyl-[2,4′]bipyridinyl-6-yl)-piperazine-1-carboxylic acid tert-butyl ester (270.0 mg, 0.59 mmol), Pd(tBu₃P)₂ (30.0 mg, 0.059 mmol), NaOtBu (226.0 mg, 2.36 mmol), 1-methyl-1H-pyrazol-3-ylamine (0.11 mL, 1.81 mmol) and 1,4-dioxane (6.0 mL) is sparged with argon for 10 min. The vessel is then sealed and the contents heated to 130° C. for 2 h. The mixture is then allowed to cool followed by concentration. The residue is then separated via flash chromatography (SiO₂, EtOAc/hexanes gradient) to give the title compound (150 mg, 59%). MS (ESI) m/z 521.4 (M+1).

### C. 2′-(1-Methyl-1H-pyrazol-3-ylamino)-6-piperazin-1-yl-[2,4′]bipyridinyl-4-carboxylic acid isopropylamide

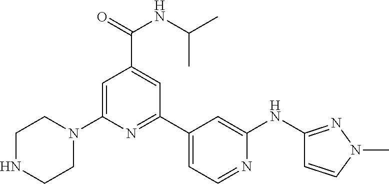

To a solution of 4-[4-isopropylcarbamoyl-2′-(1-methyl-1H-pyrazol-3-ylamine)-[2,4′]bipyridinyl-6-yl]-piperazine-1-carboxylic acid tert-butyl ester (125.0 mg, 0.24 mmol) and CH₂Cl₂ (5.0 mL) is added TFA (5.0 mL). After stirring for 2 h, the solution is concentrated. The residue is taken up in CH₂Cl₂ (10 mL) and washed with a saturated aqueous solution of NaHCO₃. The aqueous layer is further extracted with CH₂Cl₂ (2×10 mL). The combined organic layers are then dried (Na₂SO₄), filtered and concentrated. The residue is then separated via semi-prep HPLC (10-90% CH₃CN/H₂O gradient with 0.1% NH₄OH) to give the title compound (53.0 mg, 53%). MS (ESI) m/z 421.2 (M+1). ¹H NMR (400 MHz, CD₂Cl₂) δ ppm 8.14 (d, J=5.3 Hz, 1H), 7.94 (s, 1H), 7.16-7.32 (m, 3H), 6.93 (s, 1H), 6.03-6.18 (m, 2H), 5.24 (s, 1H), 4.06-4.25 (m, 1H), 3.73 (s, 3H), 3.51-3.64 (m, 4H), 2.83-2.96 (m, 4 H), 1.19 (d, J=6.4 Hz, 6H).

Compounds D and E of Example 35 can be prepared by a similar method as those above.

### D. 2′-(1-Methyl-1H-pyrazol-3-ylamino)-6-piperazin-1-yl-[2,4′]bipyridinyl-4-carboxylic acid diethylamide

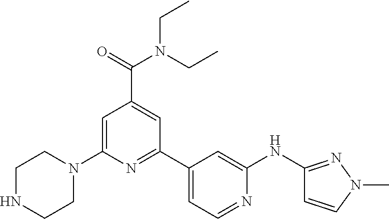

MS (ESI) m/z 435.2 (M+1). ¹H NMR (400 MHz, CD₂Cl₂) δ ppm 8.13 (d, J=5.4 Hz, 1H), 7.91 (s, 1H), 7.17-7.25 (m, 2H), 7.01 (s, 1H), 6.55 (s, 1H), 6.14 (d, J=2.3 Hz, 1H), 3.72 (s, 3H), 3.61-3.69 (m, 4H), 3.44 (q, J=6.6 Hz, 2H), 3.10-3.22 (m, 3H), 2.95-3.05 (m, 4H), 1.10-1.22 (m, 5H), 1.04 (t, J=6.9 Hz, 2H).

### E. 2′-(1-Methyl-1H-pyrazol-3-ylamino)-6-piperazin-1-yl-[2,4′]bipyridinyl-4-carboxylic acid methylamide

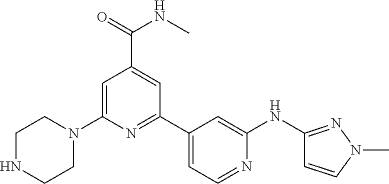

MS (ESI) m/z 395.2 (M+1). ¹H NMR (400 MHz, MeOH-d₄) δ ppm 7.87 (d, J=5.4 Hz, 1H), 7.39 (s, 1H), 7.12 (s, 1H), 7.06 (s, 1H), 7.02 (dd, J=5.6, 1.4 Hz, 1H), 3.50-3.67 (m, 5H), 2.78-2.94 (m, 7H), 1.84-2.05 (m, 2H), 1.65-1.78 (m, 2H), 1.53-1.64 (m, 1H), 1.27-1.44 (m, 2H), 1.08-1.24 (m, 3H).

### Example 36

### A. 4-(2′-Chloro-4-ethylcarbamoyl-[2,4′]bipyridinyl-6-yl)-piperazine-1-carboxylic acid tert-butyl ester

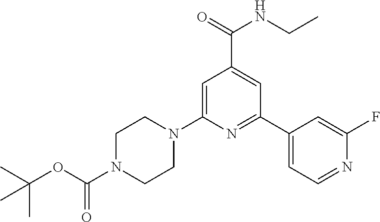

To a solution of toluene (6 mL) and trimethylaluminum (1.40 mL, 2.79 mmol) is added a 2.0 M THF solution of ethylamine (1.40 mL, 2.79 mmol). This solution is stirred at room temperature for 10 minutes before 6-(4-tert-butoxycarbonyl-piperazin-1-yl)-2′-fluoro-[2,4′]-bipyridinyl-4-carboxylic acid methyl ester (140.0 mg, 0.35 mmol) is added portion-wise. The resulting suspension is heated at 110° C. until LCMS indicated complete reaction. The reaction is cooled to ambient temperature and quenched carefully with MeOH. The gelatinous suspension is filtered and the filter cake washed well with MeOH. The organic is concentrated in vacuo and the residue purified via flash chromatography (SiO₂, EtOAc/heptanes gradient) to afford the compound as a yellow solid (81.0 mg, 54%). MS (ESI) m/z 430.2 (M+1). ¹H NMR (400 MHz, DMSO-d₆) δ ppm 8.75 (t, J=5.5 Hz, 1H), 8.42 (d, J=5.2 Hz, 1H), 8.02-8.13 (m, 1H), 7.86 (s, 1H), 7.82 (s, 1H), 7.36 (s, 1H), 3.67-3.78 (m, 4H), 3.49-3.59 (m, 4H), 3.37-3.45 (m, 2H), 1.50 (s, 9H), 1.22 (t, J=7.2 Hz, 3H).

### B. 4-[4-Ethylcarbamoyl-2′-(1-methyl-1H-pyrazol-3-ylamine)-[2,4′]bipyridinyl-6-yl]-piperazine-1-carboxylic acid tert-butyl ester

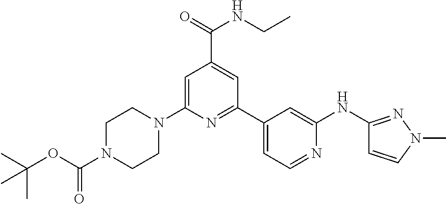

1-Methyl-1H-pyrazol-3-ylamine (65.0 uL, 0.67 mmol) is dissolved in THF (5 mL). To this is added 1.0 M THF solution of NaHMDS (1.35 mL, 1.35 mmol) followed by 4-(2′-chloro-4-ethylcarbamoyl-[2,4′]bipyridinyl-6-yl)-piperazine-1-carboxylic acid tert-butyl ester (145.0 mg, 0.33 mmol) before the reaction is heated to 80° C. for 3 h. Reaction is quenched with IPA and concentrated in vacuo. The residue is purified via flash chromatography (SiO₂, MeOH/DCM gradient) to afford the title compound as a yellow solid. MS (ESI) m/z 507.4 (M+1).

### C. 2′-(1-Methyl-1H-pyrazol-3-ylamino)-6-piperazin-1-yl-[2,4′]bipyridinyl-4-carboxylic acid ethylamide

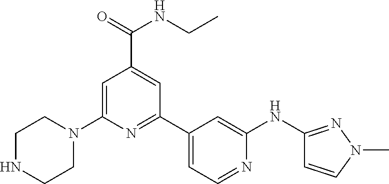

To a solution of 4-[4-ethylcarbamoyl-2′-(1-methyl-1H-pyrazol-3-ylamine)-[2,4′]bipyridinyl-6-yl]-piperazine-1-carboxylic acid tert-butyl ester (150.0 mg, 0.29 mmol) and CH₂Cl₂ (5.0 mL) is added TFA (5.0 mL). After stirring for 2 h, the solution is concentrated. The residue is taken up in CH₂Cl₂ (10 mL) and washed with a saturated aqueous solution of NaHCO₃. The aqueous layer is further extracted with CH₂Cl₂ (2×10 mL). The combined organic layers are then dried (Na₂SO₄), filtered and concentrated. The residue is then separated via semi-preparative HPLC (10-90% CH₃CN/H₂O gradient with 0.1% NH₄OH) to give the title compound (20.0 mg, 17%). MS (ESI) m/z 407.2 (M+1). ¹H NMR (400 MHz, MeOH-d₄) δ ppm 8.06 (d, J=5.4 Hz, 1H), 7.86 (s, 1H), 7.49 (s, 1H), 7.37 (d, J=2.3 Hz, 1H), 7.29 (dd, J=5.4, 1.5 Hz, 1H), 7.12 (s, 1H), 6.16 (d, J=2.4 Hz, 1H), 3.66-3.76 (m, 7H), 3.34 (q, J=7.2 Hz, 2H), 2.86-3.03 (m, 4H), 1.15 (t, J=7.3 Hz, 3H).

### Example 37

### A. 2,6-Dichloronicotinic acid ethyl ester

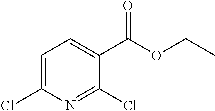

A mixture 2,6-dichloronicotinic acid (5.0 g, 26.2 mmol) and concentrated H₂SO₄ (1 mL) in EtOH (30 mL) is heated at 85° C. for 3 days. Upon cooling to room temperature, the reaction mixture is diluted with dichloromethane (200 mL), washed with saturated NaHCO₃ solution (2×100 mL) and brine (50 mL). The organic layer is dried, concentrated and the crude residue is purified by flash column with EtOAC/heptanes (2/8) to afford the desired product as a white solid (3.8 g, 67%). LC-MS ((ESI) m/z 220.0 (M+1). ¹H NMR (400 MHz, CD₂Cl₂) δ ppm 7.38 (s, 1H), 7.36 (s, 1H), 4.39 (q, J=7.1 Hz, 2H), 1.39 (t, J=7.1 Hz, 3H).

### B. 4-(6-Chloro-3-ethoxycarbonyl-pyridin-2-yl)-piperazine-1-carboxylic acid tert-butyl ester

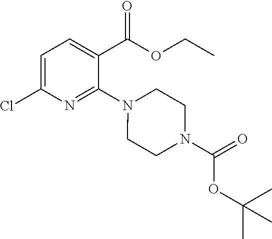

2,6-Dichloronicotinic acid ethyl ester (3.20 g, 14.5 mmol), 1-Boc-piperazine (3.25 g, 17.4 mmol) and Et₃N (4.32 mL, 29.0 mmol) are stirred in THF (60 mL) at reflux in a 250 mL round-bottom flask for 12 h. Upon cooling to room temperature the reaction mixture is extracted between EtOAc/water (600/200 mL). The organic layer is washed with brine (100 mL), dried, concentrated and purified by flash column chromatography with EtOAc/heptanes (1/3) to afford the above product as a white solid (4.4 g, 82%). ¹H-NMR (400 MHz, CD₂Cl₂) δ ppm 7.92 (d, J=7.8 Hz, 1H), 6.71 (d, J=7.8 Hz, 1H), 4.30 (q, J=7.0 Hz, 2H), 3.49-3.50 (m, 4H), 3.36-3.39 (m, 4H), 1.44 (s, 9H), 1.34 (t, J=7.0 Hz, 3H).

### C. 6-(4-tert-Butoxycarbonyl-piperazin-1-yl)-2′-fluoro-[2,4′]-bipyridinyl-5-carboxylic acid ethyl ester

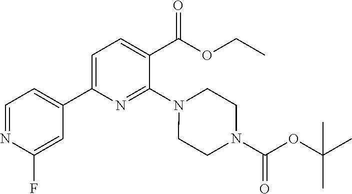

4-(6-Chloro-3-ethoxycarbonyl-pyridin-2-yl)-piperazine-1-carboxylic acid tert-butyl ester (1.11 g, 3.00 mmol) and 2-fluoropyridine-4-boronic acid (0.63 g, 4.50 mmol) are dissolved in toluene/EtOH (10:1, 30 mL). To this mixture is added 2.0 M Na₂CO₃ solution (3.0 mL, 6.0 mmol) and Pd(dppf)Cl₂ DCM complex (0.24 g, 0.30 mmol). This above suspension is heated at 110° C. for 16 h. The reaction mixture is then diluted with EtOAc (300 mL), washed with water (100 mL), dried over Na₂SO₄, and concentrated. The crude residue is purified via FCC with EtOAc/heptanes (1/2) to afford the above product as a yellow solid (0.85 g, 66%). MS (ESI) m/z 431.3 (M+1). ¹H-NMR (400 MHz, CDCl₃) δ ppm 8.31 (d, J=5.3 Hz, 1H), 8.15 (d, J=7.8 Hz, 1H), 7.76 (m, 1H), 7.55 (s, 1H), 7.28 (d, J=7.8 Hz, 1H), 4.39 (q, J=7.1 Hz, 2H), 3.60-3.62 (m, 4H), 3.49-3.52 (m, 4H), 1.49 (s, 9H), 1.41 (t, J=7.1 Hz, 3H).

### D. 6-(4-tert-Butoxycarbonyl-piperazin-1-yl)-2′-cyclohexylamino-[2,4′]bipyridinyl-5-carboxylic acid ethyl ester

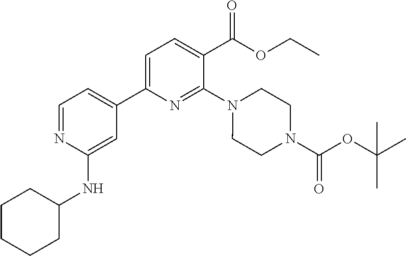

6-(4-tert-Butoxycarbonyl-piperazin-1-yl)-2′-fluoro-[2,4′]-bipyridinyl-5-carboxylic acid ethyl ester (0.29 mg, 0.67 mmol) and cyclohexylamine (0.3 mL, 2.70 mmol) are dissolved in DMSO (2 mL). After heating at 100° C. for 48 h, the reaction mixture is extracted between EtOAc and water (100/50 mL). The organic layer is dried over anhydrous Na₂SO₄, concentrated and purified by column chromatography with EtOAc/heptanes (1/2) to afford the desired product as a yellow solid (0.25 g, 73%). MS (ESI) m/z 510.4 (M+1). ¹H-NMR (400 MHz, CDCl₃) δ ppm 8.15 (d, J=5.3 Hz, 1H), 8.11 (d, J=7.8 Hz, 1H), 7.22 (d, J=7.8 Hz, 1H), 7.07 (dd, J=5.3, 1.3 Hz, 1H), 7.02 (s, 1H), 4.57 (d, J=7.8 Hz, 1H), 4.37 (q, J=8.0 Hz, 2H), 3.62-3.69 (m, 1H), 3.58-3.61 (m, 4H), 3.49-3.51 (m, 4H), 2.06-2.12 (m, 2H), 1.75-1.81 (m, 2H), 1.63-1.69 (m, 1H), 1.49 (s, 9H), 1.40 (t, J=8.0 Hz, 3H), 1.20-1.30 (m, 5H).

### E. 2′-Cyclohexylamino-6-piperazin-1-yl-[2,4′]bipyridinyl-5-carboxylic acid amide

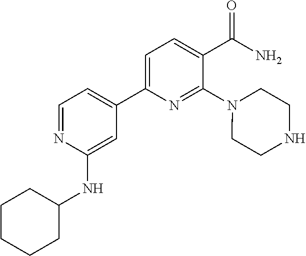

4-(5-Carbamoyl-2′-cyclohexylamino-[2,4′]bipyridinyl-6-yl)-piperazine-1-carboxylic acid tert-butyl ester (160 mg, 0.33 mmol), trifluoroacetic acid (1.3 mL) in methylene chloride (10 mL) are stirred at room temperature for 24 h. The reaction mixture is diluted with DCM (150 mL), washed with 2 M Na₂CO₃ (50 mL), dried over Na₂SO₄ and concentrated to dryness. 3 mL of MeOH is added to dissolve the crude residue and a yellow solid came out slowly. It is collected by filtration as a light yellow solid (72 mg, 57%). MS (ESI) m/z 381.2 (M+1). ¹H NMR (400 MHz, DMSO-d₆) δ ppm 8.02 (d, J=5.3 Hz, 1H), 7.90 (br, 1H), 7.82 (d, J=7.9 Hz, 1H), 7.53 (br, 1H), 7.37 (d, J=7.6 Hz, 1H), 7.14 (s, 1H), 7.02 (d, J=5.3 Hz, 1H), 6.50 (d, J=7.6 Hz, 1H), 4.08 (br, 1H), 3.69-3.76 (m, 1H), 3.30 (br, 4H), 2.82 (br, 4H), 1.91-1.96 (m, 2H), 1.70-1.75 (m, 2H), 1.57-1.62 (m, 1H), 1.15-1.35 (m, 5H).

### Example 38

### A. 6-(4-tert-Butoxycarbonyl-piperazin-1-yl)-2′-cyclohexylamino-[2,4′]bipyridinyl-5-carboxylic acid methyl ester

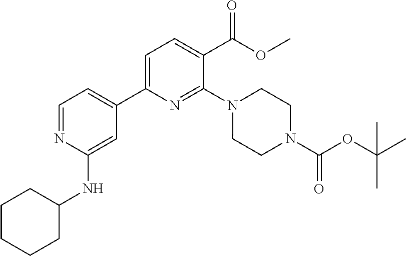

6-(4-tert-Butoxycarbonyl-piperazin-1-yl)-2′-cyclohexylamino-[2,4′]bipyridinyl-5-carboxylic acid ethyl ester (0.25 g, 0.49 mmol) and 7 M ammonia in MeOH (10 mL) are sealed in a pressure vessel and heated at 110° C. for 2 d. Upon cooling to room temperature, the solvent is removed and the crude residue is purified by column chromatography with 5% MeOH in methylene chloride to afford the title compound as a by-product. MS (ESI) m/z 496.3 (M+1).

### B. 2′-Cyclohexylamino-6-piperazin-1-yl-[2,4′]bipyridinyl-5-carboxylic acid methyl ester

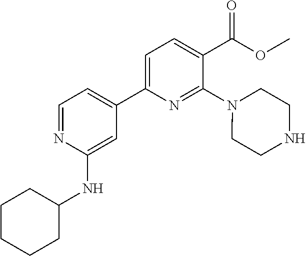

6-(4-tert-Butoxycarbonyl-piperazin-1-yl)-2′-cyclohexylamino-[2,4′]bipyridinyl-5-carboxylic acid methyl ester (65 mg, 0.13 mmol), trifluoroacetic acid (0.5 mL) in methylene chloride (3 mL) are stirred at room temperature for 24 h. The reaction mixture is diluted with DCM (50 mL), washed with 2 M Na₂CO₃ (20 mL), dried over Na₂SO₄ and concentrated to dryness. The resulting crude is purified by HPLC to afford the above product as a light yellow solid (18 mg, 35%). MS (ESI) m/z 396.2 (M+1). ¹H NMR (400 MHz, DMSO-d₆) δ ppm 8.03 (d, J=5.3 Hz, 1H), 7.99 (d, J=7.8 Hz, 1H), 7.25 (d, J=8.1 Hz, 1H), 7.13 (s, 1H), 7.01 (d, J=5.3 Hz, 1H), 6.52 (d, J=7.8 Hz, 1H), 3.82 (s, 3H), 3.69-3.75 (m, 1H), 3.34 (m, 4H), 2.79 (m, 4H), 1.91-1.95 (m, 2H), 1.69-1.74 (m, 2H), 1.57-1.62 (m, 1H), 1.15-1.35 (m, 5H).

### Example 39

### A. 4-(6-Chloro-5-ethoxycarbonyl-pyridin-2-yl)-piperazine-1-carboxylic acid tert-butyl ester

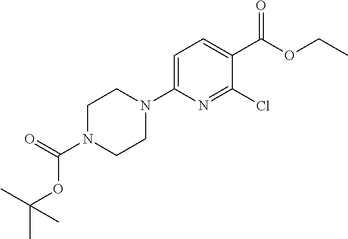

2,6-Dichloronicotinic acid ethyl ester (3.20 g, 14.5 mmol), 1-Boc-piperazine (3.25 g, 17.4 mmol) and Et₃N (4.32 mL, 29.0 mmol) are stirred in THF (60 mL) at reflux in a 250 mL round-bottom flask for 12 h. Upon cooling to room temperature the reaction mixture is extracted between EtOAc/water (600/200 mL). The organic layer is washed with brine (100 mL), dried over sodium sulfate, concentrated and purified by flash column chromatography with EtOAc/heptanes (1/3) to afford the above product as a viscous oil (0.88 g, 16%). MS (ESI) m/z 370.2 (M+1). ¹H-NMR (400 MHz, CDCl₃) δ ppm 8.03 (d, J=8.8 Hz, 1H), 6.49 (d, J=8.8 Hz, 1H), 4.33 (q, J=7.1 Hz, 2H), 3.65-3.68 (m, 4H), 3.52-3.55 (m, 4H), 1.49 (s, 9 H), 1.37 (t, J=7.1 Hz, 3H).

### B. 6-(4-tert-Butoxycarbonyl-piperazin-1-yl)-2′-fluoro-[2,4′]-bipyridinyl-3-carboxylic acid ethyl ester

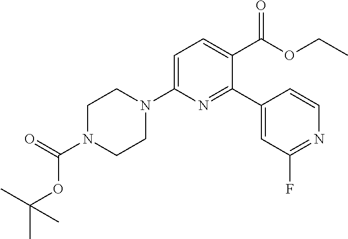

4-(6-Chloro-5-ethoxycarbonyl-pyridin-2-yl)-piperazine-1-carboxylic acid tert-butyl ester (0.82 g, 2.22 mmol) and 2-fluoropyridine-4-boronic acid (0.47 g, 3.33 mmol) are dissolved in toluene/EtOH (10/1, 22 mL). To this mixture is added 2.0 M Na₂CO₃ solution (2.2 mL, 4.40 mmol) and Pd(dppf)Cl₂ DCM complex (0.18 g, 0.22 mmol). This above suspension is heated at 110° C. for 16 h. The reaction mixture is then diluted with EtOAc (200 mL), washed with water (100 mL), dried over Na₂SO₄, and concentrated. The crude residue is purified via FCC with EtOAc/heptanes (1/2) to afford the above product as a light brown solid (0.73 g, 76%). MS (ESI) m/z 431.3 (M+1).

### C. 4-(3-Carbamoyl-2′-cyclohexylamino-[2,4′]bipyridinyl-6-yl)-piperazine-1-carbonxylic acid tert-butyl ester

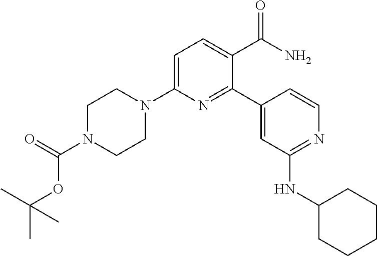

6-(4-tert-Butoxycarbonyl-piperazin-1-yl)-2′-cyclohexylamino-[2,4′]bipyridinyl-3-carboxylic acid ethyl ester (0.18 g, 0.35 mmol), ammonium chloride (30 mg) and 7 M ammonia in MeOH (12 mL) are sealed in a pressure vessel and heated at 130° C. for 4 d. Upon cooling to room temperature, the solvent is removed and the crude residue is purified by FCC with 2-5% MeOH in methylene chloride to afford the product as a yellow solid (40 mg, 24%). MS (ESI) m/z 481.2 (M+1).

### D. 2′-Cyclohexylamino-6-piperazin-1-yl-[2,4′]bipyridinyl-3-carboxylic acid ethyl ester

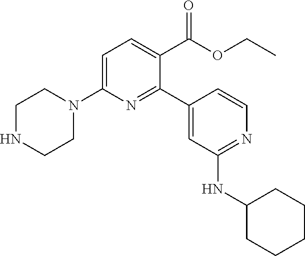

6-(4-tert-Butoxycarbonyl-piperazin-1-yl)-2′-cyclohexylamino-[2,4′]bipyridinyl-3-carboxylic acid ethyl ester (35 mg, 0.069 mmol), trifluoroacetic acid (1.0 mL) in methylene chloride (1.0 mL) are stirred at room temperature for 24 h. The reaction mixture is diluted with DCM (50 mL), washed with 2 M Na₂CO₃ (20 mL), dried over Na₂SO₄ and concentrated to dryness. The resulting crude is purified by HPLC to afford the above product as a white solid (10 mg, 35%). MS (ESI) m/z 410.2 (M+1). ¹H NMR (400 MHz, DMSO-d₆) δ ppm 7.91 (d, J=5.3 Hz, 1H), 7.89 (d, J=9.1 Hz, 1H), 6.82 (d, J=9.1 Hz, 1H), 6.43 (s, 1H), 6.39 (d, J=5.3 Hz, 1H), 6.35 (d, J=7.6 Hz, 1H), 4.04 (q, J=7.1 Hz, 2H), H), 3.54 m, 4H), 2.75 (m, 4 H), 1.88-1.95 (m, 2H), 1.68-1.73 (m, 2H), 1.55-1.61 (m, 1H), 1.26-1.36 (m, 2H), 1.12-1.22 (m, 3H), 1.03 (t, J=7.1 Hz, 3H).

### Example 40

### A. 2′-Cyclohexylamino-[2,4′]bipyridinyl-5-carboxylic acid methyl ester

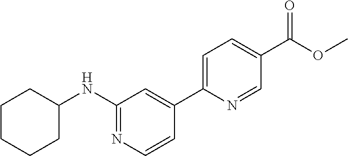

6-Chloronicotinic acid methyl ester (1.03 g, 6.00 mmol) and cyclohexyl-(4-trimethylstannanyl-pyridin-2-yl)-amine (2.13 g, 6.30 mmol) are added into toluene (60 mL) under N₂ followed by addition of trans-bis(triphenylphosphino)palladium (II) chloride (442 mg, 0.63 mmol). The reaction mixture is heated at reflux for 14 h. Then the yellow solution is diluted with EtOAc (400 mL) and washed with water (150 mL). The organic layer is dried over Na₂SO₄, concentrated and purified by column chromatography with EtOAC/heptanes (1/3) to afford a yellow solid. (1.06 g, 57%). MS (ESI) m/z 312.3 (M+1). ¹H NMR (400 MHz, CDCl₃) δ ppm 9.30 (d, J=2.0 Hz, 1H), 8.38 (dd, J=8.3, 2.3 Hz, 1H), 8.20 (d, J=5.3 Hz, 1H), 7.80 (d, J=8.3 Hz, 1H), 7.06-7.09 (m, 2H), 4.58 (d, J=8.1 Hz, 1H), 3.99 (s, 3H), 3.68-3.75 (m, 1H), 2.05-2.12 (m, 2H), 1.74-1.82 (m, 2H), 1.63-1.69 (m, 1H), 1.40-1.51 (m, 2H), 1.21-1.32 (m, 3H).

### B. 2′-Cyclohexylamino-[2,4′]bipyridinyl-5-carboxylic acid

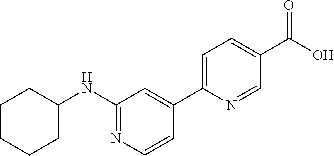

2′-Cyclohexylamino-[2,4′]bipyridinyl-5-carboxylic acid methyl ester (1.04 g, 3.34 mmol) is added into a flask with THF/H₂O (20/10 mL) followed by lithium hydroxide (0.28 g, 6.68 mmol). The reaction mixture is stirred at rt for 3 h. Then 3.3 mL of 2 N HCl aqueous solution is added to neutralize the reaction mixture. The solvent is removed and the resulting white solid is freeze-dried to remove remaining water. It is used directly in the next step without further purification. MS (ESI) m/z 298.3 (M+1).

### Example 41

### A. 2′-Cyclohexylamino-6-piperazin-1-yl-[2,4′]bipyridinyl-5-carboxylic acid

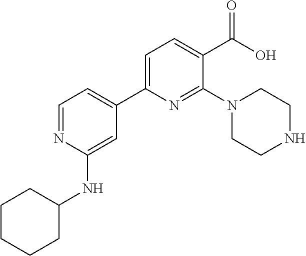

6-(4-tert-Butoxycarbonyl-piperazin-1-yl)-2′-cyclohexylamino-[2,4′]bipyridinyl-5-carboxylic acid ethyl ester (Example 37D, 650 mg, 1.28 mmol), trifluoroacetic acid (2.0 mL) in methylene chloride (4.0 mL) are stirred at room temperature for 12 h. The reaction mixture is diluted with methylene chloride (100 mL), washed with saturated Na₂CO₃ (30 mL), dried over Na₂SO₄ and concentrated to dryness to afford a yellow solid (620 mg). MS (ESI) m/z 410.3 (M+1).

The yellow solid obtained above is added into THF/water (10/5 mL) followed by lithium hydroxide (216 mg, 5.12 mmol). The reaction mixture is stirred at rt for 5 d and then the solvent is removed completely. The yellow solid (1.0 g) is used directly in the next step without further purification. MS (ESI) m/z 382.3 (M+1).

### B. 2′-Cyclohexylamino-6-piperazin-1-yl-[2,4′]bipyridinyl-5-carboxylic acid isopropylamide

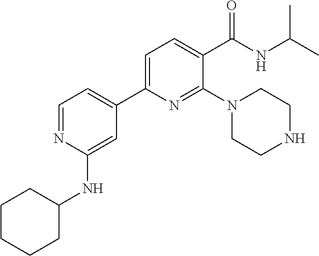

2′-Cyclohexylamino-6-piperazin-1-yl-[2,4′]bipyridinyl-5-carboxylic acid (167 mg, 0.21 mmol), isopropyl amine (54 ul, 0.63 mmol), Hunig's base (45 ul, 0.25 mmol) and HATU (285 mg, 0.74 mmol) are added into DMF (2 mL) in sequence. The reaction mixture is stirred at rt for 4 d and then purified by reverse phase HPLC to afford the above product as a off-white solid (13 mg, 15%). MS (ESI) m/z 423.4 (M+1). ¹H NMR (400 MHz, CDCl₃) δ ppm 8.55 (d, J=7.6 Hz, 1H), 8.39 (d, J=7.8 Hz, 1H), 8.17 (d, J=5.6 Hz, 1H), 7.53 (d, J=7.8 Hz, 1H), 7.12 (dd, J=5.6, 1.5 Hz, 1H), 7.06 (s, 1H), 4.62 (d, J=8.1 Hz, 1H), 4.25-4.34 (m, 1H), 3.67 (m, 1 H), 3.29-3.31 (m, 4H), 3.10-3.12 (m, 4H), 2.07-2.11 (m, 2H), 1.76-1.82 (m, 2H), 1.64-1.69 (m, 1H), 1.38-1.49 (m, 2H), 1.30 (d, J=6.6 Hz, 6H), 1.21-1.31 (m, 3H).

Compounds C-E of Example 41 can be prepared by a similar method as those above.

### C. 2′-Cyclohexylamino-6-piperazin-1-yl-[2,4′]bipyridinyl-5-carbonxylic acid methylamide

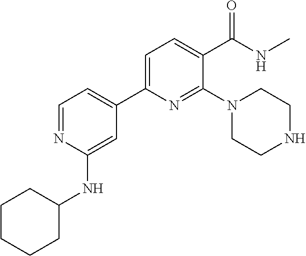

MS (ESI) m/z 395.3 (M+1). ¹H NMR (400 MHz, CDCl₃) δ ppm 8.54 (d, J=4.3 Hz, 1H), 8.36 (d, J=7.8 Hz, 1H), 8.16 (d, J=5.3 Hz, 1H), 7.52 (d, J=7.8 Hz, 1H), 7.12 (dd, J=5.6, 1.5 Hz, 1H), 7.06 (s, 1H), 4.64 (d, J=7.6 Hz, 1H), 3.65 (m, 1H), 3.27-3.29 (m, 4H), 3.07-3.09 (m, 4H), 3.04 (d, J=4.3 Hz, 3H), 2.06-2.12 (m, 2H), 1.64-1.81 (m, 3H), 1.38-1.49 (m, 2H), 1.21-1.31 (m, 3H).

### D. 2′-Cyclohexylamino-6-piperazin-1-yl-[2,4′]bipyridinyl-5-carbonxylic acid cyanomethyl-amide

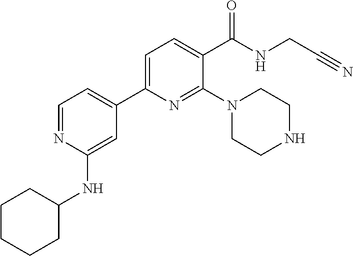

MS (ESI) m/z 420.2 (M+1). ¹H NMR (400 MHz, CDCl₃) δ ppm 9.73 (t, J=5.3 Hz, 1H), 8.44 (d, J=8.1 Hz, 1H), 8.19 (d, J=5.3 Hz, 1H), 7.60 (d, J=8.1 Hz, 1H), 7.12 (d, J=5.3 Hz, 1H), 7.06 (s, 1H), 4.58 (d, J=8.1 Hz, 1H), 4.42 (d, J=5.3 Hz, 2H), 3.66-3.73 (m, 1H), 3.26-3.29 (m, 4H), 3.12-3.14 (m, 4H), 2.06-2.13 (m, 2H), 1.76-1.83 (m, 2H), 1.66-1.70 (m, 1H), 1.40-1.50 (m, 2H), 1.22-1.32 (m, 3H).

### E. 2′-Cyclohexylamino-6-piperazin-1-yl-[2,4′]bipyridinyl-5-carbonxylic acid (cyano-methylmethyl)-amide

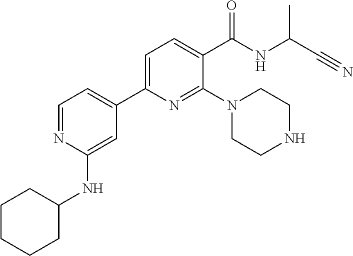

MS (ESI) m/z 434.3 (M+1). ¹H NMR (400 MHz, CDCl₃) δ ppm 9.65 (d, J=7.8 Hz, 1H), 8.44 (d, J=7.8 Hz, 1H), 8.17 (d, J=5.8 Hz, 1H), 7.60 (d, J=8.1 Hz, 1H), 7.12 (d, J=5.6 Hz, 1H), 7.07 (s, 1H), 5.13-5.20 (m, 1H), 4.17 (br, 1H), 3.65-3.74 (m, 1H), 3.29-3.36 (m, 4H), 3.12-3.23 (m, 4H), 2.07-2.12 (m, 2H), 1.76-1.82 (m, 2H), 1.71 (d, J=7.3 Hz, 3H) 1.65-1.71 (m, 1 H), 1.41-1.50 (m, 2H), 1.25-1.33 (m, 3H).

### Example 42

### A. 4-(6-Bromo-4-nitro-pyridin-2-yl)-piperazine-1-carboxylic acid tert-butyl ester

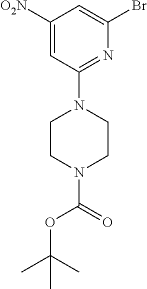

A mixture of 2,6-dibromo-4-nitro-pyridine (5.0 g, 17.8 mmol), piperazine-1-carboxylic acid tert-butyl ester (4.0 g, 21.4 mmol), triethylamine (5 mL, 35.6 mmol) and dioxane (60 mL) is heated to 110° C. for 4 h. The mixture is then allowed to cool to room temperature, diluted with CH₂Cl₂ and washed with saturated NaHCO₃, brine and then dried (Na₂SO₄), filtered and concentrated. The residue is then separated via flash chromatography (SiO₂, 10-30% EtOAc/heptane gradient) to give the title compound 4-(6-bromo-4-nitro-pyridin-2-yl)-piperazine-1-carboxylic acid tert-butyl ester. MS (ESI) m/z 386.9, 388.9 (M+1). ¹H NMR (400 MHz, CDCl₃) δ ppm 7.42 (d, J=1.5 Hz, 1H), 7.22 (d, J=1.5 Hz, 1H), 3.64-3.69 (m, 4 H), 3.55-3.60 (m, 4H), 1.50 (s, 9H).

### B. 4-(2′-Fluoro-4-nitro-[2,4′]bipyridinyl-6-yl)-piperazine-1-carboxylic acid tert-butyl ester

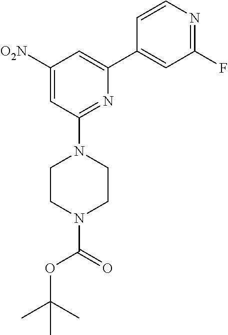

A mixture of 4-(6-bromo-4-nitro-pyridin-2-yl)-piperazine-1-carboxylic acid tert-butyl ester (1.9 g, 4.9 mmol), 2-fluoropyridine-4-boronic acid (0.9 g, 6.37 mmol), Pd(dppf)Cl₂. CH₂Cl₂ (0.2 g, 0.245 mmol), aqueous solution of Na₂CO₃ (5.0 mL, 2.0 M) and DME (45 mL) is sparged with argon for 10 min and then heated to 90° C. for 3 h under argon. The mixture is then allowed to cool to room temperature, diluted with CH₂Cl₂ and washed with saturated NaHCO₃ (×2), and dried (Na₂SO₄), filtered and concentrated. The residue is then separated via flash chromatography (SiO₂, 20-30% EtOAc/heptane gradient) to give the title compound 4-(2′-Fluoro-4-nitro-[2,4′]bipyridinyl-6-yl)-piperazine-1-carboxylic acid tert-butyl ester. MS (ESI) m/z 404.0 (M+1). ¹H NMR (400 MHz, CDCl₃) δ ppm 8.36 (d, J=5.3 Hz, 1H), 7.75-7.81 (m, 2H), 7.56-7.60 (m, 1H), 3.78 (dd, J=6.3, 4.0 Hz, 4H), 3.60-3.67 (m, 4 H), 1.51 (s, 9H).

Compound C of Example 42 can be prepared by a similar method as those above.

### C. 4-(2′-Chloro-4-nitro-[2,4′]bipyridinyl-6-yl)-piperazine-1-carboxylic acid tert-butyl ester

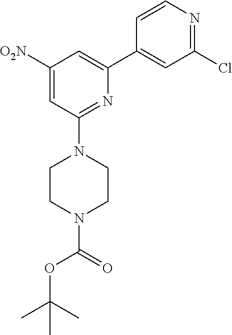

The title compound is prepared with similar method to Example 42A. MS (ESI) m/z 420.0, 422.0 (M+1). ¹H NMR (400 MHz, CDCl₃) δ ppm 8.52 (dd, J=5.2, 0.6 Hz, 1H), 7.95 (dd, J=1.5, 0.6 Hz, 1H), 7.81 (dd, J=5.3, 1.5 Hz, 1H), 7.77 (d, J=1.5 Hz, 1H), 7.42 (d, J=1.5 Hz, 1H), 3.75-3.80 (m, 4H), 3.61-3.66 (m, 4H), 1.51 (s, 9H).

### Example 43

### A. 4-(2′-Cyclohexylamino-4-nitro-[2,4′]bipyridinyl-6-yl)-piperazine-1-carboxylic acid tert-butyl ester

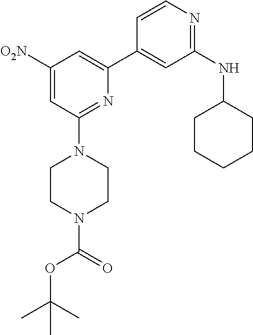

A mixture of 4-(2′-fluoro-4-nitro-[2,4′]bipyridinyl-6-yl)-piperazine-1-carboxylic acid tert-butyl ester (2.5 g, 6.2 mmol) and cyclohexylamine (250 mL) is heated to 107° C. for 62 h. The mixture is then allowed to cool followed by concentration. The residue is then separated via flash chromatography (SiO₂, 30-40% EtOAc/heptane gradient) to give the title compound 4-(2′-cyclohexylamino-4-nitro-[2,4′]bipyridinyl-6-yl)-piperazine-1-carboxylic acid tert-butyl ester. MS (ESI) m/z 483.1 (M+1). ¹H NMR (400 MHz, CDCl₃) δ ppm 8.18-8.21 (m, 1H), 7.72 (d, J=1.5 Hz, 1H), 7.35 (d, J=1.5 Hz, 1H), 7.10 (dd, J=5.3, 1.5 Hz, 1H), 6.96-6.99 (m, 1H), 4.57 (d, J=8.3 Hz, 1H), 3.65-3.78 (m, 5H), 3.62 (dd, J=6.3, 4.0 Hz, 4H), 2.06-2.15 (m, 2 H), 1.74-1.85 (m, 2H), 1.62-1.73 (m, 1H), 1.51 (s, 9H), 1.38-1.49 (m, 2H), 1.19-1.37 (m, 3H).

### B. 4-[2′-(tert-Butoxycarbonylcyclohexylamino)-4-nitro-[2,4′]bipyridinyl-6-yl]-piperazine-1-carboxylic acid tert-butyl ester

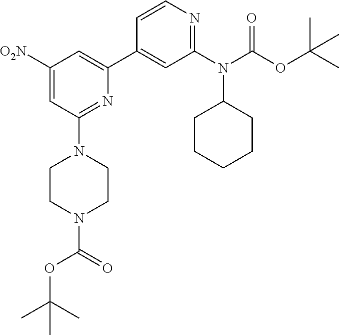

A mixture of 4-(2′-cyclohexylamino-4-nitro-[2,4′]bipyridinyl-6-yl)-piperazine-1-carboxylic acid tert-butyl ester (1.2 g, 2.49 mmol), Boc anhydride (2.72 g, 12.4 mmol), DMAP (0.061 g, 0.498 mmol), CH3CN (50 mL) and CH₂Cl₂ (5 mL) is heated to 85° C. for 4.5 h. The mixture is then allowed to cool followed by concentration. The residue is taken up in CH₂Cl₂ and washed with saturated NaHCO₃ and brine respectively and then dried (Na₂SO₄), filtered and concentrated. The residue is then separated via flash chromatography (SiO₂, 0-25% EtOAc/hexanes gradient) to give the title compound 4-[2′-(tert-Butoxycarbonyl-cyclohexyl-amino)-4-nitro-[2,4′]bipyridinyl-6-yl]-piperazine-1-carboxylic acid tert-butyl ester. MS (ESI) m/z 583.2 (M+1). ¹H NMR (400 MHz, CDCl₃) δ ppm 8.58-8.62 (m, 1H), 7.72-7.79 (m, 3H), 7.39 (d, J=1.5 Hz, 1H), 4.09-4.20 (m, 1H), 3.77 (dd, J=6.3, 4.0 Hz, 4H), 3.62 (dd, J=6.3, 4.0 Hz, 4H), 1.91-2.00 (m, 2H), 1.73-1.83 (m, 2H), 1.54-1.65 (m, 3H), 1.48-1.54 (m, 9H), 1.42-1.45 (m, 9H), 1.25-1.42 (m, 2H), 0.98-1.12 (m, 1H).

### C. 4-[2′-(tert-Butoxycarbonylcyclohexylamino)-4-amino-[2,4′]bipyridinyl-6-yl]-piperazine-1-carboxylic acid tert-butyl ester

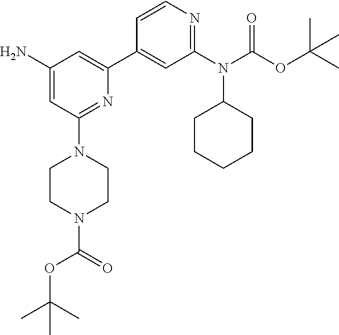

A mixture of 4-[2′-(tert-butoxycarbonyl-cyclohexyl-amino)-4-nitro-[2,4′]bipyridinyl-6-yl]-piperazine-1-carboxylic acid tert-butyl ester (1.6 g, 2.75 mmol), ammonium formate (0.9 g, 13.75 mmol), Pd/C (5% wt) (0.16 g), EtOH (250 mL) is heated to 83° C. for 1 h. The mixture is then allowed to cool followed by filtration and concentration. The residue is taken up in CH₂Cl₂ and filtered and concentrated to give the title compound 4-[2′-(tert-butoxycarbonyl-cyclohexyl-amino)-4-amino-[2,4′]bipyridinyl-6-yl]-piperazine-1-carboxylic acid tert-butyl ester. MS (ESI) m/z 553.3 (M+1). ¹H NMR (400 MHz, CDCl₃) δ ppm 8.47-8.50 (m, 1H), 7.69 (dd, J=5.2, 1.6 Hz, 1H), 7.60-7.63 (m, 1H), 6.51 (d, J=1.5 Hz, 1H), 5.90 (d, J=1.5 Hz, 1H), 4.26 (br. s., 2H), 4.03-4.16 (m, 1H), 3.54 (br. s., 8H), 1.88-1.98 (m, 2H), 1.68-1.78 (m, 2H), 1.52-1.60 (m, 1H), 1.48 (s, 9H), 1.40-1.46 (m, 1H), 1.39 (s, 9H), 1.23-1.36 (m, 3H), 0.92-1.07 (m, 1H).

### D. N*2′*-Cyclohexyl-6-piperazin-1-yl-[2,4′]bipyridinyl-4,2′-diamine

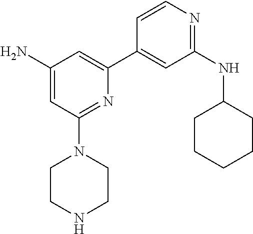

A mixture of 4-[2′-(tert-butoxycarbonyl-cyclohexyl-amino)-4-amino-[2,4′]bipyridinyl-6-yl]-piperazine-1-carboxylic acid tert-butyl ester (0.05 g, 0.091 mmol) and 50% TFA/CH₂Cl₂ is stirred at room temperature for 3 h and concentrated. The residue is mixed with 2 N NH₃ in MeOH and concentrated again, and then separated via semi-prep HPLC (8-43% CH₃CN/H₂O gradient with 0.1% NH₄OH in 17 minutes) to give the title compound N*2′*-Cyclohexyl-6-piperazin-1-yl-[2,4′]bipyridinyl-4,2′-diamine. MS (ESI) m/z 353.1 (M+1). ¹H NMR (400 MHz, CDCl₃) δ ppm 8.10 (d, J=5.3 Hz, 1H), 7.02 (dd, J=5.4, 1.5 Hz, 1H), 6.98-7.00 (m, 1H), 6.48 (d, J=1.6 Hz, 1H), 5.91 (d, J=1.6 Hz, 1H), 4.48 (d, J=7.8 Hz, 1H), 4.04 (s, 2H), 3.60-3.71 (m, 1H), 3.52-3.58 (m, 4H), 2.97-3.04 (m, 4H), 2.04-2.15 (m, 2H), 1.72-1.83 (m, 2H), 1.65-1.70 (m, 1H), 1.36-1.50 (m, 2H), 1.16-1.32 (m, 3H).

### Example 44

### Cyclohexyl-(4-nitro-6-piperazin-1-yl-[2,4′]bipyridinyl-2′-yl)-amine

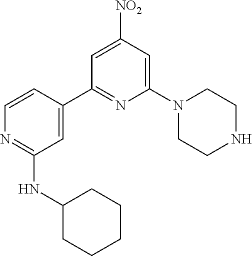

To a solution of 4-(2′-cyclohexylamino-4-nitro-[2,4′]bipyridinyl-6-yl)-piperazine-1-carboxylic acid tert-butyl ester (0.065 g, 0.135 mmol), Example 44A and CH₂Cl₂ (2 mL) is added TFA (0.5 mL). After stirring for 1 h, the solution is concentrated. The residue is then separated via semi-preparative HPLC (10-90% CH₃CN/H₂O gradient with 0.1% NH₄OH) to give the title compound cyclohexyl-(4-nitro-6-piperazin-1-yl-[2,4′]bipyridinyl-2′-yl)-amine. MS (ESI) m/z 383.0 (M+1). ¹H NMR (400 MHz, CDCl₃) δ ppm 8.18 (d, J=5.8 Hz, 1H), 7.68 (d, J=1.5 Hz, 1H), 7.33 (d, J=1.5 Hz, 1H), 7.10 (dd, J=5.6, 1.5 Hz, 1H), 6.98 (s, 1H), 4.51-4.61 (m, 1H), 3.69-3.75 (m, 4H), 3.63-3.70 (m, 1H), 2.99-3.07 (m, 4H), 2.04-2.15 (m, 2H), 1.73-1.84 (m, 2H), 1.61-1.70 (m, 1H), 1.37-1.50 (m, 2H), 1.17-1.33 (m, 3H).

### Example 45

### A. N*4*-(3-Chloro-4-fluorophenyl)-N*2′*-cyclohexyl-6-piperazin-1-yl-[2,4′]bipyridinyl-4,2′-diamine

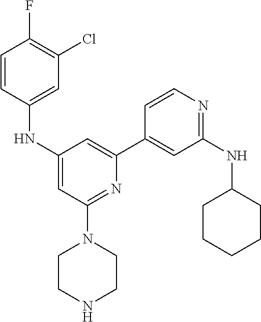

A mixture of 4-[2′-(tert-butoxycarbonyl-cyclohexyl-amino)-4-amino-[2,4′]bipyridinyl-6-yl]-piperazine-1-carboxylic acid tert-butyl ester (0.13 g, 0.236 mmol), 3-chloro-4-fluorophenylboronic acid (0.165 g, 0.944 mmol), copper (II) acetate (0.107 g, 0.59 mmol), triethylamine (0.2 mL, 1.42 mmol), 3 Å molecule sieves (10-12 beads) and DCE (5 mL) is stirred at room temperature open to the air. Reaction is monitored by LC-MS. Additional boronic acid, copper acetate and triethylamine are added. After 4 days, reaction is diluted with CH₂Cl₂, and then filtered followed by concentration. The residue is then separated via flash chromatography (SiO₂, 20-40% EtOAc/hexanes gradient) to give an intermediate of Boc protected the title compound. The intermediate is then treated with 50% TFA in CH₂Cl₂ for 1.5 h at room temperature and concentrated. The resulting residue is mixed with 2 N NH₃ in MeOH and concentrated again, and then separated via semi-prep HPLC (8-43% CH₃CN/H₂O gradient with 0.1% NH₄OH in 17 minutes). The title compound N*4*-(3-chloro-4-fluorophenyl)-N*2′*-cyclohexyl-6-piperazin-1-yl-[2,4′]bipyridinyl-4,2′-diamine is obtained. MS (ESI) m/z 481.1 (M+1). ¹H NMR (400 MHz, DMSO-d₆) δ ppm 8.72 (br. s., 1H), 7.98 (d, J=5.4 Hz, 1H), 7.38 (t, J=9.0 Hz, 1H), 7.30 (dd, J=6.6, 2.7 Hz, 1H), 7.19-7.25 (m, 1H), 7.03 (br. s., 1H), 6.87 (dd, J=5.4, 1.5 Hz, 1H), 6.73 (d, J=1.3 Hz, 1H), 6.44 (d, J=7.6 Hz, 1H), 6.24 (d, 1H), 3.66-3.79 (m, 1H), 3.39-3.44 (m, 4H), 2.76-2.83 (m, 4H), 1.89-1.98 (m, 2H), 1.67-1.77 (m, 2H), 1.56-1.65 (m, 1H), 1.26-1.39 (m, 2H), 1.13-1.26 (m, 3H).

Compounds B-G of Example 45 can be prepared by a similar method as those above.

### B. N*4*-(4-Chlorophenyl)-N*2′*-cyclohexyl-6-piperazin-1-yl-[2,4′]bipyridinyl-4,2′-diamine

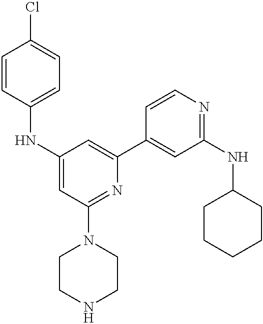

MS (ESI) m/z 463.2 (M+1). ¹H NMR (400 MHz, DMSO-d₆) δ ppm 8.75 (s, 1H), 7.97 (d, J=5.3 Hz, 1H), 7.28 (dd, J=63.2, 8.6 Hz, 4H), 7.02 (s, 1H), 6.87 (d, J=5.1 Hz, 1H), 6.77 (s, 1H), 6.43 (d, J=7.6 Hz, 1H), 6.27 (s, 1H), 3.73 (br. s., 1H), 3.38-3.45 (m, 4H), 2.79 (br. s., 4H), 1.88-1.97 (m, 2H), 1.67-1.77 (m, 2H), 1.55-1.64 (m, 1H), 1.24-1.39 (m, 2 H), 1.09-1.24 (m, 3H).

### C. N*2′*-Cyclohexyl-N*4*-(4-fluoro-phenyl)-6-piperazin-1-yl-[2,4′]bipyridinyl-4,2′-diamine

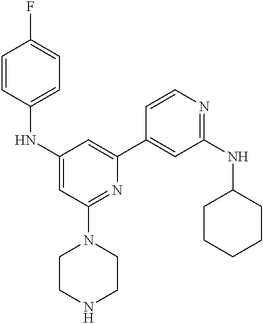

MS (ESI) m/z 447.1 (M+1). ¹H NMR (400 MHz, DMSO-d₆) δ ppm 8.59 (br. s., 1H), 7.96 (d, 1H), 7.15-7.25 (m, 4H), 6.99 (br. s., 1H), 6.86 (dd, J=5.4, 1.5 Hz, 1H), 6.72 (d, J=1.3 Hz, 1H), 6.42 (d, J=7.7 Hz, 1H), 6.21 (d, J=1.3 Hz, 1H), 3.65-3.78 (m, 1H), 3.43-3.51 (m, 4H), 2.86-2.93 (m, 4H), 1.88-1.96 (m, 2H), 1.67-1.77 (m, 2H), 1.55-1.64 (m, 1H), 1.25-1.38 (m, 2H), 1.11-1.24 (m, 3H).

### D. N*2′*-Cyclohexyl-N*4*-(3-fluoro-phenyl)-6-piperazin-1-yl-[2,4′]bipyridinyl-4,2′-diamine

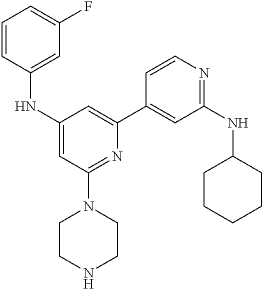

MS (ESI) m/z 447.1 (M+1). ¹H NMR (400 MHz, DMSO-d₆) δ ppm 8.91 (s, 1H), 7.98-8.00 (m, 1H), 7.32-7.39 (m, 1H), 7.02-7.06 (m, 1H), 6.95-7.01 (m, 2H), 6.90 (dd, J=5.4, 1.5 Hz, 1H), 6.86-6.87 (m, 1H), 6.76-6.84 (m, 1H), 6.40-6.45 (m, 2H), 3.70-3.76 (m, 1H), 3.64-3.70 (m, 4H), 3.12-3.19 (m, 4H), 1.88-1.97 (m, 2H), 1.68-1.77 (m, 2H), 1.56-1.64 (m, 1H), 1.25-1.37 (m, 2H), 1.12-1.24 (m, 3H).

### E. 3-(2′-Cyclohexylamino-6-piperazin-1-yl-[2,4′]bipyridinyl-4-ylamino)-N-methyl-benzamide

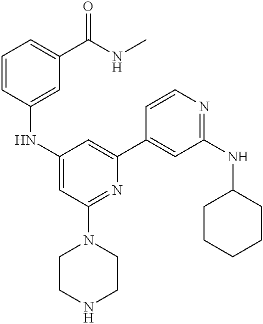

MS (ESI) m/z 486.2 (M+1). ¹H NMR (400 MHz, DMSO-d₆) δ ppm 8.76 (br. s., 1H), 8.37-8.45 (m, 1H), 7.97 (d, J=5.6 Hz, 1H), 7.66 (br. s., 1H), 7.37-7.48 (m, 2H), 7.31-7.37 (m, 1H), 7.02 (br. s., 1H), 6.83-6.88 (m, 1H), 6.78 (br. s., 1H), 6.43 (d, J=7.6 Hz, 1H), 6.29 (br. s., 1H), 3.66-3.79 (m, 1H), 3.38-3.45 (m, 4H), 2.74-2.85 (m, 7H), 1.88-1.98 (m, 2H), 1.66-1.77 (m, 2H), 1.55-1.65 (m, 1H), 1.24-1.40 (m, 2H), 1.11-1.25 (m, 3H).

### F. N*2′*-Cyclohexyl-6-piperazin-1-yl-N*4*-(4-trifluoromethylphenyl)-[2,4′]bipyridinyl-4,2′-diamine

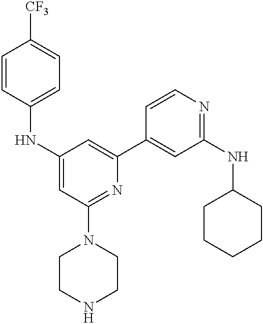

MS (ESI) m/z 497.2 (M+1). ¹H NMR (400 MHz, DMSO-d₆) δ ppm 9.08 (s, 1H), 9.08 (s, 1H), 7.98 (d, J=5.3 Hz, 1H), 7.64 (d, J=8.7 Hz, 2H), 7.33 (d, J=8.5 Hz, 2H), 7.05 (br. s., 1H), 6.90 (dd, J=5.4, 1.5 Hz, 1H), 6.88 (d, J=1.3 Hz, 1H), 6.43 (d, J=7.7 Hz, 1H), 6.40 (d, J=1.0 Hz, 1H), 3.66-3.79 (m, 1H), 3.40-3.48 (m, 4H), 2.77-2.84 (m, 4H), 1.88-1.98 (m, 2H), 1.67-1.78 (m, 2H), 1.54-1.65 (m, 1H), 1.25-1.39 (m, 2H), 1.10-1.25 (m, 3H).

### G. N*2′*-Cyclohexyl-6-piperazin-1-yl-N*4*-(3-trifluoromethylphenyl)-[2,4′]bipyridinyl-4,2′-diamine

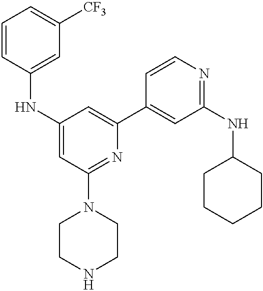

MS (ESI) m/z 497.1 (M+1). ¹H NMR (400 MHz, DMSO-d₆) δ ppm 8.94 (s, 1H), 7.97 (d, J=5.4 Hz, 1H), 7.49-7.59 (m, 2H), 7.38-7.43 (m, 1H), 7.26-7.31 (m, 1H), 7.04 (s, 1 H), 6.86 (dd, J=5.4, 1.5 Hz, 1H), 6.80 (d, J=1.4 Hz, 1H), 6.44 (d, J=7.7 Hz, 1H), 6.33 (d, J=1.4 Hz, 1H), 3.64-3.78 (m, 1H), 3.40-3.46 (m, 4H), 2.77-2.83 (m, 4H), 1.88-1.97 (m, 2H), 1.67-1.76 (m, 2H), 1.55-1.64 (m, 1H), 1.25-1.39 (m, 2H), 1.10-1.25 (m, 3H).

### Example 46

### A. N-(2′-Cyclohexylamino-6-piperazin-1-yl-[2,4′]bipyridinyl-4-yl)-methane sulfonamide

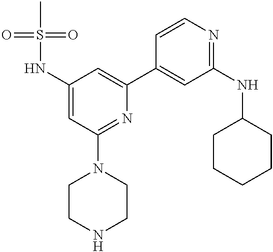

To a solution of 4-[2′-(tert-butoxycarbonyl-cyclohexyl-amino)-4-amino-[2,4′]bipyridinyl-6-yl]-piperazine-1-carboxylic acid tert-butyl ester (0.113 g, 0.205 mmol) in CH₂Cl₂ (3 mL) and triethylamine (0.145 mL, 1.02 mmol) methanesulfonyl chloride (0.04 mL, 0.512 mmol) is added at 0° C. The mixture is allowed to warm to room temperature, stirred for 1 h and then diluted with CH₂Cl₂, washed with saturated NaHCO₃ (×2), dried (Na₂SO₄), filtered and concentrated. The residue [MS (ESI) m/z 709.2 (M+1)] is obtained and is taken up in MeOH/THF (1:1, 14 mL) and treated with K₂CO₃ (1.3 g) at room temperature for 0.5 h [Ref. Tetrahedron 61 (2005) 12330]. The mixture is filtered, concentrated. The residue is taken up in CH₂Cl₂ and filtered again followed by concentration. The residue [N-(2′-(tert-butoxycarbonyl-cyclohexylamino-6-piperazin-1-yl-[2,4′]bipyridinyl-4-yl)-methanesulfonamide, MS (ESI) m/z 631.2 (M+1)] is treated with 50% TFA in CH₂Cl₂ for 2 h. After concentration, the residue is mixed with 2 N NH₃ in MeOH and concentrated again, and then separated via semi-preparative HPLC (8-43% CH₃CN/H₂O gradient with 0.1% NH₄OH in 17 minutes) to give the title compound, N-(2′-cyclohexylamino-6-piperazin-1-yl-[2,4′]bipyridinyl-4-yl)-methanesulfonamide. MS (ESI) m/z 431.0 (M+1). ¹H NMR (400 MHz, CDCl₃) δ ppm 8.12 (d, J=5.6 Hz, 1H), 7.01 (dd, J=5.4, 1.4 Hz, 1H), 6.96 (br. s., 1H), 6.80 (d, J=1.5 Hz, 1H), 6.48 (d, J=1.5 Hz, 1H), 4.62 (br. s., 1H), 3.56-3.72 (m, 5H), 3.12 (s, 3H), 2.97-3.04 (m, 4H), 2.02-2.15 (m, 3H), 1.73-1.84 (m, 3H), 1.60-1.70 (m, 1H), 1.36-1.50 (m, 2H), 1.18-1.32 (m, 3H).

### Example 47

### A. N-(2′-Cyclohexylamino-6-piperazin-1-yl-[2,4′]bipyridinyl-4-yl)-acetamide

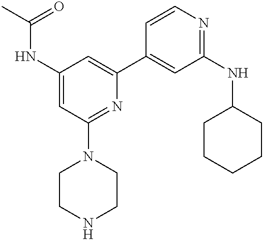

To a solution of 4-[2′-(tert-butoxycarbonyl-cyclohexyl-amino)-4-amino-[2,4′]bipyridinyl-6-yl]-piperazine-1-carboxylic acid tert-butyl ester (0.105 g, 0.19 mmol) in CH₂Cl₂ (5 mL) and triethylamine (0.140 mL, 0.95 mmol) acetic anhydride (0.055 mL, 0.571 mmol) is added followed by DMAP (0.003 g, 0.019 mmol). After the mixture is heated to 45° C. for 8 h, additional acetic anhydride is added and stirred at 41° C. for additional 9 h. And then diluted with CH₂Cl₂, washed with saturated NaHCO₃ (×2), dried (Na₂SO₄), filtered and concentrated. The residue is separated via flash chromatography (SiO₂, 35-65% EtOAc/hexanes gradient) to give an intermediate Boc protected the title compound that is then treated with 50% TFA in CH₂Cl₂ for 40 minutes. After concentration, the residue is mixed with 2 N NH₃ in MeOH and concentrated again, and then separated via semi-prep HPLC (6-36% CH₃CN/H₂O gradient with 0.1% NH₄OH in 17 minutes) to give the title compound N-(2′-cyclohexylamino-6-piperazin-1-yl-[2,4′]bipyridinyl-4-yl)-acetamide. MS (ESI) m/z 395.0 (M+1). ¹H NMR (400 MHz, DMSO-d₆) δ ppm 10.15 (s, 1H), 7.99 (d, J=5.3 Hz, 1H), 7.32 (br. s., 1H), 7.05 (d, J=5.6 Hz, 2H), 6.85 (dd, J=5.4, 1.1 Hz, 1H), 6.50 (d, J=7.6 Hz, 1H), 3.66-3.81 (m, 1H), 3.39-3.48 (m, 4H), 2.76-2.86 (m, 4H), 2.07 (s, 3H), 1.88-1.97 (m, 2H), 1.67-1.77 (m, 2H), 1.55-1.64 (m, 1H), 1.25-1.39 (m, 2H), 1.13-1.25 (m, 3H).

### Example 48

### A. Cyclohexyl-(6-piperazin-1-yl-4-tetrazol-1-yl-[2,4′]bipyridinyl-2′-yl)-amine

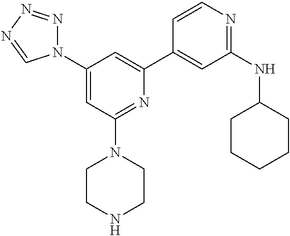

To a mixture of 4-[2′-(tert-butoxycarbonyl-cyclohexyl-amino)-4-amino-[2,4′]bipyridinyl-6-yl]-piperazine-1-carboxylic acid tert-butyl ester (0.147 g, 0.266 mmol) and NaN₃ (0.053 g, 0.815 mmol) in trimethyl orthoformate (0.714 mL) acetic acid (4 mL) is added. The mixture is stirred at room temperature over night. After concentration, the residue is taken up in CH₂Cl₂, washed with saturated NaHCO₃ (×2), brine and then dried (Na₂SO₄), filtered and concentrated. The residue [MS (ESI) m/z 606.2 (M+1)] is treated with 50% TFA in CH₂Cl₂ at room temperature for 1 h followed by concentration. The residue is mixed with 2 N NH₃ in MeOH and concentrated again, and then separated via semi-prep HPLC (8-38% CH₃CN/H₂O gradient with 0.1% NH₄OH in 17 minutes) to give the title compound cyclohexyl-(6-piperazin-1-yl-4-tetrazol-1-yl-[2,4′]bipyridinyl-2′-yl)-amine. MS (ESI) m/z 406.0 (M+1). ¹H NMR (400 MHz, DMSO-d₆) δ ppm 10.29 (s, 1H), 8.05 (d, J=5.3 Hz, 1H), 7.64 (d, J=1.1 Hz, 1H), 7.33 (d, J=1.3 Hz, 1H), 7.20 (s, 1H), 7.07 (dd, J=5.4, 1.5 Hz, 1H), 6.54 (d, J=7.7 Hz, 1H), 3.70-3.89 (m, 1H), 3.57-3.68 (m, 4H), 2.76-2.93 (m, 4H), 1.94 (dd, J=11.9, 2.5 Hz, 2H), 1.72 (dd, J=9.2, 3.7 Hz, 2H), 1.54-1.65 (m, 1H), 1.27-1.41 (m, 2H), 1.12-1.26 (m, 3H).

### Example 49

### A. N*2′*-Cyclohexyl-N*4*-(4-fluorobenzyl)-6-piperazin-1-yl-[2,4′]bipyridinyl-4,2′-diamine

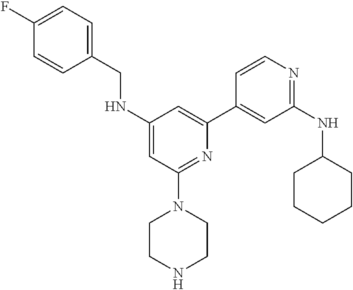

To a mixture of 4-[2′-(tert-butoxycarbonyl-cyclohexyl-amino)-4-amino-[2,4′]bipyridinyl-6-yl]-piperazine-1-carboxylic acid tert-butyl ester (0.12 g, 0.217 mmol) and 4-fluorobenzaldehyde (0.05 mL, 0.478 mmol) in CH₂Cl₂, sodium triacetoxylborohydride (0.183 g, 0.867 mmol) is added. The mixture is stirred at room temperature over night. The reaction is quenched with a mixture of ice and saturated NaHCO₃, diluted with CH₂Cl₂. The resulting organic layer is washed with saturated NaHCO₃ (×2) and brine and then dried (Na₂SO₄), filtered and concentrated. The residue [MS (ESI) m/z 661.3 (M+1)] is treated with 50% TFA in CH₂Cl₂ at room temperature for 1 h and concentrated. The resulting residue is mixed with 2 N NH₃ in MeOH and concentrated again, and then separated via semi-preparative HPLC (25-55% CH₃CN/H₂O gradient with 0.1% NH₄OH in 17 minutes) to give the title compound N*2′*-cyclohexyl-N*4*-(4-fluoro-benzyl)-6-piperazin-1-yl-[2,4′]bipyridinyl-4,2′-diamine. MS (ESI) m/z 461.1 (M+1). ¹H NMR (400 MHz, DMSO-d₆) δ ppm 7.93 (d, J=5.6 Hz, 1H), 7.36-7.43 (m, 2H), 7.12-7.19 (m, 2H), 7.00 (br. s., 1H), 6.88 (t, J=6.1 Hz, 1H), 6.82 (dd, J=5.4, 1.4 Hz, 1H), 6.50 (d, J=1.3 Hz, 1H), 6.36 (d, J=7.6 Hz, 1H), 5.86 (d, J=1.3 Hz, 1H), 4.35 (d, J=5.8 Hz, 2H), 3.63-3.76 (m, 1H), 3.37-3.43 (m, 4H), 2.79-2.85 (m, 4H), 1.87-1.96 (m, 2H), 1.67-1.76 (m, 2H), 1.55-1.63 (m, 1H), 1.24-1.38 (m, 2H), 1.09-1.24 (m, 3H).

### Example 50

### A. 4-(2′-Cyclohexylamino-4-imidazol-1-yl-[2,4′]bipyridinyl-6-yl)-piperazine-1-carboxylic acid tert-butyl ester

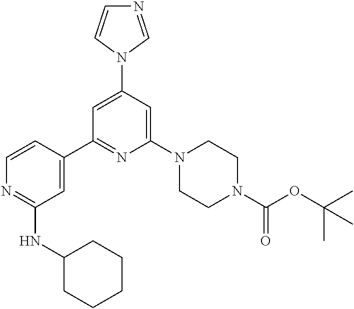

A flask under an atmosphere of argon is charged with NaH (0.200 g, 5.18 mmol) and DMSO (0.5 mL) followed by the addition of imidazole (0.352 g, 5.18 mmol) in DMSO (0.5 mL). After 5 min, a DMSO solution (1.5 mL) of 4-(2′-cyclohexylamino-4-nitro-[2,4′]bipyridinyl-6-yl)-piperazine-1-carboxylic acid tert-butyl ester Example 44A (0.250 g, 0.518 mmol) is added to the slurry. The mixture is warmed to 45° C. for 2.5 h. The mixture is then diluted with Et₂O (100 mL) and washed with saturated aqueous NaHCO₃ (100 mL). The aqueous layer is further extracted with Et₂O (2×100 mL). The combined organic layers are dried (MgSO₄), filtered and concentrated. The residue is then separated via flash chromatography (SiO₂, 0-5% MeOH/EtOAc) to give the title compound 4-(2′-cyclohexylamino-4-imidazol-1-yl-[2,4′]bipyridinyl-6-yl)-piperazine-1-carboxylic acid tert-butyl ester. MS (ESI) m/z 504.3 (M+1). ¹H NMR (400 MHz, CDCl₃) δ ppm 8.16 (d, J=5.6 Hz, 1H), 8.03 (s, 1H), 8.00 (s, 1H), 7.38 (s, 1H), 7.26 (s, 1H), 7.09 (d, J=1.5 Hz, 1H), 7.06 (dd, J=5.3, 1.5 Hz, 1H), 7.00 (s, 1H), 6.59 (d, J=1.3 Hz, 1H), 3.69-3.75 (m, 4H), 3.64-3.69 (m, 1H), 3.58-3.65 (m, 4H), 2.06-2.15 (m, 2H), 1.74-1.84 (m, 2H), 1.62-1.74 (m, 1H), 1.51 (s, 9H), 1.37-1.49 (m, 2H), 1.27 (d, J=37.4 Hz, 3H).

### B. Cyclohexyl-(4-imidazol-1-yl-6-piperazin-1-yl-[2,4′]bipyridinyl-2′-yl)-amine

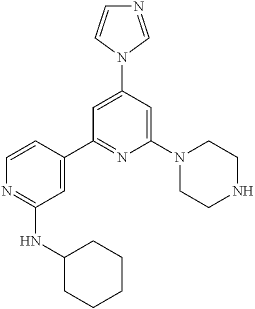

To a solution of cyclohexylamino-4-imidazol-1-yl-[2,4′]bipyridinyl-6-yl)-piperazine-1-carboxylic acid tert-butyl ester (0.091 g, 0.181 mmol) and CH₂Cl₂ (4 mL) is added TFA (2 mL). After stirring for 1 h, the solution is concentrated. The residue is taken up in MeOH and neutralized with NH₄OH to pH 7 and then separated via semi-preparative HPLC (10-65% CH₃CN/H₂O gradient with 0.1% NH₄OH) to give the title compound cyclohexyl-(4-imidazol-1-yl-6-piperazin-1-yl-[2,4′]bipyridinyl-2′-yl)-amine. MS (ESI) m/z 404.3 (M+1). ¹H NMR (400 MHz, DMSO-d₆) δ ppm 8.58 (s, 1H), 8.00-8.05 (m, 2H), 7.44 (d, J=1.5 Hz, 1H), 7.20 (s, 1H), 7.14-7.15 (m, 1H), 7.12 (dd, J=5.3, 1.5 Hz, 1H), 7.04 (d, J=1.5 Hz, 1H), 6.46 (d, J=7.6 Hz, 1H), 3.69-3.80 (m, 1H), 3.58-3.63 (m, 4H), 2.82 (d, J=9.9 Hz, 4H), 2.34-2.47 (m, 1H), 1.88-1.98 (m, 2H), 1.68-1.77 (m, 2H), 1.55-1.65 (m, 1H), 1.27-1.40 (m, 2H), 1.20 (d, J=38.1 Hz, 3H).

### Example 51

### A. 3-(2′-Cyclohexylamino-6-piperazin-1-yl-[2,4′]bipyridinyl-4-yl)-3H-imidazole-4-carboxylic acid ethyl ester

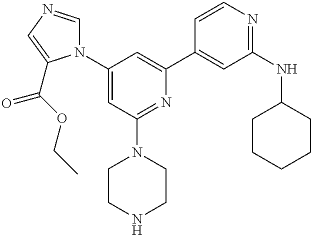

A mixture of 4-[2′-(tert-butoxycarbonyl-cyclohexyl-amino)-4-amino-[2,4′]bipyridinyl-6-yl]-piperazine-1-carboxylic acid tert-butyl ester (0.2 g, 0.362 mmol), 50% ethyl glyoxalate in toluene (0.143 mL, 1.45 mmol) 3 A molecular seives and CH₂Cl₂ (3 mL) is stirred at room temperature over night, filtered and concentrated to dryness. The residue is then mixed with (toluene-4-sulfonyl)acetonitrile, K₂CO₃ (0.15 g, 1.09 mmol) and EtOH (3 mL) and heated to 50° C. for 3 h and then concentrated. The residue is taken up in CH₂Cl₂, washed with saturated NaHCO₃ and brine and then dried (Na₂SO₄), filtered and concentrated. The residue is separated via flash chromatography (SiO₂, 50-100% EtOAc/hexanes gradient) to give an intermediate of Boc protected the title compound [MS (ESI) m/z 676.3 (M+1). The yielded intermediate is treated with 50% TFA in CH₂Cl₂ at room temperature for 1 h and concentrated. The resulting residue is mixed with 2 N NH₃ in MeOH and concentrated again, and then separated via semi-prep HPLC (10-55% CH₃CN/H₂O gradient with 0.1% NH₄OH in 17 minutes) to give the title compound 3-(2′-cyclohexylamino-6-piperazin-1-yl-[2,4′]bipyridinyl-4-yl)-3H-imidazole-4-carboxylic acid ethyl ester. MS (ESI) m/z 476.1 (M+1). ¹H NMR (400 MHz, DMSO-d₆) δ ppm 8.21 (d, J=0.9 Hz, 1H), 8.00 (d, J=5.3 Hz, 1H), 7.82 (d, J=0.9 Hz, 1H), 7.20 (d, J=1.1 Hz, 1H), 7.17 (d, J=0.6 Hz, 1H), 7.02 (dd, J=5.4, 1.4 Hz, 1H), 6.95 (d, J=1.1 Hz, 1H), 6.45 (d, J=7.8 Hz, 1H), 4.17 (q, J=7.1 Hz, 2H), 3.67-3.81 (m, 1 H), 3.55-3.61 (m, 4H), 2.79-2.85 (m, 4H), 1.88-1.97 (m, 2H), 1.67-1.77 (m, 2H), 1.54-1.63 (m, 1H), 1.25-1.40 (m, 2H), 1.10-1.25 (m, 6H).

### Example 52

### A. 4-[2′-(tert-Butoxycarbonyl-cyclohexyl-amino)-4-hydroxy-[2,4′]bipyridinyl-6-yl]-piperazine-1-carboxylic acid tert-butyl ester

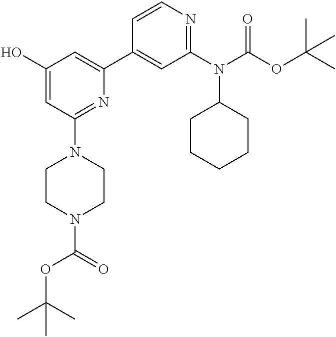

A mixture of 4-[2′-(tert-butoxycarbonyl-cyclohexyl-amino)-4-nitro-[2,4′]bipyridinyl-6-yl]-piperazine-1-carboxylic acid tert-butyl ester (1.9 g, 3.26 mmol), KOH (1.8 g, 32.6 mmol) and DMSO (65 mL) is stirred at room temperature for 1 h and then diluted with CH₂Cl₂, washed with H₂O (2×), brine, dried (Na₂SO₄), filtered and concentrated. The residue is separated via flash chromatography (SiO₂, 0-50% EtOAc/heptane gradient) to give the title compound 4-[2′-(tert-butoxycarbonyl-cyclohexyl-amino)-4-hydroxy-[2,4′]bipyridinyl-6-yl]-piperazine-1-carboxylic acid tert-butyl ester. MS (ESI) m/z 554.2 (M+1). ¹H NMR (400 MHz, CDCl₃) δ ppm 8.49 (d, J=5.3 Hz, 1H), 7.65-7.70 (m, 1H), 7.61-7.65 (m, 1H), 6.58-6.61 (m, 1H), 6.01-6.05 (m, 1H), 4.00-4.14 (m, 1H), 3.49-3.59 (m, 8H), 1.92-2.01 (m, 2H), 1.70-1.80 (m, 2H), 1.54-1.63 (m, 1H), 1.50 (s, 9H), 1.22-1.48 (m, 14H), 0.91-1.09 (m, 1H).

### B. 2′-Cyclohexylamino-6-piperazin-1-yl-[2,4′]bipyridinyl-4-ol

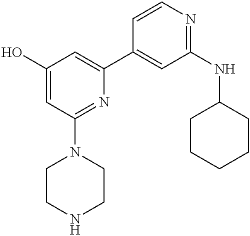

The mixture of 4-[2′-(tert-butoxycarbonyl-cyclohexyl-amino)-4-hydroxy-[2,4′]bipyridinyl-6-yl]-piperazine-1-carboxylic acid tert-butyl ester and 50% TFA in CH₂Cl₂ is stirred at room temperature for 1 h and concentrated. The resulting residue is mixed with 2 N NH₃ in MeOH and concentrated again, and then separated via semi-preparative HPLC (6-30% CH₃CN/H₂O gradient with 0.1% NH₄OH in 17 minutes) to give the title compound 2′-cyclohexylamino-6-piperazin-1-yl-[2,4′]bipyridinyl-4-ol. MS (ESI) m/z 354.0 (M+1). ¹H NMR (400 MHz, DMSO-d₆) δ ppm 7.96 (d, J=5.3 Hz, 1H), 7.01-7.07 (m, 1H), 6.88 (dd, J=5.4, 1.4 Hz, 1H), 6.60 (d, J=1.5 Hz, 1H), 6.42 (d, J=7.8 Hz, 1H), 6.11 (d, J=1.5 Hz, 1H), 3.65-3.78 (m, 1H), 3.39-3.44 (m, 4H), 2.77-2.82 (m, 4H), 1.88-1.96 (m, 2H), 1.66-1.77 (m, 2H), 1.54-1.64 (m, 1H), 1.25-1.39 (m, 2H), 1.10-1.25 (m, 3H).

### C. 2′-iso-Propylamino-6-piperazin-1-yl-[2,4′]bipyridinyl-4-ol

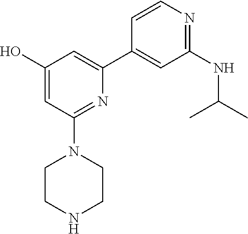

The title compound is prepared in similar method to compound Example 52B. MS (ESI) m/z 314.2 (M+1). ¹H NMR (400 MHz, DMSO-d₆) δ ppm 7.99 (d, J=5.4 Hz, 1H), 7.02 (s, 1H), 6.91 (dd, J=5.4, 0.9 Hz, 1H), 6.66 (d, J=1.0 Hz, 1H), 6.38 (d, J=7.6 Hz, 1H), 6.19 (d, J=0.9 Hz, 1H), 3.97-4.12 (m, 1H), 3.54-3.59 (m, 4H), 2.97-3.02 (m, 4H), 1.15 (d, J=6.4 Hz, 6H).

### Example 53

### A. tert-Butyl 4-(2′-[(tert-butoxycarbonyl)(cyclohexyl)amino]-4-{[(trifluoromethyl)sulfonyl]oxy}-2,4′-bipyridin-6-yl)piperazine-1-carboxylate

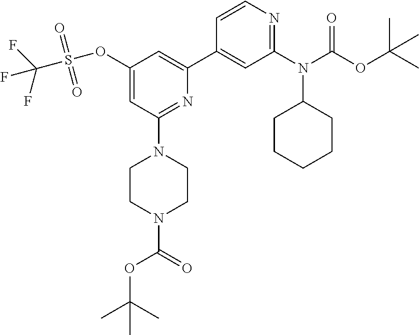

To a solution of 4-[2′-(tert-butoxycarbonyl-cyclohexyl-amino)-4-hydroxy-[2,4′]bipyridinyl-6-yl]-piperazine-1-carboxylic acid tert-butyl ester (0.8 g, 1.45 mmol) and triethylamine (1 mL, 7.25 mmol) in CH₂Cl₂ (35 mL), 2-(N,N-bis(trifluoromethylsulfonyl)amino)pyridine (0.675 g, 1.89 mmol) is added portionwise at 0° C. The mixture is allowed to warm to room temperature and stirred for 6 h and concentrated. The residue is diluted with CH₂Cl₂, washed with saturated NaHCO₃ (2×) and brine and then dried (Na₂SO₄), filtered and concentrated. The title compound tert-butyl 4-(2′-[(tert-butoxycarbonyl)(cyclohexyl)amino]-4-{[(trifluoromethyl)sulfonyl]oxy}-2,4′-bipyridin-6-yl)piperazine-1-carboxylate (1.15 g) is obtained as a crude intermediate. MS (ESI) m/z 686.2 (M+1), ¹H NMR (400 MHz, CDCl₃) δ ppm 8.56-8.58 (m, 1H), 7.66-7.68 (m, 1H), 7.64-7.65 (m, 1H), 6.98-7.00 (m, 1H), 6.50 (d, J=1.6 Hz, 1H), 4.08-4.18 (m, 1H), 3.65-3.71 (m, 4H), 3.55-3.62 (m, 4H), 1.90-1.98 (m, 2H), 1.71-1.81 (m, 2H), 1.55-1.64 (m, 1H), 1.50 (s, 9H), 1.42 (s, 9H), 1.30-1.37 (m, 4H), 0.97-1.11 (m, 1H).

### B. Cyclohexyl-(6-piperazin-1-yl-4-pyrimidin-5-yl-[2,4′]bipyridinyl-2′-yl)-amine

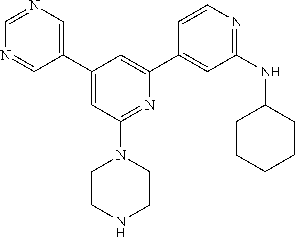

After a mixture of tert-butyl 4-(2′-[(tert-butoxycarbonyl)(cyclohexyl)amino]-4-{[(trifluoromethyl)sulfonyl]oxy}-2,4′-bipyridin-6-yl)piperazine-1-carboxylate (0.080 g, 0.117 mmol), pyrimidine-5-boronic acid (0.044 mL, 0.355 mmol) and DME (2 mL) is sparged with argon Pd(dppf)Cl₂.CH₂Cl₂ (0.0080 g, 0.01 mmol) is added followed by 2 M Na₂CO₃ (0.25 mL). The vessel is sealed and treated with microwave at 130° C. for 20 minutes. The mixture is filtered and concentrated. The residue is separated via flash chromatography (SiO₂, 50-70% EtOAc/heptane gradient) to give an intermediate of Boc protected the title compound [MS (ESI) m/z 616.3 (M+1)]. The intermediate is then treated with 50% TFA in CH₂Cl₂ at room temperature for 1 h and concentrated. The resulting residue is mixed with 2 N NH₃ in MeOH and concentrated again, and then separated via semi-prep HPLC (10-55% CH₃CN/H₂O gradient with 0.1% NH₄OH in 17 minutes) to give the title compound cyclohexyl-(6-piperazin-1-yl-4-pyrimidin-5-yl-[2,4′]bipyridinyl-2′-yl)-amine. MS (ESI) m/z 416.2 (M+1). ¹H NMR (400 MHz, DMSO-d₆) δ ppm 9.30 (s, 2H), 9.26 (s, 1H), 8.02 (d, J=5.3 Hz, 1H), 7.54 (d, J=0.9 Hz, 1H), 7.19-7.24 (m, 2H), 7.15 (dd, J=5.5, 1.5 Hz, 1H), 6.43 (d, J=7.8 Hz, 1H), 3.69-3.80 (m, 1H), 3.59-3.65 (m, 4H), 2.80-2.86 (m, 4H), 1.89-1.98 (m, 2H), 1.68-1.77 (m, 2H), 1.55-1.64 (m, 1H), 1.26-1.39 (m, 2H), 1.12-1.26 (m, 3H).

Compounds C—H of Example 53 can be prepared by a similar method as those above.

### C. Cyclohexyl-(6′-piperazin-1-yl-[3,4′;2′,4″]terpyridin-2″-yl)-amine

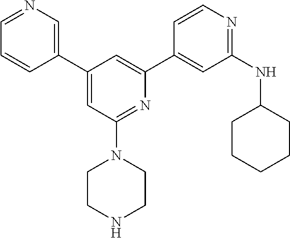

MS (ESI) m/z 415.3 (M+1). ¹H NMR (400 MHz, DMSO-d₆) δ ppm 9.03-9.09 (m, 1 H), 8.66 (dd, J=4.8, 1.5 Hz, 1H), 8.23-8.28 (m, 1H), 8.03 (d, J=5.4 Hz, 1H), 7.52-7.56 (m, 1H), 7.45-7.48 (m, 1H), 7.21-7.24 (m, 1H), 7.12-7.17 (m, 2H), 6.43 (d, J=7.7 Hz, 1H), 3.71-3.83 (m, 1H), 3.59-3.65 (m, 4H), 2.81-2.87 (m, 4H), 1.91-1.99 (m, 2H), 1.69-1.78 (m, 2H), 1.56-1.65 (m, 1H), 1.28-1.41 (m, 2H), 1.12-1.27 (m, 3H).

### D. N-[3-(2′-Cyclohexylamino-6-piperazin-1-yl-[2,4′]bipyridinyl-4-yl)-phenyl]-acetamide

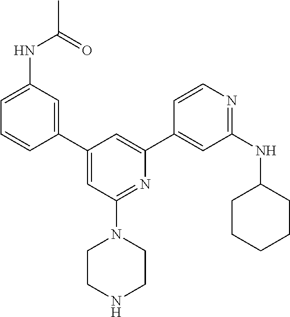

MS (ESI) m/z 471.3 (M+1). ¹H NMR (400 MHz, DMSO-d₆) δ ppm 10.05 (br. s., 1H), 8.01 (d, J=5.4 Hz, 1H), 7.90-7.92 (m, 1H), 7.69-7.73 (m, 1H), 7.39-7.50 (m, 2H), 7.31 (br. s., 1H), 7.17-7.19 (m, 1H), 7.06 (dd, J=5.4, 1.4 Hz, 1H), 6.95 (br. s., 1H), 6.46 (d, J=7.7 Hz, 1H), 3.69-3.81 (m, 1H), 3.55-3.61 (m, 4H), 2.81-2.86 (m, 4H), 2.07 (s, 3H), 1.90-1.98 (m, 2H), 1.68-1.77 (m, 2H), 1.56-1.64 (m, 1H), 1.26-1.40 (m, 2H), 1.12-1.26 (m, 3H).

### E. Cyclohexyl-[4-(3,5-dimethyl-isoxazol-4-yl)-6-piperazin-1-yl-[2,4′]bipyridinyl-2′-yl]-amine

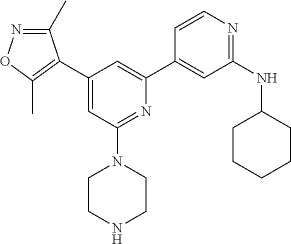

MS (ESI) m/z 433.1 (M+1). ¹H NMR (400 MHz, DMSO-d₆) δ ppm 8.00 (d, J=5.3 Hz, 1H), 7.15 (s, 1H), 7.09 (s, 1H), 7.03 (dd, J=5.4, 1.4 Hz, 1H), 6.76 (s, 1H), 6.43 (d, J=7.6 Hz, 1H), 3.68-3.79 (m, 1H), 3.53-3.58 (m, 4H), 2.79-2.85 (m, 4H), 2.47 (s, 3H), 2.29 (s, 3H), 1.88-1.98 (m, 2H), 1.67-1.78 (m, 2H), 1.56-1.65 (m, 1H), 1.26-1.40 (m, 2H), 1.13-1.25 (m, 3H).

### F. Cyclohexyl-[4-(4-fluorophenyl)-6-piperazin-1-yl-[2,4′]bipyridinyl-2′-yl]-amine

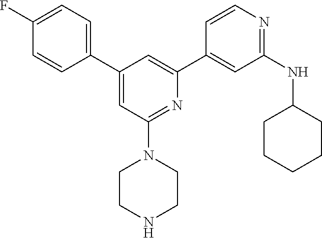

MS (ESI) m/z 432.2 (M+1). ¹H NMR (400 MHz, DMSO-d₆) δ ppm 8.01 (d, J=5.4 Hz, 1H), 7.87-7.93 (m, 2H), 7.37-7.39 (m, 1H), 7.30-7.36 (m, 2H), 7.18-7.21 (m, 1H), 7.11 (dd, J=5.4, 1.5 Hz, 1H), 7.03 (d, J=0.8 Hz, 1H), 6.42 (d, J=7.8 Hz, 1H), 3.69-3.80 (m, 1H), 3.55-3.62 (m, 4H), 2.80-2.86 (m, 4H), 1.89-1.98 (m, 2H), 1.67-1.77 (m, 2H), 1.55-1.63 (m, 1H), 1.26-1.39 (m, 2H), 1.11-1.26 (m, 3H).

### G. Cyclohexyl-[4-(3,5-dimethyl-1H-pyrazol-4-yl)-6-piperazin-1-yl-[2,4′]bipyridinyl-2′-yl]-amine

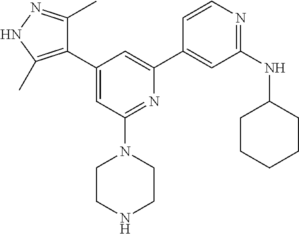

MS (ESI) m/z 432.2 (M+1). ¹H NMR (400 MHz, DMSO-d₆) δ ppm 12.45 (br. s., 1H), 7.99 (d, J=5.4 Hz, 1H), 7.13 (s, 1H), 6.99-7.05 (m, 2H), 6.64 (s, 1H), 6.42 (d, J=7.6 Hz, 1H), 3.67-3.80 (m, 1H), 3.47-3.56 (m, 4H), 2.78-2.86 (m, 4H), 2.27 (s, 6H), 1.89-1.99 (m, 2H), 1.67-1.78 (m, 2H), 1.54-1.65 (m, 1H), 1.25-1.40 (m, 2H), 1.11-1.25 (m, 3H).

### H. 3-(2′-Cyclohexylamino-6-piperazin-1-yl-[2,4′]bipyridinyl-4-yl)-benzonitrile

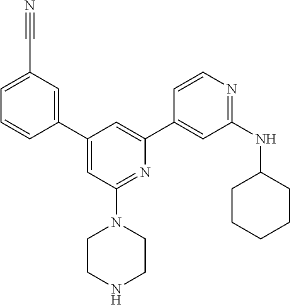

MS (ESI) m/z 439.3 (M+1). ¹H NMR (400 MHz, DMSO-d₆) δ ppm 8.40 (t, J=1.5 Hz, 1H), 8.19-8.23 (m, 1H), 8.02 (d, J=5.3 Hz, 1H), 7.90-7.94 (m, 1H), 7.71 (t, J=7.8 Hz, 1H), 7.47-7.50 (m, 1H), 7.20-7.23 (m, 1H), 7.14-7.17 (m, 2H), 6.43 (d, J=7.8 Hz, 1H), 3.69-3.82 (m, 1H), 3.59-3.64 (m, 4H), 2.81-2.86 (m, 4H), 1.89-1.98 (m, 2H), 1.67-1.77 (m, 2H), 1.55-1.65 (m, 1H), 1.26-1.42 (m, 2H), 1.12-1.26 (m, 3H).

### Example 54

### A. Cyclohexyl-(6-piperazin-1-yl-4-thiazol-5-yl-[2,4′]bipyridinyl-2′-yl)-amine

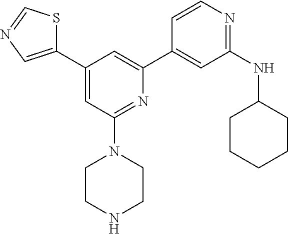

After a mixture of tert-butyl 4-(2′-[(tert-butoxycarbonyl)(cyclohexyl)amino]-4-{[(trifluoromethyl)sulfonyl]oxy}-2,4′-bipyridin-6-yl)piperazine-1-carboxylate (0.12 g, 0.175 mmol), lithium chloride (0.03 g, 0.715 mmol) and dioxane (4 mL) is sparged with argon 5-(tributylstannyl) thiazole (0.075 g, 0.2 mmol) is added followed by Pd(PPh₃)₄ (0.03 g, 0.026 mmol). The vessel is sealed and treated with microwave at 130° C. for 20 minutes. The mixture is filtered and concentrated. The residue is separated via flash chromatography (SiO₂, 30-50% EtOAc/heptane gradient) to give an intermediate of Boc protected the title compound [MS (ESI) m/z 621.2 (M+1)]. The yielded intermediate is then treated with 50% TFA in CH₂Cl₂ at room temperature for 1 h and concentrated. The resulting residue is mixed with 2 N NH₃ in MeOH and concentrated again, and then separated via semi-preparative HPLC (10-55% CH₃CN/H₂O gradient with 0.1% NH₄OH in 17 minutes) to give the title compound cyclohexyl-(6-piperazin-1-yl-4-thiazol-5-yl-[2,4′]bipyridinyl-2′-yl)-amine. MS (ESI) m/z 421.2 (M+1). ¹H NMR (400 MHz, DMSO-d₆) δ ppm 9.19-9.21 (m, 1 H), 8.62-8.65 (m, 1H), 8.02 (d, J=5.3 Hz, 1H), 7.37-7.38 (m, 1H), 7.16-7.18 (m, 1H), 7.09 (dd, J=5.4, 1.5 Hz, 1H), 7.04-7.06 (m, 1H), 6.46 (d, J=7.8 Hz, 1H), 3.70-3.84 (m, 1 H), 3.57-3.62 (m, 4H), 2.82-2.87 (m, 4H), 1.89-1.98 (m, 2H), 1.67-1.78 (m, 2H), 1.55-1.63 (m, 1H), 1.25-1.40 (m, 2H), 1.10-1.26 (m, 3H).

Compounds B-D of Example 54 can be prepared by a similar method as A.

### B. Cyclohexyl-(6-piperazin-1-yl-4-thiazol-4-yl-[2,4′]bipyridinyl-2′-yl)-amine

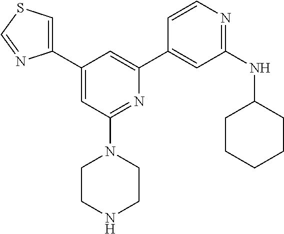

MS (ESI) m/z 421.1 (M+1). ¹H NMR (400 MHz, DMSO-d₆) δ ppm 9.25 (d, J=1.9 Hz, 1H), 8.57 (d, J=1.9 Hz, 1H), 8.02 (d, J=5.3 Hz, 1H), 7.74 (d, J=0.8 Hz, 1H), 7.38 (d, J=0.6 Hz, 1H), 7.19-7.20 (m, 1H), 7.08 (dd, J=5.4, 1.5 Hz, 1H), 6.47 (d, J=7.7 Hz, 1H), 3.69-3.80 (m, 1H), 3.56-3.61 (m, 4H), 2.82-2.87 (m, 4H), 1.89-1.99 (m, 2H), 1.68-1.77 (m, 2H), 1.55-1.64 (m, 1H), 1.26-1.38 (m, 2H), 1.11-1.26 (m, 3H).

### C. [4-(2-Aminothiazol-5-yl)-6-piperazin-1-yl-[2,4′]bipyridinyl-2′-yl]-cyclohexyl-amine

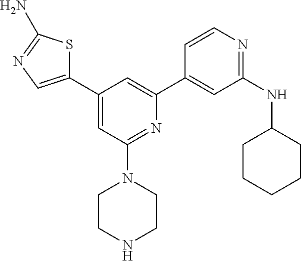

MS (ESI) m/z 436.1 (M+1). ¹H NMR (400 MHz, DMSO-d₆) δ ppm 7.99 (d, J=5.4 Hz, 1H), 7.79 (s, 1H), 7.36-7.39 (m, 2H), 7.15-7.16 (m, 1H), 7.12-7.14 (m, 1H), 7.04 (dd, J=5.4, 1.5 Hz, 1H), 6.70-6.72 (m, 1H), 6.41 (d, J=7.7 Hz, 1H), 3.69-3.79 (m, 1H), 3.51-3.56 (m, 4H), 2.81-2.87 (m, 4H), 1.88-1.99 (m, 2H), 1.67-1.78 (m, 2H), 1.55-1.64 (m, 1H), 1.25-1.39 (m, 2H), 1.12-1.25 (m, 3H).

### D. Cyclohexyl-(6-piperazin-1-yl-4-pyridazin-4-yl-[2,4′]bipyridinyl-2′-yl)-amine

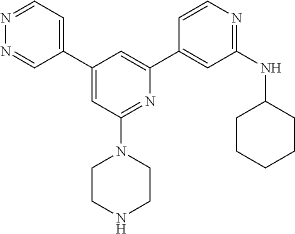

MS (ESI) m/z 416.1 (M+1). ¹H NMR (400 MHz, DMSO-d₆) δ ppm 9.78 (q, J=2.5, 1.2 Hz, 1H), 9.37 (dd, J=5.4, 1.1 Hz, 1H), 8.20 (q, J=5.4, 2.5 Hz, 1H), 8.03 (d, J=5.4 Hz, 1H), 7.60-7.62 (m, 1H), 7.30-7.32 (m, 1H), 7.21-7.23 (m, 1H), 7.16 (dd, J=5.4, 1.5 Hz, 1H), 6.44 (d, J=7.7 Hz, 1H), 3.71-3.81 (m, 1H), 3.65-3.71 (m, 4H), 2.86-2.92 (m, 4H), 1.89-1.98 (m, 2H), 1.67-1.77 (m, 2H), 1.55-1.65 (m, 1H), 1.26-1.40 (m, 2H), 1.12-1.26 (m, 3H).

### Example 55

### A. Cyclohexyl-(6-piperazin-1-yl-4-thiazol-2-yl-[2,4′]bipyridinyl-2′-yl)-amine

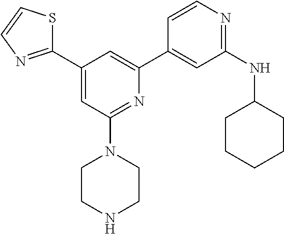

After a solution of tert-butyl 4-(2′-[(tert-butoxycarbonyl)(cyclohexyl)amino]-4-{[(trifluoromethyl)sulfonyl]oxy}-2,4′-bipyridin-6-yl)piperazine-1-carboxylate (0.12 g, 0.175 mmol) in THF (4 mL) is sparged with argon 2 M 2-thiazolylzinc bromide in THF (1.4 mL, 0.7 mmol) is added followed by Pd(PPh₃)₄ (0.016 g, 0.014 mmol). The mixture is heated at 80° C. under an argon atmosphere (balloon) for 21 h and then diluted with CH₂Cl₂, washed saturated Na₂CO₃, brine, dried (Na₂SO₄), filtered and concentrated. The residue is separated via flash chromatography (SiO₂, 25-55% EtOAc/heptane gradient) to give a mixture of mono-Boc and di-Boc protected the title compound. The yielded mixture is then treated with 50% TFA in CH₂Cl₂ at room temperature for 1 h and concentrated. The resulting residue is mixed with 2 N NH₃ in MeOH and concentrated again, and then separated via semi-prep HPLC (15-60% CH₃CN/H₂O gradient with 0.1% NH₄OH in 17 minutes) to give the title compound cyclohexyl-(6-piperazin-1-yl-4-thiazol-2-yl-[2,4′]bipyridinyl-2′-yl)-amine. MS (ESI) m/z 421.1 (M+1). ¹H NMR (400 MHz, DMSO-d₆) δ ppm 8.02-8.05 (m, 2H), 7.95 (d, J=3.2 Hz, 1H), 7.58 (s, 1H), 7.25 (s, 1H), 7.18 (s, 1H), 7.06 (dd, J=5.4, 1.3 Hz, 1H), 6.50 (d, J=7.7 Hz, 1H), 3.69-3.83 (m, 1H), 3.57-3.62 (m, 4 H), 2.82-2.87 (m, 4H), 1.90-1.99 (m, 2H), 1.67-1.77 (m, 2H), 1.55-1.64 (m, 1H), 1.26-1.39 (m, 2H), 1.13-1.26 (m, 3H).

### Example 56

### A. (4-Bromo-6-piperazin-1-yl-[2,4′]bipyridinyl-2′-yl)-cyclohexyl-amine

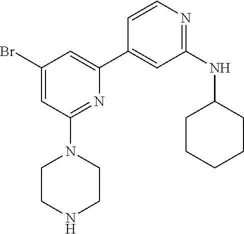

A mixture of 4-[2′-(tert-butoxycarbonyl-cyclohexyl-amino)-4-hydroxy-[2,4′]bipyridinyl-6-yl]-piperazine-1-carboxylic acid tert-butyl ester (1.2 g, 2.17 mmol) and POBr₃ (3.7 g, 13 mmol) is placed in flask with HBr receiver and heated to 130° C. for 1 h. The reaction is cooled to 0° C., quenched with MeOH and concentrated. The resulting slurry is taken up to saturated NaHCO₃, extracted with CH₂Cl₂ (×5). the combined organic layer is concentrated. The residue is then separated via semi-preparative HPLC (25-55% CH₃CN/H₂O gradient with 0.1% NH₄OH in 17 minutes) to give the title compound (4-Bromo-6-piperazin-1-yl-[2,4′]bipyridinyl-2′-yl)-cyclohexyl-amine. MS (ESI) m/z 451.9, 417.9 (M+1). ¹H NMR (400 MHz, DMSO-d₆) δ ppm 9.06 (br. s., 2H), 8.01 (d, J=5.6 Hz, 1H), 7.46 (s, 1H), 7.27 (s, 1H), 7.21 (br. s., 1H), 7.09 (br. s., 1H), 3.85-3.90 (m, 4H), 3.70-3.81 (m, 1H), 3.18-3.25 (m, 4H), 1.89-1.98 (m, 2H), 1.69-1.78 (m, 2H), 1.57-1.66 (m, 1H), 1.28-1.42 (m, 2H), 1.13-1.28 (m, 3H).

### B. tert-Butyl 4-[4-bromo-2′-(cyclohexylamino)-2,4′-bipyridin-6-yl]piperazine-1-carboxylate

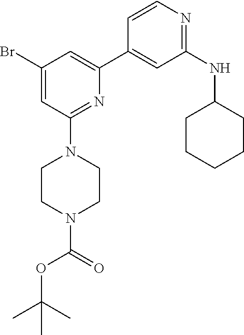

A mixture of (4-bromo-6-piperazin-1-yl-[2,4′]bipyridinyl-2′-yl)-cyclohexylamine (crude, 0.362 mmol), Boc anhydride (0.4 g, 1.81 mmol) and triethylamine (0.252 mL, 1.81 mmol) in CH₂Cl₂ (25 mL) is stirred at room temperature for 0.5 h and diluted with CH₂Cl₂, washed with saturated NaHCO₃ (×2), brine, dried (Na₂SO₄), filtered and concentrated. The residue is separated via flash chromatography (SiO₂, 15-25% EtOAc/heptane gradient) to give the title compound to give compound. ¹H NMR (400 MHz, CDCl₃) δ ppm 8.13-8.16 (m, 1H), 7.22 (d, J=1.3 Hz, 1H), 7.02 (dd, J=5.4, 1.5 Hz, 1H), 6.92-6.95 (m, 1H), 6.81 (d, J=1.3 Hz, 1H), 4.49-4.56 (m, 1H), 3.60-3.73 (m, 5H), 3.55-3.60 (m, 4H), 2.04-2.13 (m, 2H), 1.71-1.83 (m, 2H), 1.62-1.71 (m, 1H), 1.50 (s, 9H), 1.38-1.47 (m, 2H), 1.17-1.35 (m, 3H).

### Example 57

### A. Cyclohexyl-[6-piperazin-1-yl-4-(1H-pyrazol-4-yl)-[2,4′]bipyridinyl-2′-yl]-amine

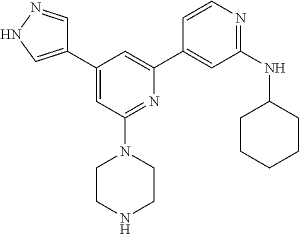

A mixture of tert-butyl 4-[4-bromo-2′-(cyclohexylamino)-2,4′-bipyridin-6-yl]piperazine-1-carboxylate (0.22 g, 0.426 mmol), 1H-pyrazole-4-boronic acid (0.29 g, 2.4 mmol), Pd(dppf)Cl₂. CH₂Cl₂ (0.007 g, 0.085 mmol), 2 M Na₂CO₃ (2.4 mL) and DME (5 mL) is sparged with argon for 10 min. The vessel is sealed and treated with microwave at 130° C. for 20 minutes. The mixture is diluted with CH₂Cl₂, washed with saturated NaHCO₃, brine, dried (Na₂SO₄), filtered and concentrated. The residue is separated via flash chromatography (SiO₂, 70-100% EtOAc/heptane gradient) to give an intermediate of Boc protected the title compound [MS (ESI) m/z 504.0 (M+1)]. The yielded intermediate is then treated with 50% TFA in CH₂Cl₂ at room temperature for 1 h and concentrated. The resulting residue is mixed with 2 N NH₃ in MeOH and concentrated again and then separated via semi-prep HPLC (10-55% CH₃CN/H₂O gradient with 0.1% NH₄OH in 17 minutes) to give the title compound cyclohexyl-[6-piperazin-1-yl-4-(1H-pyrazol-4-yl)-[2,4′]bipyridinyl-2′-yl]-amine. MS (ESI) m/z 404.0 (M+1). ¹H NMR (400 MHz, DMSO-d₆) δ ppm 13.07 (br. s., 1H), 8.45 (br. s., 1H), 8.19 (br. s., 1H), 8.00 (d, J=5.3 Hz, 1H), 7.39-7.41 (m, 1H), 7.17-7.18 (m, 1H), 7.08 (dd, J=5.4, 1.5 Hz, 1H), 7.02-7.04 (m, 1H), 6.40 (d, J=7.7 Hz, 1H), 3.69-3.79 (m, 1H), 3.53-3.58 (m, 4H), 2.80-2.85 (m, 4H), 1.89-1.99 (m, 2H), 1.68-1.77 (m, 2H), 1.55-1.65 (m, 1H), 1.26-1.39 (m, 2H), 1.13-1.26 (m, 3H).

Compound B of Example 54 can be prepared by a similar method as A.

### B. Cyclohexyl-[6-piperazin-1-yl-4-(2H-pyrazol-3-yl)-[2,4′]bipyridinyl-2′-yl]-amine

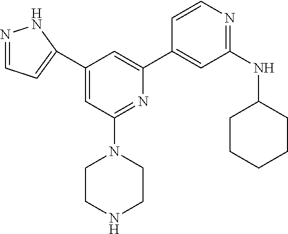

MS (ESI) m/z 404.0 (M+1). ¹H NMR (400 MHz, DMSO-d₆) δ ppm 13.09 (br. s., 1H), 8.01 (d, J=5.3 Hz, 1H), 7.84 (br. s., 1H), 7.58 (br. s., 1H), 7.16-7.21 (m, 2H), 7.06 (dd, J=5.4, 1.5 Hz, 1H), 6.97 (d, J=2.0 Hz, 1H), 6.45 (d, J=7.6 Hz, 1H), 3.69-3.82 (m, 1H), 3.53-3.61 (m, 4H), 2.82-2.88 (m, 4H), 1.90-1.99 (m, 2H), 1.67-1.78 (m, 2H), 1.54-1.65 (m, 1H), 1.26-1.41 (m, 2H), 1.11-1.26 (m, 3H).

### Example 58

### A. Cyclohexyl-(4-methoxy-6-piperazin-1-yl-[2,4′]bipyridinyl-2′-yl)-amine

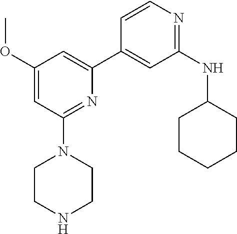

A mixture of 4-(2′-cyclohexylamino-4-nitro-[2,4′]bipyridinyl-6-yl)-piperazine-1-carboxylic acid tert-butyl ester (0.08 g, 0.166 mmol) and NaOCH₃ (0.09 g, 1.66 mmol) in DMSO (3 mL) is stirred at room temperature for 1.5 h and then diluted with CH₂Cl₂, washed with H20 (×2), brine, dried (Na₂SO₄), filtered and concentrated. The residue is separated via flash chromatography (SiO₂, 30-50% EtOAc/heptane gradient) to give a intermediate of the title compound with Boc protecting group [0.05 g, 65%, MS (ESI) m/z 468.2 (M+1), ¹H NMR (400 MHz, CDCl₃) δ ppm 8.12 (d, J=5.3 Hz, 1H), 7.04 (dd, J=5.4, 1.4 Hz, 1H), 6.98-7.00 (m, 1H), 6.73 (d, J=1.8 Hz, 1H), 6.14 (d, J=1.8 Hz, 1H), 3.89 (s, 3H), 3.55-3.72 (m, 9H), 2.05-2.14 (m, 2H), 1.73-1.83 (m, 2H), 1.61-1.71 (m, 1H), 1.50 (s, 9H), 1.37-1.46 (m, 2H), 1.19-1.33 (m, 3H)

The intermediate is mixed with 50% TFA in CH₂Cl₂ and stirred at room temperature for 2 h and concentrated. The residue is mixed with 2 N NH₃ in MeOH and concentrated again, and then separated via semi-prep HPLC (10-55% CH₃CN/H₂O gradient with 0.1% NH₄OH in 17 minutes) to give the title compound Cyclohexyl-(4-methoxy-6-piperazin-1-yl-[2,4′]bipyridinyl-2′-yl)-amine. MS (ESI) m/z 367.1 (M+1). ¹H NMR (400 MHz, DMSO-d₆) δ ppm 7.97 (d, J=5.2 Hz, 1H), 7.10 (br. s., 1H), 6.97 (dd, J=5.5, 1.5 Hz, 1H), 6.75 (d, 1H), 6.40 (d, J=7.7 Hz, 1H), 6.29 (d, J=1.5 Hz, 1H), 3.84 (s, 3H), 3.68-3.79 (m, 1H), 3.46-3.54 (m, 4H), 2.77-2.86 (m, 4H), 1.87-1.98 (m, 2H), 1.66-1.77 (m, 2H), 1.54-1.64 (m, 1H), 1.25-1.39 (m, 2H), 1.12-1.25 (m, 3H).

### Example 59

### A. 4-(2′-iso-Propylamino-4-nitro-[2,4′]bipyridinyl-6-yl)-piperazine-1-carboxylic acid tert-butyl ester

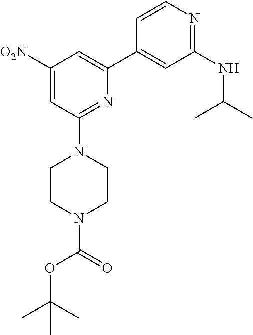

After a solution of 4-(2′-chloro-4-nitro-[2,4′]bipyridinyl-6-yl)-piperazine-1-carboxylic acid tert-butyl ester (0.8 g, 1.91 mmol) in dioxane (75 mL) is sparged with argon isopropylamine (3.25 mL, 38.14 mmol) is added followed by Pd(tBu₃P)₂ and cesium carbonate (1.87 g, 5.73 mmol). The vessel is sealed and heated at 110° C. for 5 h. The mixture is then allowed to cool and then filtered and concentrated. The residue is separated via flash chromatography (SiO₂, 25-55% EtOAc/hexanes gradient) to give the title compound 4-(2′-isopropylamino-4-nitro-[2,4′]bipyridinyl-6-yl)-piperazine-1-carboxylic acid tert-butyl ester. MS (ESI) m/z 443.1 (M+1). ¹H NMR (400 MHz, CDCl3) δ ppm 8.19 (dd, J=5.4, 0.6 Hz, 1H), 7.71 (d, J=1.6 Hz, 1H), 7.33 (d, J=1.5 Hz, 1H), 7.10 (dd, J=5.3, 1.5 Hz, 1H), 6.93-6.98 (m, 1H), 3.96-4.11 (m, 1H), 3.95-4.10 (m, 1H), 3.70-3.78 (m, 4H), 3.61 (dd, J=6.4, 4.0 Hz, 4H), 1.50 (s, 9H), 1.29 (d, J=6.3 Hz, 6H).

### B. tert-Butyl 4-{2′-[(tert-butoxycarbonyl)(isopropyl)amino]-4-nitro-2,4′-bipyridin-6-yl}piperazine-1-carboxylate

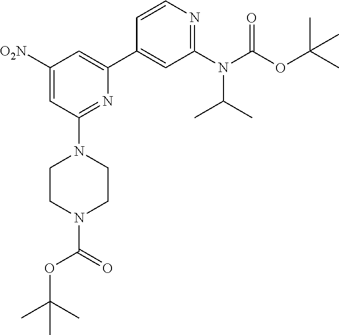

The title compound is prepared in similar method to compound Example 43B. MS (ESI) m/z 543.3 (M+1). ¹H NMR (400 MHz, CDCl₃) δ ppm 8.58 (dd, J=5.2, 0.6 Hz, 1H), 7.76-7.80 (m, 2H), 7.73 (dd, J=5.2, 1.6 Hz, 1H), 7.38 (d, J=1.5 Hz, 1H), 4.53-4.65 (m, 1H), 3.74-3.79 (m, 4H), 3.62 (dd, J=6.3, 4.0 Hz, 4H), 1.51 (s, 9H), 1.46 (s, 9H), 1.32 (d, J=6.8 Hz, 6H).

### C. tert-Butyl 4-{2′-[(tert-butoxycarbonyl)(isopropyl)amino]-4-hydroxy-2,4′-bipyridin-6-yl}piperazine-1-carboxylate

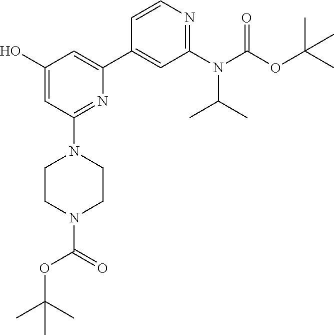

The title compound is prepared in similar method to Example 52A. MS (ESI) m/z 514.3 (M+1). The crude title compound is taken on without further purification.

### D. tert-Butyl 4-(2′-[(tert-butoxycarbonyl)(isopropyl)amino]-4-{[(trifluoromethyl)sulfonyl]oxy}-2,4′-bipyridin-6-yl)piperazine-1-carboxylate

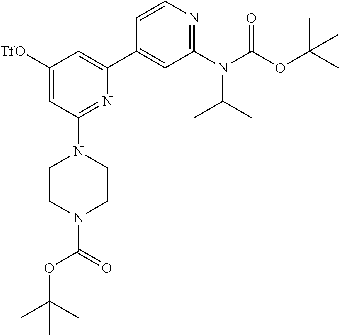

The title compound is prepared in similar method to Example 53A. MS (ESI) m/z 646.2 (M+1). ¹H NMR (400 MHz, CDCl₃) δ ppm 8.55 (dd, J=5.2, 0.5 Hz, 1H), 7.71 (d, J=0.9 Hz, 1H), 7.65 (dd, J=5.2, 1.6 Hz, 1H), 7.00 (d, J=1.6 Hz, 1H), 6.50 (d, J=1.8 Hz, 1H), 4.53-4.65 (m, 1H), 3.66-3.71 (m, 4H), 3.57-3.63 (m, 4H), 1.50 (s, 9H), 1.45 (s, 9H), 1.31 (d, J=6.8 Hz, 6H).

### E. iso-Propyl-[6-piperazin-1-yl-4-(1H-pyrazol-4-yl)-[2,4′]bipyridinyl-2′-yl]-amine

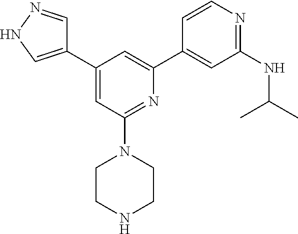

The title compound is prepared in similar method to compound Example 53B. MS (ESI) m/z 364.2 (M+1). ¹H NMR (400 MHz, DMSO-d₆) δ ppm 13.11 (br. s., 1H), 8.41 (br. s., 2H), 8.02 (d, J=5.3 Hz, 1H), 7.41 (s, 1H), 7.16 (s, 1H), 7.10 (dd, J=5.3, 1.3 Hz, 1H), 7.04 (s, 1H), 6.37 (d, J=7.6 Hz, 1H), 3.98-4.13 (m, 1H), 3.52-3.59 (m, 4H), 2.80-2.87 (m, 4H), 1.16 (d, J=6.6 Hz, 6H).

### F. [6-piperazin-1-yl-4-(1H-pyrazol-4-yl)-[2,4′]bipyridinyl-2′-yl]-(tetrahydropyran-4-yl)-amine

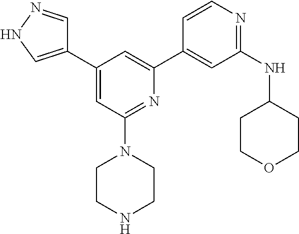

The title compound is prepared in similar method to compound Example 59E. MS (ESI) m/z 406.2 (M+1). ¹H NMR (400 MHz, DMSO-d₆) δ ppm 13.17 (br. s., 1H), 8.99 (br. s., 2H), 8.49 (br. s., 1H), 8.21 (br. s., 1H), 8.03 (d, J=5.6 Hz, 1H), 7.55 (s, 1H), 7.25 (br. s., 1 H), 7.17-7.23 (m, 2H), 3.94-4.06 (m, 1H), 3.85-3.93 (m, 6H), 3.39-3.47 (m, 2H), 3.21-3.26 (m, 4H), 1.91 (d, J=15.3 Hz, 2H), 1.39-1.53 (m, 2H).

### Example 60

### A. 2,6-Dibromo-isonicotinamide

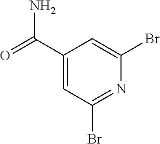

A mixture of citrazinic acid (10.0 g, 64.4 mmol) and POBr₃ (64.0 g, 225.4 mmol) is placed in a flask with HBr receiver (a saturated KOH reservoir is attached with condenser through a funnel) and heated at 130° C. for 3 h. The mixture is allowed to cool to 0° C. and the reaction is carefully quenched with NH₄OH (about 350 mL). The mixture is stirred at room temperature over night. The solid is collected through filtration to give 1^{st} batch of title compound (5.6 g). [(ESI) m/z 280.8 (M+1), ¹H NMR (400 MHz, CDCl₃) δ ppm 7.80 (s, 2H), 5.65-6.23 (m, 2H). The filtrate is extracted with CH₂Cl₂ (×2). Combined organic layer is washed with brine, dried (Na₂SO₄), filtered and concentrated to 2^{nd} batch of title compound (2.76 g). [(ESI) m/z 278.8, 280.8, 282.8 (M+1)]. The total 8.4 g of title compound 2,6-dibromo-isonicotinamide is obtained in 47% yield.

### B. tert-Butyl[1-(6-bromo-4-carbamoylpyridin-2-yl)piperidin-4-yl]carbamate

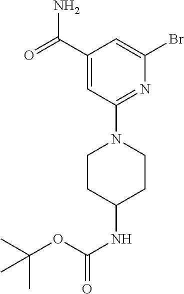

A mixture of 2,6-dibromo-isonicotinamide (1.0 g, 3.57 mmol), 4-Boc-aminopiperidine (0.72 g, 3.57 mmol), triethylamine (0.5 mL, 3.57 mmol) and dioxane (20 mL) is heated to 130° C. for 18 h in a sealed vessel, and then allowed to cool to room temperature, diluted with CH₂Cl₂, washed with saturated NaHCO₃. Combined aqueous layer is extracted with CH₂Cl₂. Combined organic layer is washed with brine, dried (Na₂SO₄), filtered and concentrated. The resulting solid is triturated with a mixture of ether and CH₂Cl₂ and filtered to give the title compound (1.1 g, 74%). MS (ESI) m/z 398.9, 400.9 (M+1). ¹H NMR (400 MHz, CDCl₃) δ ppm 6.99 (d, J=1.0 Hz, 1H), 6.90 (d, J=1.0 Hz, 1H), 5.95 (br. s., 1H), 5.58 (br. s., 1H), 4.39-4.56 (m, 1H), 4.22-4.32 (m, 2H), 3.62-3.82 (m, 1H), 2.98-3.11 (m, 2H), 2.00-2.11 (m, 2H), 1.43-1.50 (m, 9H), 1.34-1.43 (m, 2H).

### C. tert-Butyl[1-(4-carbamoyl-2′-fluoro-2,4′-bipyridin-6-yl)piperidin-4-yl]carbamate

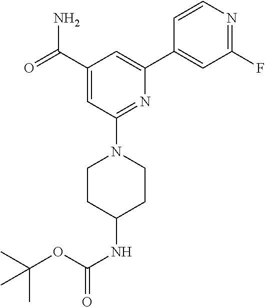

The title compound is prepared in similar method to Example 42B. MS (ESI) m/z 416.1 (M+1). ¹H NMR (400 MHz, MeOD) δ ppm 8.27 (d, J=5.3 Hz, 1H), 7.94-7.98 (m, 1 H), 7.71 (s, 1H), 7.63-7.65 (m, 1H), 7.31-7.33 (m, 1H), 7.06-7.10 (m, 1H), 4.44-4.52 (m, 2H), 3.56-3.77 (m, 1H), 2.98-3.19 (m, 3H), 1.93-2.04 (m, 2H), 1.42-1.54 (m, 11 H).

### D. 4-Amino-2″-cyclohexylamino-3,4,5,6-tetrahydro-2H-[1,2′;6′,4″]terpyridine-4′-carboxylic acid amide

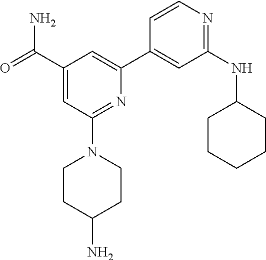

A mixture of tert-butyl[1-(4-carbamoyl-2′-fluoro-2,4′-bipyridin-6-yl)piperidin-4-yl]carbamate (0.08 g, 0.193 mmol) and cyclohexylamine (2.5 mL) is heated to 110° C. for 30 h and then concentrated. The residue is triturated with ether. The solid is collected and then treated with 50% TFA in CH₂Cl₂ at room temperature for 20 minutes. The resulting residue is mixed with 2 N NH₃ in MeOH and concentrated again and then separated via semi-preparative HPLC (10-55% CH₃CN/H₂O gradient with 0.1% NH₄OH in 17 minutes) to give the title compound 4-amino-2″-cyclohexylamino-3,4,5,6-tetrahydro-2H-[1,2′;6′,4″]terpyridine-4′-carboxylic acid amide. MS (ESI) m/z 395.2 (M+1). ¹H NMR (400 MHz, MeOD-d₄) δ ppm ¹H NMR (400 MHz, MeOD) δ ppm 7.97 (d, J=5.3 Hz, 1H), 7.50 (d, J=0.8 Hz, 1H), 7.21-7.26 (m, 2H), 7.13 (dd, J=5.6, 1.5 Hz, 1H), 4.49-4.59 (m, 2H), 3.62-3.72 (m, 1H), 2.91-3.08 (m, 3H), 2.00-2.09 (m, 2H), 1.91-1.99 (m, 2H), 1.75-1.85 (m, 2H), 1.63-1.73 (m, 1H), 1.35-1.53 (m, 4H), 1.21-1.34 (m, 3H).

Compounds E-K of Example 60 can be prepared by a similar method as those above.

### E. 4-Aminomethyl-2″-cyclohexylamino-3,4,5,6-tetrahydro-2H-[1,2′;6′,4″]terpyridine-4′-carboxylic acid amide

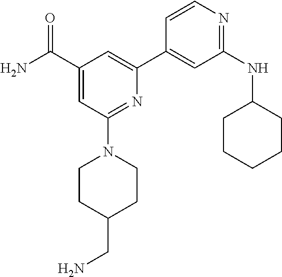

MS (ESI) m/z 409.2 (M+1). ¹H NMR (400 MHz, MeOD) δ ppm 7.94-7.98 (m, 1H), 7.47-7.50 (m, 1H), 7.18-7.25 (m, 2H), 7.13 (dd, J=5.7, 1.5 Hz, 1H), 4.54-4.65 (m, 2H), 3.62-3.74 (m, 1H), 2.89-3.02 (m, 2H), 2.64 (d, J=6.8 Hz, 2H), 1.99-2.10 (m, 2H), 1.84-1.92 (m, 2H), 1.63-1.84 (m, 4H), 1.38-1.54 (m, 2H), 1.21-1.35 (m, 5H)

### F. 2′-Cyclohexylamino-6-(R)-hexahydro-pyrrolo[1,2-a]pyrazin-2-yl-[2,4′]bipyridinyl-4-carboxylic acid amide

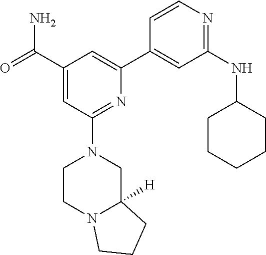

MS (ESI) m/z 421.2 (M+1). ¹H NMR (400 MHz, DMSO-d₆) δ ppm 8.18 (br. s., 1H), 8.02 (d, J=5.6 Hz, 1H), 7.61 (br. s., 1H), 7.52 (s, 1H), 7.25 (s, 1H), 7.15 (s, 1H), 7.03 (dd, 1H), 6.48 (d, J=7.6 Hz, 1H), 4.50-4.59 (m, 1H), 4.37-4.47 (m, 1H), 3.66-3.80 (m, 1H), 3.00-3.16 (m, 2H), 2.90-3.00 (m, 1H), 2.58-2.65 (m, 1H), 2.13-2.22 (m, 1H), 2.04-2.13 (m, 1H), 1.83-2.03 (m, 4H), 1.67-1.79 (m, 4H), 1.55-1.65 (m, 1H), 1.37-1.48 (m, 1H), 1.26-1.36 (m, 2H), 1.13-1.26 (m, 3H).

### G. 2′-Cyclohexylamino-6-(3,5-dimethyl-piperazin-1-yl)-[2,4′]bipyridinyl-4-carboxylic acid amide

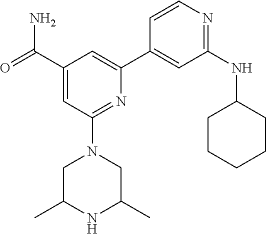

MS (ESI) m/z 409.1 (M+1). ¹H NMR (400 MHz, DMSO-d₆) δ ppm 8.16 (br. s., 1H), 8.02 (d, J=5.3 Hz, 1H), 7.61 (br. s., 1H), 7.49 (s, 1H), 7.20 (s, 1H), 7.12 (s, 1H), 7.02 (dd, J=5.4, 1.5 Hz, 1H), 6.44 (d, J=7.8 Hz, 1H), 4.27-4.35 (m, 2H), 3.65-3.80 (m, 1H), 2.73-2.87 (m, 2H), 2.29-2.42 (m, 2H), 1.89-1.99 (m, 2H), 1.68-1.78 (m, 2H), 1.55-1.65 (m, 1H), 1.26-1.40 (m, 2H), 1.13-1.26 (m, 3H), 1.08 (d, J=6.2 Hz, 6H).

### H. 3-Amino-2″-cyclohexylamino-3,4,5,6-tetrahydro-2H-[1,2′;6′,4″]terpyridine-4′-carboxylic acid amide

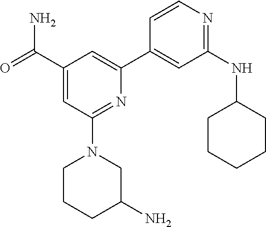

MS (ESI) m/z 395.1 (M+1). ¹H NMR (400 MHz, CDCl₃) δ ppm 8.15 (d, J=5.3 Hz, 1H), 7.20-7.25 (m, 1H), 7.05-7.12 (m, 2H), 7.02 (br. s., 1H), 6.18 (br. s., 1H), 5.68 (br. s., 1H), 4.56 (d, J=7.8 Hz, 1H), 4.31-4.38 (m, 1H), 4.15-4.25 (m, 1H), 3.61-3.77 (m, 1H), 3.06-3.16 (m, 1H), 2.82-2.99 (m, 2H), 1.99-2.15 (m, 3H), 1.73-1.91 (m, 3H), 1.60-1.72 (m, 2H), 1.32-1.48 (m, 3H), 1.18-1.32 (m, 3H)

### I. 2′-Cyclohexylamino-6-(4-methyl-piperazin-1-yl)-[2,4′]bipyridinyl-4-carboxylic acid amide

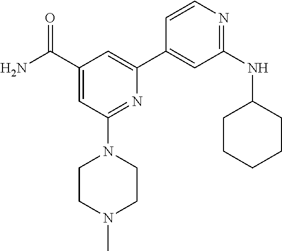

MS (ESI) m/z 493.1 (M+1). ¹H NMR (400 MHz, DMSO-d₆) δ ppm 8.17 (br. s., 1H), 8.01 (d, J=5.3 Hz, 1H), 7.60 (br. s., 1H), 7.53 (s, 1H), 7.23 (s, 1H), 7.15 (s, 1H), 7.02 (dd, J=5.4, 1.4 Hz, 1H), 3.69-3.80 (m, 1H), 3.60-3.66 (m, 4H), 2.41-2.47 (m, 4H), 2.24 (s, 3 H), 1.89-1.97 (m, 2H), 1.67-1.78 (m, 2H), 1.54-1.65 (m, 1H), 1.25-1.40 (m, 2H), 1.11-1.26 (m, 3H).

### J. 2′-Cyclohexylamino-[2,4′]bipyridinyl-4-carboxylic acid amide

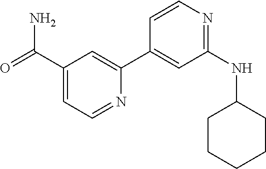

MS (ESI) m/z 297.1 (M+1). ¹H NMR (400 MHz, MeOD-d₄) δ ppm 8.79 (dd, J=5.1, 0.8 Hz, 1H), 8.25 (dd, J=1.5, 0.8 Hz, 1H), 8.04 (dd, J=5.6, 0.6 Hz, 1H), 7.80 (dd, J=5.1, 1.6 Hz, 1H), 7.14-7.15 (m, 1H), 7.12 (dd, 1H), 3.65-3.75 (m, 1H), 2.01-2.09 (m, 2H), 1.76-1.85 (m, 2H), 1.64-1.72 (m, 1H), 1.39-1.54 (m, 2H), 1.21-1.34 (m, 3H).

### K. 2′-iso-Propylamino-[2,4′]bipyridinyl-4-carboxylic acid amide

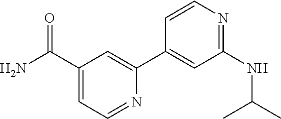

MS (ESI) m/z 257.1 (M+1). ¹H NMR (400 MHz, MeOD-d₄) δ ppm 8.79 (dd, 1H), 8.23-8.26 (m, 1H), 8.05 (dd, J=5.2, 1.1 Hz, 1H), 7.78-7.82 (m, 1H), 7.12-7.15 (m, 2H), 4.00-4.10 (m, 1H), 1.22-1.27 (m, 6H).

### Example 61

### A. tert-Butyl({1-[4-carbamoyl-2′-(cyclohexylamino)-2,4′-bipyridin-6-yl]piperidin-4-yl}methyl)carbamate

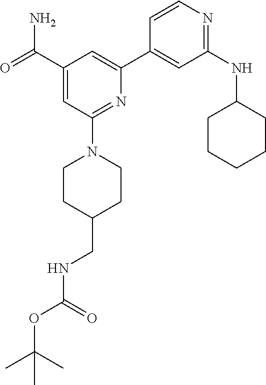

A mixture of tert-butyl {[1-(4-carbamoyl-2′-fluoro-2,4′-bipyridin-6-yl)piperidin-4-yl]methyl}carbamate (0.12 g, 0.28 mmol) and cyclohexylamine (4 mL) is heated to 120° C. for 24 h and then concentrated. The residue is purified via flash chromatography (SiO₂, 70-100% EtOAc/hexanes gradient) to give title compound MS (ESI) m/z 509.1 (M+1).

### B. 4-Aminomethyl-2″-cyclohexylamino-3,4,5,6-tetrahydro-2H-[1,2′;6′,4″]terpyridine-4′-carbonitrile trifluoroactic acid salt

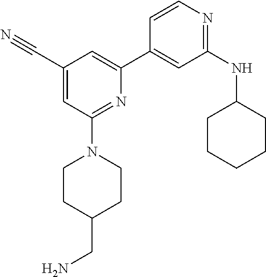

To a mixture of tert-butyl({1-[4-carbamoyl-2′-(cyclohexylamino)-2,4′-bipyridin-6-yl]piperidin-4-yl}methyl)carbamate (0.078 g, 0.153 mmol) and triethylamine (0.2 mL, 1.44 mmol) in CH₂Cl₂ (5 mL), trifluoroacetic anhydride is added at 0° C. and stirred for 10 min. The mixture is allowed to warm to room temperature and stirred for 3 h. The resulting mixture is diluted with CH₂Cl₂, washed with saturated NaHCO₃ (×2), brine, dried (Na₂SO₄), filtered and concentrated. The residue (0.09 g) is taken on without further purification.

The residue from above is mixed with K₂CO₃ (0.064 g, 0.461 mmol) in MeOH/H₂O (3:1, 24 mL). The mixture is stirred at room temperature for 0.5 h and then concentrated. The residue is taken up to CH₂Cl₂, washed with saturated NaHCO₃ (×2), brine, dried (Na₂SO₄), filtered and concentrated. The residue is separated via flash chromatography (SiO₂, 10-35% EtOAc/hexanes gradient) to give an intermediate of Boc protected the title compound [MS (ESI) m/z 491.1 (M+1).

The intermediate from above is treated 50% TFA in CH₂Cl₂ at room temperature for 1 h. The resulting solution is concentrated. The residue is then separated via semi-prep HPLC (3-28% CH₃CN/H₂O gradient with 0.1% TFA in 20 minutes) to give the title compound 4-aminomethyl-2″-cyclohexylamino-3,4,5,6-tetrahydro-2H-[1,2′;6′,4″]terpyridine-4′-carbonitrile trifluoroactic acid salt. MS (ESI) m/z 391.1 (M+1). ¹H NMR (400 MHz, DMSO-d₆) δ ppm 8.01 (d, J=6.4 Hz, 1H), 7.73-7.90 (m, 2H), 7.68 (br. s., 1H), 7.58-7.66 (m, 1H), 7.55 (br. s., 1H), 7.32-7.40 (m, 1H), 4.45-4.59 (m, 2H), 3.65-3.75 (m, 1H), 2.91-3.02 (m, 2H), 2.72-2.80 (m, 2H), 2.38-2.48 (m, 1H), 1.71-2.00 (m, 7H), 1.59-1.67 (m, 1H), 1.13-1.44 (m, 7H).

### C. 3-Amino-2″-cyclohexylamino-3,4,5,6-tetrahydro-2H-[1,2′;6′,4″]terpyridine-4′-carbonitrile trifluoroactic acid salt

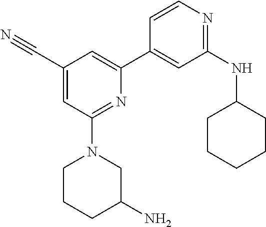

The title compound is prepared with similar method to Example 61A. MS (ESI) m/z 377.0 (M+1). ¹H NMR (400 MHz, 100° C., DMSO-d₆) δ ppm 8.03 (d, J=5.9 Hz, 1H), 7.50 (s, 1H), 7.35 (br. s., 1H), 7.30 (s, 1H), 7.21 (dd, J=5.9, 1.5 Hz, 1H), 4.28-4.37 (m, 1H), 3.93-4.03 (m, 1H), 3.73-3.84 (m, 1H), 3.25-3.48 (m, 3H), 2.02-2.13 (m, 1H), 1.93-2.02 (m, 2H), 1.82-1.93 (m, 1H), 1.70-1.82 (m, 3H), 1.58-1.70 (m, 2H), 1.20-1.48 (m, 5H).

### Example 62

### A. 2″-Cyclohexylamino-3,4,5,6-tetrahydro-2H-[1,2′;6′,4″]terpyridine-4,4′-dicarboxylic acid 4′-amide 4-(isobutylamide)

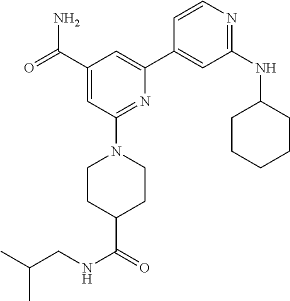

A mixture of 2,6-dibromo-isonicotinamide (0.5 g, 1.79 mmol), piperidine-4-carboxylic acid methyl ester (0.619 mL, 4.5 mmol), triethylamine (1.25 mL, 9 mmol) and MeOH (12 mL) is heated to 85° C. for 1.5 h and then 110° C. for 1.5 h, and then concentrated. The residue is taken on without further purification.

To a mixture of the residue from above, 2-fluoropyridine-4-boronic acid (0.61 g, 4.3 mmol), aqueous solution of Na₂CO₃ (5.4 mL, 2.0 M) and CH₃CN (12 mL), sparged with argon, Pd(PPh₃)₄ (0.414 g, 0.356 mmol) is added. The mixture is heated to 100° C. for 6 h. The mixture is then allowed to cool, diluted with CH₂Cl₂ and washed with saturated NaHCO₃ (×2). Combined aqueous layer is extracted with CH₂Cl₂. Combined organic layer is dried (Na₂SO₄), filtered and concentrated. The residue is then separated via flash chromatography (SiO₂, 60-95% EtOAc/hexanes gradient) to give an intermediate which is taken on to next step.

A mixture of the intermediate from above (0.2 g, 0.558 mmol), 2 M LiOH (1 mL, 1.12 mmol), and 3:1 of THF/H₂O (60 mL) is stirred at room temperature for 3 h and then concentrated. The residue is taken on without further purification.

A mixture of the residue from above, isobutylamine (0.167 mL, 1.674 mmol), HOBt (0.222 g, 1.674 mmol), PyBop (0.87 g, 1.674 mmol), DIEA (0.3 mL, 1.674 mmol) and DMA (10 mL) is stirred at room temperature over night and then diluted with CH₂Cl₂, washed with saturated NaHCO₃, 10% LiCl, brine, dried (Na₂SO₄), filtered and concentrated. The residue is then separated via flash chromatography (SiO₂, 70-100% EtOAc/hexanes gradient) to give an intermediate MS (ESI) m/z 400.1 (M+1).

A mixture of the intermediate (0.06 g, 0.15 mmol) from above, cyclohexylamine (5 mL) and dioxane (2 mL) is heated at 110° C. for 104 h and then concentrated. The residue is separated via semi-prep HPLC (20-65% CH₃CN/H₂O gradient with 0.1% NH₄OH in 17 minutes) to give the title compound 2″-cyclohexylamino-3,4,5,6-tetrahydro-2H-[1,2′;6′,4″]terpyridine-4,4′-dicarboxylic acid 4′-amide 4-(isobutylamide). MS (ESI) m/z 479.1 (M+1). ¹H NMR (400 MHz, DMSO-d₆) δ ppm 8.18 (br. s., 1H), 8.01 (d, J=5.3 Hz, 1H), 7.80 (t, J=5.7 Hz, 1H), 7.61 (br. s., 1H), 7.51 (s, 1H), 7.25 (s, 1H), 7.20 (br. s., 1H), 7.03-7.10 (m, 1H), 6.47-6.76 (m, 1H), 4.43-4.53 (m, 2H), 3.65-3.80 (m, 1H), 2.90-3.00 (m, 2H), 2.87 (t, J=6.3 Hz, 2H), 2.38-2.47 (m, 1H), 1.89-1.99 (m, 2H), 1.49-1.83 (m, 8H), 1.26-1.41 (m, 2H), 1.12-1.26 (m, 3H), 0.83 (d, J=6.7 Hz, 6H).

### Example 63

### A. N-iso-Propyl-2-methyl-3-(6-piperazin-1-yl-[2,4′]bipyridinyl-2′-ylamino)-benzamide

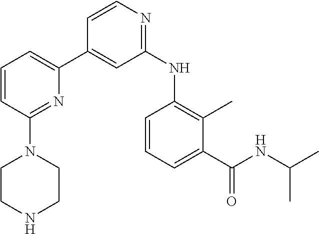

A mixture of 4-[2′-(3-methoxycarbonyl-2-methyl-phenylamino)-[2,4′]bipyridinyl-6-yl]-piperazine-1-carboxylic acid tert-butyl ester (0.142 g, 0.284 mmol), 2 M KOH (0.284 ml, 0.568 mmol), THF (12 mL) and H₂O (4 mL) is refluxed at 110° C. for 3 h. Additional 2 M KOH (0.2 mL) is added followed by THF (4 mL). The mixture is heated at 130° C. for additional 5 h. The mixture of desired product and starting material is obtained. The mixture is transferred to a sealed vessel and heated to 110° C. for additional 3 h and then concentrated to dryness to give residue (0.192 g) taken on without further purification.

The title compound is prepared with similar method to Example 62A. N-Isopropyl-2-methyl-3-(6-piperazin-1-yl-[2,4′]bipyridinyl-2′-ylamino)-benzamide. MS (ESI) m/z 431.1 (M+1). ¹H NMR (400 MHz, MeOD) δ ppm 7.93-7.95 (m, 1H), 7.81 (dd, J=8.6, 7.6 Hz, 1H), 7.74-7.76 (m, 1H), 7.63 (dd, J=6.8, 1.5 Hz, 1H), 7.40-7.50 (m, 4H), 7.12 (d, J=8.6 Hz, 1H), 4.15-4.24 (m, 1H), 3.88-3.93 (m, 4H), 3.33-3.37 (m, 4H), 2.32 (s, 3H), 1.27 (d, J=6.6 Hz, 6H).

### B. 2-Methyl-3-(6-piperazin-1-yl-[2,4′]bipyridinyl-2′-ylamino)-N-(tetrahydropyran-4-yl)-benzamide

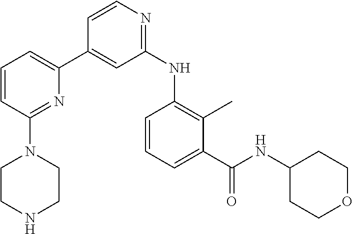

The title compound is prepared with similar method to Example 63A. ¹H NMR (400 MHz, DMSO-d₆) δ ppm 8.34 (s, 1H), 8.26 (d, J=7.8 Hz, 1H), 8.10 (d, J=5.6 Hz, 1H), 7.59-7.68 (m, 2H), 7.44 (br. s., 1H), 7.25 (dd, J=5.3, 1.3 Hz, 1H), 7.15-7.21 (m, 2H), 6.98-7.02 (m, 1H), 6.86 (d, J=8.6 Hz, 1H), 5.75 (s, 1H), 3.91-4.04 (m, 1H), 3.83-3.90 (m, 2 H), 3.49-3.55 (m, 4H), 3.35-3.43 (m, 2H), 2.81-2.87 (m, 4H), 2.21 (s, 3H), 1.74-1.82 (m, 2H), 1.45-1.58 (m, 2H).

### Example 64

### A. N*2″*-Cyclohexyl-3,4,5,6-tetrahydro-2H-[1,2′;6′,4″]terpyridine-4,2″-diamine

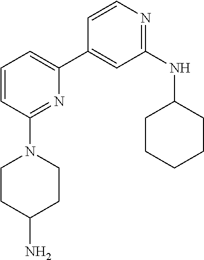

A mixture of 2,6-dibromo-pyridine (3.5 g, 14.77 mmol) and piperidin-4-yl-carbamic acid tert-butyl ester (1.478 g, 7.38 mmol) is heated to 130° C. in a sealed vessel for 6.5 h and 160° C. for 3 h. The residue is cooled and dissolved in CH₂Cl₂, washed with saturated NaHCO₃ (×2), brine, dried (Na₂SO₄), filtered and concentrated. The residue is then separated via flash chromatography (SiO₂, 10-30% EtOAc/hexanes gradient) to give an intermediate, (6′-bromo-3,4,5,6-tetrahydro-2H-[1,2′]bipyridinyl-4-yl)-carbamic acid tert-butyl ester (0.83 g, 32%). MS (ESI) m/z 356.0, 358.0 (M+1), which is taken on to next step.

The title compound is prepared with similar method to Example 1C. N*2″*-cyclohexyl-3,4,5,6-tetrahydro-2H-[1,2′;6′,4″]terpyridine-4,2″-diamine. MS (ESI) m/z 352.1 (M+1). ¹H NMR (400 MHz, CDCl₃) δ ppm 8.12 (d, J=5.3 Hz, 1H), 7.54 (dd, J=8.3, 7.6 Hz, 1H), 7.03-7.11 (m, 3H), 6.70 (d, J=8.3 Hz, 1H), 4.35-4.55 (m, 3H), 3.58-3.72 (m, 1H), 2.88-3.03 (m, 3H), 2.05-2.16 (m, 2H), 1.89-1.98 (m, 2H), 1.74-1.84 (m, 2H), 1.62-1.71 (m, 1H), 1.35-1.49 (m, 5H), 1.17-1.33 (m, 3H).

### B. 2″-Cyclohexylamino-3,4,5,6-tetrahydro-2H-[1,2′;6′,4″]terpyridine-4-carboxylic acid amide

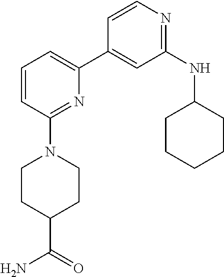

The title compound is prepared with similar method to Example 64A. MS (ESI) m/z 380.1 (M+1). ¹H NMR (400 MHz, MeOD) δ ppm 7.78-7.84 (m, 1H), 7.63-7.72 (m, 2H), 7.37-7.43 (m, 1H), 7.23-7.29 (m, 1H), 6.95-7.00 (m, 1H), 4.50-4.59 (m, 2H), 3.54-3.65 (m, 1H), 2.91-3.03 (m, 2H), 2.47-2.61 (m, 1H), 2.01-2.10 (m, 2H), 1.82-1.94 (m, 4H), 1.67-1.77 (m, 3H), 1.27-1.53 (m, 5H).

### Example 65

### A. 3-Bromomethyl-2,6-dichloro-isonicotinic acid ethyl ester

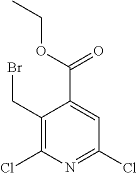

To a mixture of 2,6-dichloro-3-methylisonicotinic acid ethyl ester (10.0 g, 42.7 mmol), Example 7E and acetic acid (2.69 g, 44.9 mmol) in carbon tetrachloride (147 mL) are added N-bromosuccinimide (8.36 g, 47.0 mmol) and then benzoyl peroxide (1.03 g, 4.27 mmol). The mixture is stirred in oil bath at 60° C. under heat lamp for 5 h. The mixture is then cooled to room temperature. About half of the solvent is removed by rotary evaporation. The white succinimide solid is removed by filtration. The overweight filtrate (17 g for a theoretical 13.4 g, 42.7 mmol) is concentrated under reduced pressure and used as a crude immediately for the next step. MS (ESI) m/z 313.99. ¹H NMR (400 MHz, CDCl₃) δ ppm 7.72 (s, 1H), 4.99 (s, 2H), 4.48 (q, J=7.16 Hz, 2H), 1.46 (t, J=7.07 Hz, 3H).

### B. 4,6-Dichloro-2,3-dihydro-pyrrolo[3,4-c]pyridin-1-one

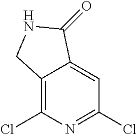

A mixture of 3-bromomethyl-2,6-dichloroisonicotinic acid ethyl ester (13.4 g, 42.7 mmol), tetrahydrofuran (100 mL), and ammonium hydroxide (300 mL of 28-30% ammonia) is stirred at room temperature for 18 h. The solvents are then removed by rotary evaporation. The nearly dry solid is treated with a small amount of water. The salmon-colored solid is isolated by filtration, washed with small amounts of water and then diethyl ether, and dried under vacuum. Filtration of the cooled filtrate yields additional salmon-colored solid (7.47 g, 36.8 mmol, 86%). MS (ESI) m/z 203.2 (M+1). ¹H NMR (400 MHz, DMSO-d₆) δ ppm 9.29 (br. s., 1H), 7.83 (s, 1H), 4.45 (s, 2H).

### C. 4-(6-Chloro-1-oxo-2,3-dihydro-1H-pyrrolo[3,4-c]pyridin-4-yl)-piperazine-1-carboxylic acid tert-butyl ester

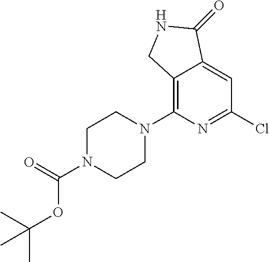

4,6-Dichloro-2,3-dihydro-pyrrolo[3,4-c]pyridin-1-one (5.63 g, 27.7 mmol), tert-butyl piperazine-1-carboxylate (7.75 g, 41.6 mmol), triethylamine (14.0 g, 139 mmol), and dioxane (50 mL) are stirred at 120° C. in a 350 mL sealed tube for 16 h. To the cooled down reaction mixture are then added more tert-butyl piperazine-1-carboxylate (5.2 g, 27.7 mmol) and triethylamine (2.83 g, 28.0 mmol). The vessel is sealed again and stirred at 120° C. for 24 h. The reaction mixture is then cooled to ambient temperature, and a light-pink solid is isolated by filtration (6.18 g, 17.5 mmol, 63%). MS (ESI) m/z 353.15 (M+1). ¹H NMR (400 MHz, DMSO-d₆) δ ppm 9.04 (s, 1H), 6.89 (s, 1H), 4.57 (s, 2H), 3.61-3.54 (m, 4H), 3.47-3.41 (m, 4H), 1.42 (s, 9H).

### D. 4-(4-Chloro-1-oxo-2,3-dihydro-1H-pyrrolo[3,4-c]pyridin-6-yl)-piperazine-1-carboxylic acid tert-butyl ester

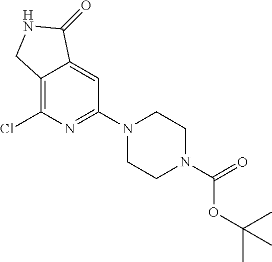

The title compound is typically obtained from the dioxane filtrate after isolation of 4-(6-chloro-1-oxo-2,3-dihydro-1H-pyrrolo[3,4-c]pyridin-4-yl)-piperazine-1-carboxylic acid tert-butyl ester. The dioxane is removed by rotary evaporation. Treatment with methanol yields a light yellow solid which is isolated by filtration. MS (ESI) m/z 353.30 (M+1). ¹H NMR (400 MHz, DMSO-d₆) δ ppm 8.93 (s, 1H), 7.01 (s, 1H), 4.28 (s, 2H), 3.58-3.53 (m, 4H), 3.45-3.40 (m, 4H), 1.42 (s, 9H).

### E. 4-[6-(2-Cyclohexylamino-pyridin-4-yl)-1-oxo-2,3-dihydro-1H-pyrrolo[3,4-c]pyridin-4-yl]-piperazine-1-carboxylic acid tert-butyl ester

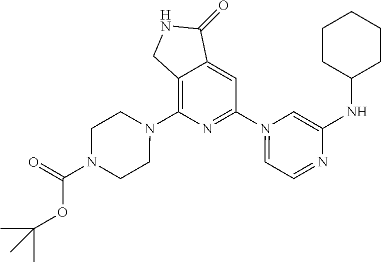

To an argon-degassed mixture of 4-(6-chloro-1-oxo-2,3-dihydro-1H-pyrrolo[3,4-c]pyridin-4-yl)-piperazine-1-carboxylic acid tert-butyl ester (0.997 g, 2.83 mmol), cyclohexyl-(4-trimethylstannanyl-pyridin-2-yl)-amine (1.15 g, 3.39 mmol), and cesium fluoride (0.988 g, 6.50 mmol) in dioxane (100 mL) is added bis(tri-tert-butylphosphine)palladium(0) (0.116 g, 0.226 mmol). The reaction mixture is stirred at 100° C. for 18 h. The room temperature reaction mixture is then filtered through Celite® and concentrated down to dryness. Treatment of the brown residue with diethyl ether (50 mL) yields an off-white solid which is isolated by filtration (1.37 g, 2.78 mmol, 98%). The yield is slightly inflated because the product is less than 95% pure. MS (ESI) m/z 493.29 (M+1). ¹H NMR (400 MHz, DMSO-d₆) δ ppm 8.97 (s, 1H), 8.02 (d, J=5.56 Hz, 1H), 7.44 (s, 1H), 7.20 (s, 1H), 7.08 (d, J=7.07 Hz, 1H), 6.42 (d, J=7.83 Hz, 1H), 4.61 (s, 2H), 3.85-3.70 (m, 1H), 3.70-3.58 (m, 4H), 3.57-3.43 (m, 4H), 2.02-1.87 (m, 2H), 1.81-1.66 (m, 2H), 1.65-1.55 (m, 1H), 1.44 (s, 9H), 1.40-1.27 (m, 2H), 1.27-1.13 (m, 3H).

### F. 6-(2-Cyclohexylamino-pyridin-4-yl)-4-piperazin-1-yl-2,3-dihydro-pyrrolo[3,4-c]pyridin-1-one

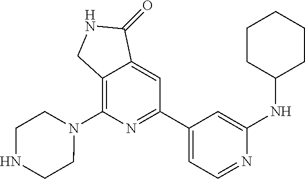

To a suspension of 4-[6-(2-cyclohexylamino-pyridin-4-yl)-1-oxo-2,3-dihydro-1H-pyrrolo[3,4-c]pyridin-4-yl]-piperazine-1-carboxylic acid tert-butyl ester (1.37 g, 2.78 mmol) in dichloromethane (20 mL) is added trifluoroacetic acid (5 mL, 7.4 g, 65 mmol). The solution is stirred at ambient temperature for 2 h. The solvents are removed by rotary evaporation. The crude residue is treated with triethylamine (5 mL) and dichloromethane (200 mL), and then washed with water (40 mL). After separation of the layers, the organic layer is dried over sodium sulfate, filtered, and concentrated down to dryness by rotary evaporation. The tan solid is then treated with hot 2-propanol (50 mL). After cooling of the suspension to room temperature, a light yellow solid is isolated by filtration and dried under reduced pressure (0.616 g, 1.57 mmol, 56%). MS (ESI) m/z 393.24 (M+1). ¹H NMR (400 MHz, DMSO-d₆) δ ppm 8.98 (s, 1H), 8.02 (d, J=5.30 Hz, 1H), 7.45 (s, 1H), 7.17 (s, 1H), 7.09 (d, J=5.56 Hz, 1H), 6.42 (d, J=8.08 Hz, 1H), 4.60 (s, 2H), 3.88-3.59 (m, 5H), 3.15-2.94 (m, 4H), 2.03-1.86 (m, 2H), 1.80-1.67 (m, 2H), 1.65-1.54 (m, 1H), 1.42-1.26 (m, 2H), 1.26-1.13 (m, 3H).

### Example 66

### A. 4-[4-(2-Cyclohexylamino-pyridin-4-yl)-1-oxo-2,3-dihydro-1H-pyrrolo[3,4-c]pyridin-6-yl]-piperazine-1-carboxylic acid tert-butyl ester

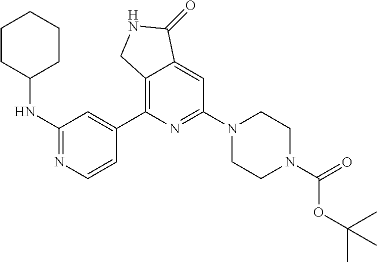

To a nitrogen-degassed mixture of 4-(4-chloro-1-oxo-2,3-dihydro-1H-pyrrolo[3,4-c]pyridin-6-yl)-piperazine-1-carboxylic acid tert-butyl ester (0.103 g, 0.2919 mmol) and cyclohexyl-(4-trimethylstannanyl-pyridin-2-yl)-amine (0.114 g, 0.336 mmol) in toluene (10 mL) is added trans-dichlorobis(triphenylphosphine)palladium(II) (0.021 g, 0.029 mmol). The reaction mixture is stirred under nitrogen at 110° C. for 16 h. The reaction is cooled to room temperature and the solvent is removed by rotary evaporation. The crude is purified through two successive silica gel columns (first solvent system: ethyl acetate, then 95:5 ethyl acetate methanol; second solvent system: 98:2:0.5, then 96:4:0.9 dichloromethane/methanol/ammonium hydroxide). A third column is used for contaminated fractions. A yellow solid is obtained as a result (0.043 g, 0.087 mmol, 30%). MS (ESI) m/z 493.33 (M+1). ¹H NMR (400 MHz, DMSO-d₆) δ ppm 8.94 (s, 1H), 8.04 (d, J=5.43 Hz, 1H), 7.08 (s, 1H), 6.98 (s, 1H), 6.95 (d, J=5.43 Hz, 1H), 6.50 (d, J=7.58 Hz, 1H), 4.59 (s, 2H), 3.77-3.69 (m, 1H), 3.67-3.59 (m, 4H), 3.50-3.44 (m, 4H), 1.95 (d, J=10.11 Hz, 2H), 1.77-1.69 (m, 2H), 1.64-1.56 (m, 1H), 1.43 (s, 9H), 1.39-1.31 (m, 2H), 1.25-1.17 (m, 3H).

### B. 4-(2-Cyclohexylamino-pyridin-4-yl)-6-piperazin-1-yl-2,3-dihydro-pyrrolo[3,4-c]pyridin-1-one

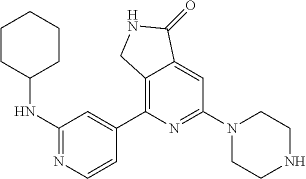

A solution of 4-[4-(2-cyclohexylamino-pyridin-4-yl)-1-oxo-2,3-dihydro-1H-pyrrolo[3,4-c]pyridin-6-yl]-piperazine-1-carboxylic acid tert-butyl ester (0.042 g, 0.085 mmol) in dichloromethane (5 mL) and trifluoroacetic acid (3.0 mL, 4.4 g, 39 mmol) is stirred for 2 h. The solvents are removed by rotary evaporation. The residue is then dissolved into dichloromethane and washed with 2 N aqueous sodium hydroxide solution and then brine. The aqueous layers are extracted three times with fresh dichloromethane. The combined organic layers are dried over sodium sulfate, filtered, concentrated by rotary evaporation, and dried in vacuo, to yield a yellow solid (0.030 g, 0.077 mmol, 90%). MS (ESI) m/z 393.24 (M+1). ¹H NMR (400 MHz, DMSO-d₆) δ ppm 8.94 (s, 1H), 8.04 (d, J=4.55 Hz, 1H), 7.05 (s, 1H), 6.97 (s, 1H), 6.94 (d, J=4.29 Hz, 1H), 6.52 (d, J=7.58 Hz, 1H), 4.59 (s, 2H), 3.73 (br. s., 1H), 3.62 (s, 4H), 2.92 (s, 4H), 1.94 (d, J=9.85 Hz, 2H), 1.72 (d, J=10.36 Hz, 2H), 1.59 (d, J=9.35 Hz, 1H), 1.36-1.27 (m, 2H), 1.25-1.14 (m, 3H).

### Example 67

### A. 6-Chloro-4-(5,6-dihydro-8H-[1,2,4]triazolo[4,3-a]pyrazin-7-yl)-2,3-dihydro-pyrrolo[3,4-c]pyridin-1-one

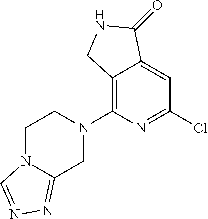

A mixture of 4,6-dichloro-2,3-dihydro-pyrrolo[3,4-c]pyridin-1-one (0.655 g, 3.23 mmol), triethylamine (2.61 g, 25.8 mmol), and 5,6,7,8-tetrahydro-[1,2,4]triazolo[4,3-a]pyrazine hydrochloride (0.544 g, 3.39 mmol) in dioxane (7.5 mL) is stirred in a 48 mL sealed tube at 120° C. for 16 h. Additional 5,6,7,8-tetrahydro-[1,2,4]triazolo[4,3-a]pyrazine hydrochloride (0.500 g, 3.11 mmol) is added to the mixture, which is heated again at 120° C. for 24 h. A light yellow powder is isolated by filtration of the room temperature reaction mixture (0.611 g, 2.10 mmol, 65%). MS (ESI) m/z 291.18 (M+1). ¹H NMR (400 MHz, DMSO-d₆) δ ppm 9.11 (s, 1H), 8.50 (s, 1H), 7.01 (s, 1H), 4.97 (s, 2H), 4.68 (s, 2H), 4.19 (t, J=5.31 Hz, 2H), 4.01 (t, J=5.43 Hz, 2H).

### B. 6-(2-Cyclohexylamino-pyridin-4-yl)-4-(5,6-dihydro-8H-[1,2,4]triazolo[4,3-a]pyrazin-7-yl)-2,3-dihydro-pyrrolo[3,4-c]pyridin-1-one

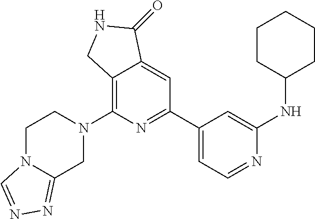

To a nitrogen-degassed mixture of 6-chloro-4-(5,6-dihydro-8H-[1,2,4]triazolo[4,3-a]pyrazin-7-yl)-2,3-dihydro-pyrrolo[3,4-c]pyridin-1-one (0.146 g, 0.503 mmol) and cyclohexyl-(4-trimethylstannanyl-pyridin-2-yl)-amine (0.204 g, 0.603 mmol) in N,N-dimethylformamide (7 mL) is added trans-dichlorobis(triphenylphosphine)palladium(II) (0.053 g, 0.075 mmol). The reaction mixture is stirred under nitrogen at 100° C. for 18 h. The reaction is cooled to room temperature. The solvent is removed by keeping under vacuum over 72 h. Treatment with methanol (about 10 mL) is followed by removal of white solid impurity through filtration. The filtrate is concentrated down to dryness by rotary evaporation and then treated with dioxane (first hot, then at room temperature). A tan solid isolated by filtration and weighing in excess of 0.2 g is determined to be a mixture of starting chloride and desired product. This mixture is purified on a Gilson system to yield a light yellow powder (15 mg, 0.035 mmol, 7%). MS (ESI) m/z 431.23 (M+1). ¹H NMR (400 MHz, DMSO-d₆) δ ppm 9.04 (s, 1H), 8.52 (s, 1 H), 8.04 (d, J=5.31 Hz, 1H), 7.53 (s, 1H), 7.23 (s, 1H), 7.11 (d, J=5.56 Hz, 1H), 6.52 (d, J=6.82 Hz, 1H), 5.06 (s, 2H), 4.72 (s, 2H), 4.30-4.19 (m, 2H), 4.07 (t, J=4.93 Hz, 2H), 3.83-3.69 (m, 1H), 2.01-1.88 (m, 2H), 1.80-1.67 (m, 2H), 1.65-1.55 (m, 1H), 1.42-1.27 (m, 2H), 1.27-1.14 (m, 3H).

### Example 68

### A. 4-iso-Butylcarbamoyl-piperidine-1-carboxylic acid tert-butyl ester

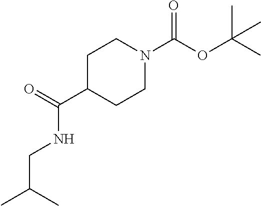

Thionyl chloride (0.38 mL, 5.23 mmol) is added to a solution of 1-BOC-piperidine-4-carboxylic acid (1.00 g, 4.36 mmol), pyridine (0.88 mL, 10.9 mmol) and CH₂Cl₂ (10 mL) at room temperature. After 25 min, a solution of isobutylamine, Et₃N (2.1 mL, 15.3 mmol) and CH₂Cl₂ (10 mL) is added. After an additional 2 h the solution is poured into 2 M HCl (100 mL) and extracted with Et₂O (100 mL). The organic layer is then washed with 2 M HCl (100 mL) and then 2 M Na₂CO₃ (100 mL). The ether layer is separated and then dried (Na₂SO₄) before being filtered and concentrated to give 4-Isobutylcarbamoyl-piperidine-1-carboxylic acid tert-butyl ester. ¹H NMR (400 MHz, CDCl₃) δ ppm 5.48 (br. s., 1H), 4.06-4.23 (m, 2 H), 3.05-3.12 (m, 2H), 2.67-2.81 (m, 2H), 2.16-2.27 (m, 1H), 1.71-1.85 (m, 3H), 1.55-1.69 (m, 2H), 1.46 (s, 9H), 0.91 (d, J=6.8 Hz, 6H).

### B. 1-(6-Chloro-1-oxo-2,3-dihydro-1H-pyrrolo[3,4-c]pyridin-4-yl)-piperidine-4-carboxylic acid isobutyl-amide

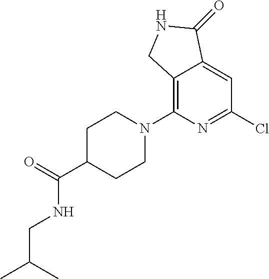

A mixture of 4,6-dichloro-2,3-dihydro-pyrrolo[3,4-c]pyridin-1-one (1.19 g, 5.87 mmol), triethylamine (2.97 g, 29.3 mmol), and crude trifluoroacetic acid salt of piperidine-4-carboxylic acid isobutyl amide [obtained by stirring 4-isobutylcarbamoyl-piperidine-1-carboxylic acid tert-butyl ester (2.63 g, 8.80 mmol) in dichloromethane (20 mL) and trifluoroacetic acid (6 mL) for 1.5 hours, then removing solvents by rotary evaporation] in dioxane (10 mL) is heated in a 75 mL sealed tube at 120° C. for 68 h. Filtration of room temperature mixture does not lead to separation of regioisomers. The filtrate is concentrated down to dryness by rotary evaporation, dissolved into ethyl acetate, and then washed with water and brine. The organic layer is dried over sodium sulfate, filtered and concentrated down to dryness by rotary evaporation. This residue is then combined with solids from first filtration and eluted through a silica gel column (80:20 ethyl acetate/heptane, then 100% ethyl acetate, then 95:5 ethyl acetate/methanol). The more polar regioisomer (TLC solvents: 90:10 ethyl acetate/methanol) is concentrated down to dryness by rotary evaporation, then treated with a small amount of methanol. The pink solid is isolated by filtration (0.647 g, 1.84 mmol, 31%). MS (ESI) m/z 351.20 (M+1). ¹H NMR (400 MHz, DMSO-d₆) δ ppm 9.00 (s, 1H), 7.79 (t, J=5.81 Hz, 1H), 6.84 (s, 1H), 4.54 (s, 2H), 4.19 (d, J=13.39 Hz, 2H), 3.09-2.92 (m, 2H), 2.86 (dd, J=6.57, 6.06 Hz, 2H), 2.48-2.36 (m, 1H), 1.79-1.71 (m, 2H), 1.71-1.52 (m, 3H), 0.82 (d, J=6.82 Hz, 6H).

### C. 1-[6-(2-Cyclohexylamino-pyridin-4-yl)-1-oxo-2,3-dihydro-1H-pyrrolo[3,4-c]pyridin-4-yl]-piperidine-4-carboxylic acid isobutyl-amide

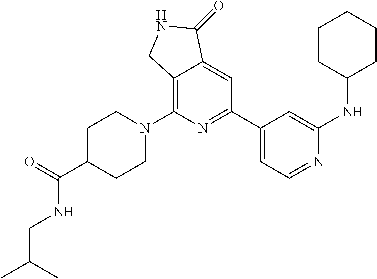

To an argon-degassed mixture of 1-(6-chloro-1-oxo-2,3-dihydro-1H-pyrrolo[3,4-c]pyridin-4-yl)-piperidine-4-carboxylic acid isobutyl-amide (0.217 g, 0.619 mmol), cyclohexyl-(4-trimethylstannanyl-pyridin-2-yl)-amine (0.252 g, 0.742 mmol), and cesium fluoride (0.216 g, 1.42 mmol) in dioxane (22 mL) is added bis(tri-tert-butylphosphine)palladium(0) (0.026 g, 0.049 mmol). The reaction mixture is stirred at 100° C. for 24 h. The reaction mixture is cooled to room temperature, and a dark solid is removed by filtration. The filtrate is concentrated down to dryness by rotary evaporation and then treated with a mixture of mainly diethyl ether and a small amount of methanol. A dark yellow solid is isolated by filtration (0.13 g, 0.26 mmol, 43%). MS (ESI) m/z 491.31 (M+1). ¹H NMR (400 MHz, DMSO-d₆) δ ppm 8.92 (s, 1H), 8.02 (d, J=5.56 Hz, 1H), 7.80 (t, J=5.94 Hz, 1H), 7.38 (s, 1H), 7.17 (s, 1H), 7.07 (d, J=5.56 Hz, 1H), 6.43 (d, J=7.58 Hz, 1H), 4.58 (s, 2H), 4.35 (d, J=12.88 Hz, 2H), 3.81-3.62 (m, 1H), 3.10-2.98 (m, 2H), 2.87 (dd, J=6.57, 6.06 Hz, 2H), 2.00-1.87 (m, 2H), 1.82-1.55 (m, 9H), 1.39-1.26 (m, 2H), 1.26-1.12 (m, 3H), 0.83 (d, J=6.57 Hz, 6H).

### Example 71

### In-vitro Assay for PKD Inhibition

The assay to measure protein kinase D1 (PKD1) activity is a time-resolved fluorescence resonance transfer (TR-FRET) assay using PerkinElmer's LANCE™ technology. In this case, a biotinylated syntide-2 peptide is used as the substrate in this reaction. Phosphorylation of the syntide-2 substrate is detected by a specific antibody that recognizes the phosphorylated peptide. A second fluorophore, APC, is conjugated to streptavidin that binds the biotinylated syntide-2 peptide. For detection, the europium fluorophore can be excited by 340 nM light Which then emits at 615 nM. Therefore, when the europium labeled secondary antibody binds on the phosphorylated peptide, it is brought into close contact with the APC and excites this fluorophore. The APC emission is at 665 nM and the 665 nM:615 nM ratio is a readout of PKD1 activity.

This assay is performed with full length wild-type enzyme that is expressed and purified from Sf9 insect cells. The reaction buffer consists of 35 mM Tris-HCl pH7.5, 5 mM MgCl₂, 0.02% Tween-20, 20 μM ATP, 1 mM DTT and 0.2 μg/mL PKD1 enzyme. The enzyme reaction is initiated by the addition of 2 μM syntide-2 peptide substrate and the reaction carried out for 50 minutes at room temperature. The reaction is stopped by a stop/detection Buffer consisting of 50 mM EDTA, 0.18 mg/mL rabbit polyclonal anti-phospho Syntide-2 antibody, 0.5 nM europium labeled anti-rabbit IgG and 10 nM streptavidin conjugated APC. After a one hour incubation with the stop/detection buffer, the reaction is read on an Envision 2100 Reader using a LANCE™ Eu/APC dual protocol. as described above, a 665 nM:615 nM ratio is determined to measure substrate phosphorylation and enzyme activity. Compounds are typically tested in an 11 point dose response fashion in triplicate for each concentration used. IC₅₀ values are calculated using an Activity Base (IDBS) software program.

The assay to measure protein kinase D2 (PKD2) activity is a time-resolved fluorescence resonance transfer (TR-FRET) assay using PerkinElmer's LANCE™ technology. In this case, a biotinylated syntide-2 peptide is used as the substrate in this reaction. Phosphorylation of the syntide-2 substrate is detected by a specific antibody that recognizes the phosphorylated peptide. A second fluorophore, APC, is conjugated to streptavidin that binds the biotinylated syntide-2 peptide. For detection, the europium fluorophore can be excited by 340 nM light which then emits at 615 nM. Therefore, when the europium labeled secondary antibody binds on the phosphorylated peptide, it is brought into close contact with the APC and excites this fluorophore. The APC emission Is at 665 nM and the 665 nM:615 nM ratio is a readout of PKD2 activity.

This assay is performed with full length wild-type enzyme purchase from Invitrogen. The reaction buffer consists of 35 mM Tris-HCl pH7.5, 5 mM MgCl₂, 0.02% Tween-20, 20 μM ATP, 1 mM DTT and 0.2 μg/mL PKD2 enzyme. The enzyme reaction is initiated by the addition of 2 μM syntide-2 peptide substrate and the reaction carried out for 50 minutes at room temperature. The reaction is stopped by a stop/detection Buffer consisting of 50 mM EDTA, 0.18 mg/mL rabbit polyclonal anti-phospho Syntide-2 antibody, 0.5 nM europium labeled anti-rabbit IgG and 10 nM streptavidin conjugated APC. After a one hour incubation with the stop/detection buffer, the reaction is read on an Envision 2100 Reader using a LANCE™ Eu/APC dual protocol. As described above, a 665 nM:615 nM ratio is determined to measure substrate phosphorylation and enzyme activity. Compounds are typically tested in an 11 point dose response fashion in triplicate for each concentration used. IC₅₀ values are calculated using an Activity Base (IDBS) software program.

The assay to measure protein kinase D3 (PKD3) activity is a time-resolved fluorescence resonance transfer (TR-FRET) assay using PerkinElmer's LANCE™ technology. In this case, a biotinylated syntide-2 peptide is used as the substrate in this reaction. Phosphorylation of the syntide-2 substrate is detected by a specific antibody that recognizes the phosphorylated peptide. A second fluorophore, APC, is conjugated to streptavidin that binds the biotinylated syntide-2 peptide. For detection, the europium fluorophore can be excited by 340 nM light which then emits at 615 nM. Therefore, when the europium labeled secondary antibody binds on the phosphorylated peptide, it is brought into close contact with the APC and excites this fluorophore. The APC emission is at 665 nM and the 665 nM:615 nM ratio is a readout of PKD3 activity.

This assay is performed with full length wild-type enzyme that is purchased from Invitrogen. The reaction buffer consists of 35 mM Tris-HCl pH7.5, 5 mM MgCl₂, 0.02% Tween-20, 20 μM ATP, 1 mM DTT and 0.2 μg/mL PKD3 enzyme. The enzyme reaction is initiated by the addition of 2 μM syntide-2 peptide substrate and the reaction carried out for 50 minutes at room temperature. The reaction is stopped by a stop/detection buffer consisting of 50 mM EDTA, 0.18 mg/mL rabbit polyclonal anti-phospho Syntide-2 antibody, 0.5 nM europium labeled anti-rabbit IgG and 10 nM streptavidin conjugated APC. After a one hour incubation with the stop/detection buffer, the reaction is read on an Envision 2100 Reader using a LANCE™ Eu/APC dual protocol. AS described above, a 665 nM:615 nM ratio is determined to measure substrate phosphorylation and enzyme activity. Compounds are typically tested in an 11 point dose response fashion in triplicate for each concentration used. IC₅₀ values are calculated using an Activity Base (IDBS) software program.

Compounds are evaluated in the HDAC5 nuclear export assay, a 384-well plate-based assay that enables high throughput screening (HTS) to identify small molecules that block agonist-dependent nuclear export of HDAC5. This assay employs the Cellomics High Content Imaging platform (Giuliano & Taylor 1998) and adenovirus encoding green fluorescent protein (GFP) tagged HDAC5. Neonatal rat ventricular myocytes (NRVMs) are infected with GFP-HDAC5 encoding virus and plated on gelatin-coated 384-well dishes. Cells are exposed to compound and stimulated with an prostaglandin (PGF2α), which is a potent stimulus for HDAC5 nuclear export. Following two hours of stimulation, cells are fixed and GFP-HDAC5 localization quantified using the Cellomics system, which provides a read-out of relative fluorescence intensity in the cytoplasmic versus nuclear compartment.

TABLE 2
Inhibitory Activity of Compounds

| | PKD1 | PKD2 | PKD3 | HDAC |
|---|---|---|---|---|
| Compound | (nM) | (nM) | (nM) | (nM) |
| 2′-(2-Chlorobenzylamino-6-piperazin-1-yl- | 25 | 368 | 31 | n.d. |
| [2,4′]bipyridinyl-4-carboxylic acid amide | | | | |
| iso-Propyl-[6-piperazin-1-yl-4-(1H-pyrazol- | <1 | 3 | <1 | 92 |
| 4-yl)-[2,4′]bipyridinyl-2′-yl]-amine | | | | |
| Cyclohexyl-[3-(4-fluorophenyl)-6-piperazin- | 21 | 157 | 24 | 384 |
| 1-yl-[2,4′]bipyridinyl-2′-yl]-amine | | | | |
| 2′-Cyclohexylamino-6-(R)-hexahydro- | 35 | 297 | 20 | 616 |
| pyrrolo[1,2-a]pyrazin-2-yl-[2,4′]bipyridinyl-4- | | | | |
| carboxylic acid amide | | | | |
| Cyclohexyl-(4-nitro-6-piperazin-1-yl- | 4 | 62 | 5 | 235 |
| [2,4′]bipyridinyl-2′-yl)-amine | | | | |
| 2′-[2-(4-Hydroxyphenyl)ethylamino]-6- | 2 | 402 | 2 | n.d. |
| piperazin-1-yl-[2,4′]bipyridinyl-4-carboxylic | | | | |
| acid amide | | | | |
| 2′-(1-Methyl-1H-pyrazol-3-ylamino)-6- | 8 | 76 | 8 | >1000 |
| piperazin-1-yl-[2,4′]bipyridinyl-4-carbonitrile. | | | | |
| 2′-Cyclohexylamino-6-((R)-3- | 6 | 60 | 6 | 679 |
| methylpiperazin-1-yl)-[2,4′]bipyridinyl-4- | | | | |
| carboxylic acid amide | | | | |
| Cyclohexyl-(6-piperazin-1-yl-4- | 20 | 101 | 13 | 342 |
| trifluoromethyl-[2,4′]bipyridinyl-2′-yl)amine | | | | |
| 6-(2-Cyclohexylamino-pyridin-4yl)-4-(5,6- | 56 | 466 | 74 | >1000 |
| dihydro-8H-[1,2,4]triazolo[4,3-a]pyrazin-7- | | | | |
| yl)-2,3-dihydro-pyrrolo[3,4-c]pyridin-1-one | | | | |
| 2′-Cyclohexylamino-6-piperazin-1-yl- | 60 | 281 | 114 | >1000 |
| [2,4′]bipyridinyl-5-carbonxylic acid | | | | |
| cyanomethyl-amide | | | | |
| 2-Methyl-N-(2-morpholin-4-yl-ethyl)-3-(6- | 22 | 186 | 58 | 288 |
| piperazin-1-yl-[2,4′]bipyridinyl-2′-ylamino)- | | | | |
| benzamide | | | | |
| 2-Methyl-N-(2-morpholin-4-yl-ethyl)-3-(6- | <1 | 1 | <1 | 25 |
| piperazin-1-yl-[2,4′]bipyridinyl-2′-ylamino)- | | | | |
| benzamide | | | | |
| N*2′*-Cyclohexyl-6-piperazin-1-yl- | 10 | 361 | 38 | 421 |
| [2,4′]bipyridinyl-4,2′-diamine | | | | |
| 2′-Cyclohexylamino-6-piperazin-1-yl- | 83 | 500 | 166 | >1000 |
| [2,4′]bipyridinyl-5-carboxylic acid methyl | | | | |
| ester | | | | |
| Cyclohexyl-(4-[1,3,4]oxadiazol-2-yl-6- | 12 | 214 | 14 | 126 |
| piperazin-1-yl-[2,4′]bipyridinyl-2′-yl)-amine | | | | |
| N*2′*-Cyclohexyl-N*4*-(4-fluoro-phenyl)-6- | 182 | 1156 | 252 | 423 |
| piperazin-1-yl-[2,4′]bipyridinyl-4,2′-diamine | | | | |
| Cyclohexyl-(4-methanesulfonyl-6-piperazin- | 63 | 735 | 85 | >1000 |
| 1-yl-[2,4′]bipyridinyl-2′-yl)-amine | | | | |

### EQUIVALENTS

Those skilled in the art will recognize, or be able to ascertain using no more than routine experimentation, numerous equivalents to the specific procedures described herein. Such equivalents are considered to be within the scope of the present invention and are covered by the following claims. The appropriate components, processes, and methods of those patents, applications and other documents may be selected for the present invention and embodiments thereof.

1. A compound of Formula I: 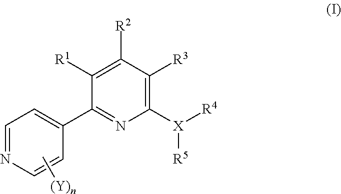
wherein
R¹, R², and R³ are each independently hydrogen, halogen, cyano, nitro, hydroxy, alkyl, alkoxy, alkoxycarbonyl, —C(O)NR⁷R⁸, hydroxycarbonyl, —NR⁹R¹⁰, alkylsulfonyl, heterocyclyl, heteroaryl, or aryl; or R² may be linked with R¹ to form a lactam ring, or R² may be linked with R³ to form a lactam ring;
X is nitrogen;
R⁴ and R⁵ are each independently hydrogen, heterocyclyl, alkyl, or R⁴ and R⁵ are linked together to form a heterocyclic or heteroaryl ring;
R⁷ and R⁸ are each independently hydrogen, alkyl, or cycloalkyl;
R⁹ and R¹⁰ are each independently hydrogen, alkoxycarbonyl, arylaminocarbonyl, sulfonyl, acyl, or aryl;
Y is independently selected for each occurrence from halogen, cyano, nitro, hydroxy, aryl, alkyl, alkoxy, or —NR¹¹R¹², provided that at least one Y is —NR¹¹R¹²;
R¹¹ and R¹² are each independently hydrogen, cycloalkyl, heterocyclyl, aryl, arylamino, heteroaryl, or alkyl;
n is an integer selected from 0, 1, 2, 3, or 4; and pharmaceutically acceptable salts, polymorphs, rotamers, prodrugs, enantiomers, hydrates, and solvates thereof.

2. The compound according to claim 1, wherein R⁴ is hydrogen and R⁵ is heterocyclyl; or
R⁴ and R⁵ are linked together to form the following heterocyclic ring: 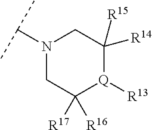
wherein
Q is nitrogen, oxygen, or —CH;
R¹³ is hydrogen, alkyl, acyl, aminocarbonyl, hydroxycarbonyl, amino, alkylaminocarbonyl, alkoxycarbonyl, or absent when Q is oxygen, or when linked with R¹⁶ may be a heterocycle; and
R¹⁴, R¹⁵, R¹⁶, and R¹⁷ are each independently hydrogen, alkyl, amino, or R¹⁴ and R¹⁵ may optionally be linked to form a ring, or R¹⁶ and R¹⁷ may optionally be linked to form a ring; or a pharmaceutically acceptable salt thereof.

3. The compound according to claim 1, wherein
R¹ and R³ are hydrogen;
R² is hydrogen, cyano, nitro, hydroxy, —C(O)NH₂, or heteroaryl; or R² may be linked with R¹ to form a lactam ring, or R² may be linked with R³ to form a lactam ring;
Y is NR¹¹R¹²; and
R¹¹ and R¹² are each independently hydrogen, alkyl, cycloalkyl, heterocyclyl, aryl, or heteroaryl; or a pharmaceutically acceptable salt thereof.

4. The compound according to claim 1, wherein
R¹ is hydrogen;
R² is hydrogen, nitro, —C(O)NH₂, or pyrazolyl;
R³ is hydrogen, or R² and R³ may optionally be linked to form a lactam ring;
X is 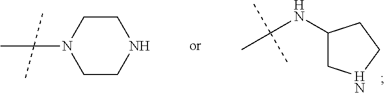
Y is —NHR¹² and Y is in the 2 position; and
R¹² is isopropyl, cyclohexyl, phenyl, benzyl, pyranyl, pyrazolyl, or —C(O)(CH₂)₂; or a pharmaceutically acceptable salt thereof.

5. The compound according to claim 1, wherein R¹² is benzyl substituted with hydroxy; or a pharmaceutically acceptable salt thereof.

6. The compound according to claim 1, wherein R¹² is phenyl substituted with methyl, fluorine, or methoxy; or a pharmaceutically acceptable salt thereof.

7. The compound according to claim 1, wherein R¹² is —C(O)(CH₂)₂-pyrrolidinyl; or a pharmaceutically acceptable salt thereof.

8. The compound according to claim 1, wherein R¹² is N-methyl-pyrazolyl; or a pharmaceutically acceptable salt thereof.

9. (canceled)

10. A pharmaceutical composition comprising a compound according to claim 1; or a pharmaceutically acceptable salt thereof; and a pharmaceutically acceptable excipient or carrier.

11. A method of treating a PKD associated disorder or disease in a subject, comprising administering to said subject a therapeutically effective amount of the compound according to claim 1; or a pharmaceutically acceptable salt thereof, such that said PKD associated disorder or disease in said subject is treated.

12-14. (canceled)

15. The method according to claim 11 wherein the disease or disorder is heart failure, colorectal cancer, regulation of cell growth, autoimmune disorders, or hyperproliferative skin disorders.

16. The method of claim 11, wherein said subject is human.

17. The method according to claim 11 further comprising administering a second agent, such that said subject is treated for said PKD associated disorder or disease.

18. The method of claim 17, wherein said second agent is an HMG-Co-A reductase inhibitor, an angiotensin II receptor antagonist, angiotensin converting enzyme (ACE) Inhibitor, a calcium channel blocker (CCB), a dual angiotensin converting enzyme/neutral endopeptidase (ACE/NEP) inhibitor, an endothelin antagonist, a renin inhibitor, a diuretic, an ApoA-I mimic, an anti-diabetic agent, an obesity-reducing agent, an aldosterone receptor blocker, an endothelin receptor blocker, or a CETP inhibitor.

19. (canceled)

20. The pharmaceutical composition according to claim 10 further comprising a second agent.

21. The pharmaceutical composition of claim 20, wherein said second agent is an HMG-Co-A reductase inhibitor, an angiotensin II receptor antagonist, angiotensin converting enzyme (ACE) Inhibitor, a calcium channel blocker (CCB), a dual angiotensin converting enzyme/neutral endopeptidase (ACE/NEP) inhibitor, an endothelin antagonist, a renin inhibitor, a diuretic, an ApoA-I mimic, an anti-diabetic agent, an obesity-reducing agent, an aldosterone receptor blocker, an endothelin receptor blocker, or a CETP inhibitor.
